(19) United States
(12) Patent Application Publication
(10) Pub. No.: US 2023/0227515 A1
(43) Pub. Date: Jul. 20, 2023

(54) OPTIMIZED GENE THERAPY FOR TARGETING MUSCLE IN MUSCLE DISEASES

(71) Applicant: RESEARCH INSTITUTE AT NATIONWIDE CHILDREN'S HOSPITAL, Columbus, OH (US)

(72) Inventor: Paul Taylor Martin, Bexley, OH (US)

(21) Appl. No.: 17/786,721

(22) PCT Filed: Dec. 21, 2020
(86) PCT No.: PCT/US2020/066477

§ 371 Date: Jun. 17, 2022

Related U.S. Application Data
(60) Provisional application No. 62/951,564, filed on Dec. 20, 2019.

(51) Int. Cl. C07K 14/47; A61K 48/00; C12N 15/86; A61P 21/00

(52) U.S. Cl. CPC C07K 14/4707; A61K 48/005; C12N 15/86; A61P 21/00

(57) ABSTRACT

The disclosure provides gene therapy vectors, such as adeno-associated virus (AAV), optimized for delivering a transgene to muscles. The optimized vectors contain constitutive or a muscle-specific promoter to deliver whole body or skeletal/heart muscle-specific transgene expression, respectively, in combination with a transgene cDNA to replace the gene mutation found in a muscle disease with a normal copy of the gene, an internal ribosomal entry site (IRES) to allow for production of a second protein from the same transcript, and a muscle growth factor, to build new muscle growth and strength. For example, the invention provides The disclosure provides gene therapy vectors, such as recombinant adeno-associated vims (rAAV), designed for treatment of GNE myopathy in which the rAAV expresses UDP-GlcNAc-epimerase/ManNAc-6 alone or in combination with a muscle growth factor or muscle transdifferentation factor. The provided AAV replace the mutated GNE gene expression while expressing proteins that stimulate muscle growth.

This application claims priority benefit to U.S. Provisional Patent Application No. 62/951,564, filed Dec. 20, 2019, which is incorporated by reference herein in its entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 54649_Sqlisting.txt; Size: 233,379 bytes, created; Dec. 21, 2020.

### FIELD OF INVENTION

The disclosure provides gene therapy vectors, such as adeno-associated virus (AAV), optimized for delivering a transgene to muscles. The optimized vectors contain constitutive or a muscle-specific promoter to deliver whole body or skeletal/heart muscle-specific transgene expression, respectively, in combination with a transgene cDNA to replace the gene mutation found in a muscle disease with a normal copy of the gene, an internal ribosomal entry site (IRES) to allow for production of a second protein from the same transcript, and a muscle growth factor, to build new muscle growth and strength. The transgene and the muscle growth factor gene are expressed from the same mRNA, which expresses both proteins due to the presence of an Internal Ribosomal Entry Site (or IRES) from the Fibroblast Growth Factor 1A gene sequence, which allows for the second protein to be made from the single mRNA.

### BACKGROUND

GNE Myopathy is an adult onset autosomal recessive genetic disease characterized by progressive muscle weakness that that can lead to loss of ambulation and loss of independent living. As its name implies, GNE myopathy is caused by loss of function pathogenic variants or mutations in the GNE gene. This disease is also known as hereditary inclusion body myopathy, quadriceps sparing myopathy, distal myopathy with rimmed vacuoles, and Nonaka myopathy. The GNE gene encodes a bifunctional UDP-GIcNAc-epimerase/ManNAc-6 kinase, whose enzymatic activities are essential in sialic acid biosynthetic pathway.

Sialic acid is an acidic monosaccharide that modifies non-reducing terminal carbohydrate chains on glycoproteins and glycolipids and plays an important role in different processes such as cell-adhesion and cellular interactions. Sialic acid has been implicated in health and disease and is found in terminal sugar chains of proteins modulating their cellular functions. As UDP-N-acetylglucosamine 2-epimerase/N-acetylmannosamine kinase (GNE) is the key enzyme for the biosynthesis of sialic acid. Moreover, it has been demonstrated that GNE expression is induced when myofibers are damaged or regenerating, and that GNE plays a role in muscle regeneration. Myoblasts carrying a mutated GNE gene show a reduction in their epimerase activity, whereby only the cells carrying a homozygous epimerase mutation also present with a significant reduction in the overall membrane bound sialic acid. (Pogoryleva et al., Orphanet J Rare Dis. 13: 70, 2018).

GNE myopathy leads to weakness and wasting of muscles in legs and arms. First symptoms usually occur in young adults (usually in the third decade of life), but a later onset has also been observed in some patients. A diagnosis of GNE myopathy should be considered primarily in patients presenting with distal weakness (foot drop) in early adulthood (other onset symptoms are possible too). The disease slowly progresses to involve other lower and upper extremities' muscles, typically with marked sparing of the quadriceps. Characteristic findings found in biopsies of affected muscles include “rimmed” (autophagic) vacuoles, aggregation of various proteins, and fiber size variation.

Despite the fact that mutations in the GNE gene were shown to cause GNE myopathy in 2001, there are as yet no effective therapies for this disease. Attempts to develop slow release sialic acid therapy failed in a phase 3 clinical trial, and ManNAc glycan therapy is currently being investigated. While development of a gene therapy approach for GNE gene replacement might seem straightforward, it is in fact complicated by a number of unresolved issues in GNE Myopathy research: First and foremost is the lack of a robust and reproducible model for the disease. While Noguchi and Nishino published several papers on a transgenic GNED176VTg Gne^{-/-} mouse model showing clear aspects of disease pathology, other groups, have failed to see the same phenotypes with subsequent breeding, likely the result of genetic drift in the founder transgenic line (see Nishino et al., J. Neurol. Neurosurg, Psychiatry 86(4): 385-392). A GNE_{M712T} variant knock-in mouse model showed premature death in the first few weeks of life due to kidney disease, a clinical phenotype that is not present in GNE Myopathy patients. Other lines of the same model were bred out to show no phenotype at all despite having the same genetic mutation. Second is a lack of measurable natural history data from the rare and geographically diverse patient population. Third, because of the late onset of disease on the highly variable disease progression, it is quite difficult to show clinical effects in GNE myopathy trials with only gene replacement, which will only slow or arrest disease progression.

Cells deficient in GNE activity can be rescued by addition of sialic acid (SA), or by addition of ManNAc, which can also be converted to ManNAc-6 phosphate, the end product of GNE activity, through GlcNAc-6 kinase activity that is not mutated in the disease. Some glycan therapies have shown efficacy in the GNE_{D176V}^{Tg}Gne^{-/-} mouse and the Gne_{M712T} knock-in mouse. This has led to two sets of clinical trials, one using slow release SA (phase 3 completed)(Lochmuller et al., Neurology 92(18): e2109-e17, 2019) and one using ManNAc (phase 1 completed) (Xu et al., Mol. Genet. Metab., 12291-2: 126-34, 2017). While SA and ManNAc were shown to have significant therapeutic effects in mice, slow release SA therapy (ACE-ER) met no clinical milestones in a phase 3 clinical trial of GNE myopathy patients[16]. There was no significant change from placebo for any clinical measure.

The lack of efficacy for glycan therapy in GNE myopathy patients makes gene therapy a very attractive alternative. There is, however, still a major problem, that is the slow and variable progression of the human disease and the lack of robust short-term clinical milestones. For example, a current phase 3 clinical trial was 48 weeks in duration[16]. In that time, there was not a significant drop in any of the strength measures for the patient population from pre-treatment baseline, though some measures did trend lower.

The goal of the GNE therapeutic methods provided herein is to create a tandem gene therapy—to utilize a muscle-specific IRES to create a bicistronic gene therapy vector that expresses both the normal GNE gene and a known muscle growth factor. Such an AAV vector will both correct the genetic defect of GNE myopathy and increase muscle strength, thus reversing rather than just arresting the decline of muscle strength clinical measures. A therapy that builds new muscle and muscle strength while also preventing further disease by adding back the normal GNE gene will be of greater benefit to patients with GNE myopathy and will provide an easier means of demonstrating clinical improvement.

Given the pathophysiology of the disease, recent clinical trials have evaluated the use of sialic acid or ManNAc (a precursor of sialic acid) in patients with GNE myopathy as well as early gene therapy trials. For example, AAV8 viral vectors carrying wild type human GNE cDNA have been shown to transduce murine muscle cells and human GNE myopathy-derived muscle cells in culture and to express the transgene in these cells (Mitrani-Rosenbaum et al., Neuromuscul. Disord. 22(11): 1015-24, 2012). The gene therapies in the prior art only focus on delivering wild-type GNE gene and do not utilize the dual function bicistronic technology disclosed herein.

The disclosure provides for gene therapies which increase muscle strength at the same time that they provide a transgene for gene replacement to prevent further muscle injury or to promote muscle growth are desired. For example, gene therapy vectors that provide GNE gene replacement are likely to be one of the only ways to prove clinical effectiveness for GNE myopathy in a period shorter than 5 years, as the natural history of disease progression is slow and quite variable. It will also be the one of the only ways to show clinical efficacy in all GNE myopathy patients, many of which have lost ambulation not long after diagnosis but that can still show significant arm function, for example self-feeding, which could still be preserved or improved by such a therapy. Because this disease is a myopathy and not a dystrophy, muscle, once repaired, should remain in place permanently.

### SUMMARY OF INVENTION

The disclosure provides gene therapy vectors, such as adeno-associated virus (AAV), optimized for delivering a transgene to muscles. The optimized vectors contain constitutive or a muscle-specific promoter to deliver whole body or skeletal/heart muscle-specific transgene expression, respectively, in combination with a transgene cDNA to replace the gene mutation found in a muscle disease with a normal copy of the gene (or a surrogate gene replacement), an internal ribosomal entry site (IRES) to allow for production of a second protein from the same transcript, and a muscle growth factor, to build new muscle growth and strength. The transgene and the muscle growth factor gene are expressed from the same mRNA, which expresses both proteins due to the presence of an Internal Ribosomal Entry Site (or IRES) from the Fibroblast Growth Factor 1A gene sequence, which allows for the second protein to be made from the single mRNA. For example, the disclosure provides gene therapy vectors designed for treatment of GNE myopathy. The AAV expresses the GNE gene, which encodes a bifunctional UDP-GlcNAc-epimerase/ManNAc-6 kinase enzyme alone or in combination with muscle growth factors such as follistatin (FST), a heparin binding-modified Insulin-like growth factor 1 (HB-IGF), native IGF1 or SMAD7. In this scenario, the provided AAV replace the mutated GNE gene expression in GNE myopathy patients with the normal GNE gene while simultaneously expressing proteins that stimulate muscle growth and strength, which can offset and even reverse the course of the disease. The unique aspect of the tandem vector is that it delivers two necessary therapeutic elements at the same time—1, a gene replacement therapy to prevent further disease in the expressing cells or tissues, and 2, a muscle growth therapy that reverses disease by building new muscle growth and strength. For the muscular dystrophies and for myopathies, the loss of muscle tissue arises from mutations in genes that cause the disease. The therapies proposed here will not only arrest the disease in such patients by reintroducing a non-mutated version of the disease gene, but build and reverse ongoing muscle loss by co-expressing a muscle growth factor. Such growth factors may double the amount of muscle in a tissue, doubling (and thereby reversing) weakness caused in these diseases.

The disclosure also provides surrogate gene therapy vectors for the treatment of muscular dystrophy, e.g. Duchene muscular dystrophy, limb girdle muscular dystrophy 2L (LGMD2A) and congenital muscular dystrophy 1a (MDC1A). The AAV expresses the GALGT2 (B4GALNT2) gene, which encodes the GalNAc transferase (beta 1,4-N-acetylgalactosamine galactosyltransferase) alone or in combination with muscle growth factors such as follistatin (FST), a heparin binding-modified Insulin-like growth factor 1 (HB-IGF), native IGF1 or SMAD7. This is a surrogate gene therapy because rather than replacing the mutated gene, the therapy provides the enzyme that transfers a complex sugar molecule onto a specific protein, such as dystroglycan.

Provided herein are AAV having a genome comprising a constitutive or a muscle specific promoter which drives expression of a nucleotide sequence encoding a transgene of interest in combination with a nucleotide sequence encoding a muscle growth factor, such as a protein that induces muscle growth and a muscle-specific IRES such as the FGF IRES, or a muscle transdifferentiation factor, such as myoD. This gene therapy approach is useful for treating any disease that requires gene replacement in combination with the need to increase muscle growth or muscle strength such as GNE myopathy, limb girdle muscular dystrophies, congenital muscular dystrophy 1A and Duchene muscular dystrophy.

The disclosure provides for polynucleotides comprising a) a promoter element such as a constitutive or muscle specific promoter, b) a transgene, c) internal ribosomal entry site (IRES), and d) a nucleotide sequence (i.e., a second transgene) encoding a muscle growth factor or a muscle transdifferentation factor. For example, the constitutive or muscle specific promoter is operably linked to a transgene and/or the IRES is operably linked to the nucleotide sequence encoding the muscle growth factor or the muscle transdifferentation factor. The fact that the elements are linked into a single mRNA allows for both functions to be provided by a single AAV-mediated gene therapy product. Due to the great expense of AAV production and due to the safety issues in dosing with AAV, use of a single AAV vector with two gene therapies would be vastly superior to obtaining the same result by mixing together two monogenic AAV gene therapies, where twice the amount (or more) of AAV would have to be made and be delivered to the patient to achieve the same result.

The disclosure also provides for polynucleotides comprising a) one or more constitutive or muscle specific promoter elements and b) a GNE cDNA sequence or a GALGT2 cDNA sequence. For example, the polynucleotide comprises a) more or more constitutive or muscle specific promoter elements, b) a GNE cDNA sequence, c) internal ribosomal entry site (IRES), and d) a polynucleotide sequence that induces muscle growth or differentiates cells into muscles cell. In some embodiments, the muscle specific control element is operably linked to a GNE cDNA sequence and/or the IRES is operably linked to a polynucleotide that induces muscle growth. In an additional example, the polynucleotide comprises a) more or more constitutive or muscle specific promoter elements, b) a GALGT2 cDNA sequence, c) internal ribosomal entry site (IRES), and d) a polynucleotide sequence that induces muscle growth or differentiates cells into muscles cell.

GNE myopathy is an adult onset, slowly progressing, muscle disease. In order to demonstrate therapeutic effects within a reasonable time frame, and in order to provide the greatest benefit to patients who are already impacted by muscle weakness at the time of diagnosis, a gene therapy is needed that not only corrects the genetic deficiency in GNE gene function but also builds new muscle mass. Follistatin, IGF1, SMAD7 and HB-IGF are known to dramatically stimulate muscle growth in mice, macaques and/or humans. Follistatin does this, in part, by inhibiting repressive growth signaling by myostatin through competitive inhibition and repression of Smad2/3 signaling, while IGF1 does this, in part, by activating the muscle IGF1 Receptor and activating Akt/mTOR signaling. Provided herein are bicistronic AAV expressing with GNE using the IRES sequence from FGF1A, which is known to work most efficiently in skeletal muscle tissue. Use of a muscle-specific IRES is ideal for follistatin, as it promotes optimal muscle growth through local expression, while use of CMV promoter for GNE expression would be ideal, as GNE is normally expressed in all tissues.

Provided herein are AAV having a genome comprising a promoter element such as a constitutive promotor or a muscle specific promoter which drives expression of a GNE cDNA sequence or a GALGT2 cDNA sequence. In particular, the disclosure provides rAAV having a genome designed to promote GNE gene replacement. In these AAV, the genome comprises a) one or more muscle specific promoter elements and b) GNE cDNA sequence. In another aspect, the disclosure provides a rAAV having a genome designed to promote GALGT2 surrogate gene therapy (expression of a surrogate gene).

For example, the disclosure provides a rAAV genome comprising a polynucleotide comprising a nucleotide sequence encoding a wild type human GNE gene, e.g. the variant 2 GNE wild type human cDNA (SEQ ID NO: 1) and a muscle specific promoter such as CMV promoter (SEQ ID NO: 3), MCK promoter (SEQ ID NO: 4), MHCK7 promoter (SEQ ID NO: 5), or a miniCMV promoter (SEQ ID NO: 7), or the human GNE promoter sequence (SEQ ID NO: 6). In some embodiments, the human GNE promoter element are found between exons 1 and 2 to drive expression of the variant 2 (722 amino acid) GNE cDNA comprising the nucleic acid sequence of SEQ ID NO: 1 (thereby allowing for endogenous natural gene expression).

The disclosure also provides for polynucleotides comprising a) one or more constitutive or muscle specific promoter elements and b) a GALGT2 cDNA sequence (SEQ ID NO: 36). For example, the polynucleotide comprises a) more or more constitutive or muscle specific promoter elements, b) a GALGT2 cDNA sequence, c) internal ribosomal entry site (IRES), and d) a polynucleotide sequence that induces muscle growth or differentiates cells into muscles cell. In some embodiments, the muscle specific control element is operably linked to a GALGT2 cDNA sequence and/or the IRES is operably linked to a polynucleotide that induces muscle growth.

For example, the disclosure also provides a rAAV genome comprising a polynucleotide comprising a nucleotide sequence encoding a wild type human GALGT2 gene (SEQ ID NO: 36) and a muscle specific promoter such as MCK promoter (SEQ ID NO: 4), or MHCK7 promoter (SEQ ID NO: 5).

The disclosure also provides for rAAV having a genome designed to include a second transgene which will induce muscle growth or differentiate or convert a cell to muscle. For example, the rAAV have a genome comprising a GNE cDNA or the GALGT2 cDNA sequence, an internal ribosomal entry site (IRES) from the Fibroblast Growth Factor 1A gene, which is known to function in skeletal muscle, 3′ of the GNE cDNA or GALGT2 cDNA sequence, followed by a nucleotide sequence encoding a gene known to induce muscle growth such as a follistatin, e.g. follistatin 344 (FS344) or a variant of IGF1 e.g. HB-IGF1, prior to the poly A sequence or SMAD7. The FGF IRES comprises the nucleotide sequence of SEQ ID NO: 30 of a fragment thereof. An exemplary fragment of the FGF IRES comprises the nucleotide sequence of SEQ ID NO: 8, which is also referenced to herein as “mini-IRES”.

The present disclosure is directed to gene therapy vectors, e.g. AAV, expressing the wild type human GNE gene to skeletal muscles to reduce or to replace the defective GNE gene. The gene therapy vectors of the invention also may be AAV expressing wild type human GNE gene and a gene that induces muscle growth such as follistatin, IGF1 or SMAD7 in a single rAAV genome.

The disclosure provides for polynucleotides comprising a) one or more promoter elements such as a constitutive or muscle-specific promoter and b) GNE cDNA sequence. The disclosure also provides for polynucleotides comprising a) more or more promoter elements such as a constitutive muscle specific promoter, b) GNE cDNA sequence or a GALGT2 cDNA sequence, c) internal ribosomal entry site (IRES), and d) a nucleotide sequence that encodes a muscle growth factor or a muscle transdifferentation factor. The GNE cDNA is a nucleic acid sequence that encodes UDP-GlcNAc-epimerase/ManNAc-6. In exemplary embodiments, the GNE cDNA is a wild type variant 2 GNE cDNA which encodes UDP-GlcNAc-epimerase/ManNAc-6 kinase. The variant 2 wild type GNE cDNA sequence is set out as the nucleic acid sequence of SEQ ID NO: 1. The disclosure also provides for polynucleotides comprising a GNE promoter element found between exons 1 and 2 to drive expression of the same variant 2 (722 amino acid) GNE cDNA. The GNE promoter sequence is set out as SEQ ID NO: 6. The GALGT2 cDNA is a nucleic acid sequence that encodes GalNAc transferase. The GALGT2 cDNA sequence is set out as the nucleic acid sequence of SEQ ID NO: 36. The GalNAc transferase amino acid sequence is set out as SEQ ID NO: 37.

In some aspects, the disclosure provides polynucleotide that comprise the GNE cDNA sequence or the GALGT2 cDNA sequence and a nucleotide sequence that encodes a protein that induces muscle growth such as follistatin, an Insulin-like Growth Factor 1 (IGF1) variant or SMAD7. For example, the follistatin is follistatin 344 which is encoded by the nucleotide sequence of SEQ ID NO: 9. Another exemplary follistatin is follistatin 317 which is encoded by the nucleotide sequence of SEQ ID NO: 28 In addition, the IGF1 variant is HB-IGF which is encoded by the nucleotide sequence of SEQ ID NO: 11. The SMAD7 is encoded by the nucleotide sequence of SEQ ID NO: 39.

In some aspects, the disclosure provides a polynucleotide that comprises the GNE cDNA sequence or the GALGT2 cDNA sequence and a sequence that encodes a protein that induces differentiation of a cell to muscle (transdifferentation factor), such as myoD (SEQ ID NO: 31).

In some aspects, the polynucleotides comprise an internal ribosomal entry site (IRES) such as the IRES from the Fibroblast Growth Factor 1A gene (FGF IRES). The FGF IRES nucleotide sequence is set out as SEQ ID NO: 30 or a fragment thereof. The FGF IRES may be miniaturized such as the miniFGR IRES set out as SEQ ID NO: 8.

Another aspect of the disclosure provides for compositions comprising a nucleic acid molecule comprising the genome within the nucleotide sequence of any one of SEQ ID NOS: 12-26 and 36, rAAV having a genome within the nucleic acid sequence of SEQ ID NOS: 12-26 and 36 or, rAAV particles comprising a genome within the nucleic acid sequence of any one of SEQ ID NOS: 12-26 and 36. Any of the methods disclosed herein may be carried out with these compositions.

The disclosed AAV comprise a genome comprising a CMV promoter and a variant 2 wild type human GNE cDNA, such as the genome provided in FIG. 1A or the genome set out within SEQ ID NO: 12.

The disclosed AAV comprise a genome comprising a MCK promoter and a variant 2 wild type human GNE cDNA, such as the genome provided in FIG. 1B or the genome set out within SEQ ID NO: 13.

The disclosed AAV comprise a genome comprising a MHCK promoter and a variant 2 wild type human GNE cDNA, such as the genome provided in FIG. 1C or the genome set out within SEQ ID NO: 14.

The disclosed AAV comprise a genome comprising a GNE promoter and a variant 2 wild type human GNE cDNA, such as the genome provided in FIG. 1D or the genome set out within SEQ ID NO: 15.

The disclosed AAV comprise a genome comprising the MHCK7 promoter, a variant 2 wild type GNE cDNA, a nucleic acid sequence encoding miniFGF1 IRES and a nucleic acid sequence encoding follistatin 344, such as the genome provided in FIG. 1E or the genome set out within SEQ ID NO: 16.

The disclosed AAV comprise a genome comprising the MHCK7 promoter, a variant 2 wild type GNE cDNA, a nucleic acid sequence encoding miniFGF1 IRES and a nucleic acid sequence encoding HB-IGF1, such as the genome provided in FIG. 1F or the genome set out within SEQ ID NO: 17.

The disclosed AAV comprise a genome comprising the CMV promoter, a variant 2 wild type GNE cDNA, a nucleic acid sequence encoding FGF1 IRES and nucleic acid sequence encoding follistatin 344, such as the genome provided in FIG. 1G or the genome set out within SEQ ID NO: 18.

The disclosed AAV comprise a genome comprising the CMV promoter, a variant 2 wild type GNE cDNA, nucleic acid sequence encoding miniFGF1 IRES and nucleic acid sequence encoding HB-IGF1, such as the genome provided in FIG. 1H or the genome set out within SEQ ID NO: 19.

The disclosed AAV comprise a genome comprising the MCK promoter, a variant 2 wild type GNE cDNA, nucleic acid sequence encoding miniFGF1 IRES and a nucleic acid sequence encoding follistatin 344, such as the genome provided in FIG. 1I or the genome set out within SEQ ID NO: 20.

The disclosed AAV comprise a genome comprising the MCK promoter, a variant 2 wild type GNE cDNA, nucleic acid sequence encoding miniFGF1 IRES and nucleic acid sequence encoding HB-IGF1, such as the genome provided in FIG. 1J or the genome set out within SEQ ID NO: 21.

The disclosed AAV comprise a genome comprising the GNE promoter, a variant 2 wild type GNE cDNA, nucleic acid sequence encoding miniFGF1 IRES and a nucleic acid sequence encoding follistatin 344, such as the genome provided in FIG. 1K or the genome set out within SEQ ID NO: 22.

The disclosed AAV comprise a genome comprising the GNE promoter, a variant 2 wild type GNE cDNA, a nucleic acid sequence encoding miniFGF1 IRES and a nucleic acid sequence encoding HB-IGF1, such as the genome provided in FIG. 1L or the genome set out within SEQ ID NO: 23.

The disclosed AAV comprise a genome comprising the miniCMV promoter, a variant 2 wild type GNE cDNA, such as the genome provided in FIG. 1M or the genome set out within SEQ ID NO: 24.

The disclosed AAV comprise a genome comprising the miniCMV promoter, a variant 2 wild type GNE cDNA, nucleic acid sequence encoding FGF1 IRES and a nucleic acid sequence encoding follistatin 344, such as the genome provided in FIG. 1N or the genome set out within SEQ ID NO: 25.

The disclosed AAV comprise a genome comprising the miniCMV promoter, a variant 2 wild type GNE cDNA, nucleic acid sequence encoding FGF1 IRES and a nucleic acid sequence encoding HB-IGF1, such as the genome provided in FIG. 1O or the genome set out within SEQ ID NO: 26.

The disclosed AAV comprise a genome comprising the MHCK7 promoter, a variant 2 wild type GNE cDNA, a nucleic acid sequence encoding FGF1 IRES and a nucleic acid sequence encoding SMAD7, such as the genome provided in FIG. 1P

The disclosed AAV comprise a genome comprising the MHCK7 promoter, a variant 2 wild type GNE cDNA, a nucleic acid sequence encoding miniFGF1 IRES and a nucleic acid sequence encoding SMAD7, such as the genome provided in FIG. 1Q.

The disclosed AAV comprise a genome comprising the CMV promoter, a variant 2 wild type GNE cDNA, a nucleic acid sequence encoding FGF1 IRES and nucleic acid sequence encoding SMAD7, such as the genome provided in FIG. 1R.

The disclosed AAV comprise a genome comprising the CMV promoter, a variant 2 wild type GNE cDNA, nucleic acid sequence encoding miniFGF1 IRES and nucleic acid sequence encoding SMAD7, such as the genome provided in FIG. 1S.

The disclosed AAV comprise a genome comprising the MCK promoter, a variant 2 wild type GNE cDNA, nucleic acid sequence encoding FGF1 IRES and a nucleic acid sequence encoding SMAD7, such as the genome provided in FIG. 1T.

The disclosed AAV comprise a genome comprising the MCK promoter, a variant 2 wild type GNE cDNA, nucleic acid sequence encoding miniFGF1 IRES and nucleic acid sequence encoding SMAD7, such as the genome provided in FIG. 1U.

The disclosed AAV comprise a genome comprising the GNE promoter, a variant 2 wild type GNE cDNA, nucleic acid sequence encoding FGF1 IRES and a nucleic acid sequence encoding SMAD7 such as the genome provided in FIG. 1V.

The disclosed AAV comprise a genome comprising the GNE promoter, a variant 2 wild type GNE cDNA, a nucleic acid sequence encoding miniFGF1 IRES and a nucleic acid sequence encoding SMAD7, such as the genome provided in FIG. 1W.

The disclosed AAV comprise a genome comprising the miniCMV promoter, a variant 2 wild type GNE cDNA, nucleic acid sequence encoding FGF1 IRES and a nucleic acid sequence encoding SMAD7, such as the genome provided in FIG. 1X.

The disclosed AAV comprise a genome comprising the miniCMV promoter, a variant 2 wild type GNE cDNA, nucleic acid sequence encoding miniFGF1 IRES and a nucleic acid sequence encoding SMAD7, such as the genome provided in FIG. 1Y.

The disclosed AAV comprise a genome comprising the MCK promoter, a GALGT2 cDNA, nucleic acid sequence encoding FGF1 IRES and a nucleic acid sequence encoding follistatin 344, such as the genome provided in FIG. 1Z or the genome set out within SEQ ID NO: 38.

The disclosed AAV comprise a genome comprising the MCK promoter, a GALGT2 cDNA, nucleic acid sequence encoding FGF1 IRES and a nucleic acid sequence encoding HB-IGF1, such as the genome provided in FIG. 1AA.

The disclosed AAV comprise a genome comprising the MCK promoter, a GALGT2 cDNA, nucleic acid sequence encoding FGF1 IRES and a nucleic acid sequence encoding SMAD7, such as the genome provided in FIG. 1BB.

The disclosure provides for methods of treating GNE myopathy in a human subject in need thereof comprising the step of administering a recombinant adenovirus associated (rAAV) disclosed herein or an AAV. The method of treating GNE myopathy include methods of reducing, inhibiting or slowing the progression of the muscle weakening symptoms of GNE, muscle atrophy and/or methods of increasing muscle strength in a subject in need thereof. The subject in need may be showing the muscle weakening symptoms of GNE myopathy. The subject in need may have a mutation in the GNE gene.

The disclosure provides for methods of treating muscular dystrophy, including Duchene muscular dystrophy, LGMD2A and MDC1A, in a human subject in need thereof comprising the step of administering a recombinant adeno-virus associated (rAAV) disclosed herein or an AAV. The method of treating muscular dystrophy include methods of reducing, inhibiting or slowing the progression of the muscle weakening symptoms, muscle atrophy and/or methods of increasing muscle strength in a subject in need thereof. The subject in need may be showing the muscle weakening symptoms of GNE myopathy. The subject in need may have a mutation in the GNE gene.

In any of the methods of the disclosure, the dose of rAAV can be administered by intramuscular, intraperitoneal, intravenous, intraarterial, oral, buccal, nasal, pulmonary, intracranial, intraosseous, intraocular, rectal, or vaginal route of administration. For example, the route of administration is systemic such as by injection, infusion or implantation. For example, the dose of rAAV is administered by infusion over approximately one hour. In addition, the dose of rAAV is administered by an intravenous route through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

In any of the methods of the disclosure, the rAAV administered is of the serotype AAVrh7.4. The rAAV vectors of the disclosure may be any AAV serotype, such as the serotype AAVrh.74, Anc80, AAV1, AAV2, AAV4, AAV5, AAV6, AAV7, AAV8, AAV9, AAVrh10, AAV11, AAV12, AAV13, AAVTT, AAV7m8 and their derivatives.

In one aspect, the disclosure provides for a rAAV comprising a muscle specific control element nucleotide sequence, and a nucleotide sequence encoding the UDP-N-acetylglucosamine 2-epimerase/N-acetylmannosamine kinase. For example, the nucleotide sequence encodes a functional UDP-N-acetylglucosamine 2-epimerase/N-acetylmannosamine kinase, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 1, wherein the encoded protein retains kinase activity. In addition, the nucleotide sequence encodes a functional protein that comprises an amino acid sequence that, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 2, and retains kinase activity.

In another aspect, the disclosure provides for a rAAV comprising a muscle specific control element nucleotide sequence, and a nucleotide sequence encoding GalNAc transferase. For example, the nucleotide sequence encodes a functional GalNAc transferase, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 36, wherein the encoded protein retains transferase activity. In addition, the nucleotide sequence encodes a functional protein that comprises an amino acid sequence that, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 37, and retains transferase activity.

In another aspect, the disclosures provides for a rAAV comprising a muscle specific control element nucleotide sequence, and a nucleotide sequence encoding a follistatin, such as follistatin 344 or follistatin 317. For example, the nucleotide sequence encodes a functional follistatin wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 9 or 28, wherein the encoded protein retains follistatin activity. In addition, the nucleotide sequence encodes a functional protein that comprises an amino acid sequence that, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 10 or 29 and retains follistatin activity Follistatin activity refers to binding of follistatin to activins and thereby antagonizing activin activity. Follistatin functions by inhibiting repressive growth signaling by myostatin through competitive inhibition and repression of Smad2/3 signaling.

In one embodiment, the disclosure provides for a rAAV comprising a muscle specific promoter element nucleotide sequence, and a nucleotide sequence encoding a IGF variant, such as HB-IGF. For example, the nucleotide sequence encodes a IGF variant wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 11, wherein the encoded protein retains IGF activity. In addition, the nucleotide sequence encodes a functional protein that comprises an amino acid sequence that, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 27, and retains IGF-1 activity. IGF-1 activity refers to IGF-1 binding to and activating the IGF receptor (IGFR) and/or the insulin receptor IGF-1 functions by activating muscle IGFRs and Akt/mTOR signaling. IGF-1 activity includes stimulating cell growth and proliferation, e.g. muscle cell growth, and inhibiting programmed cell death. The disclosure also provides for rAAV wherein the nucleotide sequence comprises a nucleotide sequence that hybridizes under stringent conditions to the nucleic acid sequence of SEQ ID NO: 11, or compliments thereof, and encodes a functional IGF variant.

The disclosure also provides for rAAV wherein the nucleotide sequence comprises a nucleotide sequence that hybridizes under stringent conditions to the nucleic acid sequence of SEQ ID NO: 9 or 28, or compliments thereof, and encodes a functional follistatin.

The disclosure also provides for rAAV wherein the nucleotide sequence comprises a nucleotide sequence that hybridizes under stringent conditions to the nucleic acid sequence of SEQ ID NO: 11, or compliments thereof, and encodes a functional IGF.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

The term “muscle specific promoter element” refers to a nucleotide sequence that regulates expression of a coding sequence that is specific for expression in muscle tissue. These control elements include enhancers and promoters. The disclosure provides for polynucleotides or AAV with a genome comprising one or more of the muscle specific control elements MCKH7 promoter, the MCK promoter, or the MCK enhancer. The GNE promoter may be the promoter for the human wild type GNE gene. Other promoter elements, for example CMV, miniCMV and GNE promoter, allow for expression in almost all tissues, and will be referred to as “constitutive promoters.”

The term “constitutive promoter element” refers to an unregulated promoter that allows for continual transcription of its associated gene. Examples of constitutive promoter elements include hACTB, hEF-1α, CAG, CMV, herpes simplex virus thymidine kinase (HSV-TK), SP1, C-FOS, or C-MYC promoters.

The term “operably linked” refers to the positioning of the regulatory element nucleotide sequence, e.g. promoter nucleotide sequence, to confer expression of said nucleotide sequence by said regulatory element.

For example, the muscle specific promoter element is the MHCK7 promoter nucleotide sequence SEQ ID NO: 5, or the muscle specific promoter element is the CMV promoter nucleic acid sequence of SEQ ID NO: 3, or the muscle specific promoter element is MCK nucleotide sequence of SEQ ID NO: 4 or the muscle specific promoter element is GNE promoter nucleotide sequence of SEQ ID NO: 6 or the muscle specific promoter element is miniCMV nucleotide sequence of SEQ ID NO: 7. In addition, in any of the rAAV vectors of the disclosure, the muscle specific promoter element nucleotide sequence is operably linked to the GNE cDNA sequence. (SEQ ID NO: 1)

In a further aspect, the disclosure provides for an rAAV construct contained in the plasmid comprising the nucleotide sequence of any one of SEQ ID NO: 12-26 and 38 or a nucleotide sequence that is at least 80%, 85%, 90%, 95%, 96%, 97%, 98% or 99% identical to any of the nucleotide sequence of SEQ ID NO: 12-26.

The disclosure also provides for pharmaceutical compositions (or sometimes referred to herein as simply “compositions”) comprising any of the rAAV vectors or rAAV particles of the disclosure.

In another embodiment, the disclosure provides for methods of producing a rAAV particle comprising culturing a cell that has been transfected with any rAAV vectors disclosed herein and recovering rAAV particles from the supernatant of the transfected cells. The disclosure also provides for viral particles comprising any of the disclosed recombinant AAV vectors.

In any of the methods of treating a GNE myopathy, the level of GNE gene expression in a cell of the subject is increased after administration of the rAAV. Expression of the GNE gene in the cell is detected by measuring the UDP-N-acetylglucosamine 2-epimerase/N-acetylmannosamine kinase level by Western blot, immunohistochemistry or enzyme in various tissues (e.g. muscle, heart, liver, kidney, brain, colon assays) before and after administration of the rAAV.

### BRIEF DESCRIPTION OF THE DRAWINGS

FIG. 1A-1BB provide schematic diagrams of the AAV genomes provided herein.

FIG. 2 provides the plasmid sequence comprising the genome of rAAVrh74.CMV.GNE (variant 2) (SEQ ID NO: 12) set out in FIG. 1A.

FIG. 3 provides the plasmid sequence comprising the genome of rAAVrh74.MCK.GNE (variant 2) (SEQ ID NO: 13) set out in FIG. 1B.

FIG. 4 provides the plasmid sequence comprising the genome of rAAVrh74.MHCK7.GNE (variant 2) (SEQ ID NO: 14) set out in FIG. 1C.

FIG. 5 provides the plasmid sequence comprising the genome of rAAVrh74.GNE promoter.GNE (variant 2) (SEQ ID NO: 15) set out in FIG. 1D.

FIG. 6 provides the plasmid sequence comprising the genome of rAAVrh74.MHCK7.GNE(variant 2).FGF1IRES.FS344 (SEQ ID NO: 16) set out in FIG. 1E.

FIG. 7 provides the plasmid sequence comprising the genome of rAAVrh74.MHCK7.GNE(variant2).FGF1IRES.HB-IGF1 (SEQ ID NO: 17) set out in FIG. 1F.

FIG. 8 provides the plasmid sequence comprising the genome of rAAVrh74.CVM.GNE(variant 2).FGF1IRES.FS344 (SEQ ID NO: 18) set out in FIG. 1G.

FIG. 9 provides the plasmid sequence comprising the genome of rAAVrh74.CMV.GNE(variant 2).FGF1IRES.HB-IGF1 (SEQ ID NO: 19) set out in FIG. 1H.

FIG. 10 provides the plasmid sequence comprising the genome of rAAVrh74.MCK.GNE(variant 2).FGF1IRES.FS344 (SEQ ID NO: 20) set out in FIG. 1I.

FIG. 11 provides the plasmid sequence comprising the genome of rAAVrh74.MCK.GNE(variant2).FGF1IRES.HB-IGF1 (SEQ ID NO: 21) set out in FIG. 1J.

FIG. 12 provides the plasmid sequence comprising the genome of rAAVrh74.GNE promoter.GNE(variant 2).FGF1IRES.FS344 (SEQ ID NO: 22) set out in FIG. 1K.

FIG. 13 provides the plasmid sequence comprising the genome of rAAVrh74.GNE promoter.GNE(variant 2).FGF1IRES.HB-IGFI (SEQ ID NO: 23) set out in FIG. 1L.

FIG. 14 provides the plasmid sequence comprising the genome of rAAVrh74.mimiCMV.GNE (SEQ ID NO: 24) set out in FIG. 1M.

FIG. 15 provides the plasmid sequence comprising the genome of rAAVrh74.mimiCMV.GNE(variant 2).FGF1IRES.FS344 (SEQ ID NO: 25) set out in FIG. 1N.

FIG. 16 provides the plasmid sequence comprising the genome of rAAVrh74.miniCMV.GNE(variant 2).FGF1IRES.HB-IGF1 (SEQ ID NO: 26) set out in FIG. 1O.

FIG. 17 provides the plasmid sequence comprising the genome of rAAVrh74.MCK.GALGT2.FGF1IRES.FS344 (SEQ ID NO: 38) set out in FIG. 1Z.

FIG. 18 Sialic acid staining of liver and muscle after intramuscular injection of rAAVrh74.MCK.GNE or IP injection of rAAVrh74.LSP.GNE in GNED176V TgGne−/− mice. Bar is 100 μm

FIG. 19 provides genotyping data from founder mice having Cas9-CRISPR Gne exon 3 deletion/loxP recombination experiment. Founders CR10646-8 and -9 contain a genomic deletion in GNE exon 3.

FIG. 20 provides staining of Gne-deficient Lec3 CHO cells with rAAV.CMV.GNE.mini-IRES.GFP to show expression of a second protein using the mini-IRES sequence. GFP shows endogenous fluorescence, while Gne shows immunostaining, with DAPI in Triple exposure as a stain for nuclei.

FIG. 21 provides staining of Gne-deficient Lec3 CHO cells after transfection with rAAV.miniCMV.GNE. Full-length (FL)-IRES.GFP to show expression of a second protein using the full-length IRES sequence. GFP shows endogenous fluorescence, while Gne shows immunostaining, with DAPI in Triple exposure as a stain for nuclei.

FIG. 22 demonstrates muscle growth after intramuscular injection of IGF1, HB-IGF1 or FST344 using rAAVrh74. The tibialis anterior muscle (TA, left) was injected with 1×10¹¹ vg (vector genomes) and the gastrocnemius muscle (Gastroc, right) was injected with 5×10¹¹ vg of AAV expressing Insulin-like growth factor 1 (IGF1, muscle form Ea), HB-IGF1, or follistatin (FST) form 344. Muscles were dissected and weighed at 2 months post-injection, showing significant increases for HB-IGF1 and FST344 in the TA and for FST344 in the Gastroc compared to injection of buffer alone. Errors are SEM for n=12 muscles per group. *p<0.05, ***p<0.001.

FIG. 23 demonstrates that CMV.GNE.IRES.GFP allows for induction of sialic acid expression on the membranes of Lec3 Gne-deficient CHO cells while the IRES allows for expression of a second protein, in this case GFP. Endogenous GFP expression is shown in the green channel, while MAA staining of sialic acids is shown in red. Normal CHO cells have MAA staining because they have normal Gne function, while Lec3 cells normally do not express MAA, as they do not have functional Gne. Introduction of CMV.GNE.IRES.GFP allows for functional GNE expression in Lec3 cells and also expression of a second protein, GFP, due to the presence of the IRES. DAPI shown in Triple exposure to show nuclei stained in blue.

FIG. 24 demonstrates that muscle cells (C2C12 cells) transfected with MCK.GALGT2.IRES.FS344 (or FST) can express GALGT2 (stained green) and FST (stained red) in the same cells due to the presence of the IRES sequence in the bicistronic vector. C2 cells mock-transfected without the bicistronic DNA show low to no expression of either protein using a time-matched image.

FIG. 25 demonstrates that a change in MAA signal in Lec3 cells after infection with rAAVrh74.CMV.GNE. Maackia amurensis agglutinin (MAA) coupled to horseradish peroxidase (HRP) was used to assay sialic acid expression in CHO or Lec3 cells in a 96-well ELISA plate assays using a colorimetric assay for HRP activity as the output. Lec3 cells grown in Opti-MEM for 3 days show reduced MAA binding relative to CHO cells, and this binding could be partially rescued by addition of rAAVrh74.CMV.GNE for two days. Errors are SD for n=2 per group. MOI, multiplicity of infection, OD, optical density, **p<0.01.

FIG. 26 demonstrates GNE enzyme activity in CHO cells, Lec3 cells, and Lec3 cells transfected with pAAV.CMV.GNE. Cells were lysed and 0.3 mg of total protein was used per sample to measure UDP-GlcNAc epimerase activity. A colorimetric assay was used to measure ManNAc, and samples were compared to a ManNAc standard curve. CHO cells show significantly more UDP-GlcNAc epimerase activity than Lec3 cells, which lack functional Gne enzyme. Lec3 cells transfected with pAAV.CMV.GNE show GNE enzyme activity that exceeds levels found in CHO cells. Errors are SD for n=2 per group. **p<0.01, ***p<0.001

FIG. 27 demonstrates the function of bistronic GALGT2 and Follistatin344 (FST) gene therapy in mdx mice. FIG. 27A demonstrates injection of TA muscle with 1×10¹¹ vg of rAAVrh74.MCK.GALGT2.IRES.FST or the single gene vector rAAVrh74.MCK.FST at the same dose led to an increase in muscle size, measured as muscle weight relative to total body weight (mg/g). Errors are SD for n=4/grp. *p<0.05, **p<0.01. FIG. 27B provides images of the TA muscle stained with antibodies to FST and WFA (to recognize GalNAc made by GALGT2) after injection.

### DETAILED DESCRIPTION

The disclosure provides gene therapy vectors, such as adeno-associated virus (AAV), optimized for delivering a transgene to muscles. The optimized vectors contain constitutive or a muscle-specific promoter to deliver whole body or skeletal/heart muscle-specific transgene expression, respectively, in combination with a transgene cDNA to replace the gene mutation found in a muscle disease with a normal copy of the gene or to provide a surrogate gene therapy, an internal ribosomal entry site (IRES) to allow for production of a second protein from the same transcript, and a muscle growth factor, to build new muscle growth and strength. The transgene and the muscle growth factor gene are expressed from the same mRNA, which expresses both proteins due to the presence of an Internal Ribosomal Entry Site (or IRES) from the Fibroblast Growth Factor 1A gene sequence, which allows for the second protein to be made from the single mRNA.

The disclosure provides gene therapy vectors, such as adeno-associated virus (AAV), designed for treatment of GNE myopathy. The AAV express UDP-GlcNAc-epimerase/ManNAc-6 alone or in combination with follistatin or IGF1. The provided AAV replace the mutated GNE gene expression while expressing proteins that stimulate muscle growth. The strategy of combining gene replacement functionality (either direct gene replacement or replacement with a surrogate gene function), which will prevent further disease, with muscle growth or muscle transdifferentiation therapy, which will build new muscle mass and strength, has the potential not only to arrest the disease process but to reverse it by simultaneously stopping disease pathogenesis while stimulating new muscle growth and strength.

Provided herein are gene therapy vectors that are 1) provide a transgene for gene replacement or as a surrogate gene therapy and 2) provide the gene encoding a growth factor that induced muscle growth or increases muscle strength. This gene therapy is encoded by a single gene therapy genome, e.g. a single AAV genome. This combination therapy has the potential not only to arrest the disease process but to reverse it by simultaneously stopping disease pathogenesis while stimulating new muscle growth and strength.

The provided gene therapy is useful for treating GNE myopathies, Duchenne and Becker muscular dystrophies (DMD and BMD) and limb girdle muscular dystrophies (LGMD) such as LGMD2A (CAPN3, LGMD2C (SGCG), LGMD2D (SGCA), LGMD2E (SGCB), LGMD2F (SGCD), LGMD2G (TCAP), LGMD2H (TRIM32), LGMD2I (FKRP), LGMD2K (POMT1), LGMD2L (ANO5), LGMD2M (FKTN), LGMD2O (POMT2), LGMD2P (DAG1), LGMD2R (DES), LGMD2T (GMPPB) LGMD2U (ISPD), LGMD2X (BVES), LGMD2Y (TOR1AIP1), LGMD2Z (POGLUT1), LGMD1A (TTID, MYOT), LGMD1B (LMNA), LGMD1C (Cav3), LGMD1D (DES), LGMD1F (TNPO3), LGMD1G (HNRPDL) and MDC1A. In each instance, the first transgene may be used for gene replacement for the gene missing in the disease or a surrogate gene replacement, such as GALGT2 or B4GALNT2), while the second transgene is a muscle growth factor such as FS344, HB-IGF1, IGF1 or SMAD7 to reverse disease symptoms by building new muscle growth and strength. The gene therapy of the present disclosure also may be used to treat diseases where a surrogate gene is used to prevent disease in lieu of gene replacement as the first transgene, and would apply to therapies where muscle growth from placement of the second transgene comes not from a muscle growth factor but from a muscle transdifferentiation factor (e.g., MyoD), where muscle is built by conversion of fat or fibroblasts to muscle rather than from muscle growth factor support.

In some embodiments, wherein constructs that have space available, the AAV genome comprises a second IRES and a third transgene to provide three gene therapies at once as well.

AAV having a genome comprising a muscle specific promoter which drives expression of a nucleotide sequence encoding a transgene of interest in combination with a nucleotide sequence encoding a muscle growth factor, such as a protein that induces muscle growth and a muscle-specific IRES such as the FGF IRES. This gene therapy approach is useful for treating any disease that requires gene replacement in combination with the need to increase muscle growth or muscle strength such as GNE myopathy, limb girdle muscular dystrophies and Duchene muscular dystrophy.

### Growth Factors and Transdifferentiation Factors

Growth factors that induce muscle growth or increase muscle strength include IGF, HB-IGF, Pax7, HGF (hepatocyte growth factor), HGH (human growth hormone), FGF19 (fibroblast growth factor 19), FGF21 (fibroblast growth factor 21), VEGF (vascular endothelial growth factor), IL6 (Interleukin 6), IL15 (Interleukin 15) and SMAD7 (mothers against decapentaplegic homolog 7 (MADH7)).

Growth factors that induce muscle growth or increase muscle strength also include the follistatins. Follistatin is a secreted protein that inhibits the activity of TGF-β family members such as GDF-11/BMP-11. Follistatin-344 is a follistatin precursor that undergoes peptide cleavage to form the circulating Follistatin-315 isoform which includes a C-terminal acidic region. It circulates with myostatin propeptide in a complex that includes two other proteins, follistatin related gene (FLRG) and GDF associated serum protein (GASP-1). Follistatin-317 is another follistatin precursor that undergoes peptide cleavage to form the membrane-bound Follistatin-288 isoform.

The DNA and amino acid sequences of the follistatin-344 precursor are respectively set out in SEQ ID NOs: 9 and 10. The Follistatin-288 isoform, which lacks a C-terminal acidic region, exhibits strong affinity for heparin-sulfate-proteoglycans, is a potent suppressor of pituitary follicle stimulating hormone, is found in the follicular fluid of the ovary, and demonstrates high affinity for the granulose cells of the ovary. The testis also produce Follistatin-288. The DNA and amino acid sequences of the follistatin-317 precursor are respectively set out in SEQ ID NOs: 28 and 29. Lack of follistatin results in reduced muscle mass at birth.

Examples of follistatins are provided in Shimasaki et al., U.S. Pat. No. 5,041,538, other follistatin-like proteins are provided in U.S. Pat. Nos. 5,942,420; 6,410,232; 6,537,966; and 6,953,662), FLRG (SEQ ID NO: 33, the corresponding nucleotide sequence is SEQ ID NO: 32) is provided in Hill et al., J. Biol. Chem., 277(43): 40735-40741 (2002)] and GASP-1 (SEQ ID NO: 35, corresponding nucleotide sequence is SEQ ID NO: 34) is provided in Hill et al., Mol Endocrinol, 17: 1144-1154 (2003).

SMAD7 is known to inhibit TGF-β-activated signaling responses by associating with the active TGF-β complex, which results in reduced TGF-β signaling. Myostatin and TGF-β signaling induces SMAD7 expression establishing a negative feedback loop to inhibit TGF-β signaling. In particular, SMAD7 is known to modulate myogenesis using this negative feedback loop (Kollias et al. Mol. Cell Biol. 26(16):6248-6260, 2006. The nucleotide sequence encoding the SMAD7 protein is set out as SEQ ID NO: 39 (Genbank Accession No. NM_005904.4), and the amino acid sequence is set out as SEQ ID NO: 40 (Genbank Accession No. NP_005895).

Transdifferentiation factors are agents that convert or induce differentiation to a non-muscle cell to muscle. For example, MyoD is known to convert a number of cell types into muscle, including dermal fibroblasts, chondrocytes, smooth muscle, retinal pigmented epithelial cells, adipocytes, and melanoma, neuroblastoma, osteosarcoma, and hepatoma cells (Abraham & Tapscott, Curr. Opin. Genet. Dev. 23(5): 568-573, 2013). Other examples of transdifferentiatation factors Myocd (myocardin), Mef2C (myocyte enhancer factor 2C), Mef2B (myocyte enhancer factor 2B), Mkl1 (MKL [megakaryoblastic leukemia]/Myocd-like 1), Gata4 (GATA-binding protein 4), Gata5 (GATA-binding protein 5), Gata6 (GATA-binding protein 6), Ets1 (E26 avian leukemia oncogene 1, 5′ domain).

### GNE Myopathy

GNE myopathy is characterized by progressive muscle atrophy and weakness. The age of onset is typically in the third decade of life, beginning with weakness in the tibialis anterior (TA) and hamstring muscles and often rendering patient's wheelchair-bound by the second decade after diagnosis. Patients may ultimately require assistance with daily living functions such as eating. Muscle biopsies typically shows rimmed vacuoles and inclusion bodies. GNE myopathy is caused by mutations in the GNE gene, which encodes a bifunctional UDP-GlcNAc epimerase/ManNAc-6 kinase. GNE function is required for synthesis of all sialic acid (SA). The SA biosynthetic pathway culminates in the production of CMP-SA, which is utilized by sialyltransferases to transfer SA onto glycoproteins and glycolipids in all mammalian cells.

GNE myopathy incidence has recently been estimated to between 1 and 6 per million, a rare disease. There are, however, founder effect mutations that cause GNE myopathy to occur at much higher incidence in certain human populations, for example in patients of Japanese (D176V, D207V in the new nomenclature) and Middle Eastern (M712T, M743T in the new nomenclature) descent. Disease mutation carrier frequency in one study of 1000 Iranian Jews was found to be 1 in 11. The partial reduction in GNE activity in patients leads to reduced, but not absent, SA expression.

Diminished IGF1R signaling has been shown to be a basis for muscle stem cell death in a model of GNE myopathy, making IGF1 a possible ideal growth factor element to the gene therapy design. These tandem gene vectors are expected not only to inhibit disease progression (the function of GNE gene replacement) but also induce new muscle growth (thereby increasing muscle strength) and possibly prevent stem cell death. These vectors are highly unique, as patients with GNE myopathy lose muscle and strength over decades, and the provided AAV are expected not only to slow this progression but to actually reverse it. The provided dual function AAV will be able to show clinical efficacy, as this disease shows high clinical variability (between patient disease mutations and even amongst patients with the same disease mutation) and because it is slowly progressing (with major clinical changes occurring over decades).

### GNE Myopathy Mutations

In any of the provided methods of the subject is suffering from GNE myopathy. For the example, the subject has a mutation in the GNE gene that results in reduced expression of UDP-N-acetylglucosamine 2-epimerase/N-acetylmannosamine kinase. A diagnosis of GNE myopathy is confirmed in a subject by the presence of pathogenic (mostly missense) mutations in both alleles of the GNE gene. Table 1 provides of known mutations in the GNE gene that are associated with GNE myopathy is provided below. The subjects of the claimed methods may comprise a mutation set out in this table.

In Table 1 Bold print indicates cDNA or protein truncating variants. Italic print+dark gray highlight indicates ‘Mild’ variants. A question mark (?) indicates that the exact nomenclature could not be extracted from the reference. The DNA numbering system is based on cDNA sequence. Nucleotide numbering uses +1 as the A of the ATG translation initiation codon in the reference sequence, with the initiation codon as codon 1.

| | | Nucleotide | | GNE | | | |
|---|---|---|---|---|---|---|---|
| Amino Acid Substitution | | Substitution | Gene | protein | Severity | | |
| hGNE1 | hGNE2 | mRNA Variant1 | Exon | domain | Prediction | Ethnicity | Reference |
| E2G | p.E33G | c.98A > G | 3 | ep | Severe | European | [Saechao et al., 2010] |
| R8* | p.R39* | c.115C > T | 3 | ep | Severe | Caucasian, | [Saechao et al., |
| | | | | | | Chinese, | 2010; Lu et al., |
| | | | | | | Japanese | 2011; Mori- |
| | | | | | | | Yoshimura et al., |
| | | | | | | | 2012] |
| R11W | p.R42W | c.124C > T | 3 | ep | Severe | Indian | [Huizing et al., 2004] |
| C13S | p.C44S | c.131G > C | 3 | ep | Medium | Chinese, | [Kim et al., 2006; Lu |
| | | | | | | Japanese, | et al., 2011; Park et |
| | | | | | | Korean | al., 2012; Tomimitsu |
| | | | | | | | et al., 2004] |
| A26P | p.A57P | c.169G > C | 3 | ep | Mild | Caucasian | [Weihl et al., 2011] |
| P27L | p.P58L | c.173C > T | 3 | ep | Medium | Japanese, | [ Mori-Yoshimura et |
| | | | | | | Indian | al., 2012; Nalini et |
| | | | | | | | al., 2013] |
| P27S | p.P58S | c.172C > T | 3 | ep | Medium | Italian | [Broccolini et al., |
| | | | | | | | 2004] |
| I28M | p.I59M | c.177C > G | 3 | ep | Medium | Japanese | [Cho et al., 2013] |
| M29T | p.M60T | c.179T > C | 3 | ep | Medium | Korean, | [Kim et al., |
| | | | | | | Japanese | 2006; Cho et al., |
| | | | | | | | 2013] |
| M29R | p.M60R | c.179T > G | 3 | ep | Severe | Japanese | [Cho et al., 2013] |
| E35K | p.E66K | c.196G > A | 3 | ep | Medium | Chinese | [Li et al., 2011; Lu et |
| | | | | | | | al., 2011] |
| P36L | p.P67L | c.200C > T | 3 | ep | Severe | Italian | [Eisenberg et al., |
| | | | | | | | 2003] |
| E40K | p.E71K | c.211G > A | 3 | ep | Medium | Japanese | [Cho et al., 2013] |
| frameshift | frameshift | ins10 bp? | 3 | ep | Severe | Japanese | [Nishino et al., 2002] |
| I51M | p.I82M | c.246A > G | 3 | ep | Medium | Chinese | [Li et al., 2011; Lu et |
| | | | | | | | al., 2011] |
| exon 3 del | exon 3 del | ? | 3 | ep | Severe | Japanese | [Cho et al., 2013] |
| M60V | p.M91V | c.271A > G | 4 | ep | Mild | Portuguese | novel |
| R71W | p.R102W | c.304A > T | 4 | ep | Severe | Caucasian | [Saechao et al., 2010] |
| G89R | p.G120R | c.358G > C | 4 | ep | Severe | Thai, | [Liewluck et al., |
| | | | | | | Japanese | 2006; Cho et al., |
| | | | | | | | 2013] |
| G89S | p.G120S | c.358G > A | 4 | ep | Medium | Japanese | [Mori-Yoshimura et |
| | | | | | | | al., 2012; Cho et al., |
| | | | | | | | 2013] |
| R101C | p.R132C | c.394C > T | 4 | ep | Severe | Korean | [Park et al., 2012] |
| R101H | p.R132H | c.395G > A | 4 | ep | Medium | Japanese | [Cho et al., 2013] |
| I106T | p.I137T | c.410T > C | 4 | ep | Medium | Chinese | [Lu et al., 2011] |
| I128Ifs*6 | p.I159Ifs*6 | c.476insT | 4 | ep-NES | Severe | Japanese | [Mori-Yoshimura et |
| | | | | | | | al., 2012; Cho et al., |
| | | | | | | | 2013] |
| R129Q | p.R160Q | c.479G > A | 4 | ep-NES | Medium | Japanese | [Mori-Yoshimura et |
| | | | | | | | al., 2012] |
| R129* | p.R160* | c.478C > T | 4 | ep-NES | Severe | Indian | novel |
| H132Q | p.H163Q | c.489C > G | 4 | ep-NES | Medium | Japanese | [Nishino et al., |
| | | | | | | | 2002; Tomimitsu et |
| | | | | | | | al., 2002] |
| G135V | p.G166V | c.497G > T | 4 | ep-NES | Severe | English, | [Sparks et al., 2005] |
| | | | | | | Irish, USA | |
| G136R | p.G167R | c.501G > A | 4 | ep-NES | Severe | Japanese | [Cho et al., 2013] |
| I142T | p.I173T | c.518T > C | 4 | ep | Severe | Caucasian | [Saechao et al., 2010] |
| I150 V | p.I181V | c.541A > G | 4 | ep | Medium | European | [No et al., 2013] |
| Y156H | p.Y187H | c.559T > C | 4 | ep | Medium | Indian | novel |
| H157fs | p.H188fs | ? | 4 | ep | Severe | Korean | [Sim et al., 2013] |
| R162C | p.R193C | c.515C > T | 4 | ep | Severe | Italian, | [Del Bo et al., |
| | | | | | | Indian | 2003; Nalini et al., |
| | | | | | | | 2013] |
| M171V | p.M202V | c.604A > G | 4 | ep | Severe | Italian | [Broccolini et al., |
| | | | | | | | 2002] |
| D176V | p.D207V | c.620A > T | 4 | ep | Medium | Chinese, | [Nishino et al., |
| | | | | | | Japanese, | 2002; Tomimitsu et |
| | | | | | | Korean | al., |
| | | | | | | | 2002, 2004; Motozaki |
| | | | | | | | et al., 2007; Li et al., |
| | | | | | | | 2011; Park et al., |
| | | | | | | | 2012] |
| R177C | p.R208C | c.622C > T | 4 | ep | Severe | Japanese | [Nishino et al., |
| | | | | | | | 2002; Cho et al., |
| | | | | | | | 2013] |
| I178N | p.I209N | c.626T > A | 4 | ep | Severe | Japanese | [Cho et al., 2013] |
| I178M | p.I209M | c.627C > G | 4 | ep | Medium | Japanese | [Cho et al., 2013] |
| L179F | p.L210F | c.628C > T | 4 | ep | Medium | Italian | [Grandis et al., 2010] |
| Y186C | p.Y217C | c.650A > G | 4 | ep | Severe | Pakistani | [No et al., 2013] |
| D187G | p.D218G | c.653A > G | 4 | ep | Severe | Japanese | [Mori-Yoshimura et |
| | | | | | | | al., 2012; Cho et al., |
| | | | | | | | 2013] |
| N194Tfs*4 | p.N225Tfs*4 | c.674delA | 4 | ep | Severe | Japanese | [Cho et al., 2013] |
| I200F | p.I231F | c.691A > T | 4 | ep | Medium | USA | [Eisenberg et al., |
| | | | | | | | 2003] |
| R202L | p.R233L | c.698G > T | 4 | ep | Medium | Greek | [Huizing et al., 2004] |
| W204* | p.W235* | c.705G > A | 4 | ep | Severe | Caucasian | [Saechao et al., 2010] |
| G206S | p.G237S | c.709G > A | 4 | ep | Medium | Italian | [Broccolini et al., |
| | | | | | | | 2004] |
| splicing | splicing | c.710-4A > G | in 4 | ep | Splicing? | Japanese | [Cho et al., 2013] |
| G206Vfs*3 | p.G237Vfs*3 | c.710delG | 5 | ep | Severe | Italian | [Broccolini et al., |
| | | | | | | | 2004] |
| D208N | p.D239N | c.715G > A | 5 | ep | Mild | Korean | [Sim et al., 2013] |
| D213V | p.D244V | c.731A > T | 5 | ep | Medium | Indian | novel |
| V216A | p.V247A | c.740T > C | 5 | ep | Severe | USA, | [ Vasconcelos et al., |
| | | | | | | German, | 2002; Huizing et al., |
| | | | | | | Dutch | 2004] |
| Q219K | p.Q250K | c.748C > A | 5 | ep | Medium | Japanese | [Cho et al., 2013] |
| D225N | p.D256N | c.766G > A | 5 | ep | Medium | Bahamas | [Eisenberg et al., |
| | | | | | | | 2001] |
| F233S | p.F264S | c.791T > C | 5 | ep | Medium | Japanese | [Mori-Yoshimura et |
| | | | | | | | al., 2012] |
| I241S | p.I272S | c.815T > G | 5 | ep | Medium | Chinese, | [Ro et al., 2005; Chu |
| | | | | | | Taiwanese | et al., 2007; Li et al., |
| | | | | | | | 2011; Lu et al., 2011] |
| R246W | p.R277W | c.829C > T | 5 | ep | Severe | Caucasian, | [Darvish et al., |
| | | | | | | Chinese, | 2002; Ro et al., |
| | | | | | | Japanese, | 2005; Sparks et al., |
| | | | | | | Italian, | 2005; Saechao et al., |
| | | | | | | USA | 2010; Stober et al., |
| | | | | | | | 2010; Li et al., |
| | | | | | | | 2011; Cho et al., |
| | | | | | | | 2013] |
| R246Q | p.R277Q | c.830G > A | 5 | ep | Mild | Bahamas, | [Eisenberg et al., |
| | | | | | | Italian, | 2001; Broccolini et |
| | | | | | | Taiwanese, | al., 2004; Chu et al., |
| | | | | | | Japanese | 2007; Saechao et al., |
| | | | | | | | 2010; Chai et al., |
| | | | | | | | 2011] |
| splicing | splicing | c. 862 + 4A > G | in 5 | ep | Splicing | Japanese | [Nishino et al., |
| | | | | | | | 2002; Cho et al., |
| | | | | | | | 2013] |
| M261V | p.M292V | c.874A > G | 6 | ep-AR | Mild | Korean | [Park et al., 2012] |
| M261I | p.M292I | c.876G > ? | 6 | ep-AR | Mild | Korean | [Sim et al., 2013] |
| M265T | p.M296T | c.887T > C | 6 | ep-AR | Medium | European | [No et al., 2013] |
| I270N | p.I301N | c.902T > A | 6 | ep-AR | Medium | Japanese | [Cho et al., 2013] |
| I270T | p.I301T | c.902T > C | 6 | ep-AR | Medium | Japanese | [Cho et al., 2013] |
| R277C | p.R308C | c.922C > T | 6 | ep-AR | Medium | French, | [Behin et al., |
| | | | | | | Japanese | 2008; Cho et al., |
| | | | | | | | 2013] |
| R277G | p.R308G | c.922C > G | 6 | ep-AR | Medium | Japanese | [Cho et al., 2013] |
| P283S | p.P314S | c.940C > T | 6 | ep-AR | Medium | Japanese | [Tomimitsu et al., |
| | | | | | | | 2004] |
| H293R | p.H324R | c.971A > G | 6 | ep-AR | Medium | Indian | [Kannan et al., 2012] |
| G295D | p.G326D | c.977G > A | 6 | ep-AR | Medium | Japanese | [Mori-Yoshimura et |
| | | | | | | | al., 2012] |
| G295R | p.G326R | c.976G > C | 6 | ep-AR | Medium | Japanese | [Cho et al., 2013] |
| M297T | p.M328T | c.983T > C | 6 | ep-AR | Medium | Indian | novel |
| I298T | p.I329T | c.986T > C | 6 | ep-AR | Severe | Asian, | [Saechao et al., |
| | | | | | | Chinese, | 2010; Lu et al., 2001] |
| | | | | | | Indian | |
| N300K | p.N331K | c.993C > A | 6 | ep-AR | Severe | Italian | [Tasca et al., 2012] |
| C303V | p.C334V | c.1000_1001TG > GT | 6 | ep-AR | Medium | Japanese | [Tomimitsu et al., |
| | | | | | | | 2002] |
| C303* | p.C334* | c.1002T > A | 6 | ep-AR | Severe | Indian | [Eisenberg et al., |
| | | | | | | | 2001] |
| G304R | p.G335R | c.1003G > A | 6 | ep | Severe | Indian | [Nalini et al., 2013] |
| R306Q | p.R337Q | c.1010G > A | 6 | ep | Medium | Japanese | [Nishino et al., 2002] |
| A310P | p.A341P | c.1021G > C | 6 | ep | Severe | Chinese | [Ro et al., |
| | | | | | | | 2005; Stober et al., |
| | | | | | | | 2010] |
| V315M | p.V346M | c.1036G > A | 6 | ep | Medium | European | [No et al., 2013] |
| N317D | p.N348D | c.1042A > G | 6 | ep | Severe | European | [No et al., 2013] |
| R321C | p.R352C | c.1054C > T | 6 | ep | Severe | Japanese | [Mori-Yoshimura et |
| | | | | | | | al., 2012] |
| splicing | splicing | c.1076-1delG | in 6 | ep | Splicing? | Japanese | [Cho et al., 2013] |
| V331A | p.V362A | c.1085T > C | 7 | ep | Severe | Japanese | [Nishino et al., 2002] |
| H333R | p.H364R | c.1091A > G | 7 | ep | Medium | Caucasian | [Weihl et al., 2011] |
| R335W | p.R366W | c.1096C > T | 7 | ep | Severe | Caucasian | [Fisher et al., |
| | | | | | | | 2006; Saechao et al., |
| | | | | | | | 2010] |
| L347del; | p.L378del; | c.1132_1134 | 7 | ep | Severe | Caucasian | [Fisher et al., 2006] |
| H348N | p.H379N | del; c.1135C > A | | | | | |
| L347P | p.L378P | c.1133T > C | 7 | ep | Severe | Japanese | [Cho et al., 2013] |
| splicing | splicing | c.1163 + 2dupT | in 7 | ep | Splicing | European, | [Broccolini et al., |
| | | | | | | Italian | 2004; No et al., 2013] |
| Y361* | p.Y392* | c.1176T > G | 8 | ep | Severe | Caucasian | [Weihl et al., 2011] |
| V367I | p.V398I | c.1192G > A | 8 | ep | Medium | Iranian | [Krause et al., 2003] |
| I377Tfs*15 | p.I408Tfs*15 | c.1223delT | 8 | ep | Severe | Italian | [Broccolini et al., |
| | | | | | | | 2004] |
| D378Y | p.D409Y | c.1225G > T | 8 | UF | Severe | European, | [Nishino et al., |
| | | | | | | Irish, | 2002; Eisenberg et |
| | | | | | | Japanese, | al., 2003; No et al., |
| | | | | | | USA | 2013] |
| L379H | p.L410H | c.1229T > A | 8 | UF | Severe | Tunisian | [Amouri et al., 2005] |
| P390S | p.P421S | c.1261C > T | 8 | UF | Medium | Korean | [Sim et al., 2013] |
| R420* | p.R451* | c.1351C > T | 8 | kin | Severe | Japanese, | [Tomimitsu et al., |
| | | | | | | Indian | 2004; Nalini et al., |
| | | | | | | | 2013] |
| V421A | p.V452A | c.1355T > C | 8 | kin | Medium | Japanese | [Tomimitsu et al., |
| | | | | | | | 2004; Cai et al., |
| | | | | | | | 2013] |
| K432Rfs*16 | p.K463Rfs*16 | c.1388delA | 9 | kin | Severe | Indian | [Voermans et al., |
| | | | | | | | 2010] |
| Y434C | p.Y465C | c.1394A > G | 9 | kin | Medium | Korean | [Sim et al., 2013] |
| Q436* | p.Q467* | c.1399C > T | 9 | kin | Severe | Taiwanese | [Saechao et al., 2010] |
| C453F | p.C484F | c.1451G > T | 9 | kin | Severe | Japanese | [Cho et al., 2013] |
| A460V | p.A491V | c.1472C > T | 9 | kin | Medium | Japanese | [Kayashima et al., |
| | | | | | | | 2002] |
| .L463P | p.L494P | c.1481T > C | 9 | ep | Severe | Korean | [Sim et al., 2013] |
| G469R | p.G500R | c.1498G > A | 9 | kin | Severe | Japanese | [Cho et al., 2013] |
| splicing | splicing | c.1504 + 5G > A | in 9 | kin | Splicing | Japanese | [Cho et al., 2013] |
| splicing | splicing | c.1505 + 4G > A | in 9 | kin | Splicing? | Japanese | [Cho et al., 2013] |
| I472T | p.I503T | c.1508T > C | 10 | kin | Severe | Japanese | [Nishino et al., |
| | | | | | | | 2002; Yabe et al., |
| | | | | | | | 2003] |
| W495* | p.W526* | c.1577G > A | 10 | kin | Severe | Caucasian | [No et al., 2013] |
| L508S | p.L539S | c.1616T > C | 10 | kin | Severe | Chinese | [Li et al., 2011; Lu et |
| | | | | | | | al., 2011] |
| H509Y | p.H540Y | c.1618C > T | 10 | kin | Medium | Chinese | [Lu et al., 2011] |
| P511H | p.P542H | c.1625C > A | 10 | kin | Severe | Japanese | [Motozaki et al., |
| | | | | | | | 2007] |
| P511L | p.P542L | c.1625C > T | 10 | kin | Severe | Thai | [Liewluck et al., |
| | | | | | | | 2006] |
| W513* | p.W544* | c.1632G > A | 10 | kin | Severe | Chinese, | [Ro et al., 2005; Li et |
| | | | | | | Taiwanese, | al., 2011; Nalini et |
| | | | | | | Indian | al., 2013] |
| V514del | p.V545del | c.1634_1637del | 10 | kin | Severe | Japanese | [Cho et al., 2013] |
| N519S | p.N550S | c.1649A > G | 10 | kin | Medium | Italian | [Broccolini et al., |
| | | | | | | | 2004] |
| A524V | p.A555V | c.1664C > T | 10 | kin | Severe | French, | [Darvish et al., |
| | | | | | | Mexican, | 2002; Liewluck et al., |
| | | | | | | Thai, | 2006; Behin et al., |
| | | | | | | Japanese | 2008; Cho et al., |
| | | | | | | | 2013] |
| F528C | p.F559C | c.1676T > G | 10 | kin | Severe | German | [Eisenberg et al., |
| | | | | | | | 2003] |
| G545Efs*11 | p.G576Efs*11 | c.1727delG | 11 | kin | Severe | Korean | [Park et al., 2012] |
| L556S | p.L587S | c.1760T > C | 11 | kin | Severe | Caucasian | [Saechao et al., 2010] |
| I557T | p.I588T | c.1763T > C | 11 | kin | Medium | Italian, | [Eisenberg et al., |
| | | | | | | Japanese | 2003; Tomimitsu et |
| | | | | | | | al., 2004] |
| G559R | p.G590R | c.1768G > C | 11 | kin | Severe | Japanese, | [Huizing et al., |
| | | | | | | Greek | 2004; Cho et al., |
| | | | | | | | 2013;] |
| G559A | p.G590A | c.1769G > C | 11 | kin | Severe | Turkish | novel |
| G568S | p.G599S | c.1795G > A | 11 | kin | Severe | Japanese | [Mori-Yoshimura et |
| | | | | | | | al., 2012] |
| G568V | p.G599V | c.1796G > T | 11 | kin | Severe | Indian | [Nalini et al., 2013] |
| V572del | p.V603del | c.1806_1808del | 11 | kin | Severe | Japanese | [Cho et al., 2013] |
| V572L | p.V603L | c.1807G > C | 11 | kin | Medium | Asian, | [Kayashima et al., |
| | | | | | | Chinese, | 2002; Tomimitso et |
| | | | | | | Japanese, | al., 2002; Kim et al., |
| | | | | | | Korean | 2006; Li et al., |
| | | | | | | | 2011; Parket al., |
| | | | | | | | 2012] |
| G576E | p.G607E | c.1820G > A | 11 | kin | Severe | USA | [Eisenberg et al., |
| | | | | | | | 2001] |
| C579Y | p.C610Y | c.1829G > A | 11 | kin | Severe | Japanese | [Cho et al., 2013] |
| C581R | p.C612R | c.1834T > C | 11 | kin | Severe | Pakistani | novel |
| C586* | p.C617* | c.1850delG | 11 | kin | Severe | Japanese | [Mori-Yoshimura et |
| | | | | | | | al., 2012] |
| I587T | p.I618T | c.1853T > C | 11 | kin | Medium | Algerian, | [Tomimitsu et al., |
| | | | | | | Chinese, | 2002; Kalaydjieva et |
| | | | | | | Italian, | al., 2005; Behin et al., |
| | | | | | | Cajun, | 2008; Grandis et al., |
| | | | | | | Japanese, | 2010; Li et al., |
| | | | | | | Roma | 2011; Cho et al., |
| | | | | | | Gypsies | 2013] |
| I587N | p.I618N | c.1853T > A | 11 | kin | Medium | Japanese | [Cho et al., 2013] |
| A591T | p.A622T | c.1864G > A | 11 | kin | Severe | Chinese, | [Kim et al., 2006; Lu |
| | | | | | | Korean | et al., 2011] |
| A600E | p.A631E | c.1892C > A | 11 | kin | Severe | Japanese | [Mori-Yoshimura et |
| | | | | | | | al., 2012; Cho et al., |
| | | | | | | | 2013] |
| A600T | p.A631T | c.1891G > A | 11 | kin | Medium | Italian | [Broccolini et al., |
| | | | | | | | 2004] |
| L603F | p.L634F | c.1900C > T | 11 | kin | Medium | Japanese | [Mori-Yoshimura et |
| | | | | | | | al., 2012] |
| splicing | splicing | c.1909 + 5G > A | in 11 | kin | Splicing | Indian | [Boyden et al., 2011] |
| S615* | p.S646* | c.1937C > G | 12 | kin | Severe | Caucasian | [Saechao et al., 2010] |
| A630Lfs*12 | p.A661Lfs*12 | c.1980delA | 12 | kin | Severe | Japanese | [Cho et al., 2013] |
| A630T | p.A661T | c.1981G > A | 12 | kin | Medium | Japanese | [Nishino et al., |
| | | | | | | | 2002; Cho et al., |
| | | | | | | | 2013] |
| A631T | p.A662T | c.1984G > A | 12 | kin | Severe | Caucasian, | [Eisenberg et al., |
| | | | | | | Senegalese, | 2001; Behin et al., |
| | | | | | | USA | 2008; No et al., 2013] |
| A631V | p.A662V | c.1985C > T | 12 | kin | Severe | Caucasian, | [Nishino et al., |
| | | | | | | Korean, | 2002; Tomimitsu et |
| | | | | | | Chinese, | al., |
| | | | | | | German, | 2002; Vasconcelos et |
| | | | | | | Irish, S. | al., 2002; Eisenberg |
| | | | | | | African, | et al., 2003; Saechao |
| | | | | | | USA, | et al., 2010; Li et al., |
| | | | | | | Japanese | 2011; Weihl et al., |
| | | | | | | | 2011; Park et al., |
| | | | | | | | 2012] |
| N635K | p.N666K | c.1998T > A | 12 | kin | Severe | Japanese | [Cho et al., 2013] |
| N635K | p.N666K | c.1998T > G | 12 | kin | Severe | Japanese | [Cai et al., 2013] |
| A648V | p.A679V | c.2036C > T | 13 | kin | Medium | German | novel |
| I656N | p.I687N | c.2060T > A | 13 | kin | Severe | Thai | [Liewluck et al., |
| | | | | | | | 2006] |
| G669R | p.G700R | c.2098G > A | 13 | kin | Severe | Japanese | [Cho et al., 2013] |
| G669R | p.G700R | c.2098G > C | 13 | kin | Severe | Asian, | [No et al., 2013] |
| | | | | | | Indian, | |
| | | | | | | Portuguese | |
| Y675H | p.Y706H | c.2116T > C | 13 | kin | Medium | Caucasian | [Darvish et al., |
| | | | | | | | 2002; Saechao et al., |
| | | | | | | | 2010] |
| V679G | p.V710G | c.2129T > G | 13 | kin | Severe | French | [Behin et al., 2008] |
| V696M | p.V727M | c.2179G > A | 13 | kin | Medium | Algerian, | [Eisenberg et al., |
| | | | | | | Asian, | 2001; Huizing et al., |
| | | | | | | Chinese, | 2004; Liewluck et al., |
| | | | | | | Middle- | 2006; Behin et al., |
| | | | | | | Eastern, | 2008; Saechao et al., |
| | | | | | | Indian, | 2010; Voermans et |
| | | | | | | Pakistani, | al., 2010; Boyden et |
| | | | | | | Thai, | al., 2011; Lu et al., |
| | | | | | | Portuguese | 2011; No et al., 2013] |
| S699L | p.S730L | c.2189C > T | 13 | kin | Severe | Middle- | [No et al., 2013] |
| | | | | | | Eastern | |
| G708S | p.G739S | c.2215G > A | 13 | kin | Severe | Japanese | [Tomimitsu et al., |
| | | | | | | | 2004; Cho et al., |
| | | | | | | | 2013] |
| M712T | p.M743T | c.2228T > C | 13 | kin | Severe | Egyptian- | [Eisenberg et al., |
| | | | | | | Muslim, | 2001; Broccolini et |
| | | | | | | Persian | al., 2002; Darvish et |
| | | | | | | Jewish, | al., 2002; Noguchi et |
| | | | | | | Japanese | al., 2004; Tomimitsu |
| | | | | | | | et al., 2004; Amouri |
| | | | | | | | et al., 2005; Cho et |
| | | | | | | | al., 2013] |
| large | large deletion | del ex2-ex10 | 2-10 | ep + | Severe | Italian | [Del Bo et al., 2003] |
| deletion | | (>35.7 kb) | | kin | | | |

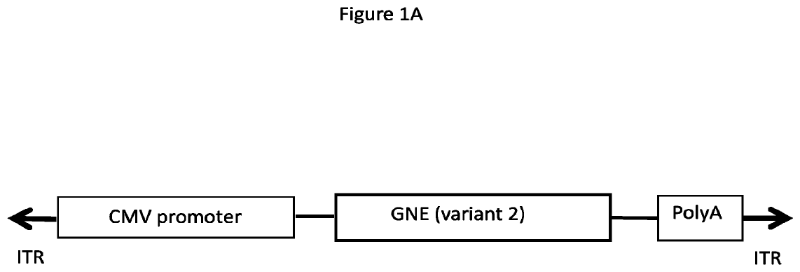

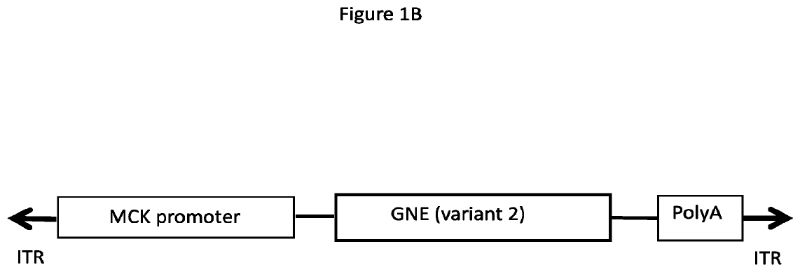

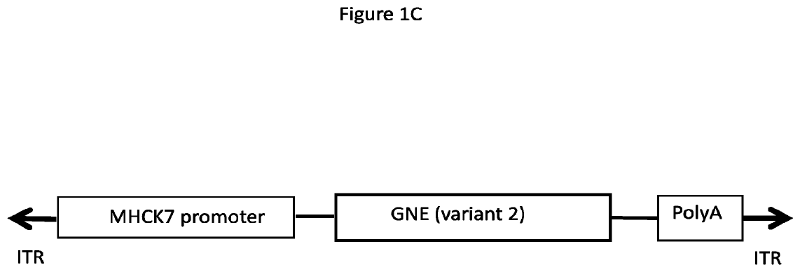

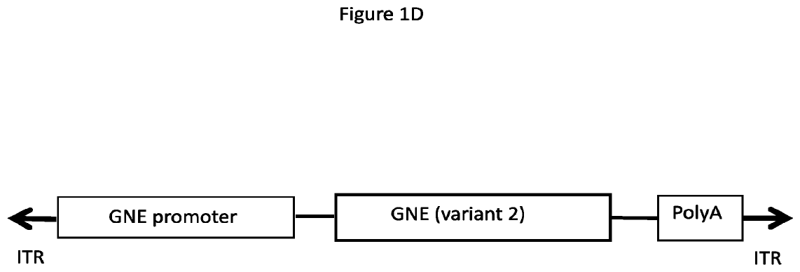

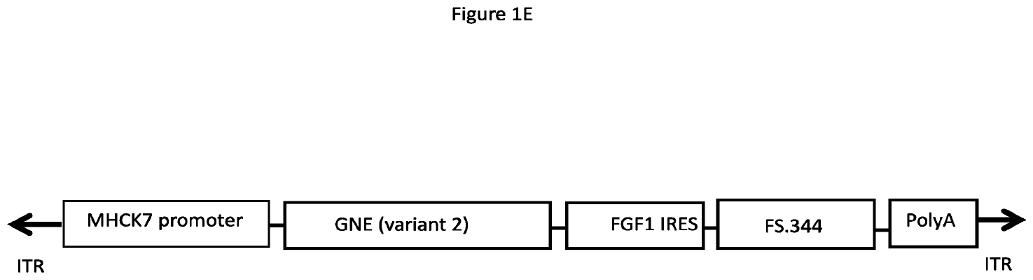

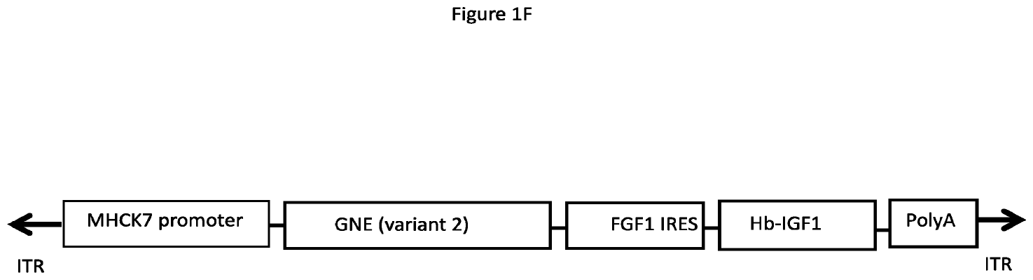

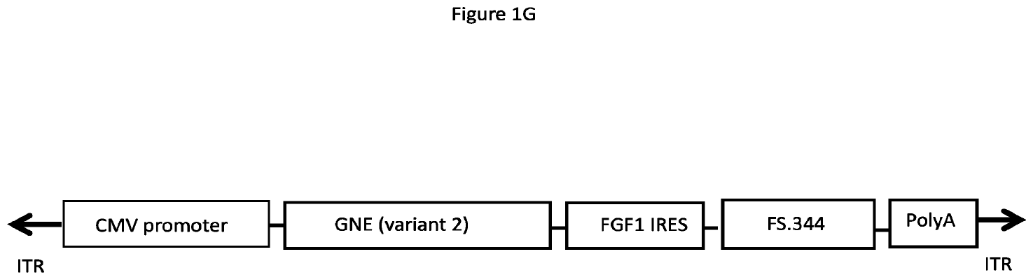

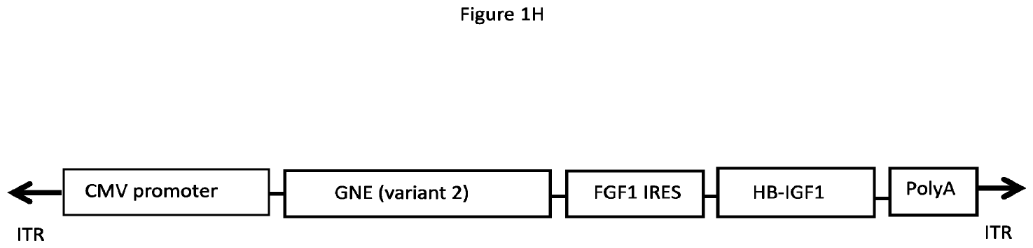

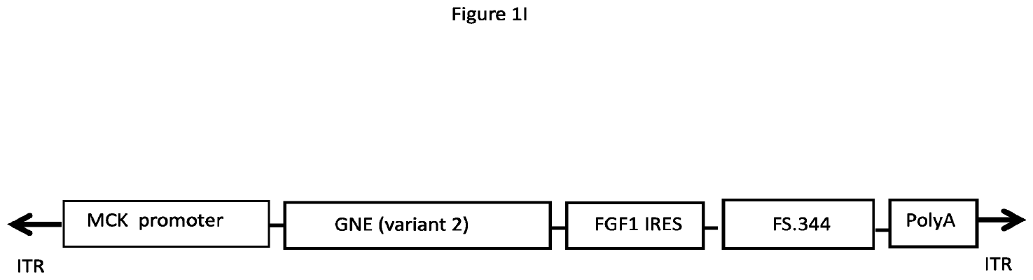

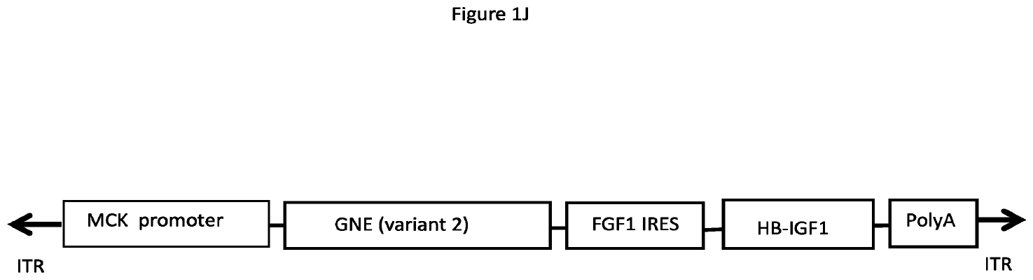

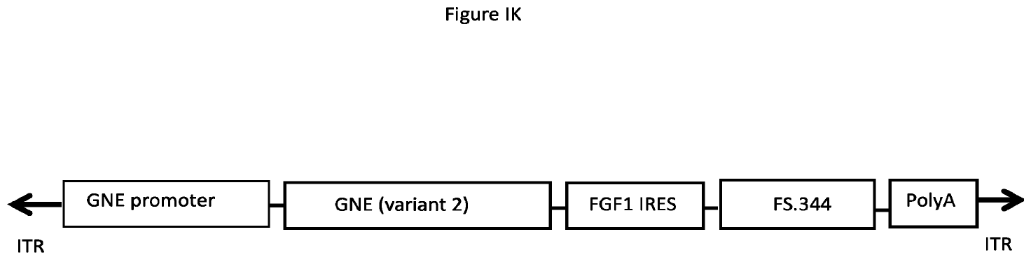

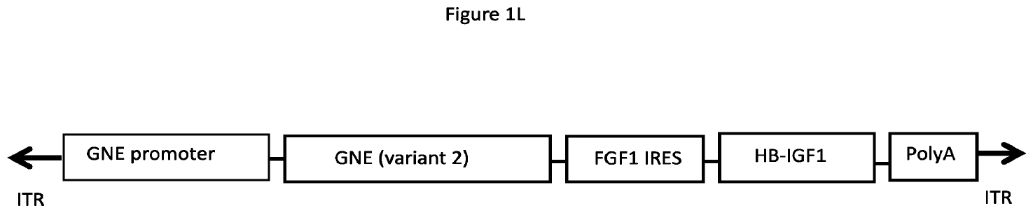

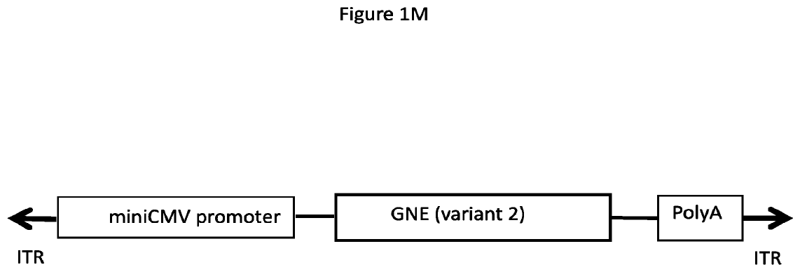

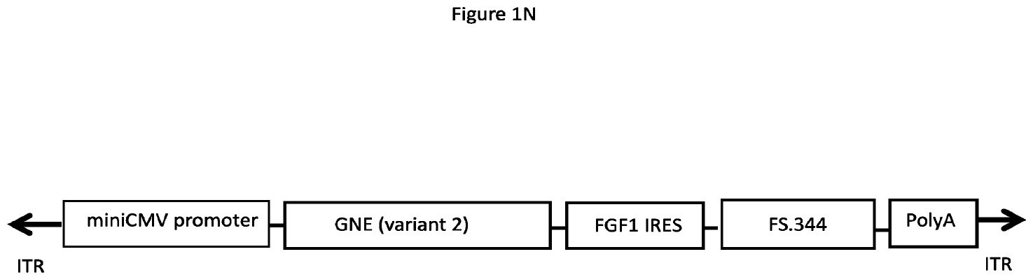

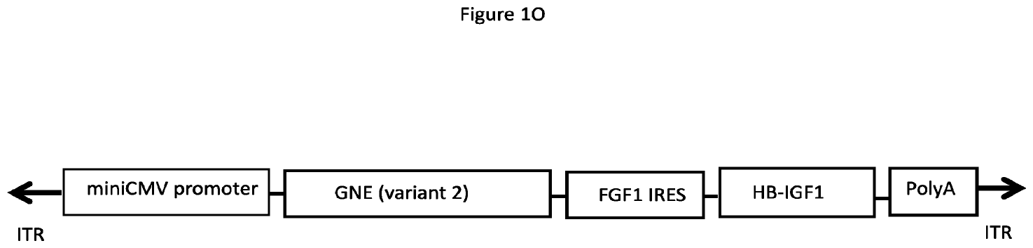

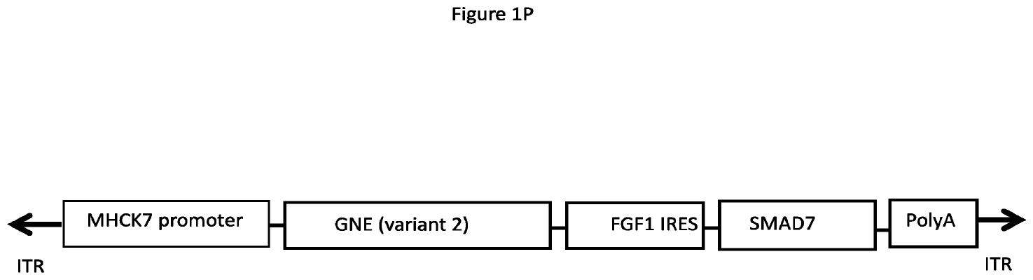

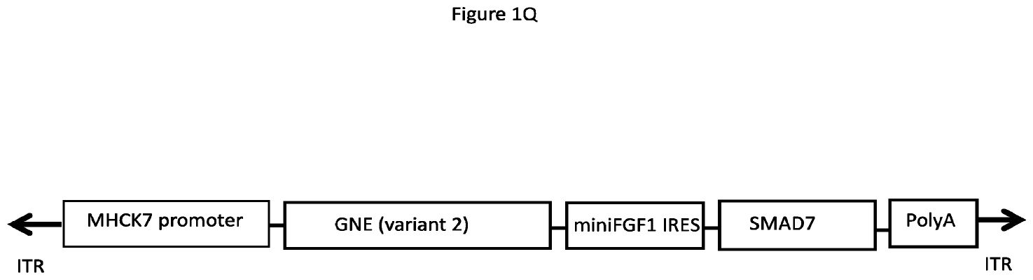

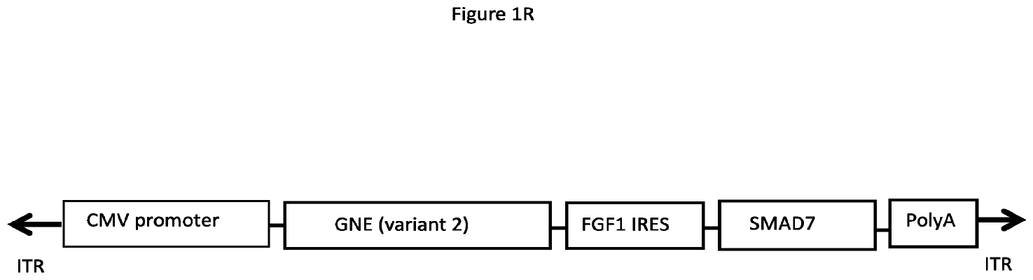

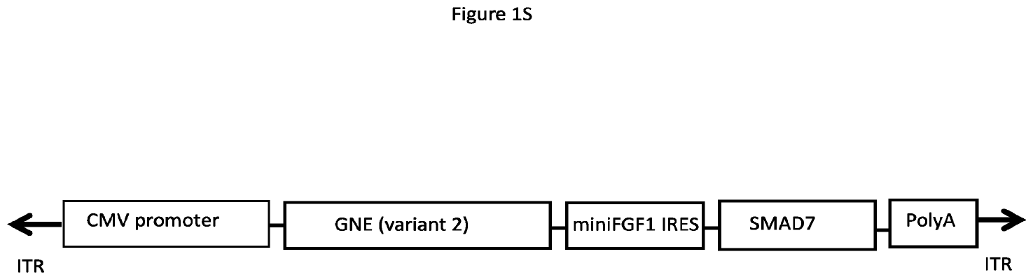

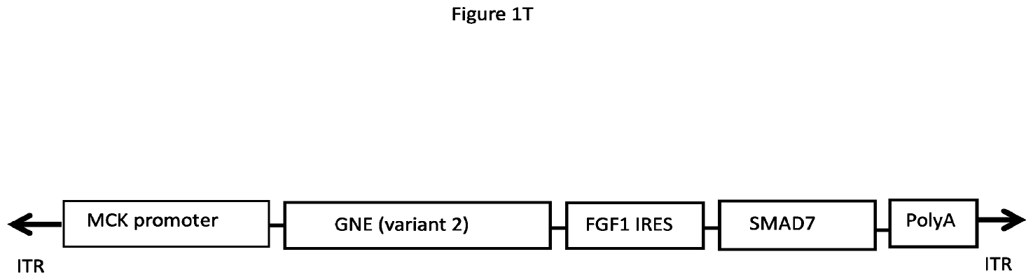

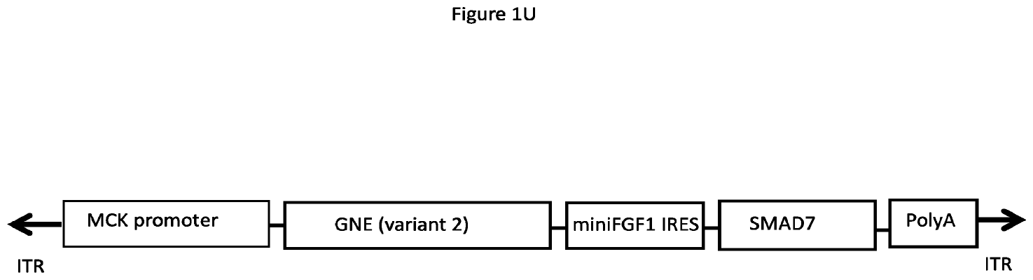

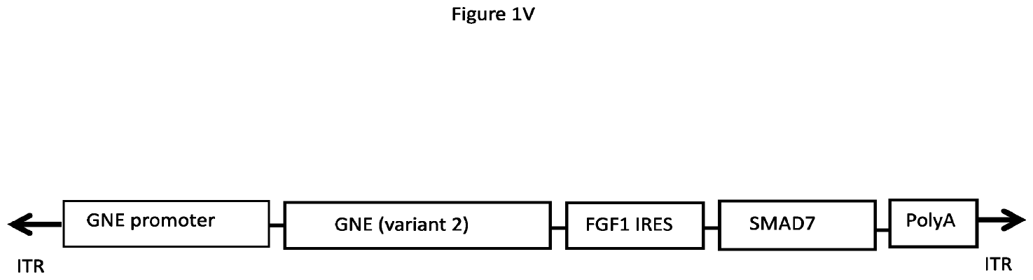

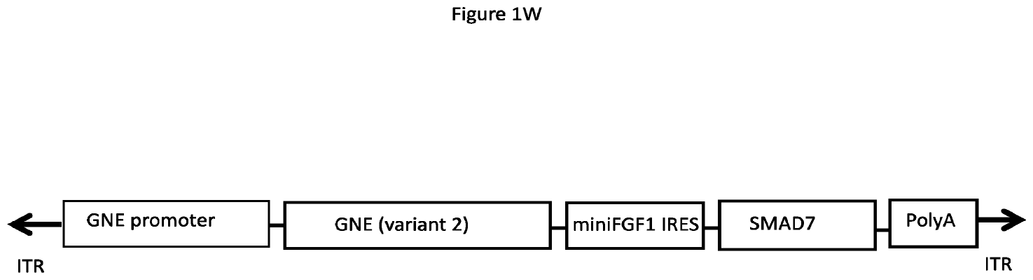

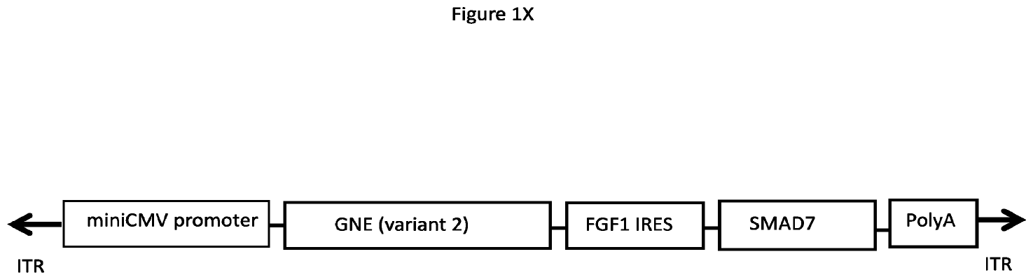

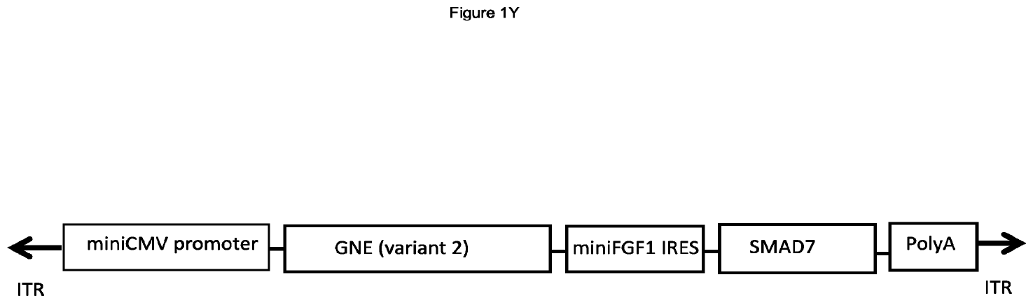

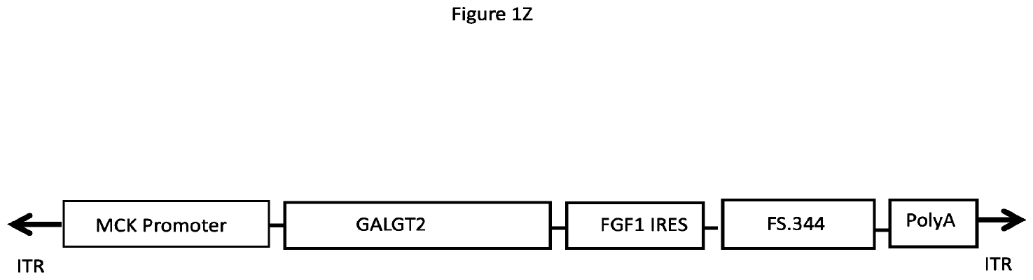

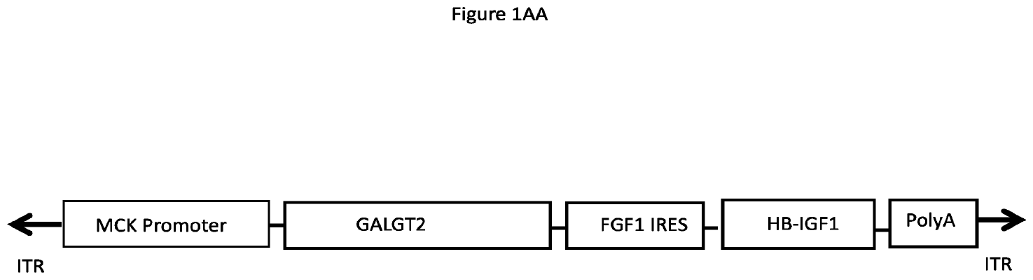

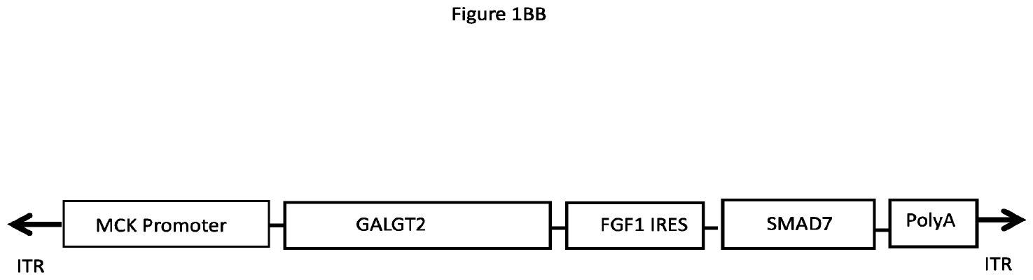

¹Amino acid substitutions are provided in the previously used hGNE1 (NP_005467.1) and in the preferred new hGNE2 (NP_001121699.1) nomenclature [Huizing et al. 2014b]. For some variants, updated nomenclature is provided extracted from the reference.
²Nucleotide variants are provided in the mRNA variant 1 nomenclature (NM_001128227.2; longest mRNA spliceform; encoding hGNE2 protein).
³Exon numbering according to genomic sequence (NC_000009.12) and as indicated in FIG. 1. in = intron.
⁴See text for details about GNE protein domains; ep = UDP-GlcNAc 2-epimerase domain; ep-NES = nuclear export signal; ep-AR: allosteric region; UF = unknown function; kin = ManNAc kinase domain; UF epimerase.
⁵Combined pathogenicity scores, Intronic variants with predicted splicing effects are listed as “splicing’, and without such effects as “splicing?”, Extracted from literature reference.

### GNE Mouse Models

Gne is an essential gene in mice; deletion causes embryonic lethality between embryonic (E) day 8.5 and 9.5. The most celebrated model for GNE myopathy was made by Malicdan et al. (Hum. Mol. Genet. 16(22): 2669-82, 2007). This model constitutively expressed a mutant human GNE_{D207V} transgene (Tg) in a mouse Gne^{-/-} background. By 30 weeks, GNE_{D207V}^{Tg}Gne^{-/-} mice were reported to show significant lifespan reductions, reduced scores in rod climbing and constant speed treadmill walking, and modest elevation in serum CK activity and muscle production of A131-42 peptide. By 42 weeks, muscles exhibited rimmed vacuoles with congophilic inclusion bodies, as well as pathology in respiratory and cardiac muscles that are not found in human GNE myopathy patients. Unfortunately, as these mice have been bred, most of these phenotypes have been lost from the line, such that we and others cannot find evidence of muscle pathology or muscle deficiencies at 64 weeks.

A second model, a knock-in of the M712T (now called M743T) Persian founder GNE mutation, showed perinatal lethality (by P3) due to kidney disease (Galeno et al., Clin. Invest. 117(6):1585-94, 2007). Again, others have found that this homozygous knock-in line can be bred to create a subpopulation of animals with no phenotype (Sela et al., Neuromuscular Med. 15(1): 180-91, 2013). Thus, the robustness of all pre-clinical data on this disease has been called into question due to the high phenotypic variability of the models used.

All of the pre-clinical data is highly complicated by the fact that all current mouse models of GNE myopathy show complicated and overly variable phenotypes. A GNE_{M743T} knock-in model showed early death due to kidney complications, which could be offset by ManNAc. Other strains of the same knock-in show no phenotype. A GNE_{D207V}^{Tg}Gne^{-/-} mouse model showed clear disease phenotypes at one year of age in early studies, none of which can be repeated with mice that are currently alive. As Gne deficiency leads to embryonic death at E8.5 to E9.5 in the mouse, pure gene deletion mice are not useful, though foxed mice to deleted genes with more precision are being made by multiple groups, including ours.

A mouse model is described herein in Example 3. This mouse model is generated using Cas9-CRISPR, which will ultimately allow for the generation of a floxed allele into exon 3 of the mouse Gne gene, and introduction of this allele is sufficient to allow for Cre-mediated deletion, yielding a GNE myopathy-like phenotype. As Gne is essential in mice, leading to lethality between E8.5 and E9, creation of a floxed allele to delete the gene in the adult mouse should allow for creation of a robust body-wide or muscle-specific phenotypes using Cre-mediated deletion. This, in turn, allows for more reproducible demonstrations of therapeutic efficacy.

### Muscular Dystrophies

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

One type of MD is Duchene muscular dystrophy (DMD). It is the most common severe childhood form of muscular dystrophy affecting 1 in 5000 newborn males. Inheritance follows an X-linked recessive pattern. DMD is caused by mutations in the DMD gene leading to absence of dystrophin protein (427 KDa) in skeletal and cardiac muscles, as well as GI tract and retina. Dystrophin not only protects the sarcolemma from eccentric contractions, but also anchors a number of signaling proteins in close proximity to sarcolemma. Clinical symptoms of DMD are usually first noted between ages 3 to 5 years, with altered gait and reduced motor skills typically leading to diagnostic evaluation. DMD is relentlessly progressive, with loss of ambulation by age twelve. Historically patients died from respiratory complications late in the second decade, but improved supportive care—and in particular judicious use of nocturnal ventilatory support—has extended life expectancy by nearly a decade. Prolonging life unmasks the nearly universal decline in cardiac function, with complications of dilated cardiomyopathy. This poses further clinical challenges and a need for recognition and medical intervention that did not previously exist. Non-progressive cognitive dysfunction may also be present in DMD. Despite virtually hundreds of clinical trials in DMD, treatment with corticosteroids remains the only treatment that has consistently demonstrated efficacy. Current standard of care for DMD involves use of prednisone or deflazacort, which can prolong ambulation by several years at the expense of significant side effects, and has limited evidence for any impact on survival.

Another type of MD is Congenital Muscular Dystrophy 1A (MCD1A). MCD1A belongs to a group of neuromuscular disorders with onset at birth or infancy characterized by hypotonia, muscle weakness and muscle wasting. MCD1A represents 30-40% of congenital muscular dystrophies, with some regional variation. Prevalence is estimated at 1/30,000. The disease presents at birth or in the first few months of life with hypotonia and muscle weakness in the limbs and trunk. Respiratory and feeding disorders can also occur. Motor development is delayed and limited (sitting or standing is only possible with help). Infants present with early rigidity of the vertebral column, scoliosis, and respiratory insufficiency. There is facial involvement with a typical elongated myopathic face and ocular ophthalmoplegia disorders can appear later. Epileptic attacks are possible, although they occur in less than a third of subjects. Intellectual development is normal. MCD1A is caused by mutations in the LAMA2 gene coding for the alpha-2 laminin chain. Transmission is autosomal recessive. Current treatment is symptomatic. It consists of a multidisciplinary approach, including physiotherapists, occupational therapists and speech-language therapists, with the objective of optimizing each subject's abilities. Seizures or other neurological complications require specific treatment. The prognosis of MDC1A is very severe as a large proportion of affected children do not reach adolescence. Currently, the prognosis can only be improved by attentive multidisciplinary (particularly orthopedic and respiratory) management.

Yet another type of MD is Limb Girdle Muscular Dystrophy (LGMD). LGMDs are rare conditions and they present differently in different people with respect to age of onset, areas of muscle weakness, heart and respiratory involvement, rate of progression and severity. LGMDs can begin in childhood, adolescence, young adulthood or even later. Both genders are affected equally. LGMDs cause weakness in the shoulder and pelvic girdle, with nearby muscles in the upper legs and arms sometimes also weakening with time. Weakness of the legs often appears before that of the arms. Facial muscles are usually unaffected. As the condition progresses, people can have problems with walking and may need to use a wheelchair over time. The involvement of shoulder and arm muscles can lead to difficulty in raising arms over head and in lifting objects. In some types of LGMD, the heart and breathing muscles may be involved.

There are at least nineteen forms of LGMD, and the forms are classified by their associated genetic defects.

| Type | Pattern of Inheritance | Gene or Chromosome |
|---|---|---|
| LGMD1A | Autosomal dominant | Myotilin gene |
| LGMD1B | Autosomal dominant | Lamin A/C gene |
| LGMD1C | Autosomal dominant | Caveolin gene |
| LGMD1D | Autosomal dominant | Chromosome 7 |
| LGMD1E | Autosomal dominant | Desmin gene |
| LGMD1F | Autosomal dominant | Chromosome 7 |
| LGMD1G | Autosomal dominant | Chromosome 4 |
| LGMD1H | Autosomal dominant | Chromosome 3 |
| LGMD2A | Autosomal recessive | Calpain-3 gene |
| LGMD2B | Autosomal recessive | Dysferlin gene |
| LGMD2C | Autosomal recessive | Gamma-sarcoglycan |
| LGMD2D | Autosomal recessive | Alpha-sarcoglycan gene |
| LGMD2E | Autosomal recessive | Beta-sarcoglycan gene |
| LGMD2F | Autosomal recessive | Delta-sarcoglycan gene |
| LGMD2G | Autosomal recessive | Telethonin gene |
| LGMD2H | Autosomal recessive | TRIM32 |
| LGMD2I | Autosomal recessive | FKRP gene |
| LGMD2J | Autosomal recessive | Titin gene |
| LGMD2K | Autosomal recessive | POMT1 gene |
| LGMD2L | Autosomal recessive | Anoctamin 5 gene |
| LGMD2M | Autosomal recessive | Fukutin gene |
| LGMD2N | Autosomal recessive | POMT2 gene |
| LGMD2O | Autosomal recessive | POMGnT1 gene |
| LGMD2Q | Autosomal recessive | Plectin gene |

Specialized tests for LGMD are now available through a national scheme for diagnosis, the National Commissioning Group (NCG).

The GALGT2 gene (otherwise known as B4GALNT2) encodes a β1-4-N-acetyl-D-galactosamine (βGalNAc) glycosyltransferase. GALGT2 overexpression has been studied in three different models of muscular dystrophy: DMD, LGMD2D and MDC1A [Xu et al., Am. J. Pathol, 175: 235-247 (2009); Xu et al., Am. J. Path., 171: 181-199 (2007); Xu et al., Neuromuscul. Disord., 17: 209-220 (2007); Martin et al., Am. J. Physiol. Cell. Physiol., 296: C476-488 (2009); and Nguyen et al., Proc. Natl. Acad. Sci. USA, 99: 5616-5621 (2002)]. GALGT2 overexpression in skeletal muscles has been reported to induce the glycosylation of alpha dystroglycan with β1-4-N-acetyl-D-galactosamine (GalNAc) carbohydrate to make the CT carbohydrate antigen (Neu5Ac/Gcα2-3[GalNAcβ1-4]Galβα1-4GlcNAcβ-). The GALGT2 glycosyltransferase and the CT carbohydrate it creates are normally confined to neuromuscular and myotendinous junctions in skeletal muscles of adult humans, non-human primates, rodents and all other mammals yet studied [Martin et al., J. Neurocytol., 32: 915-929 (2003)]. Overexpression of GALGT2 in skeletal muscle has been reported to stimulate the ectopic glycosylation of the extrasynaptic membrane, stimulating the ectopic overexpression of a scaffold of normally synaptic proteins that are orthologues or homologues of proteins missing in various forms of muscular dystrophy, including dystrophin surrogates (e.g., utrophin, Plectin1) and laminin α2 surrogates (laminin α5 and agrin) [Xu et al. 2009, supra; Xu et al, Am. J. Path. 2007, supra; Xu et al., Neuromuscul. Disord. 2007, supra; Nguyen et al., supra; Chicoine et al., Mol. Ther, 22: 713-724. (2014). As a group, the induction of such surrogates by GALGT2 has been reported to strengthen sarcolemmal membrane integrity and prevent muscle injury in dystrophin-deficient muscles as well as in wild type muscles [Martin et al., supra]. GALGT2 overexpression in skeletal muscle has been reported to prevent muscle damage and inhibit muscle disease. This is true in the mdx mouse model for DMD [Xu et al., Neuromuscul. Disord. 2007, supra; Martin et al. (2009), supra; Nguyen et al., supra], where improvement equal to that of micro-dystrophin gene transfer is noted even though only half the number of fibers were transduced Martin et al. (2009), supra]. Notably, GALGT2 gene transfer has also been reported to be preventive in the dy^{W} model for congenital muscular dystrophy 1A [Xu et al, Am. J. Path. 2007, supra] and the Sgca^{-/-} mouse model for limb girdle muscular dystrophy type 2D [Xu et al. 2009, supra].

### AAV Gene Therapy

The present disclosure provides for gene therapy vectors, e.g. rAAV vectors, expressing the GNE gene and methods of treating GNE myopathy.

As used herein, the term “AAV” is a standard abbreviation for Adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

### AAV

Adeno-associated virus (AAV) is a replication-deficient parvovirus, the single-stranded DNA genome of which is about 4.7 kb in length including two 145 nucleotide inverted terminal repeats (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of Translational Medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes AAVrh.74, AAVrh.10, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art. To promote skeletal muscle specific expression, AAV1, AAV6, AAV8, AAV9, AAVrh10, or AAVrh.74 can be used.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAVrh.10, AAV-8, AAV-9, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The provided recombinant AAV (i.e., infectious encapsidated rAAV particles) comprise a rAAV genome. In exemplary embodiments, the genomes of both rAAV lack AAV rep and cap DNA, that is, there is no AAV rep or cap DNA between the ITRs of the genomes.

In an exemplary embodiment, the recombinant AAV is produced by the triple transfection method (Xiao et al., J Virol 72, 2224-2232 (1998) using the AAV vector plasmid comprising the GNE gene and a muscle specific promoter element, pNLRep2-Caprh74 and pHelp, rAAV contains the GNE gene expression cassette flanked by AAV2 inverted terminal repeat sequences (ITR). It is this sequence that is encapsidated into AAVrh74 virions. The plasmid contains the GNE sequence and the muscle specific promoter element and core promoter elements of the muscle specific promoter to drive gene expression. The expression cassette may also contain an SV40 intron (SD/SA) to promote high-level gene expression and the bovine growth hormone polyadenylation signal is used for efficient transcription termination.

The pNLREP2-Caprh74 is an AAV helper plasmid that encodes the 4 wild-type AAV2 rep proteins and the 3 wild-type AAV VP capsid proteins from serotype rh74.

The pHELP adenovirus helper plasmid is 11,635 bp and was obtained from Applied Viromics. The plasmid contains the regions of adenovirus genome that are important for AAV replication, namely E2A, E4ORF6, and VA RNA (the adenovirus E1 functions are provided by the 293 cells). The adenovirus sequences present in this plasmid only represents ˜40% of the adenovirus genome, and does not contain the cis elements critical for replication such as the adenovirus terminal repeats. Therefore, no infectious adenovirus is expected to be generated from such a production system.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising rAAV of the present disclosure. Compositions of the disclosure comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include buffers and surfactants such as pluronics.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral vector genomes (vgs). One exemplary method of determining encapsilated vector genome titer uses quantitative PCR such as the methods described in (Pozsgai et al., Mol. Ther. 25(4): 855-869, 2017).

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the disclosure is GNE myopathy.

Combination therapies are also contemplated by the disclosure. Combination as used herein includes both simultaneous treatment and sequential treatments. Combinations of methods of the disclosure with standard medical treatments (e.g., corticosteroids) are specifically contemplated, as are combinations with novel therapies.

Administration of an effective dose of the compositions may be by routes standard in the art including, but not limited to, intramuscular, parenteral, intravenous, intraarterial, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the UDP-GlcNAc-epimerase/ManNAc-6 kinase protein and either follistatin 344, follistatin 317 or insulin-like growth factor 1.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion or implantation.

In particular, actual administration of rAAV of the present disclosure may be accomplished by using any physical method that will transport the rAAV recombinant vector into the target tissue of an animal. Administration according to the disclosure includes, but is not limited to, injection into muscle and injection into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

The dose of rAAV to be administered in methods disclosed herein will vary depending, for example, on the particular rAAV, the mode of administration, the treatment goal, the individual, and the cell type(s) being targeted, and may be determined by methods standard in the art. Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 2×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 2×10¹⁴ vg, 1×10¹⁵ vg respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1.25×10¹⁴ vg/kg, 1.5×10¹⁴ vg/kg, 1.75×10¹⁴ vg/kg, 2.0×10¹⁴ vg/kg, 2.25×10¹⁴ vg/kg, 2.5×10¹⁴ vg/kg, 2.75×10¹⁴ vg/kg, 3.0×10¹⁴ vg/kg, 3.25×10¹⁴ vg/kg, 3.5×10¹⁴ vg/kg, 3.75×10¹⁴ vg/kg, 4.0×10¹⁴ vg/kg, 1×10¹⁵ vg/kg respectively). Methods for titering AAV are described in Clark et al., Hum. Gene Ther., 10: 1031-1039 (1999).

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxypropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the disclosure results in sustained expression of the UDP-GIcNAc-epimerase/ManNAc-6 kinase protein. The present disclosure thus provides methods of administering/delivering rAAV which express UDP-GIcNAc-epimerase/ManNAc-6 kinase protein to an animal, preferably a human being. These methods include transducing tissues (including, but not limited to, tissues such as muscle, organs such as liver and brain, and glands such as salivary glands) with one or more rAAV of the present disclosure. Transduction may be carried out with gene cassettes comprising tissue specific control elements. For example, one embodiment of the disclosure provides methods of transducing muscle cells and muscle tissues directed by muscle specific promoter elements, including, but not limited to, those derived from the actin and myosin gene families, such as from the myoD gene family (See Weintraub et al., Science, 251: 761-766 (1991)), the myocyte-specific enhancer binding factor MEF-2 (Cserjesi and Olson, Mol Cell Biol 11: 4854-4862 (1991)), control elements derived from the human skeletal actin gene (Muscat et al., Mol Cell Biol, 7: 4089-4099 (1987)), the cardiac actin gene, muscle creatine kinase sequence elements (See Johnson et al., Mol Cell Biol, 9:3393-3399 (1989)) and the murine creatine kinase enhancer (MCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery, because it is not a vital organ and is easy to access. The disclosure contemplates sustained expression UDP-GIcNAc-epimerase/ManNAc-6 kinase of from transduced myofibers.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the coding region of the GNE to a recipient cell either in vivo or in vitro, via a replication-deficient rAAV of the disclosure resulting in expression of UDP-GlcNAc-epimerase/ManNAc-6 kinase by the recipient cell.

The following EXAMPLES are provided by way of illustration and not limitation. Described numerical ranges are inclusive of each integer value within each range and inclusive of the lowest and highest stated integer.

### EXAMPLES

### Example 1

### Constructs Encoding GIcNAc Epimerase/ManNAc Kinase or GalNAc Transferase Gene cDNA

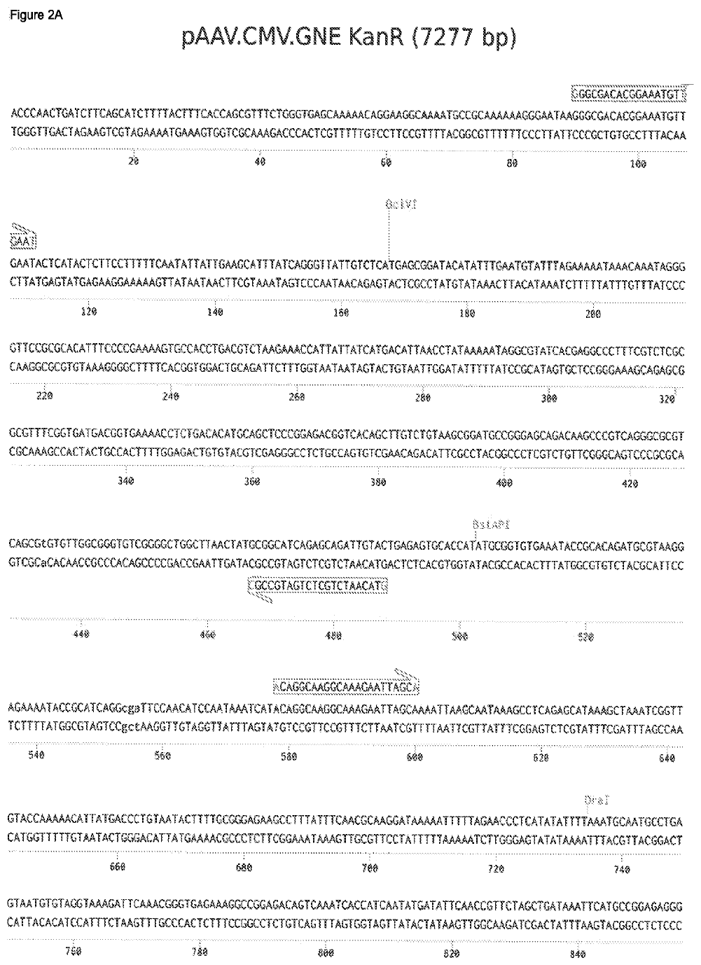

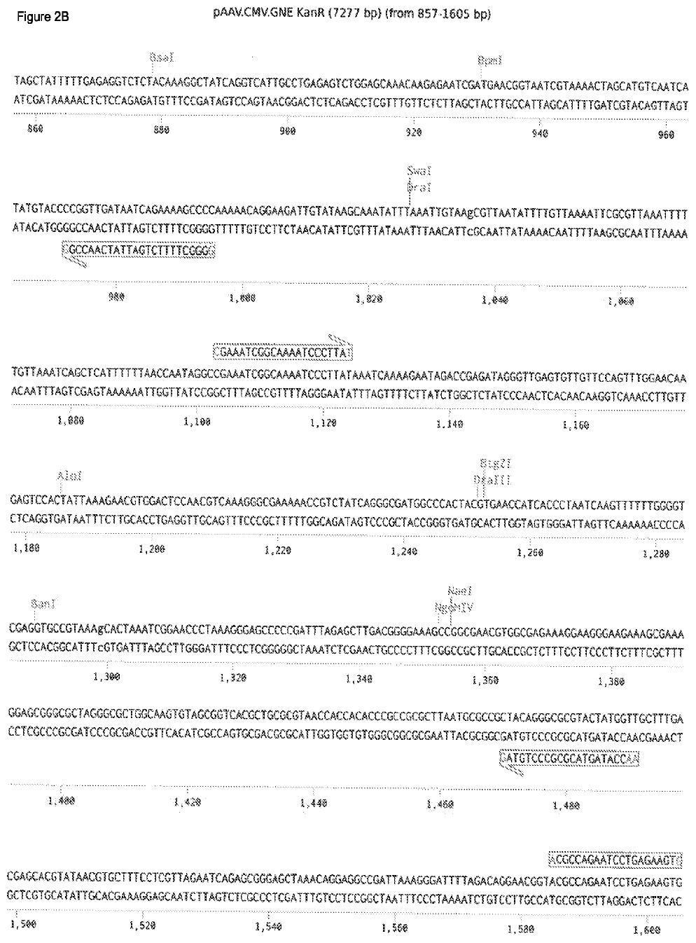

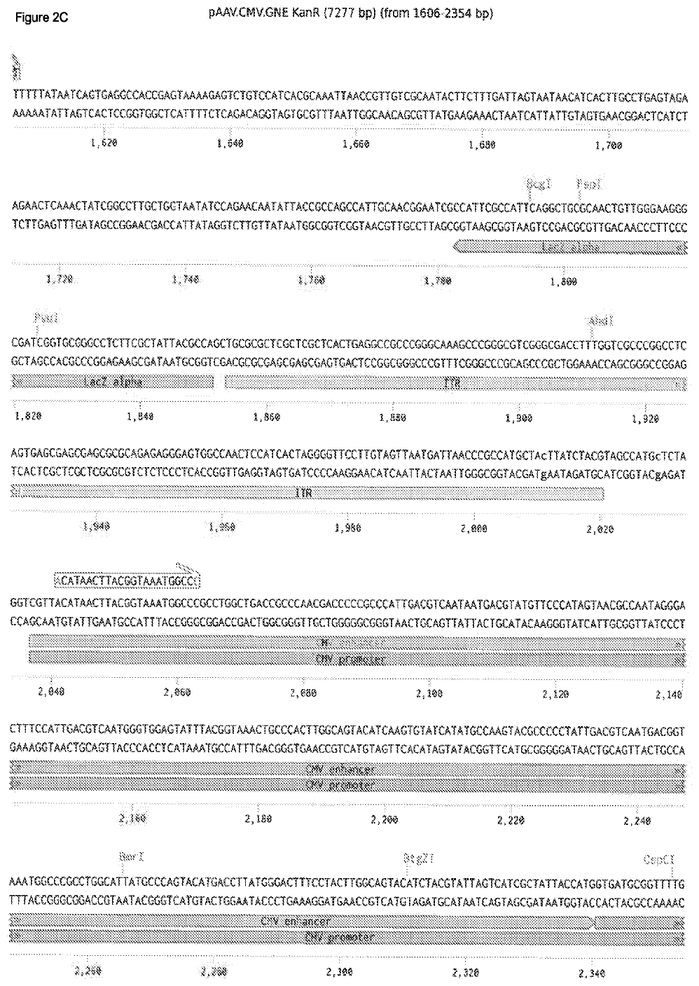

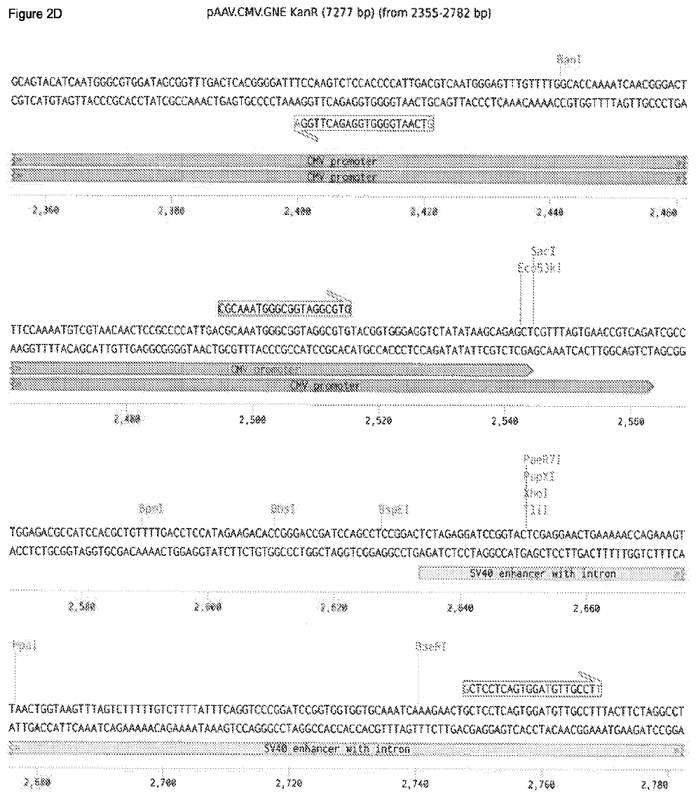

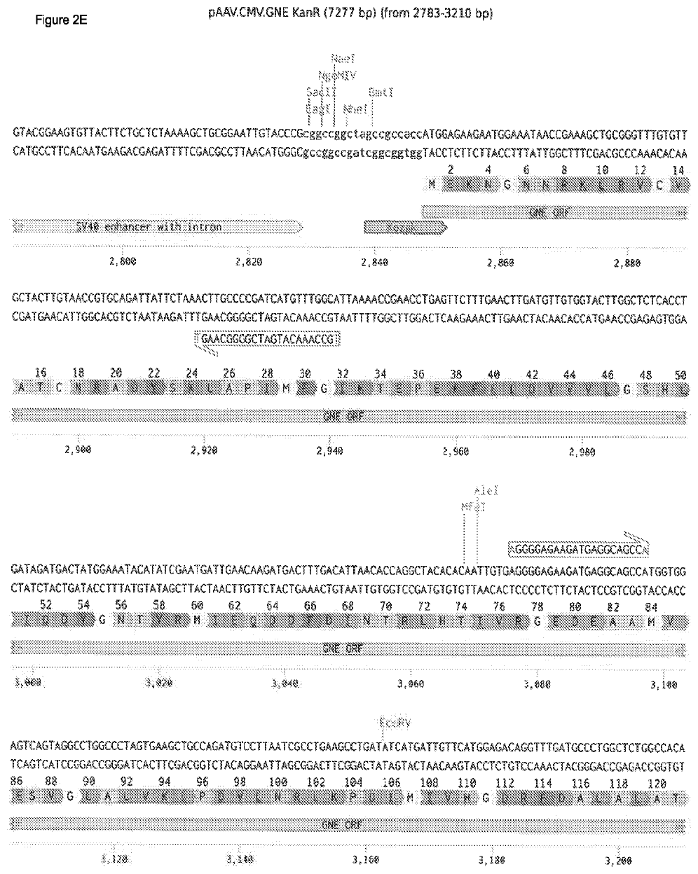

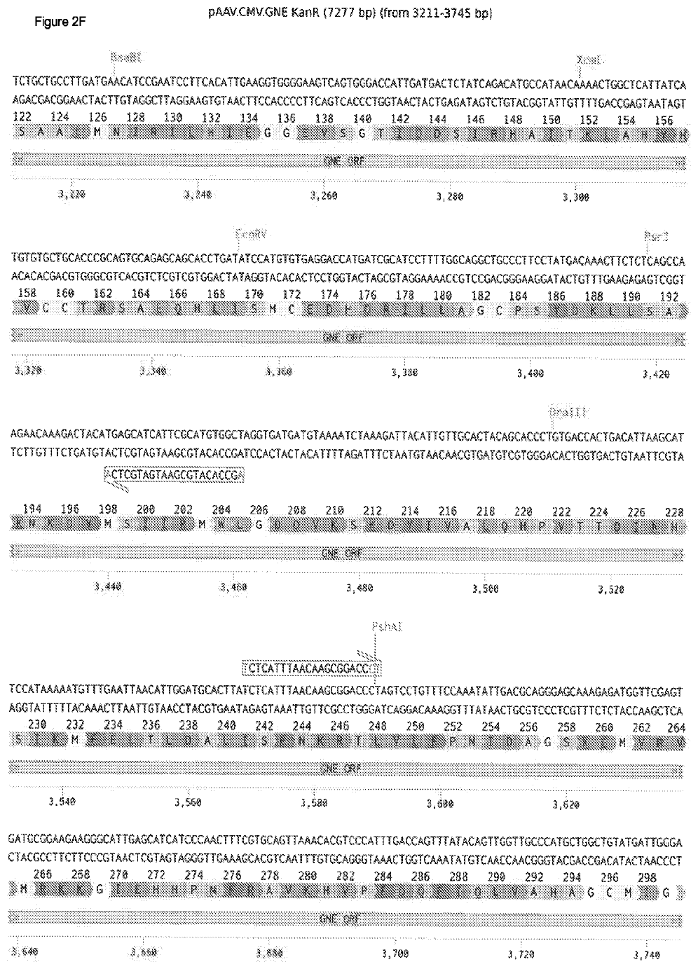

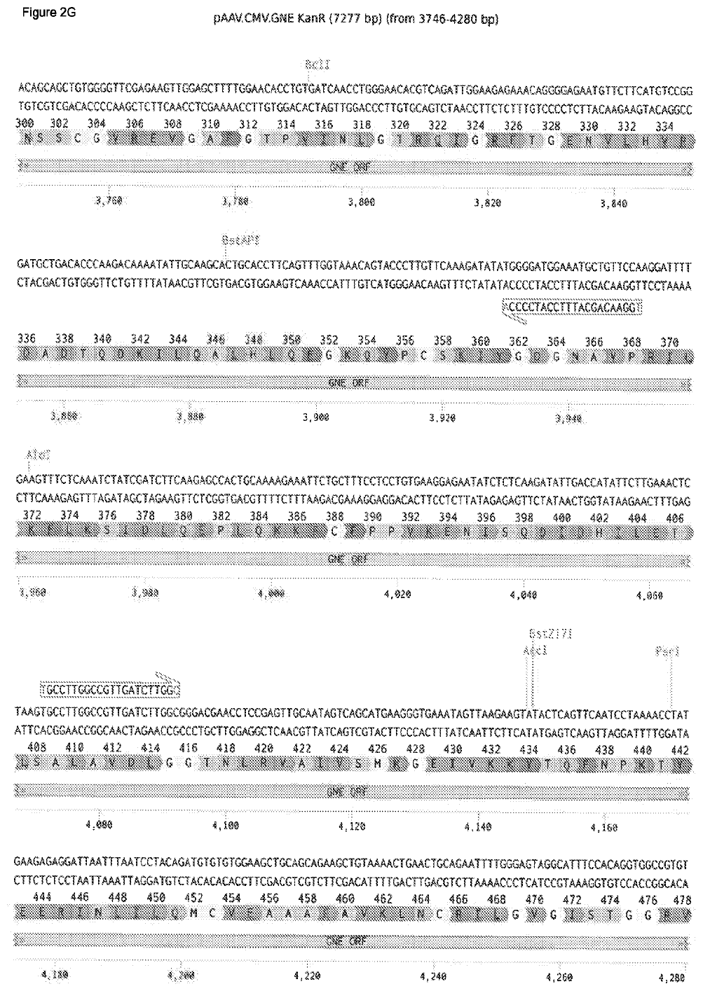

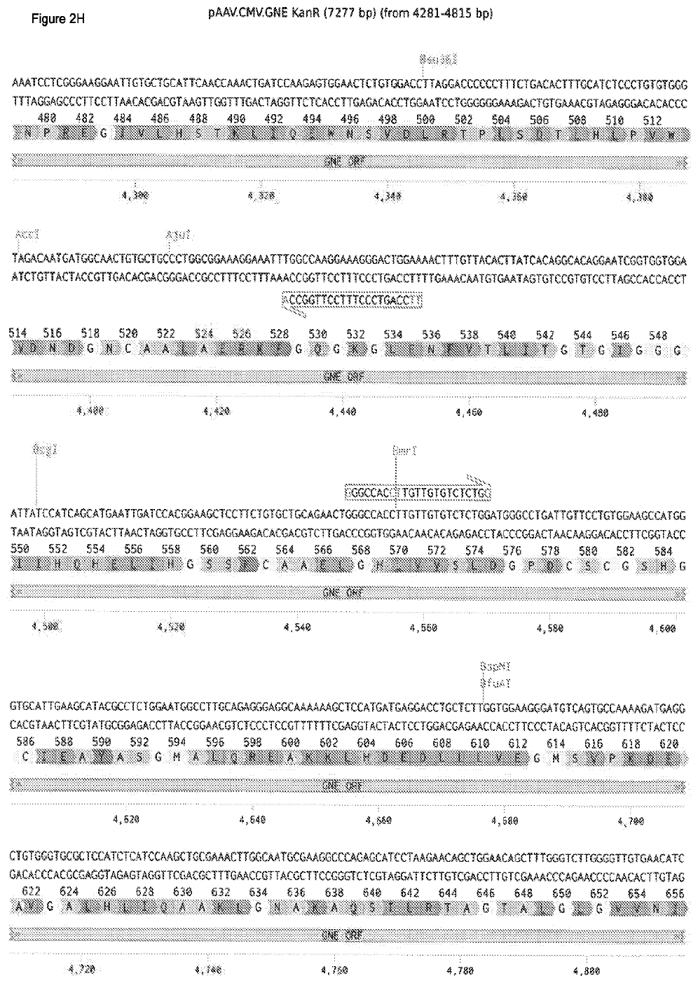

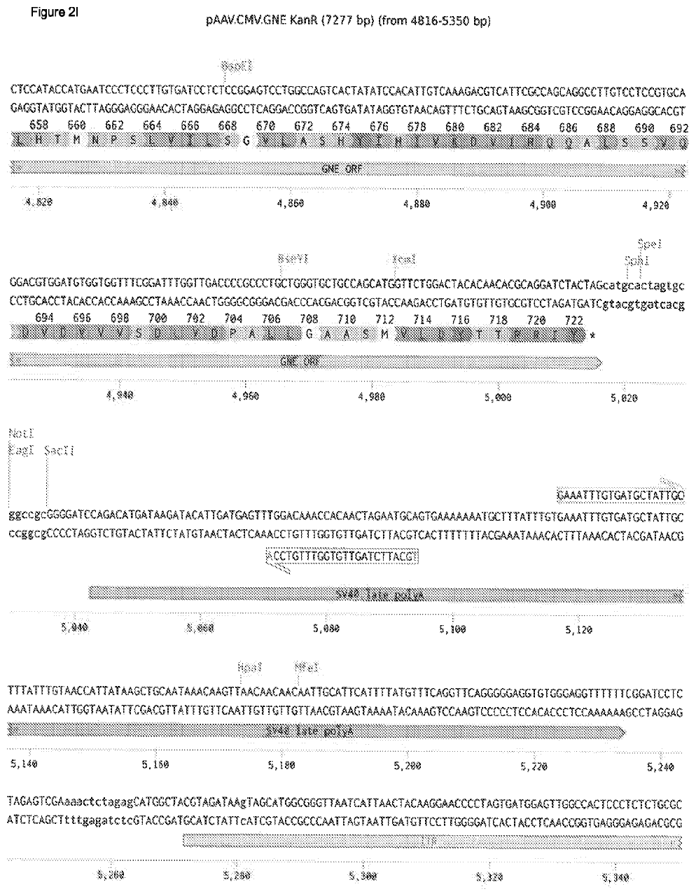

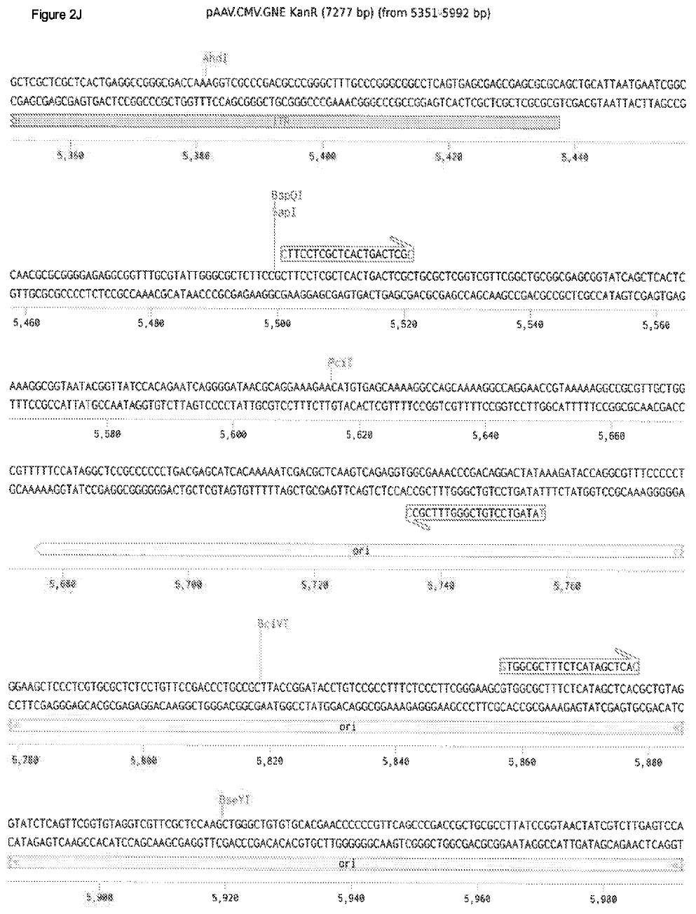

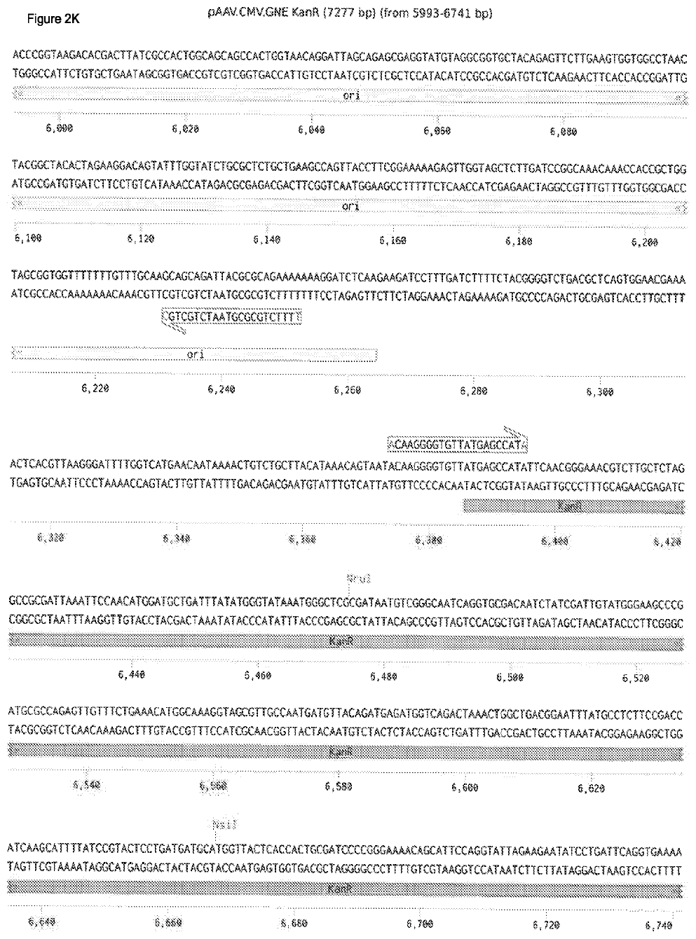

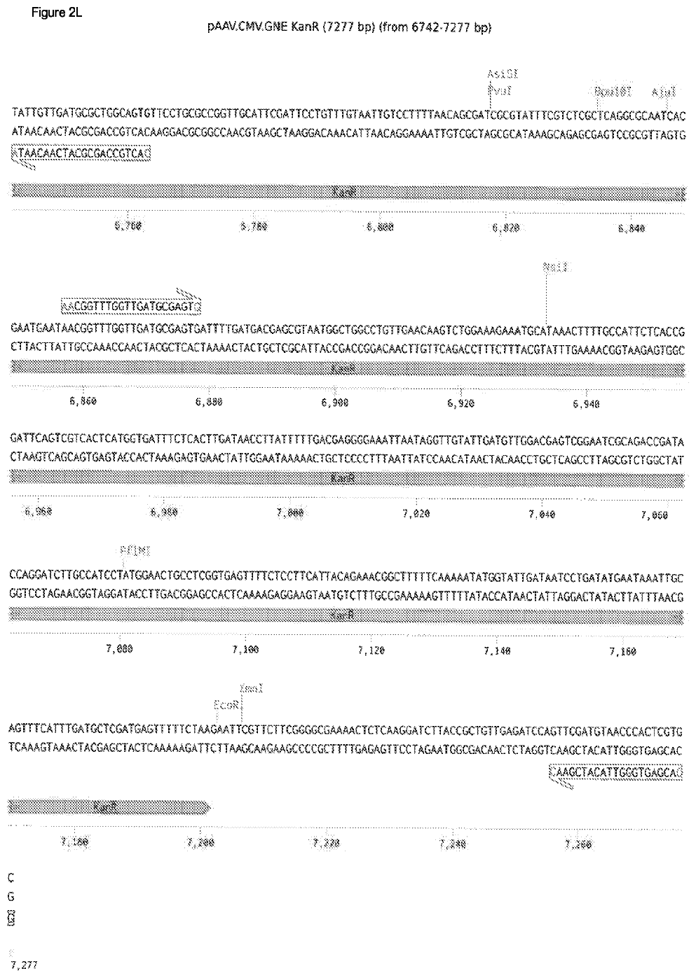

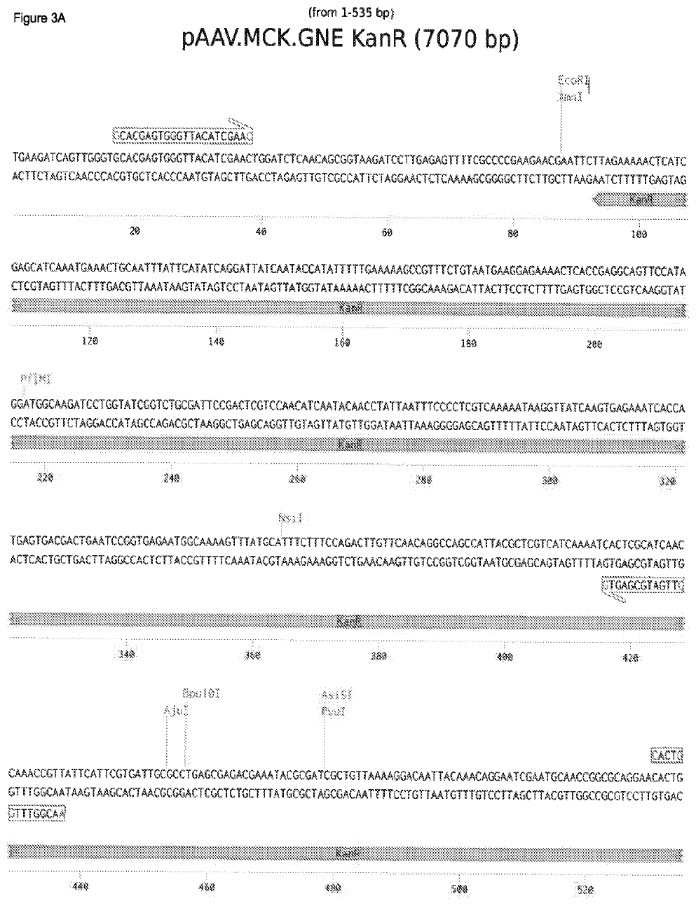

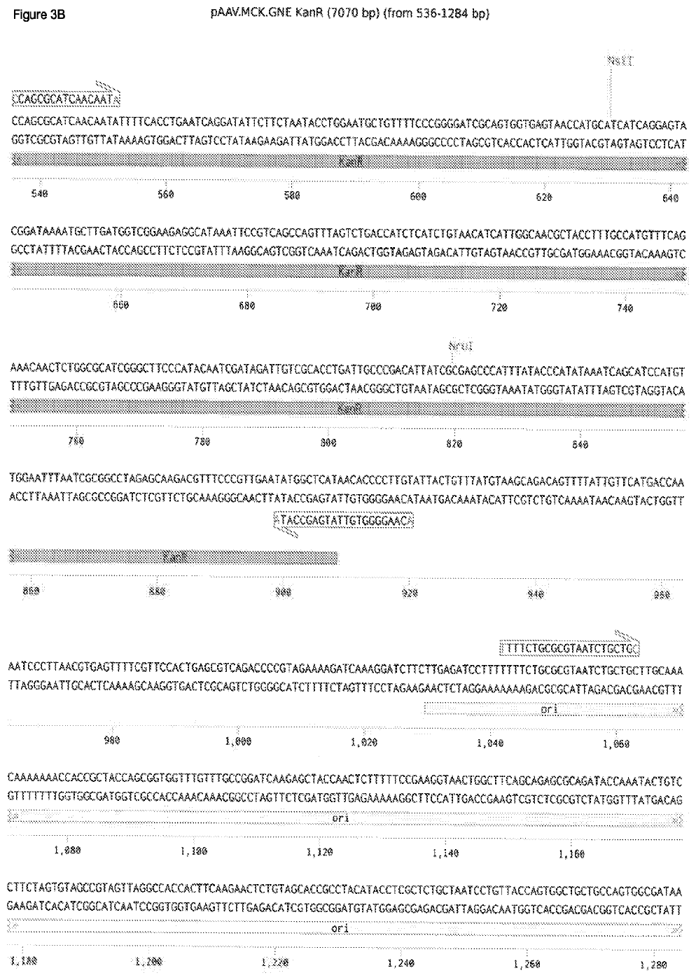

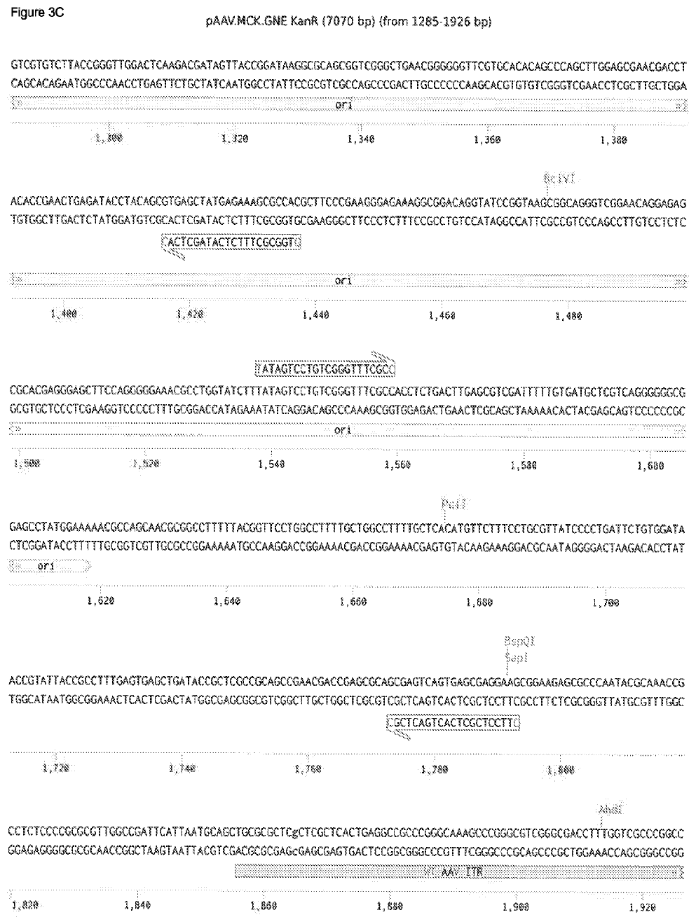

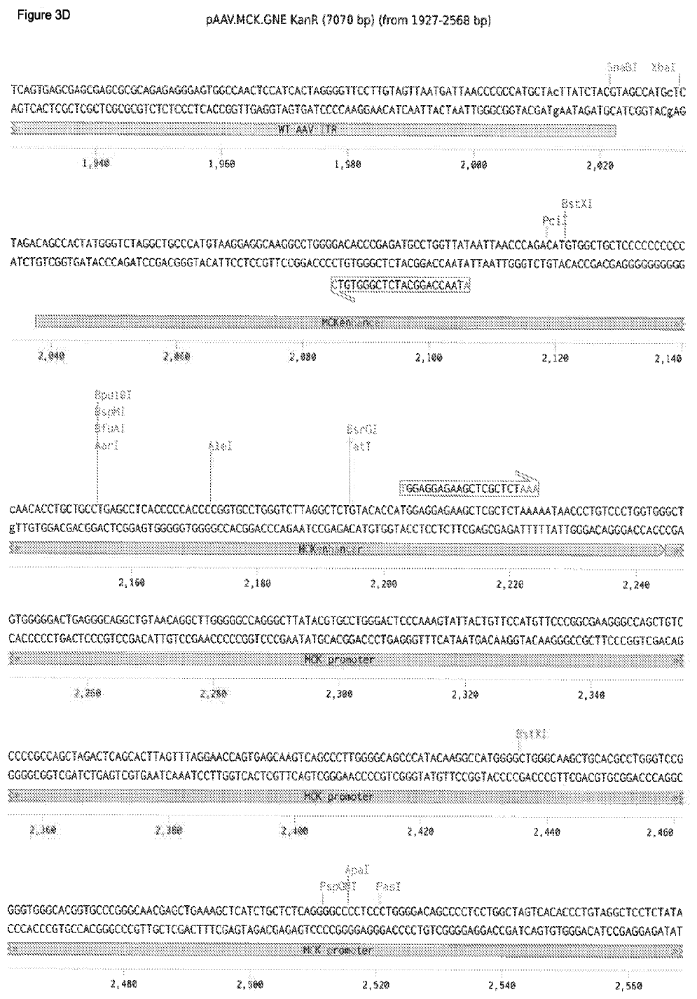

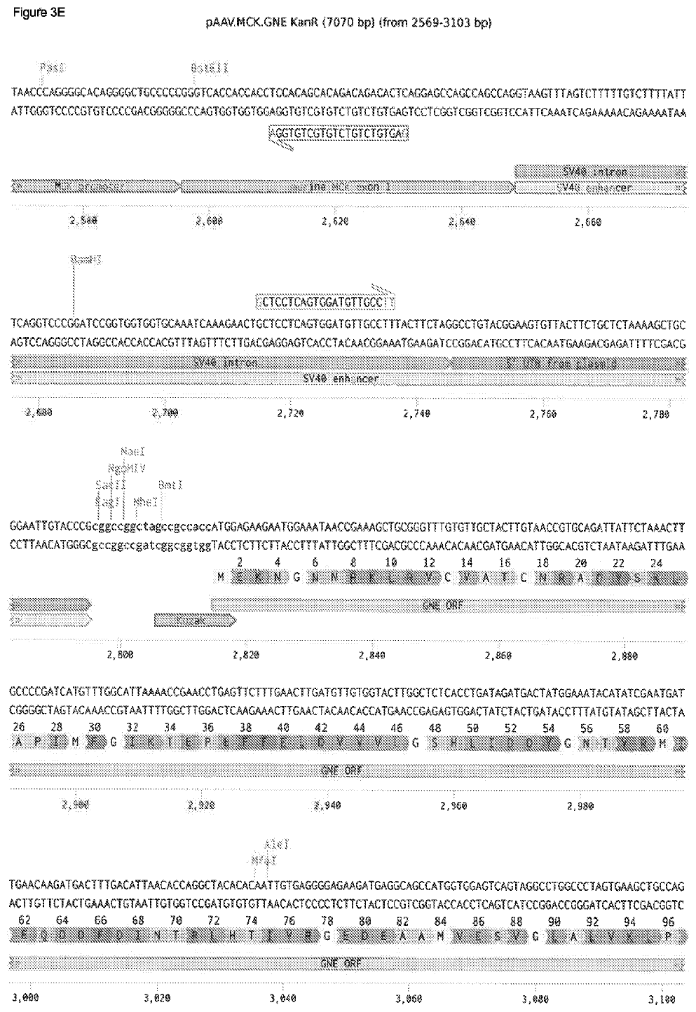

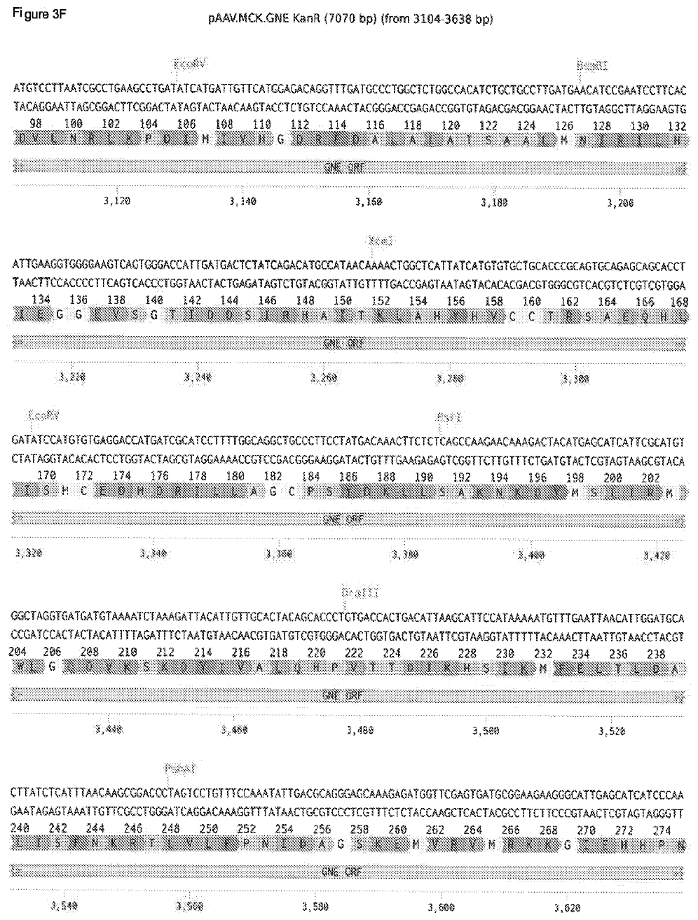

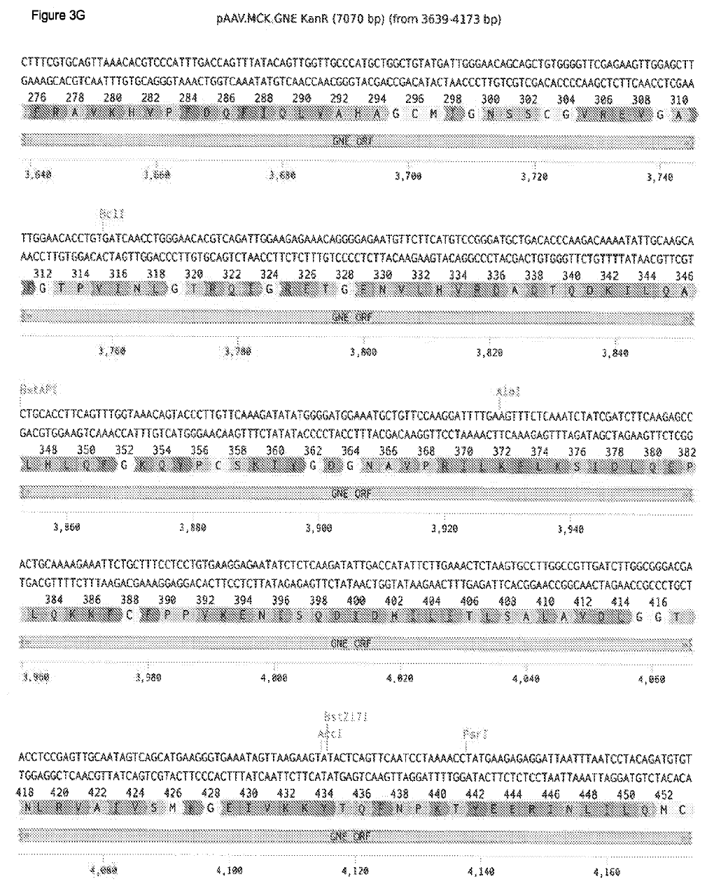

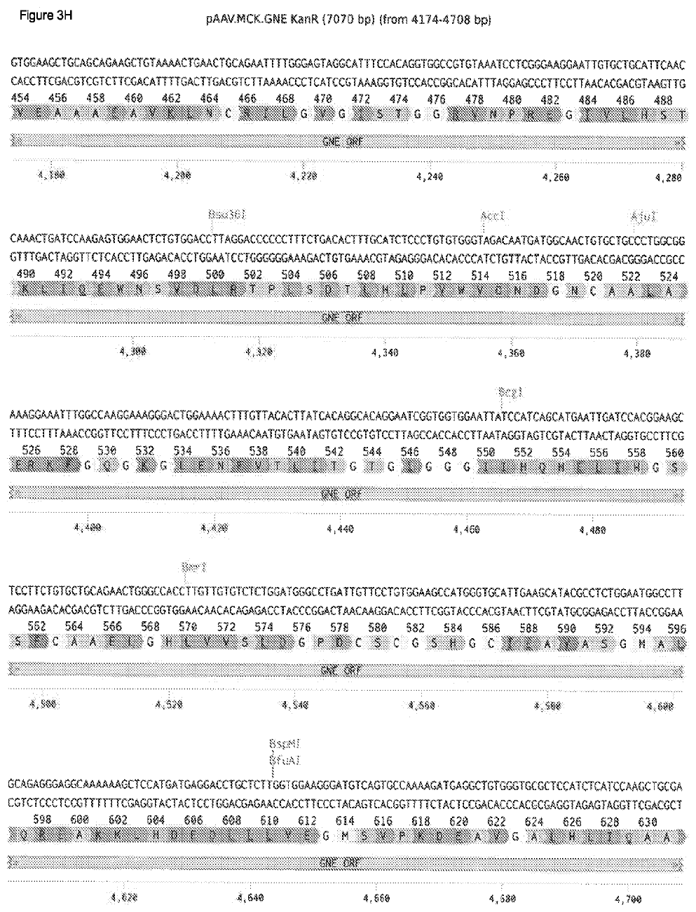

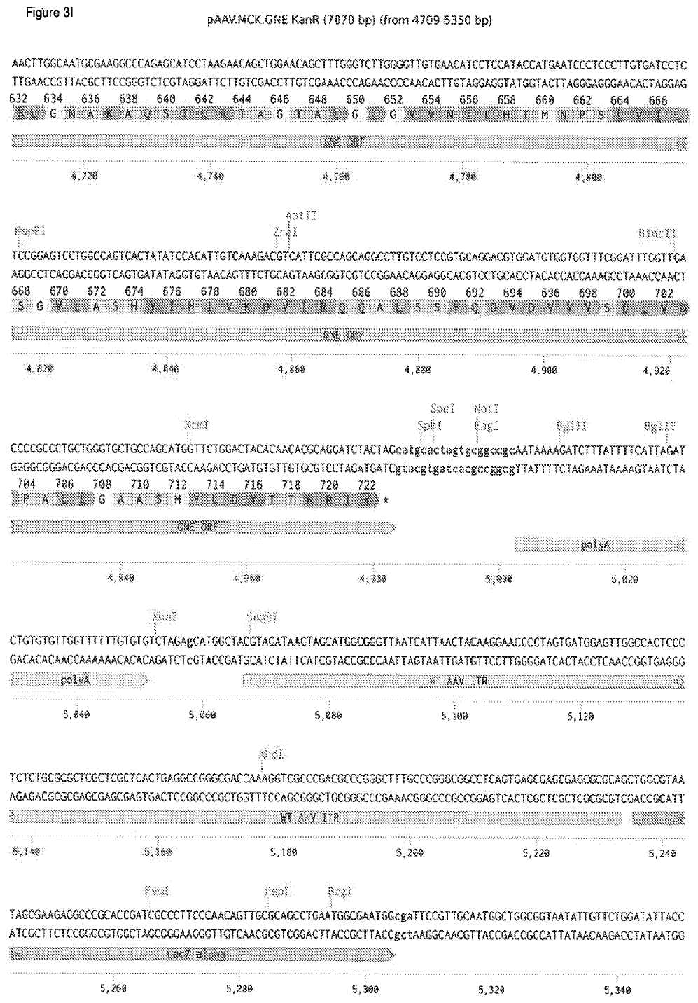

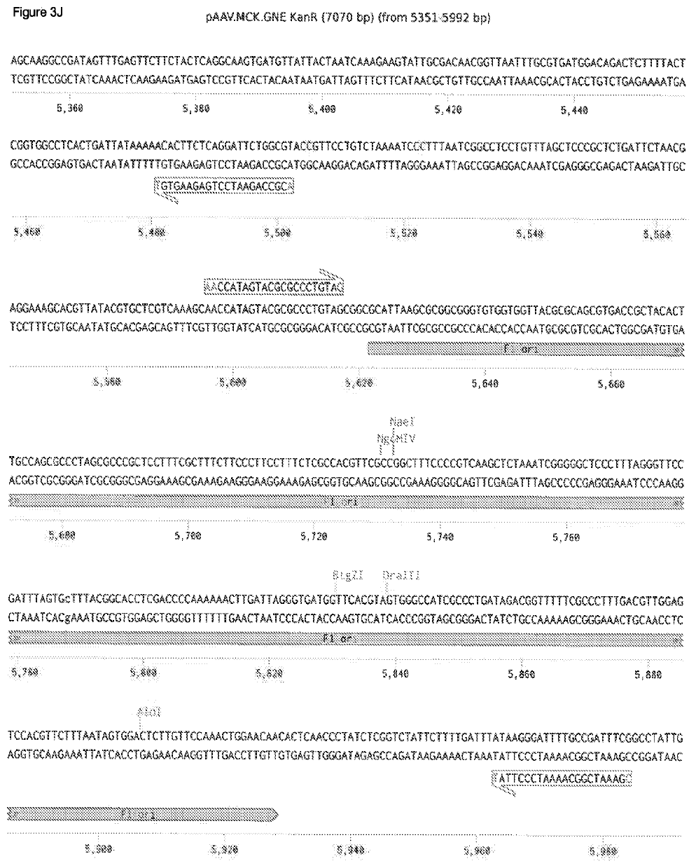

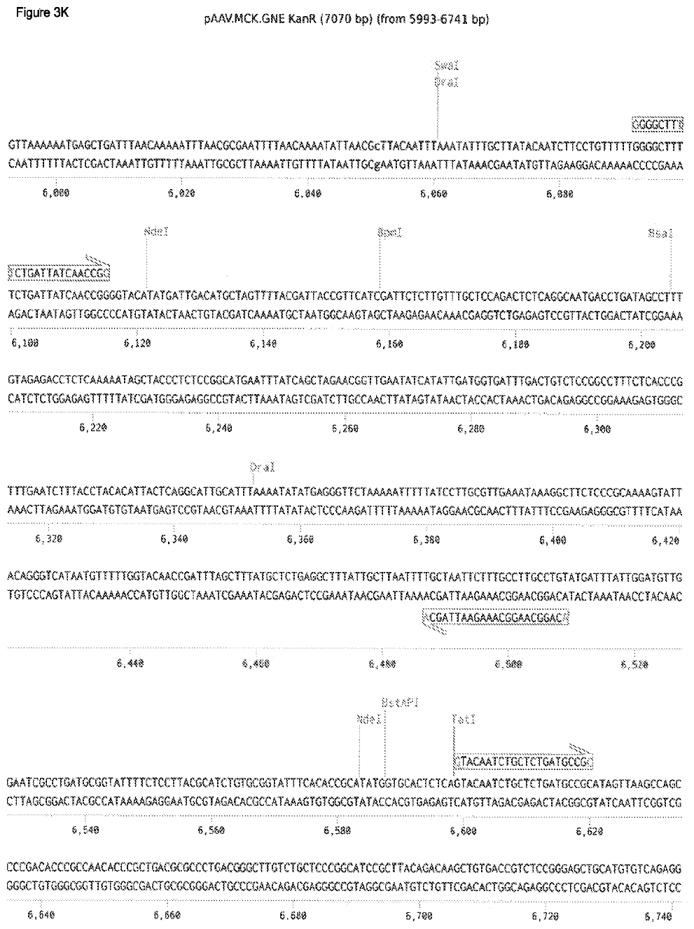

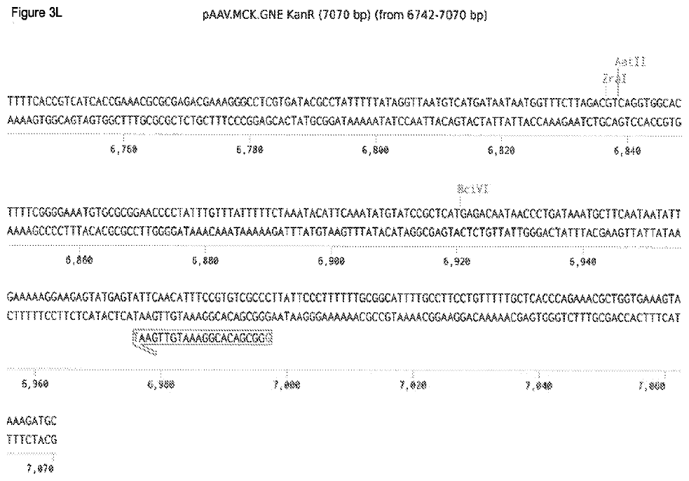

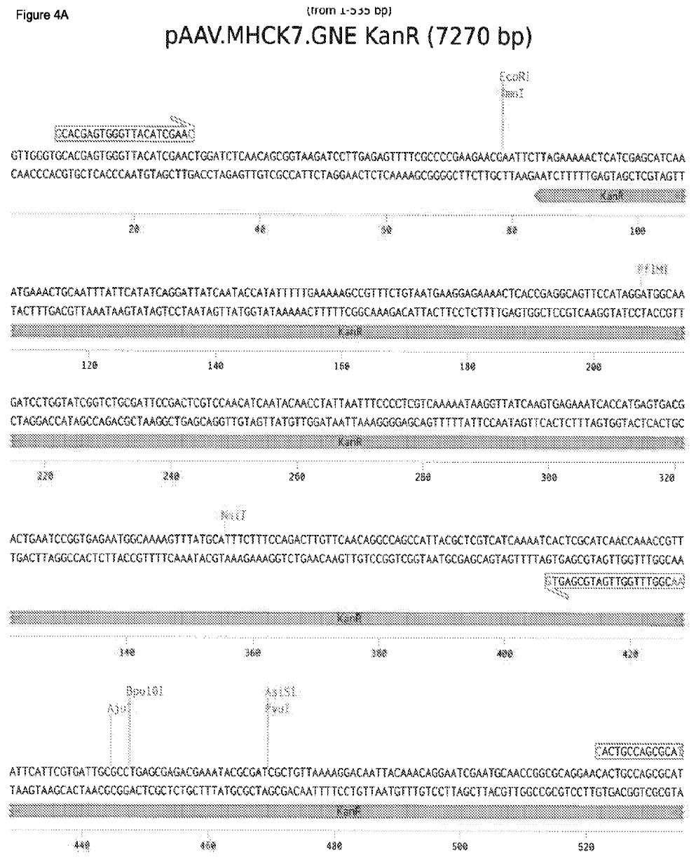

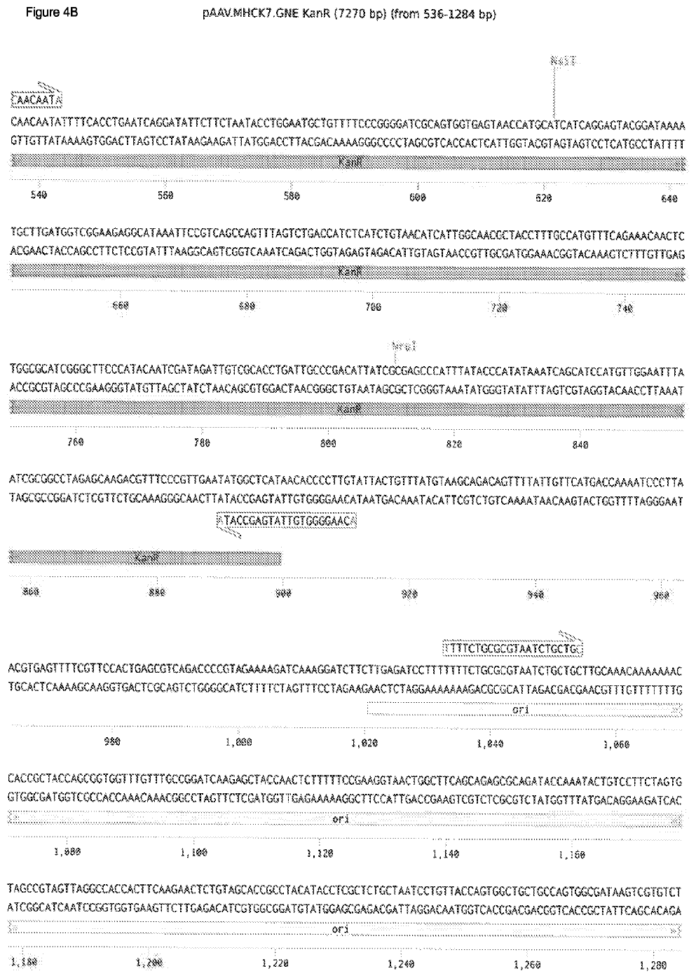

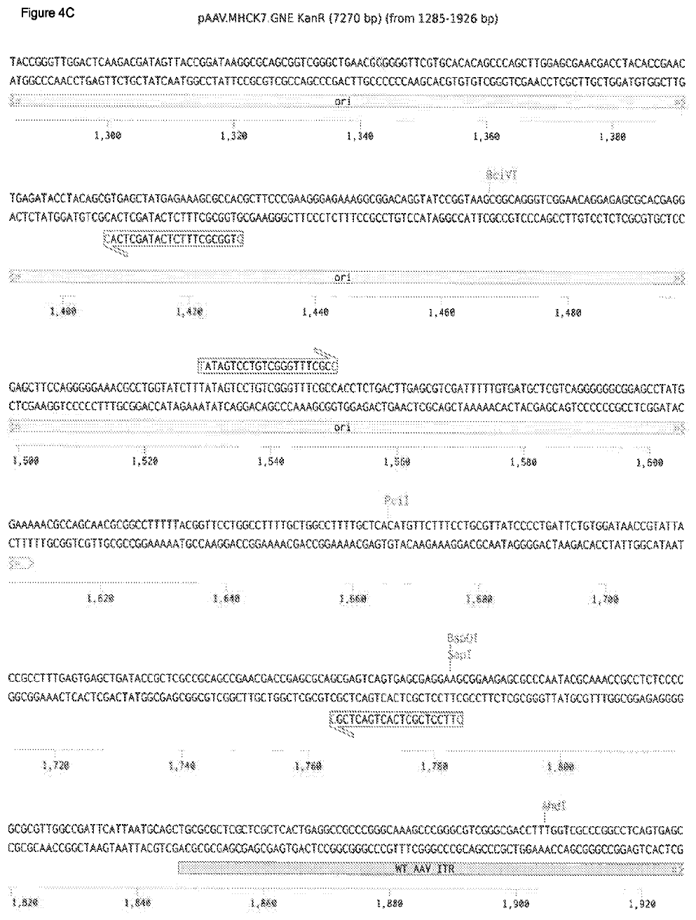

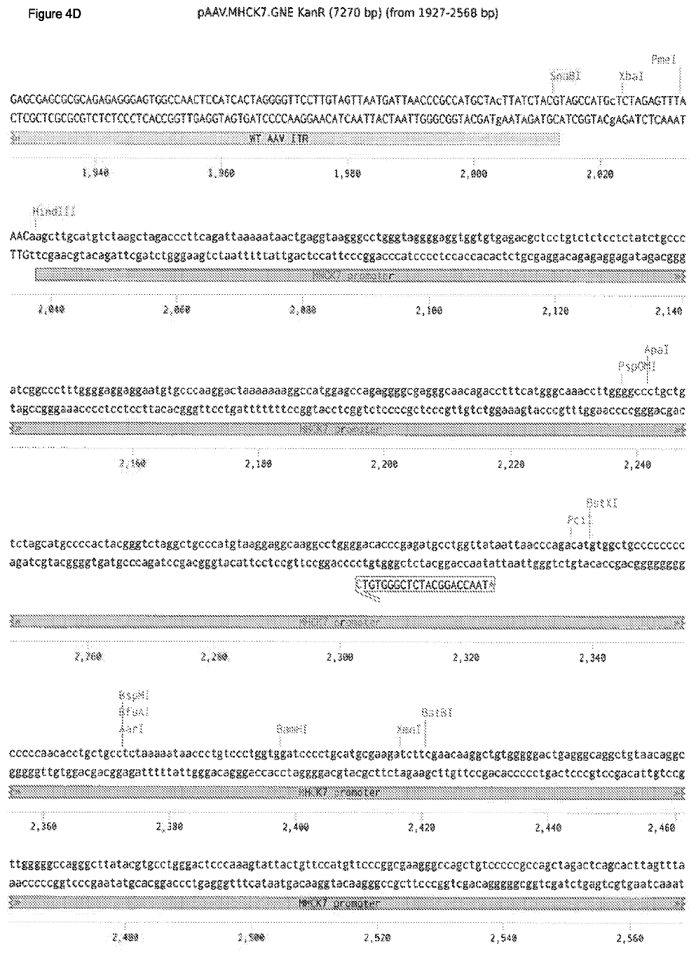

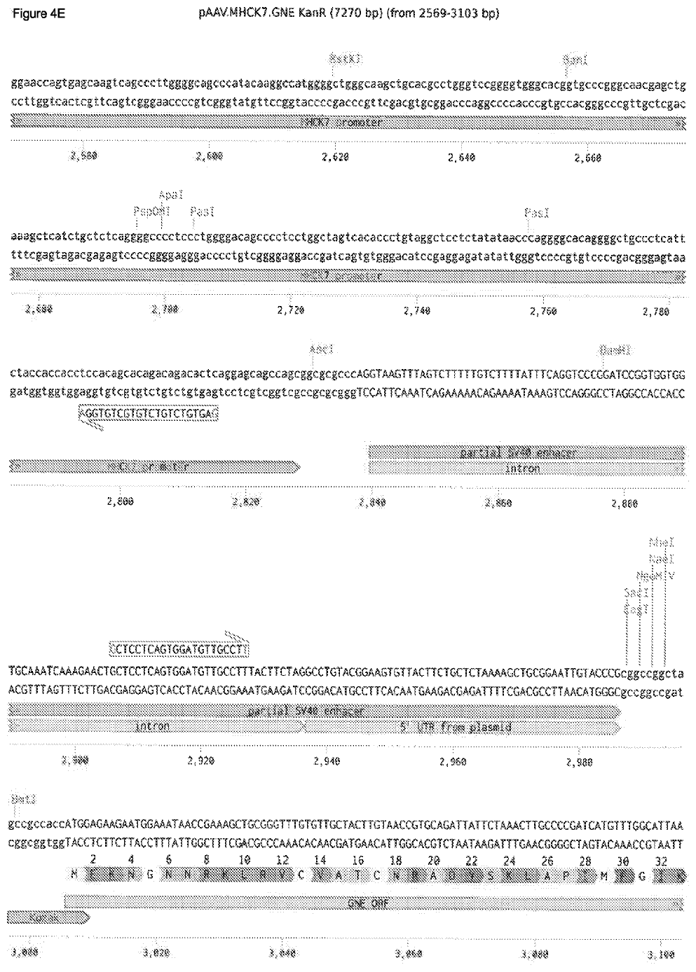

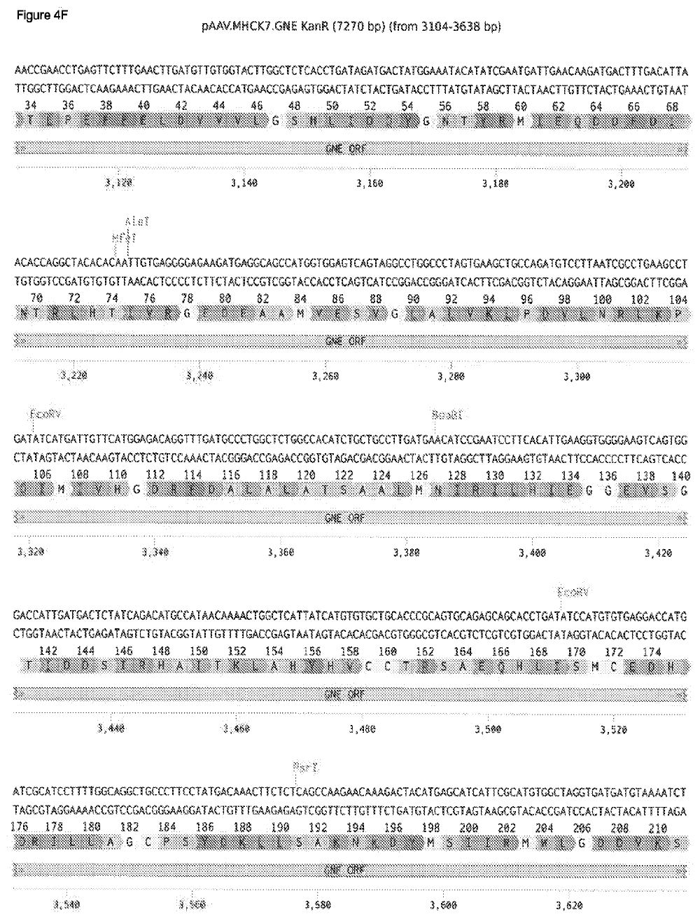

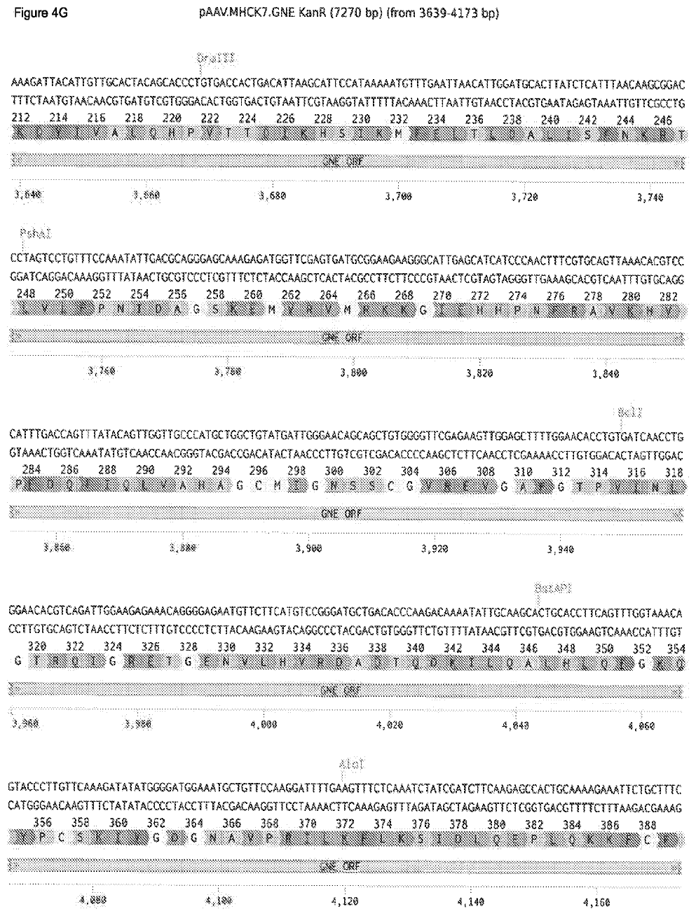

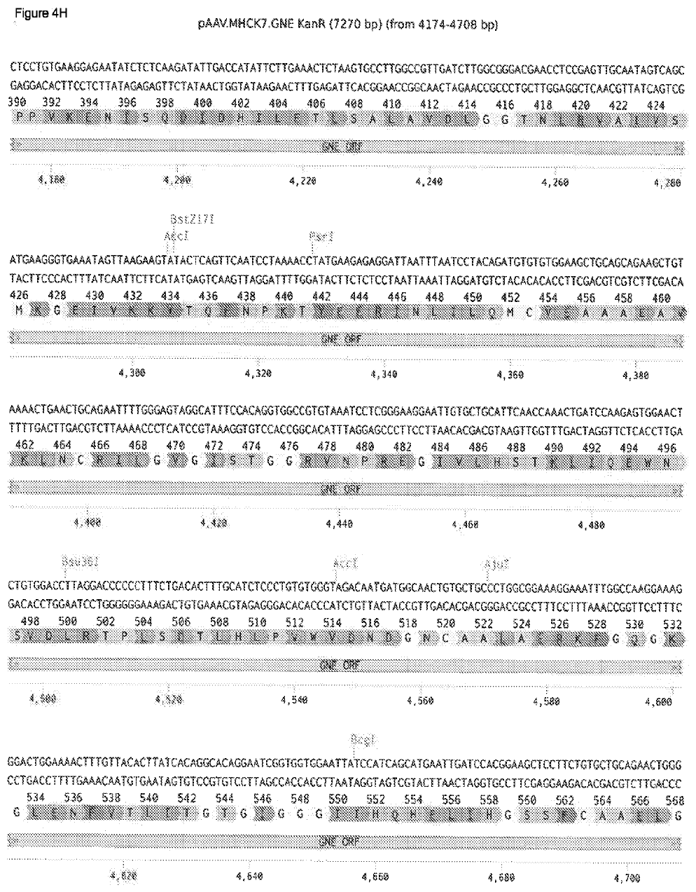

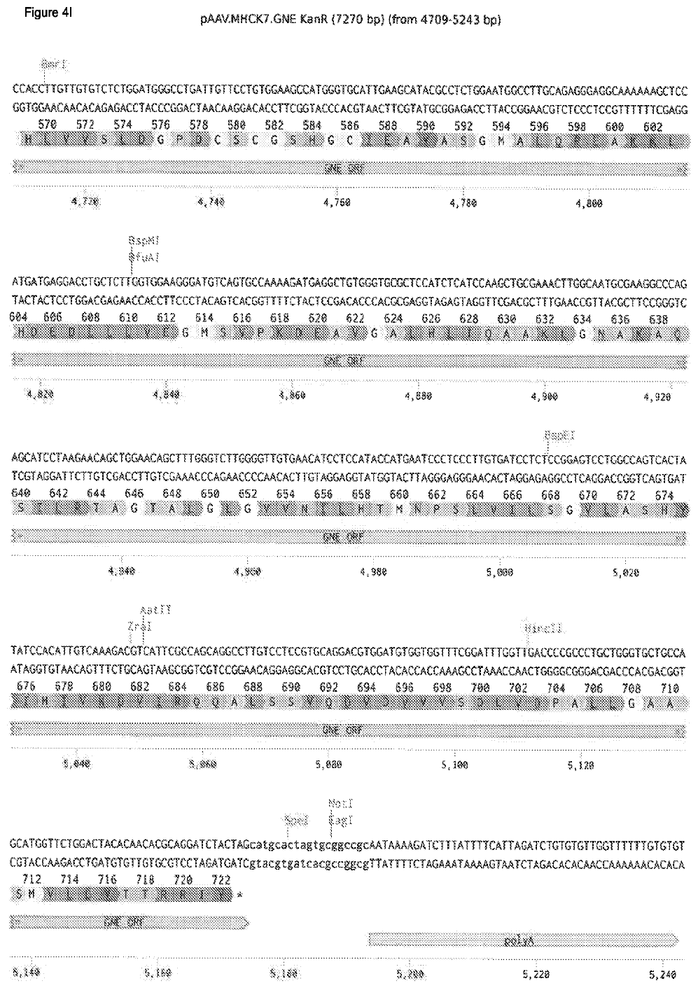

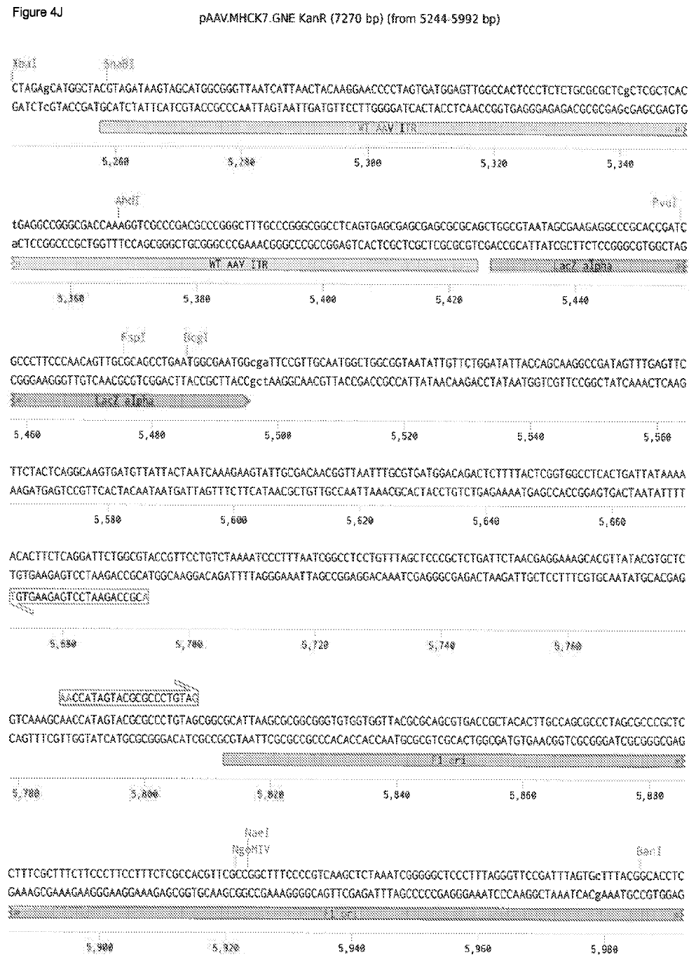

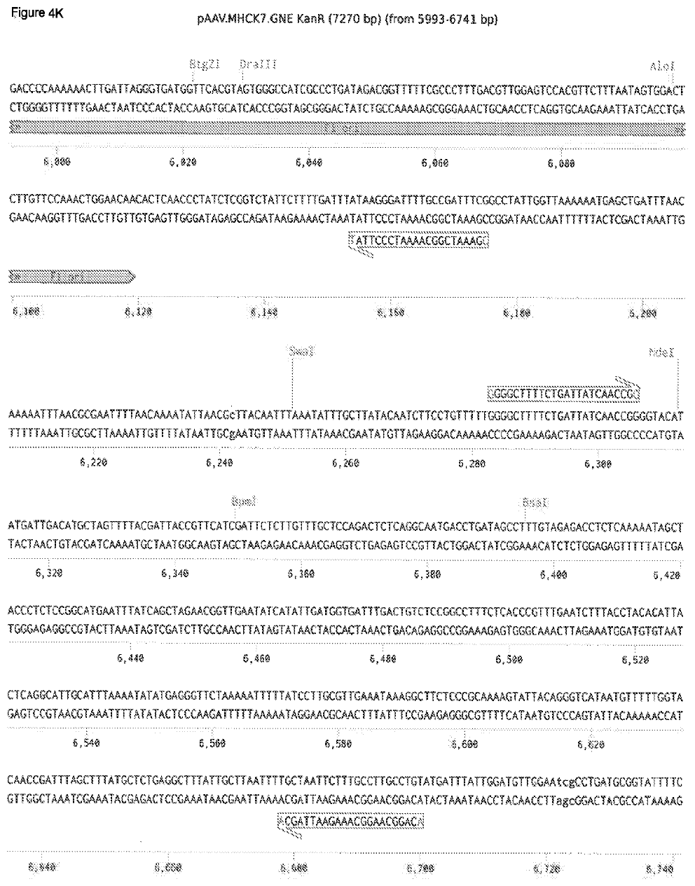

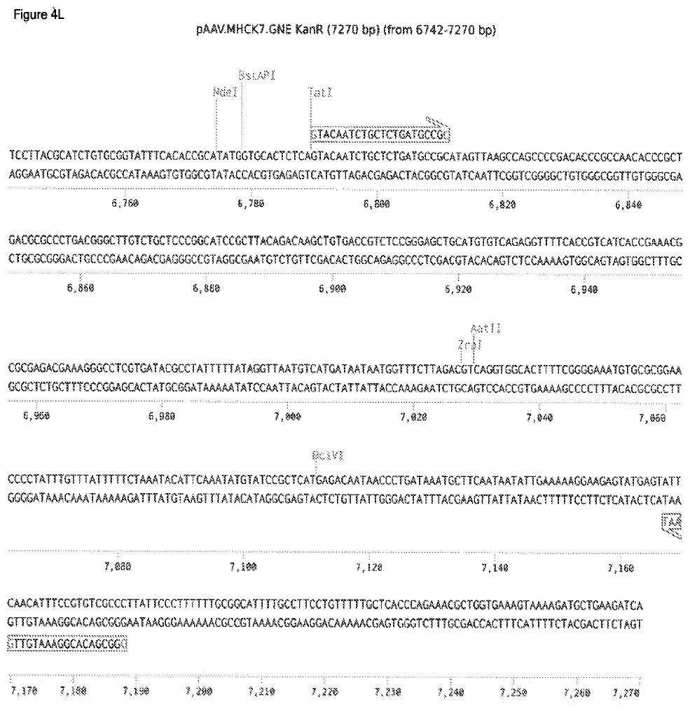

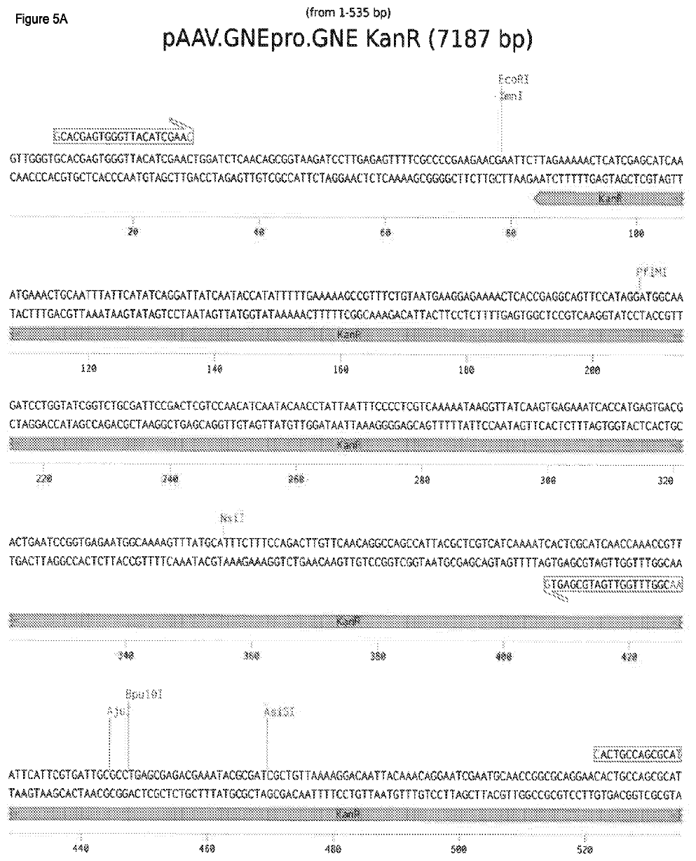

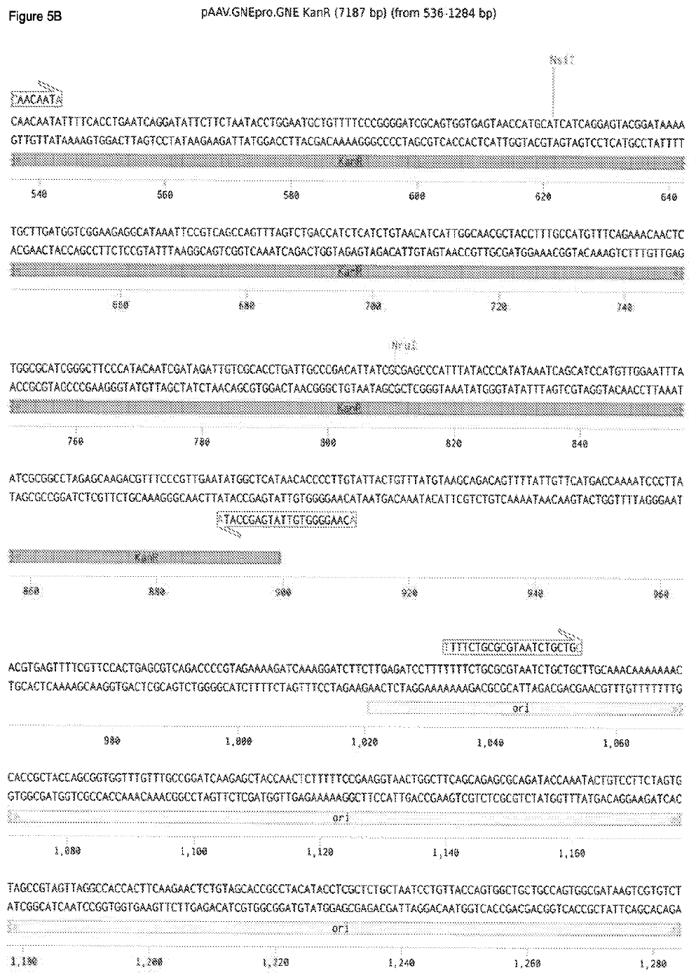

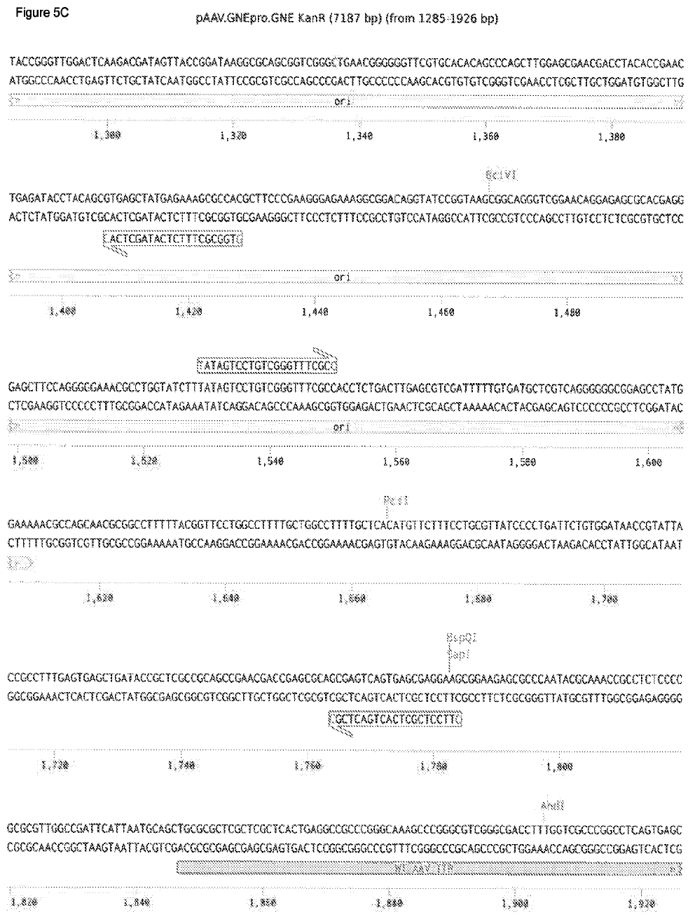

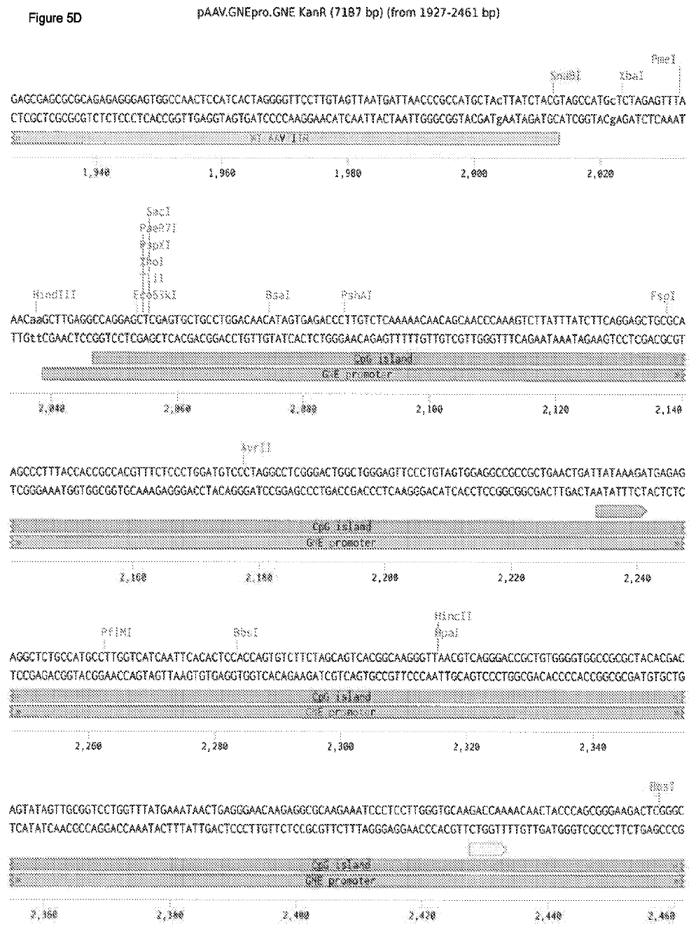

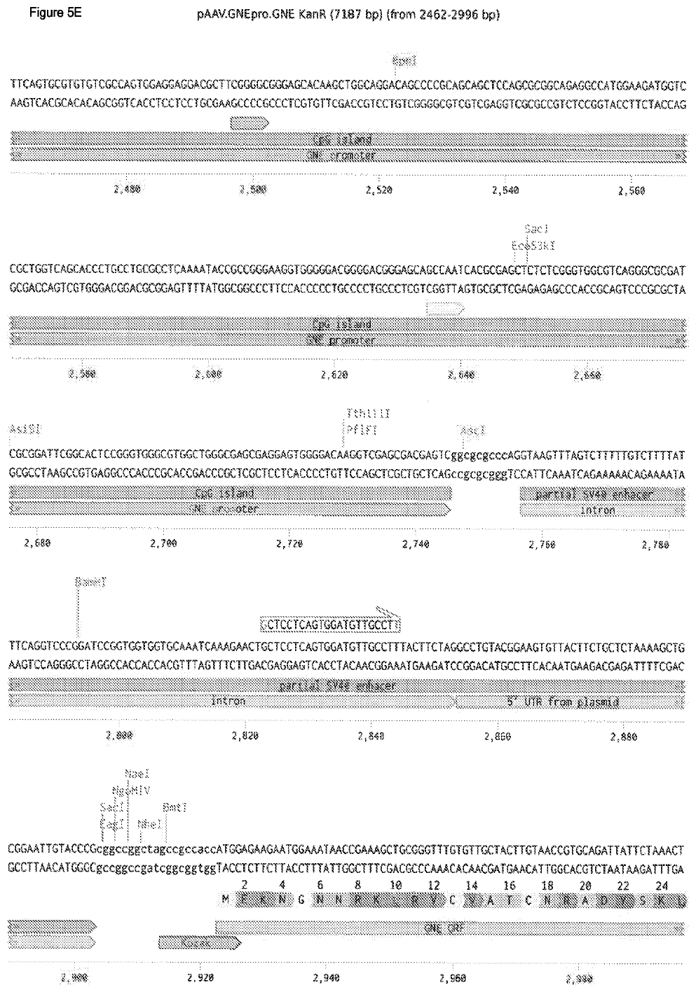

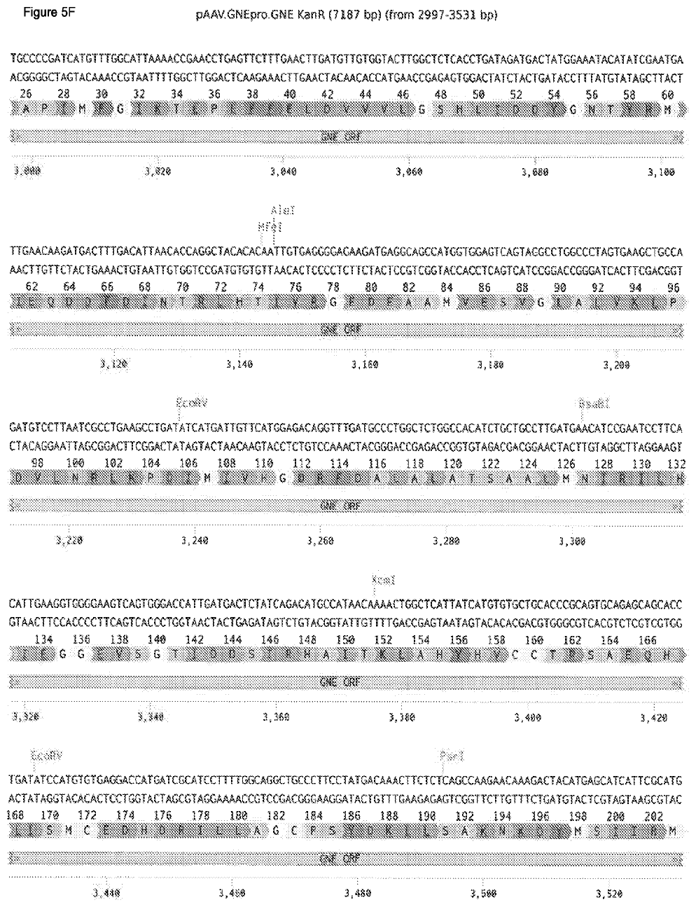

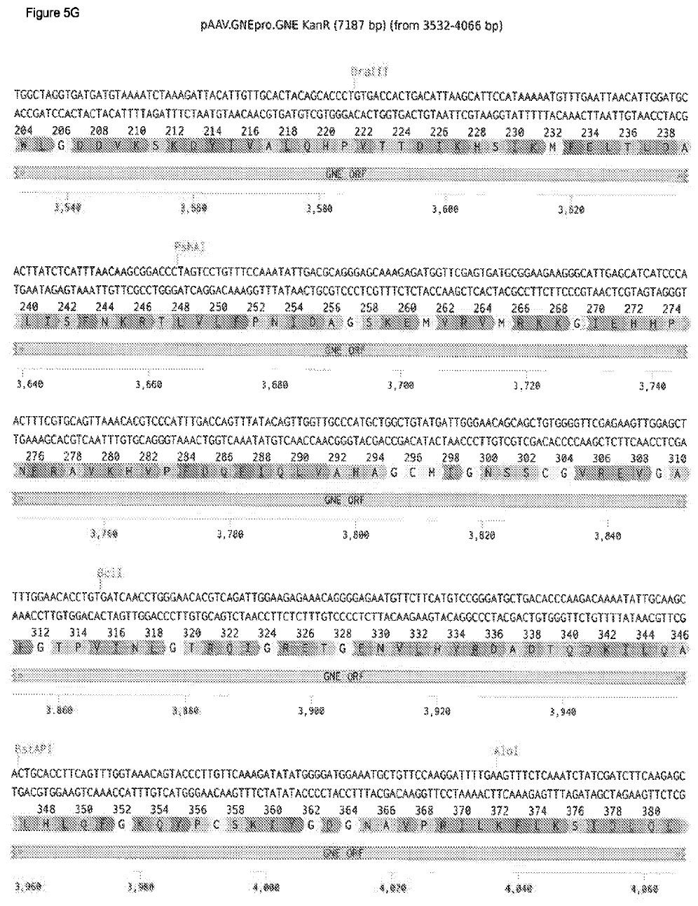

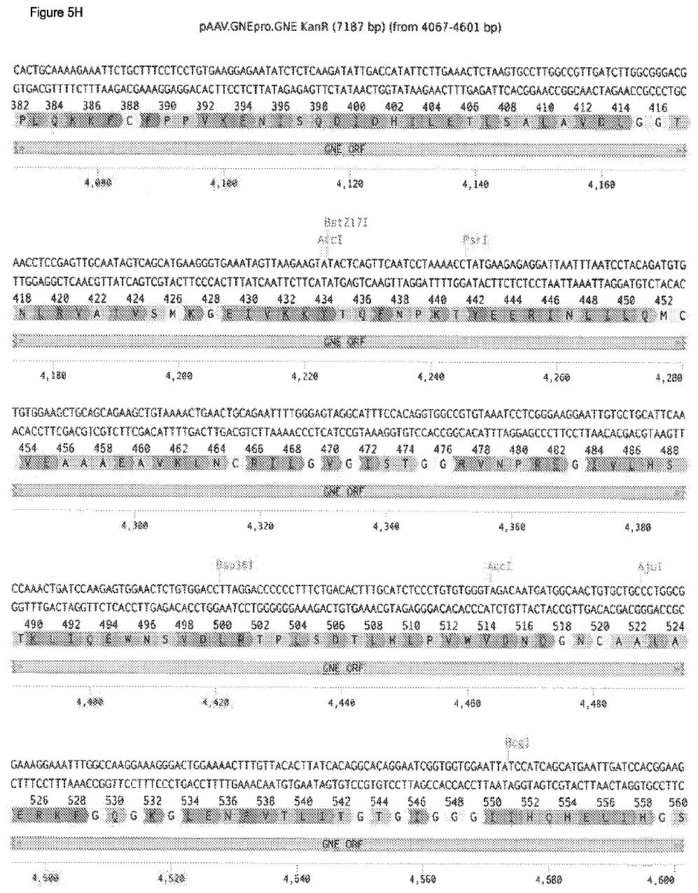

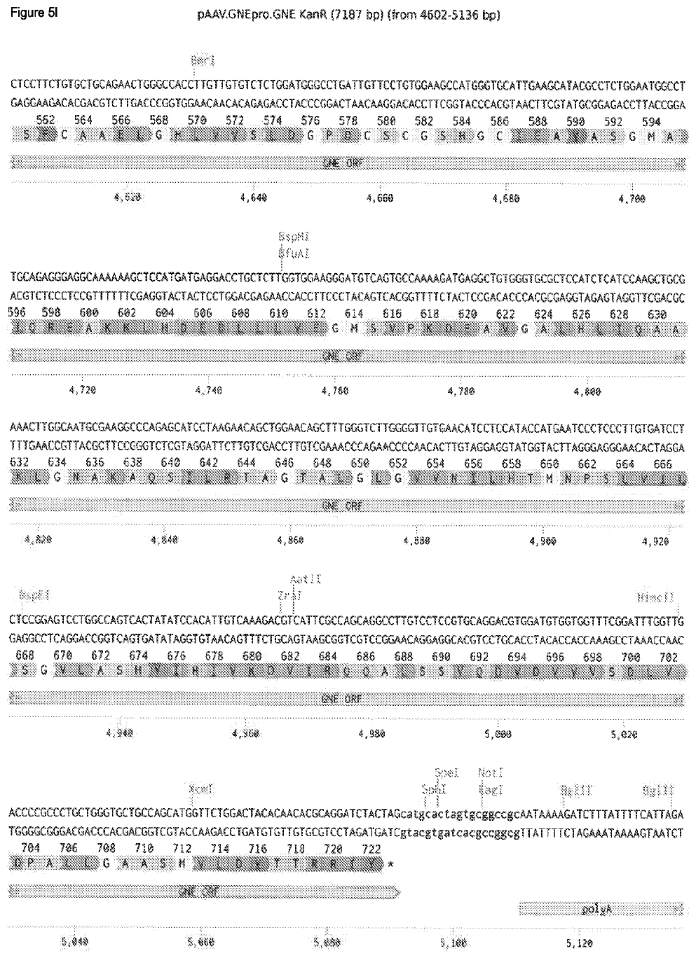

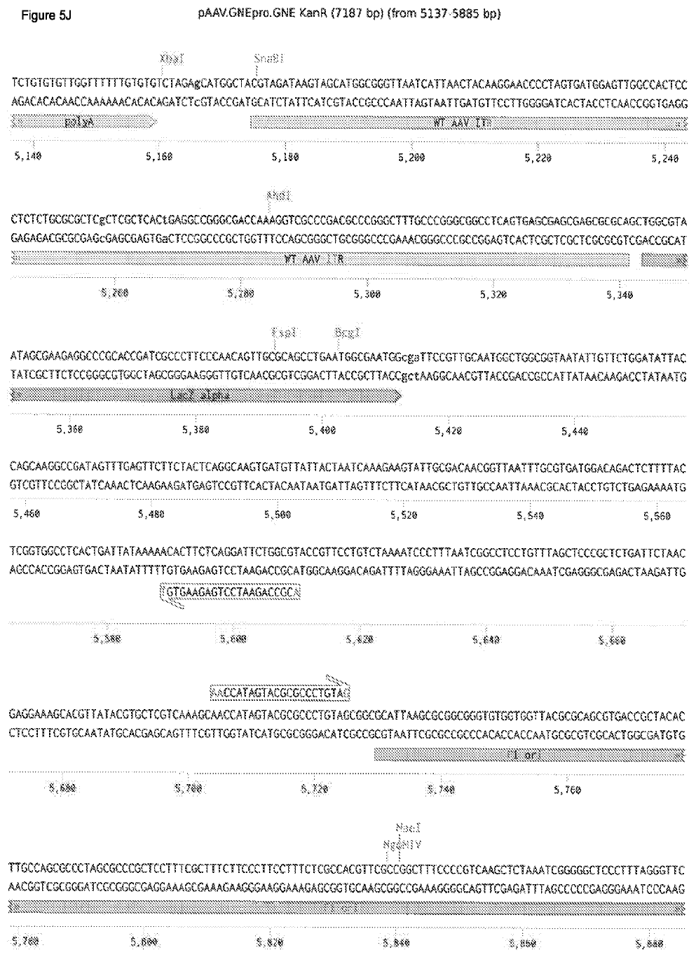

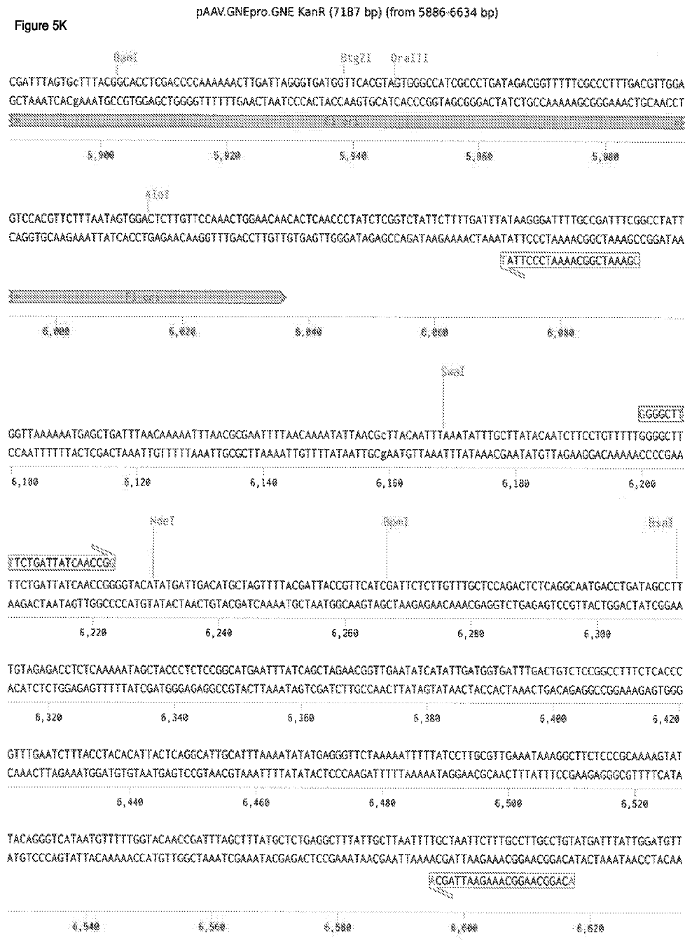

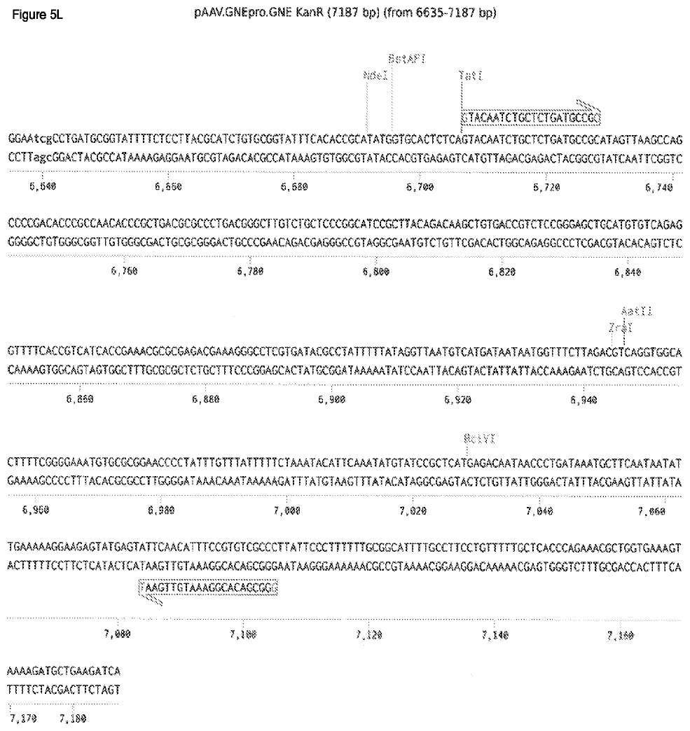

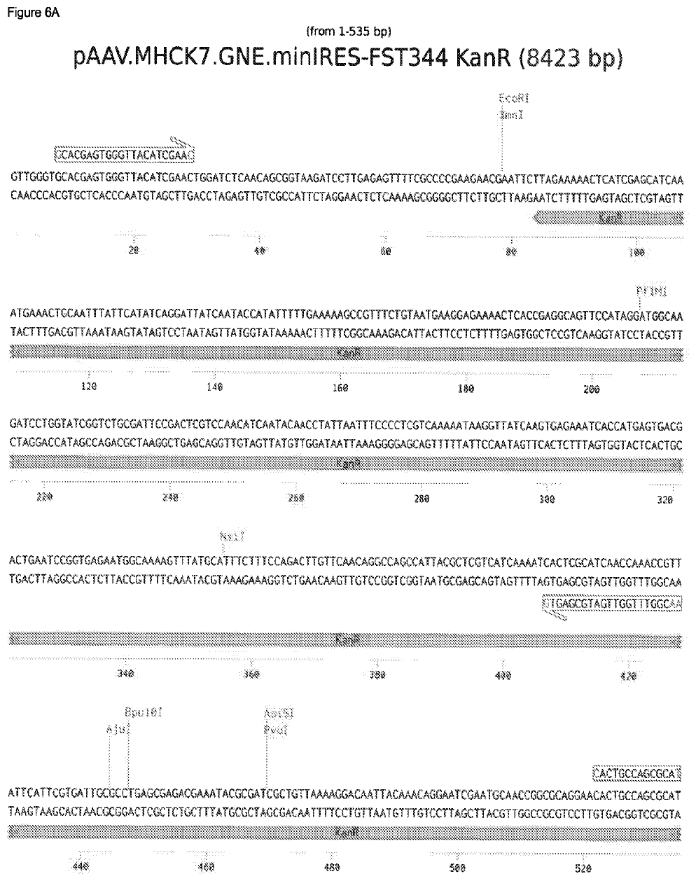

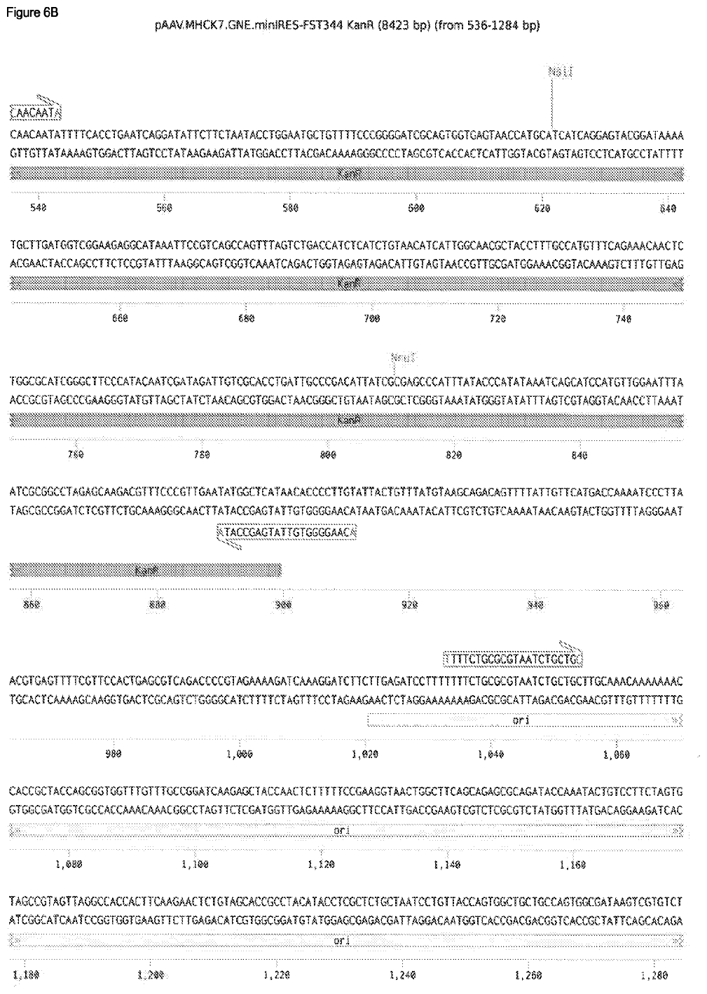

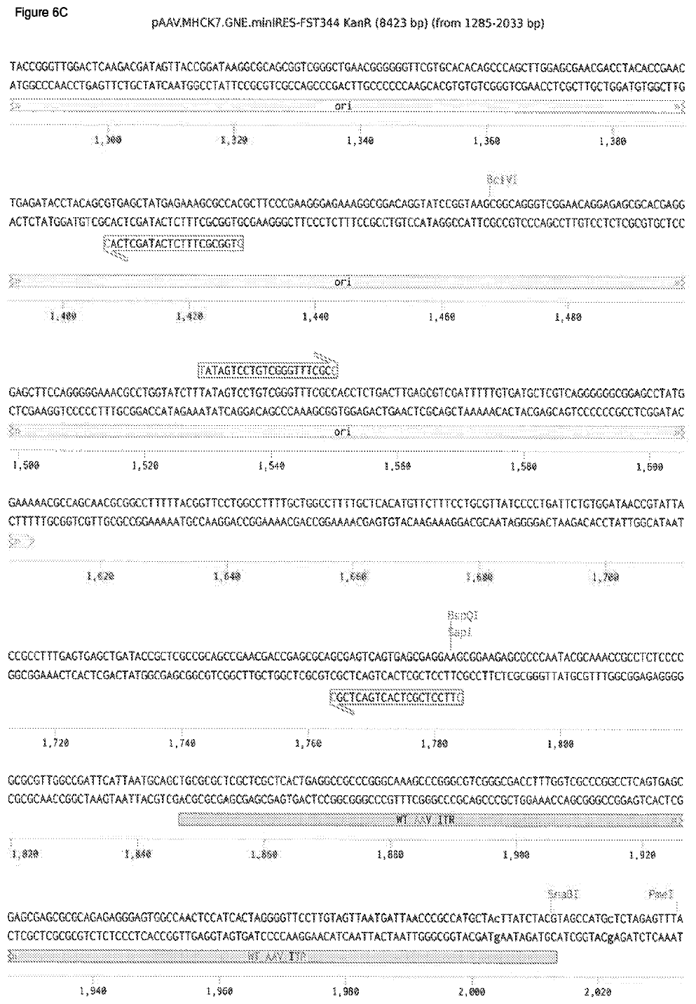

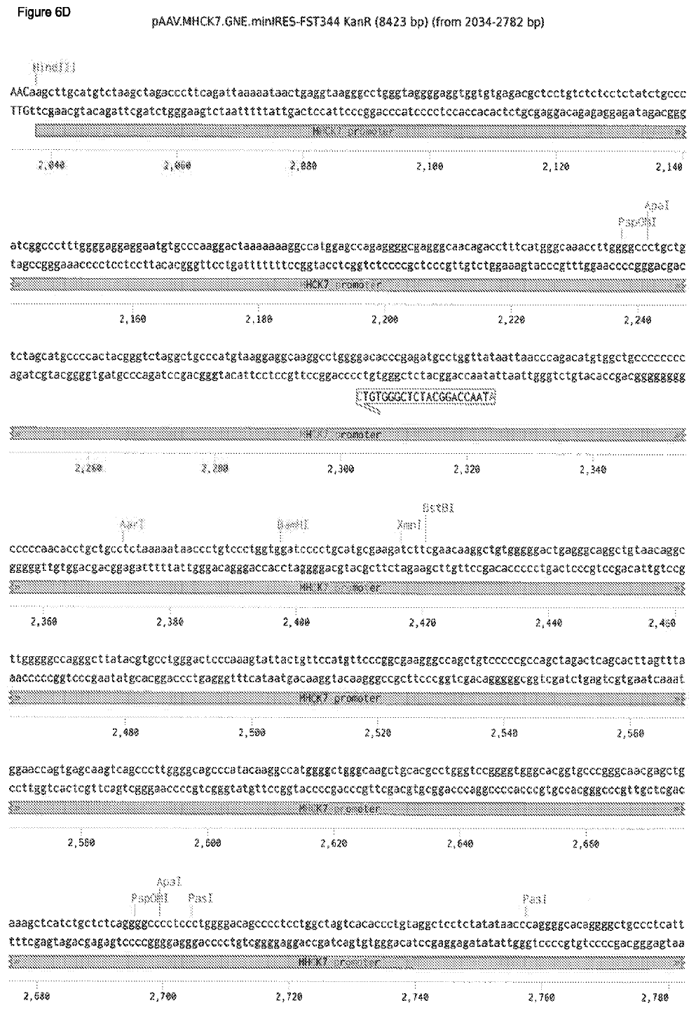

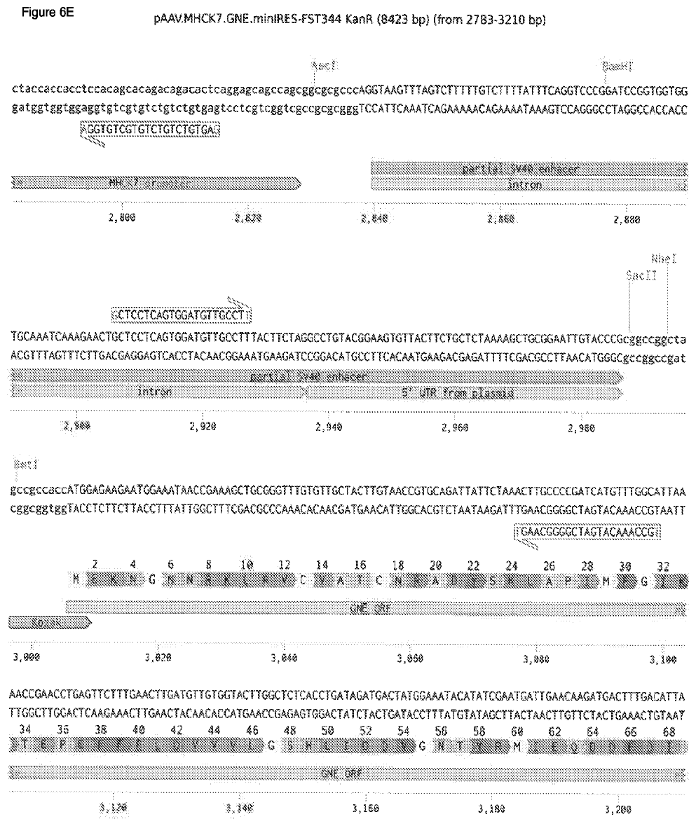

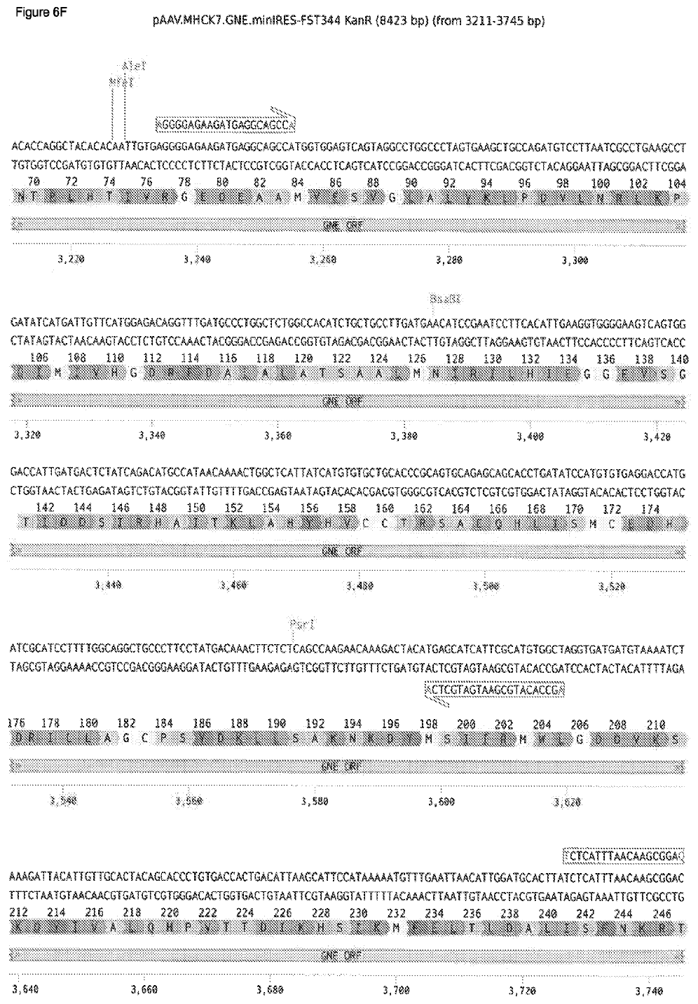

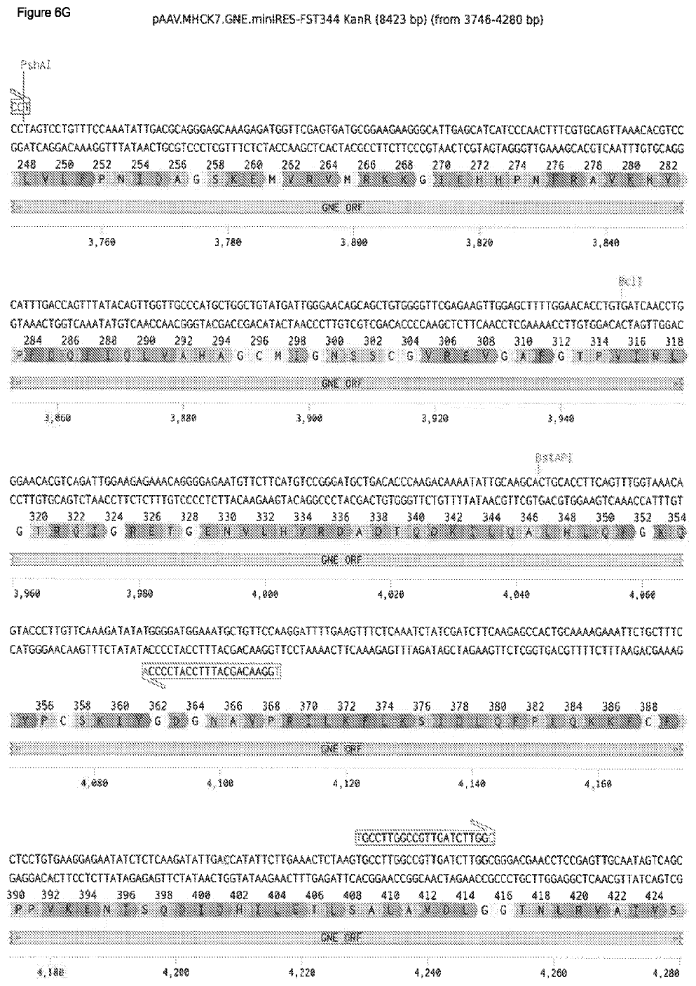

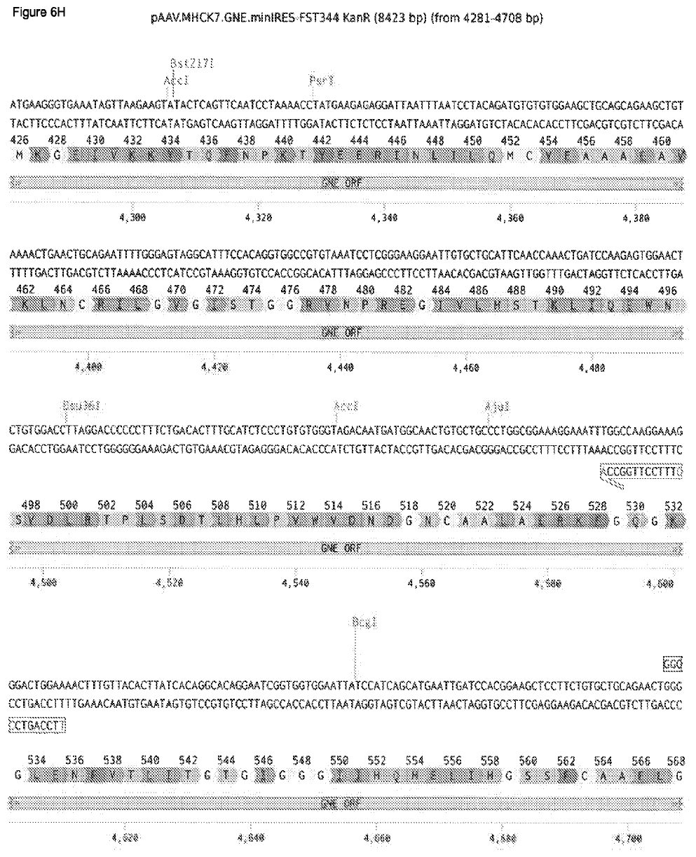

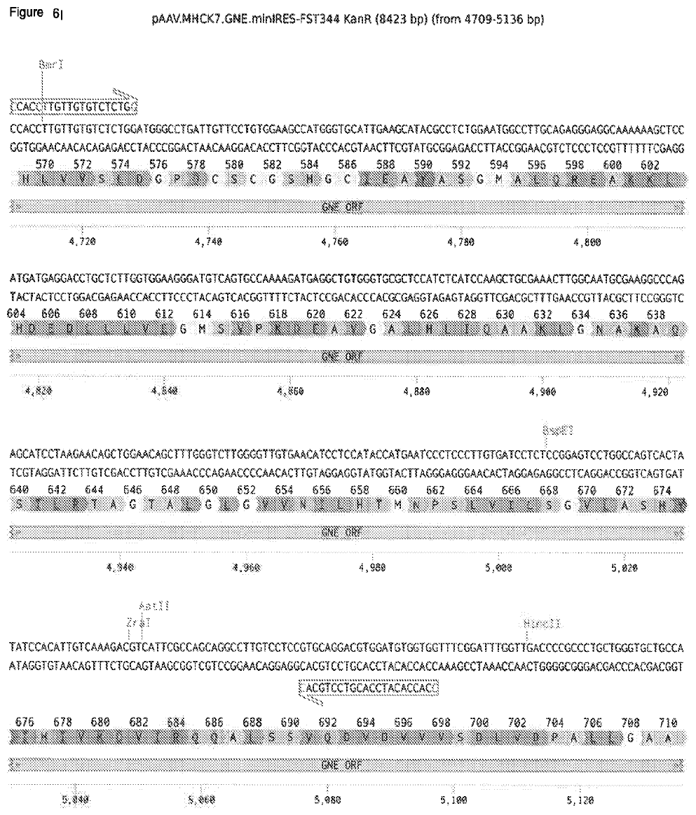

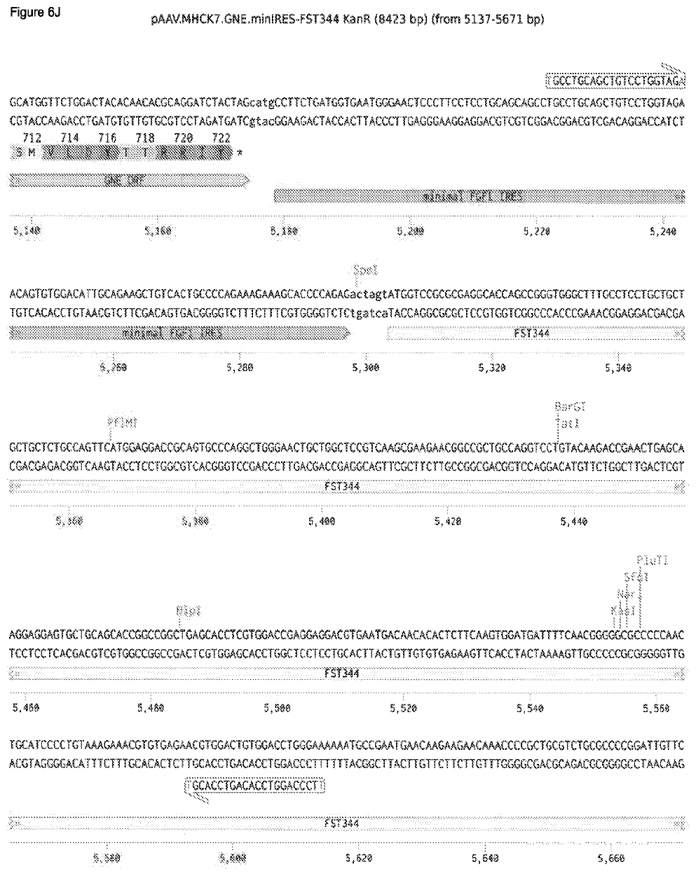

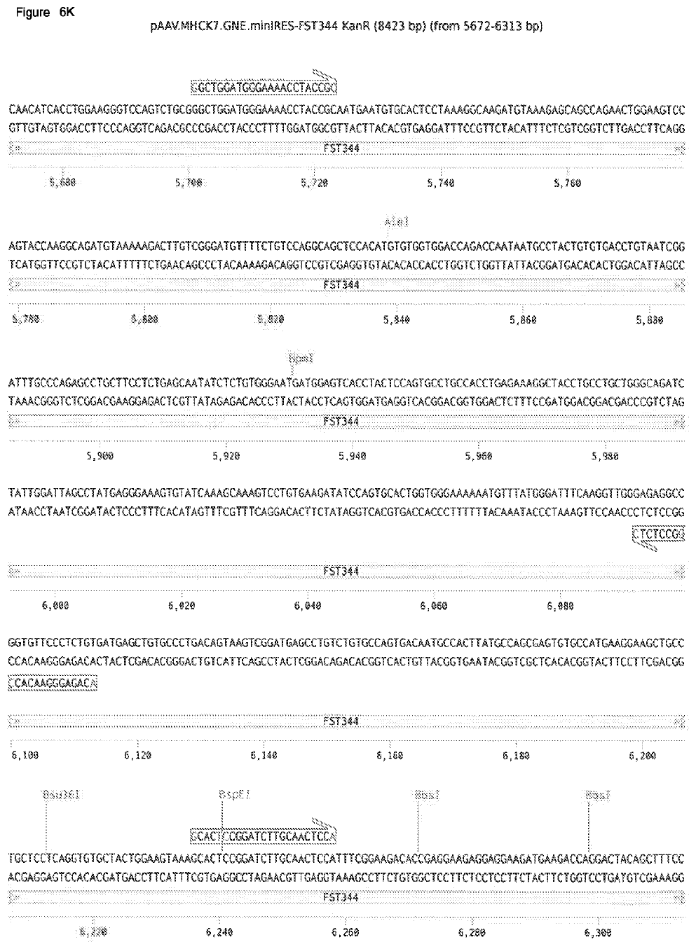

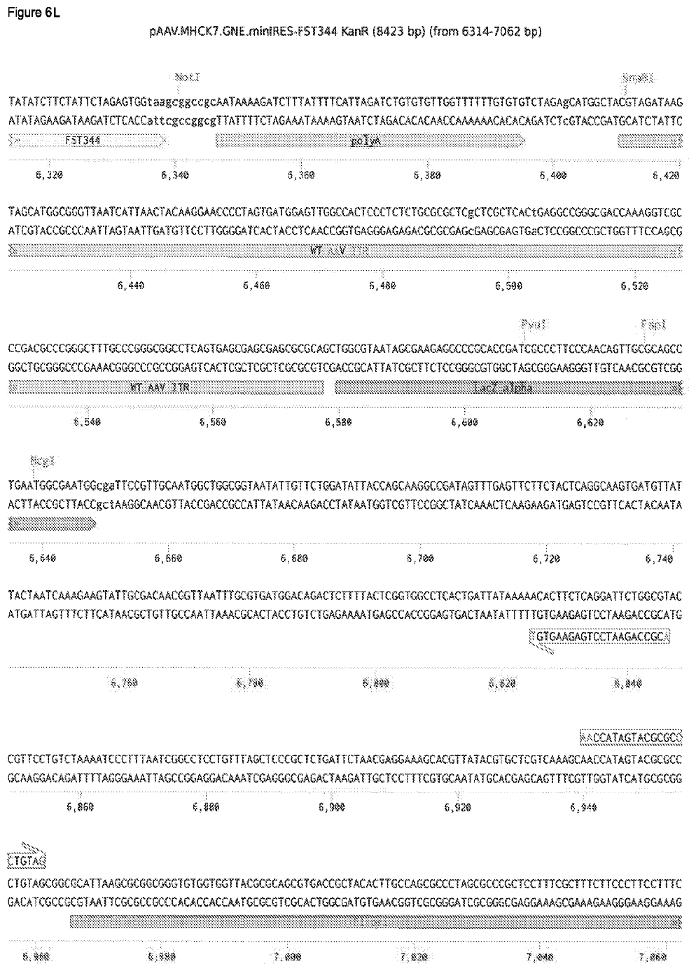

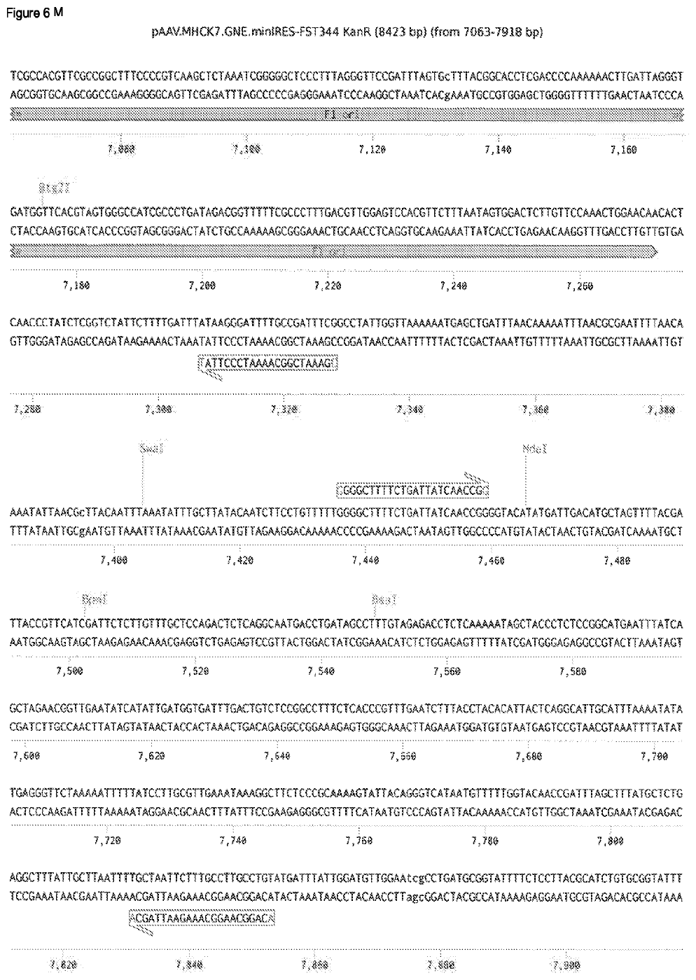

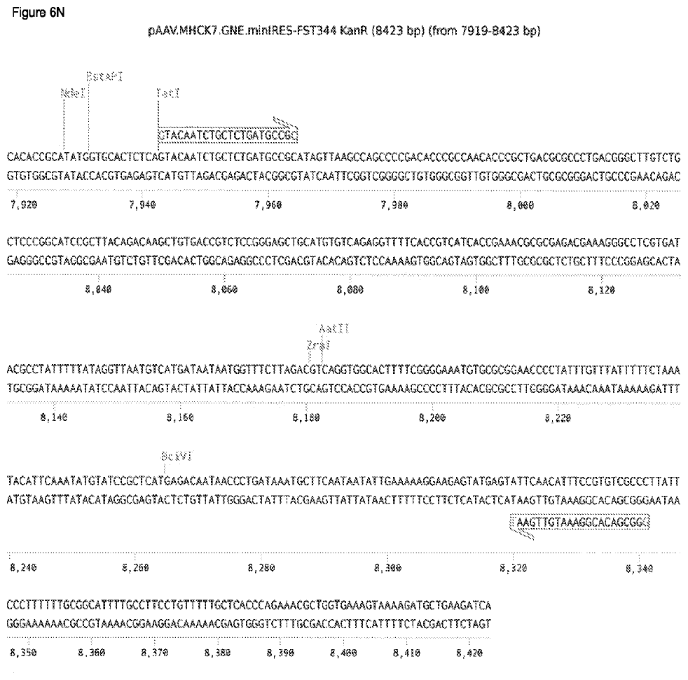

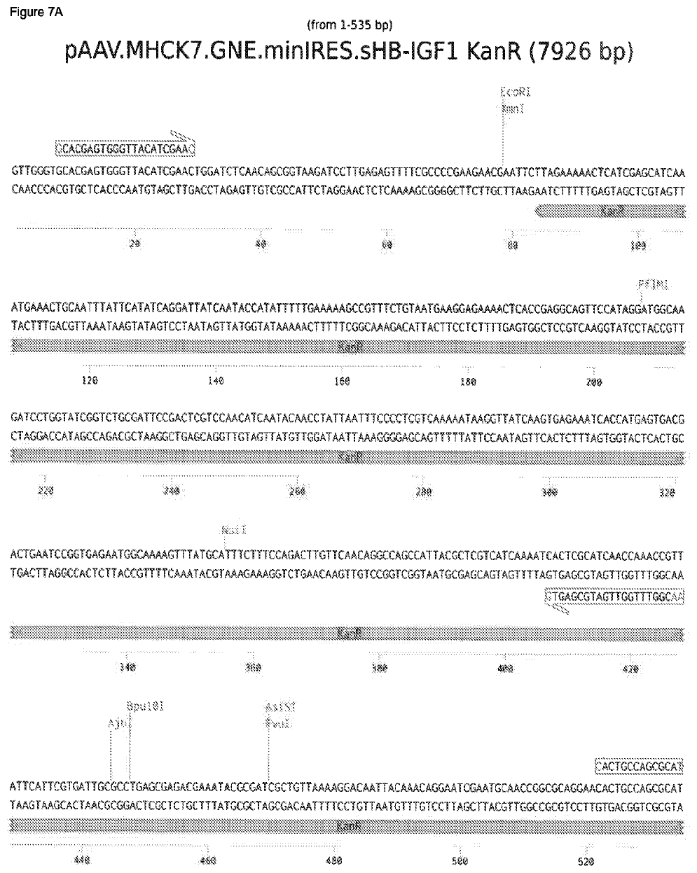

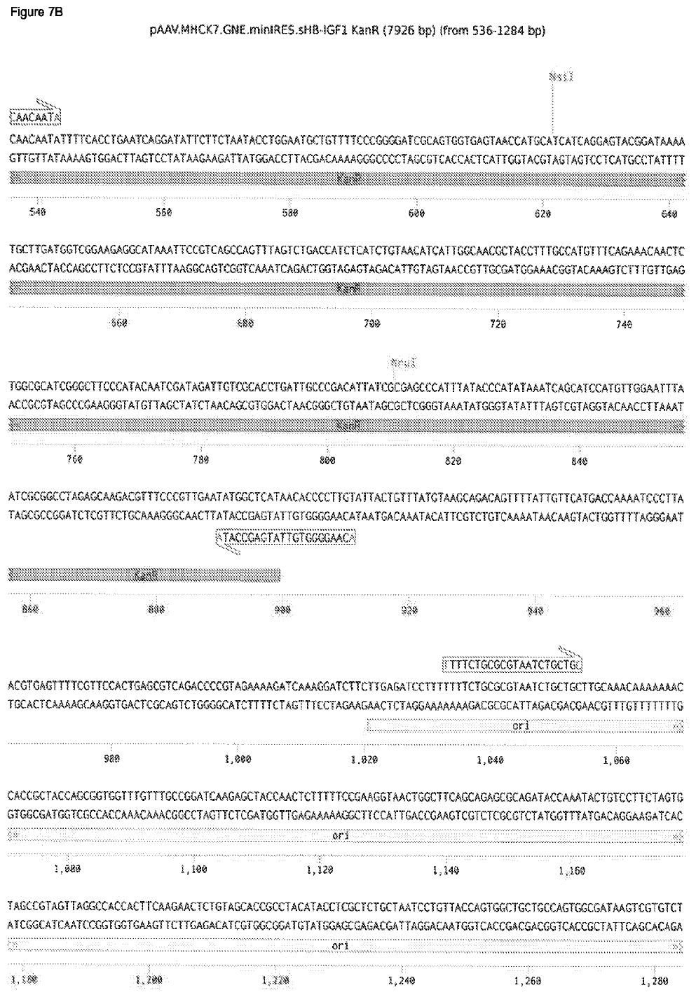

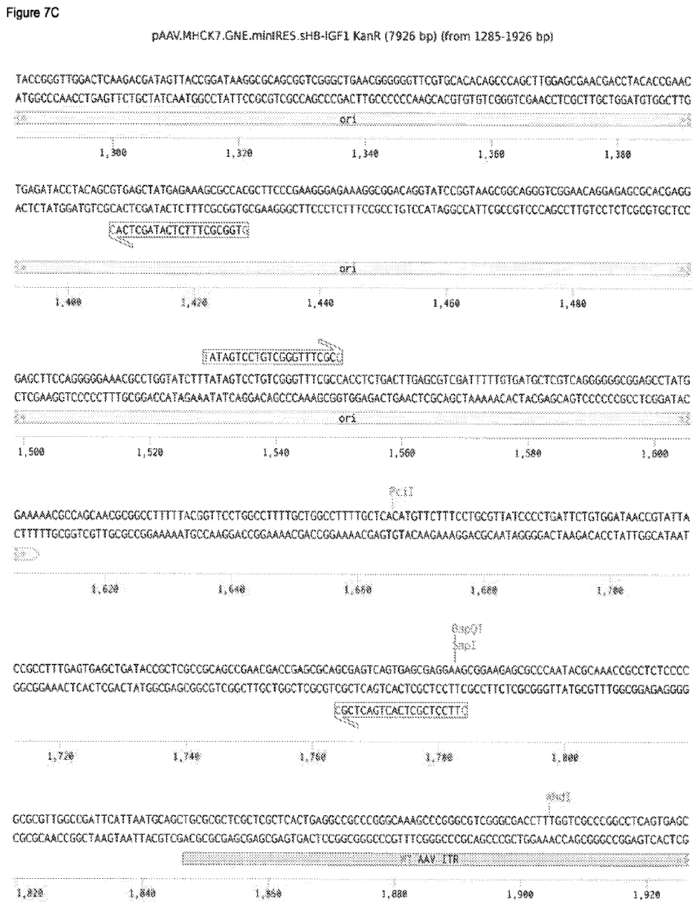

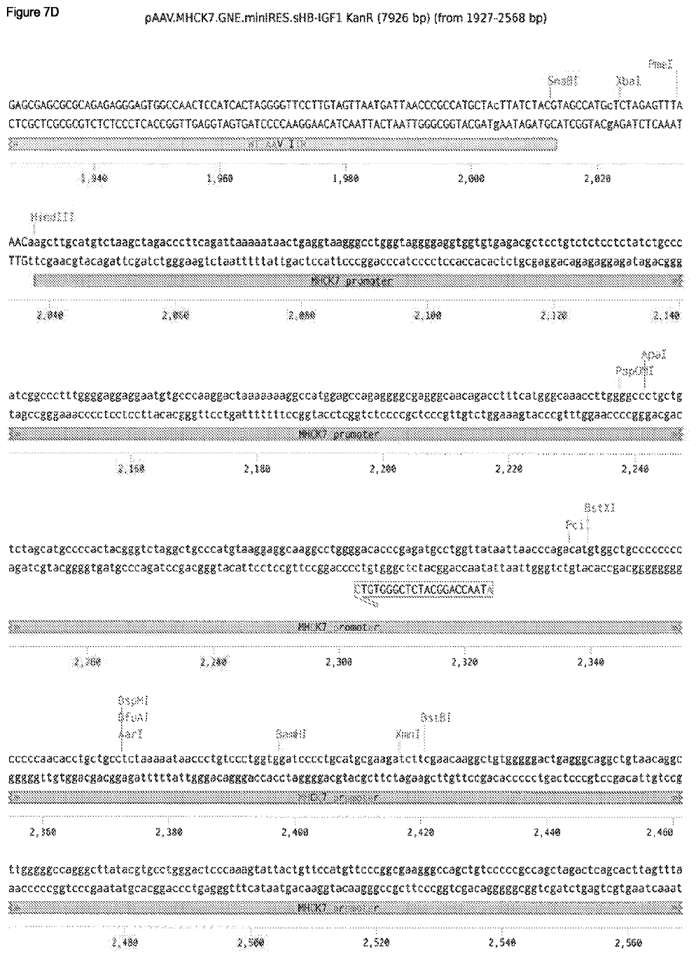

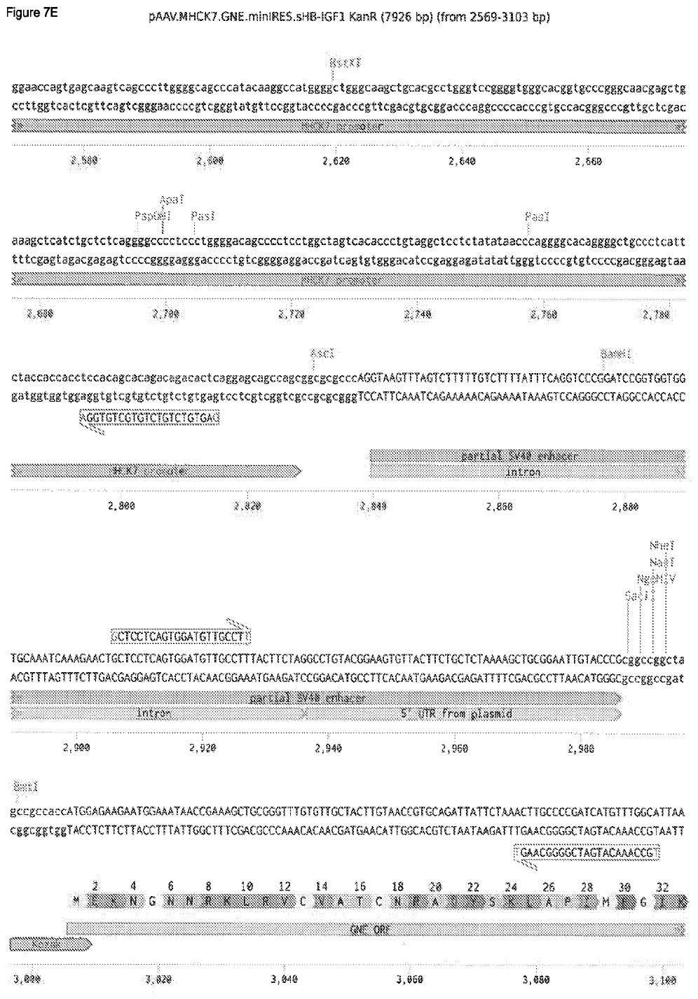

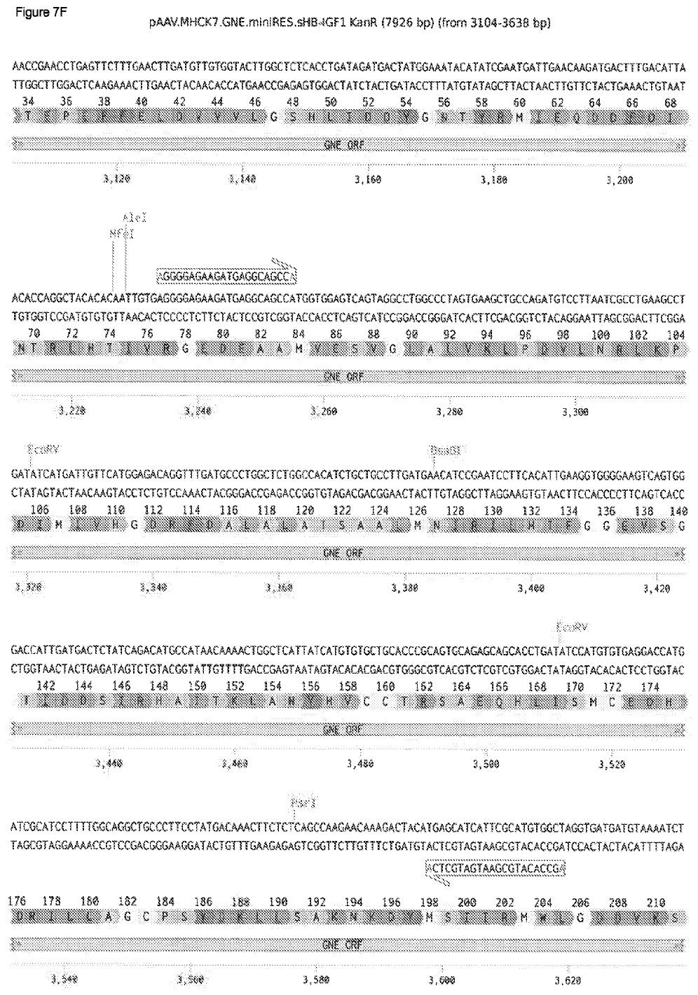

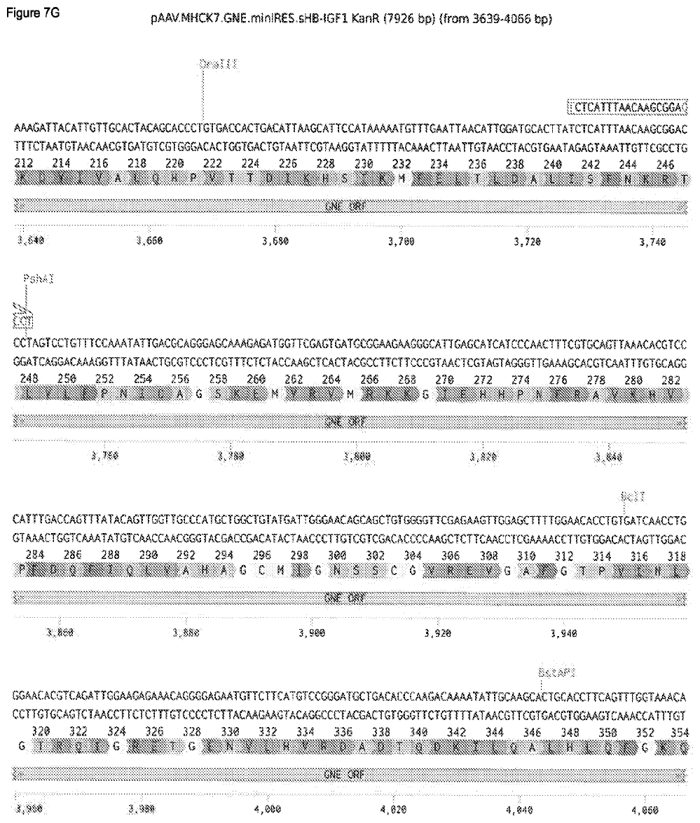

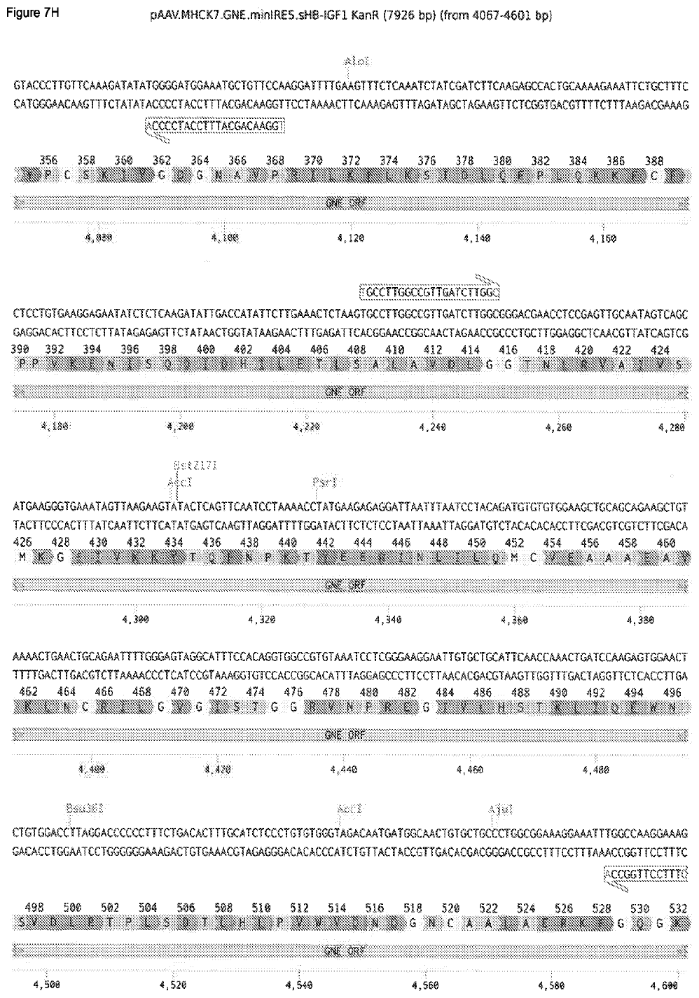

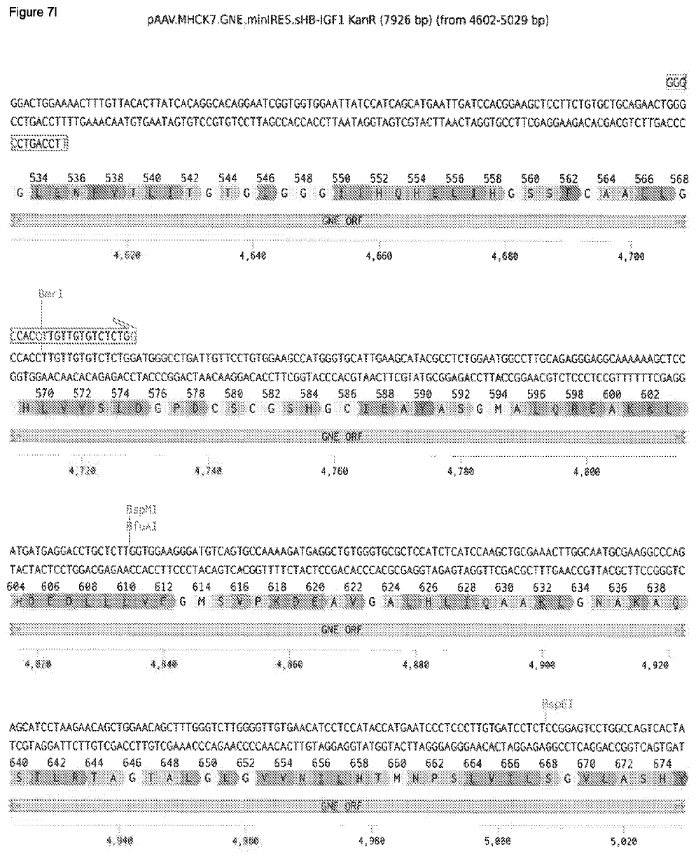

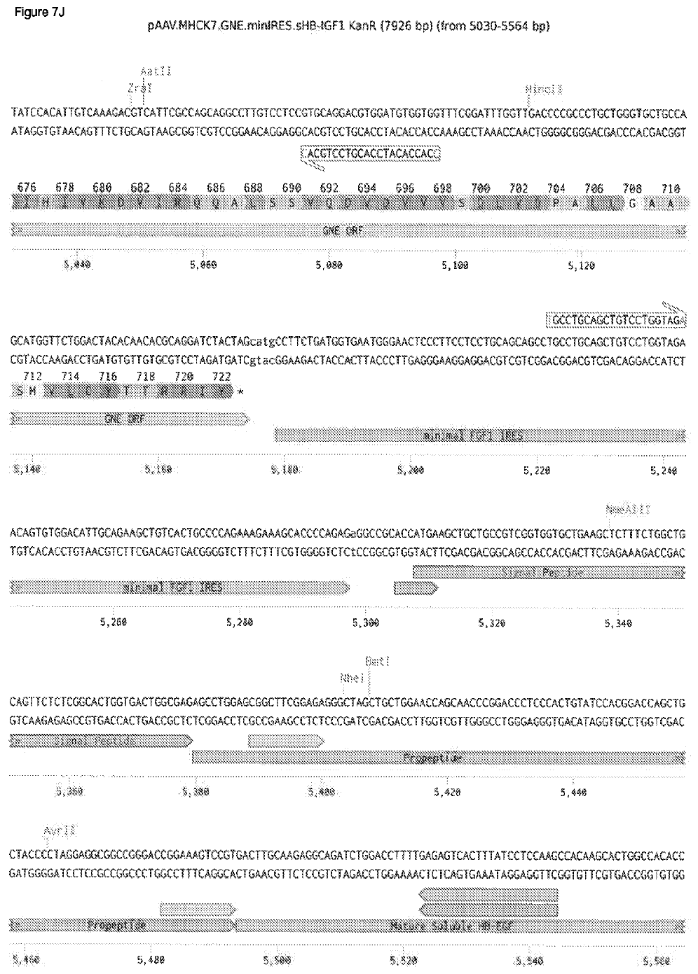

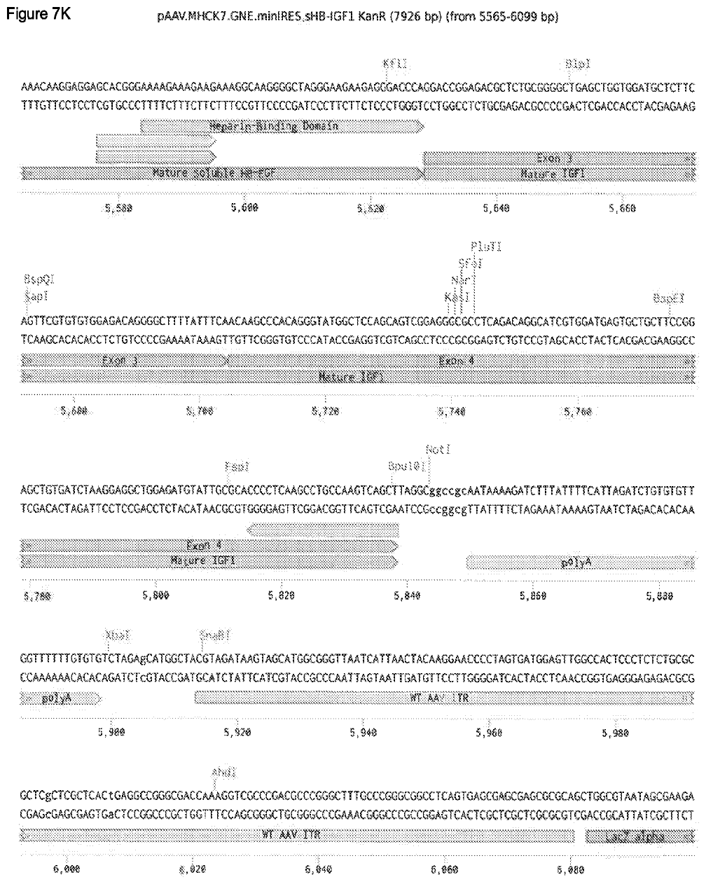

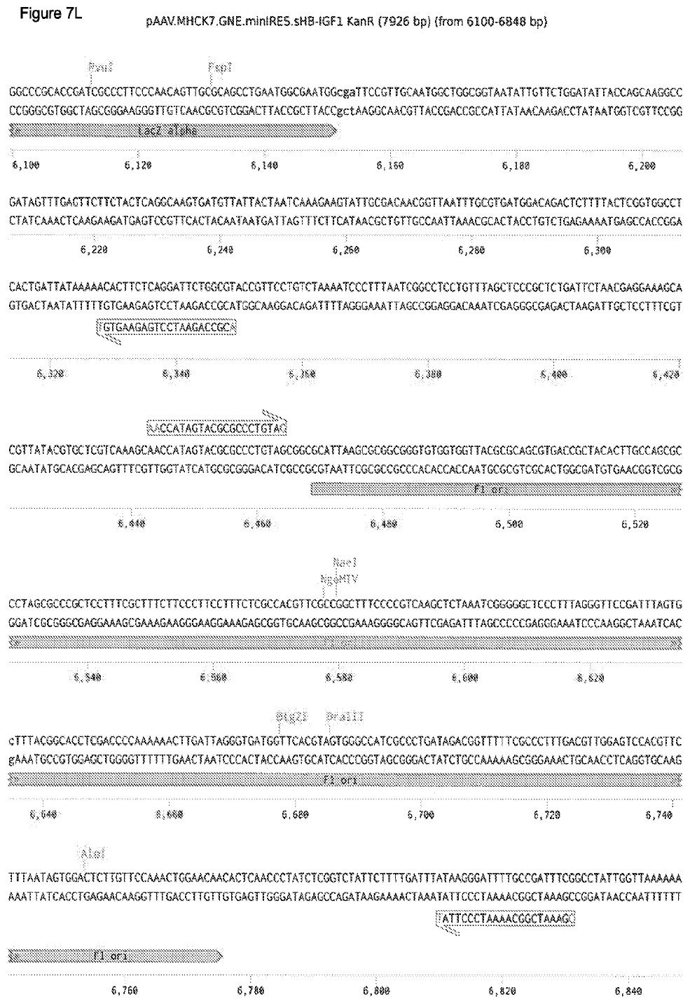

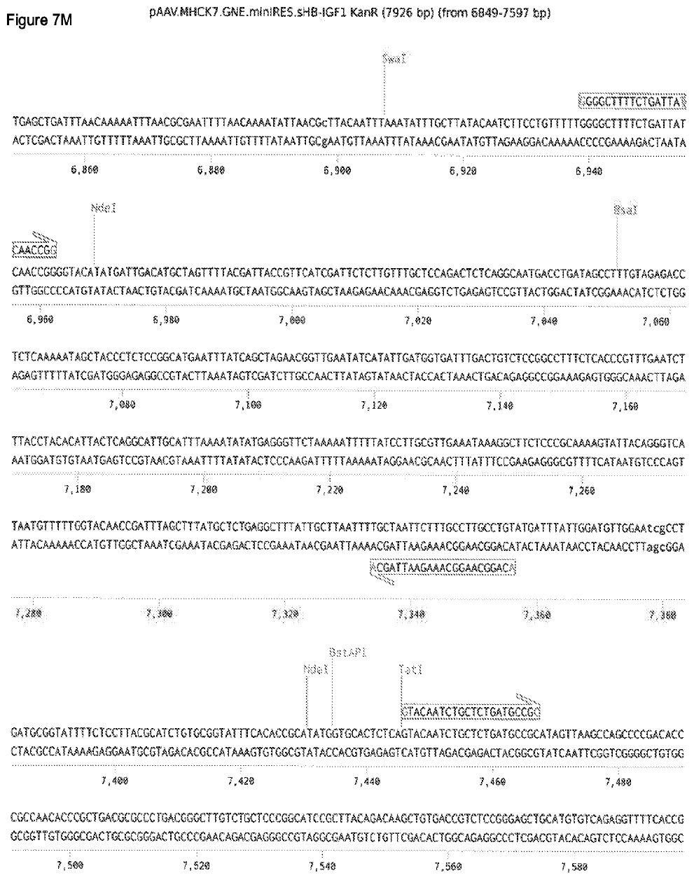

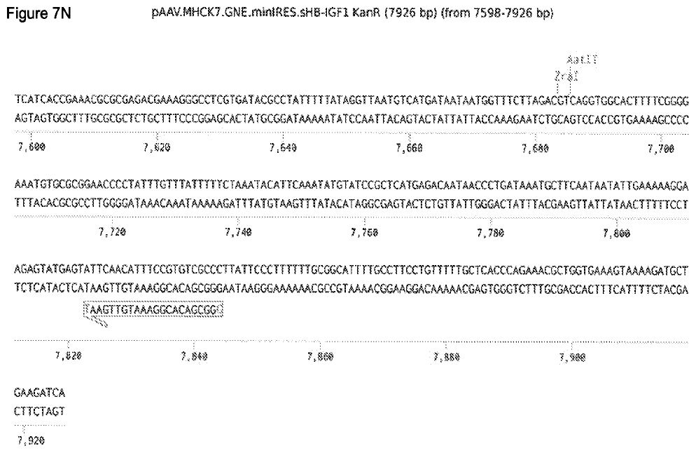

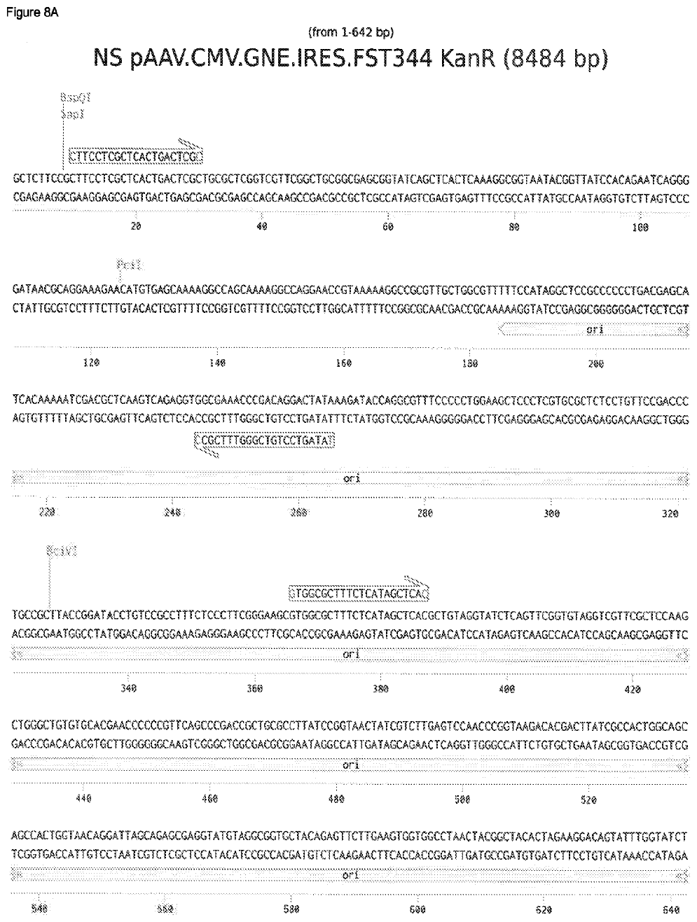

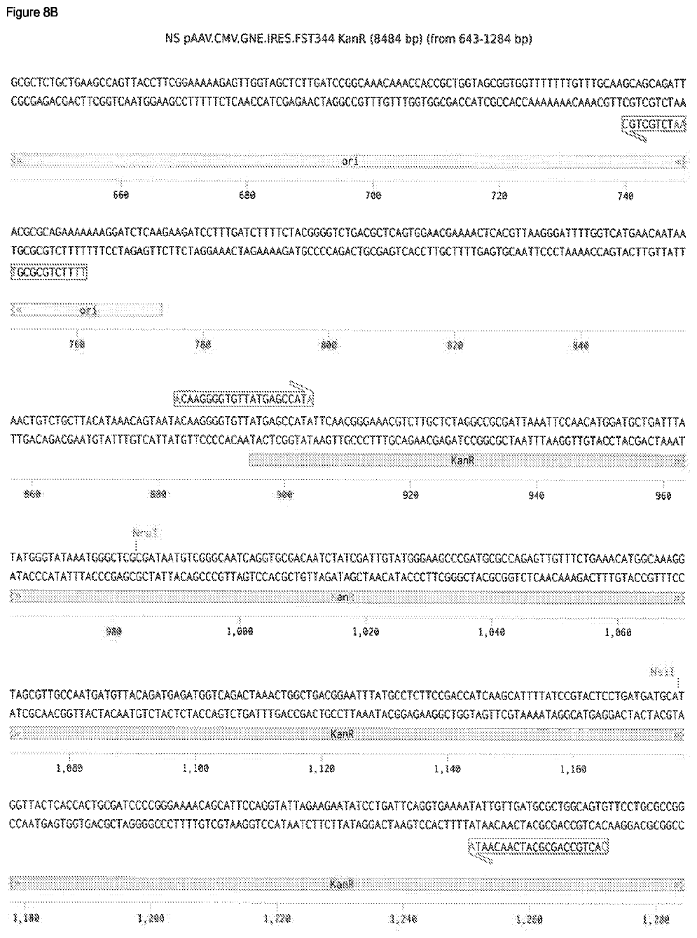

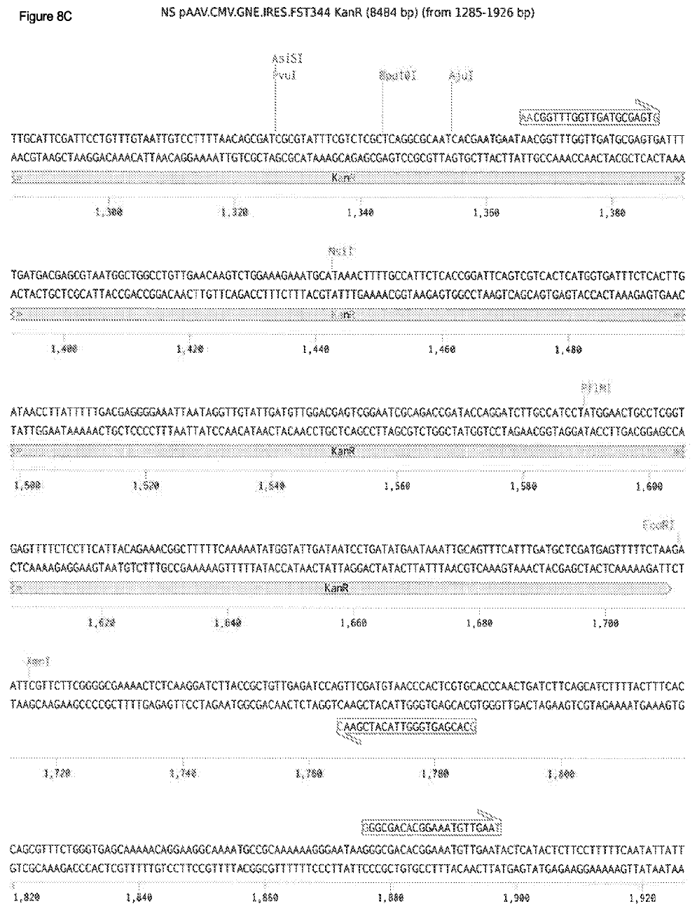

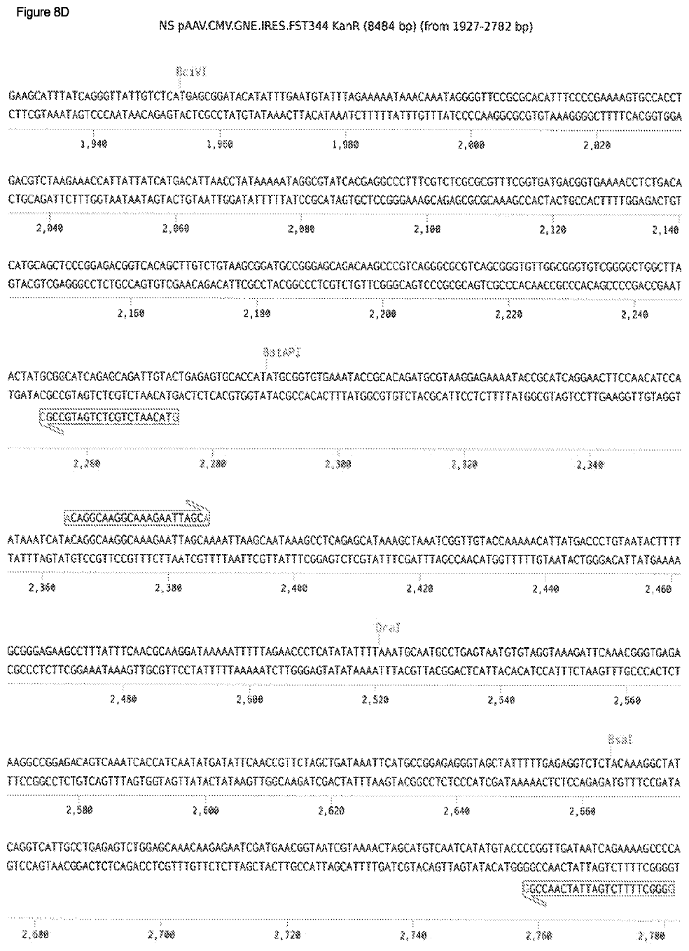

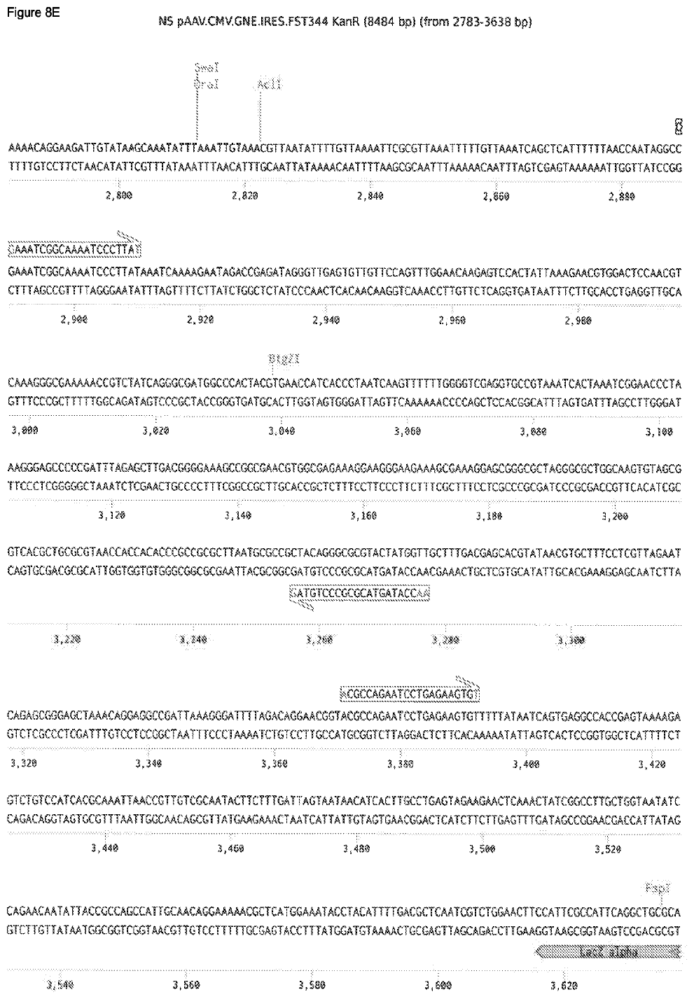

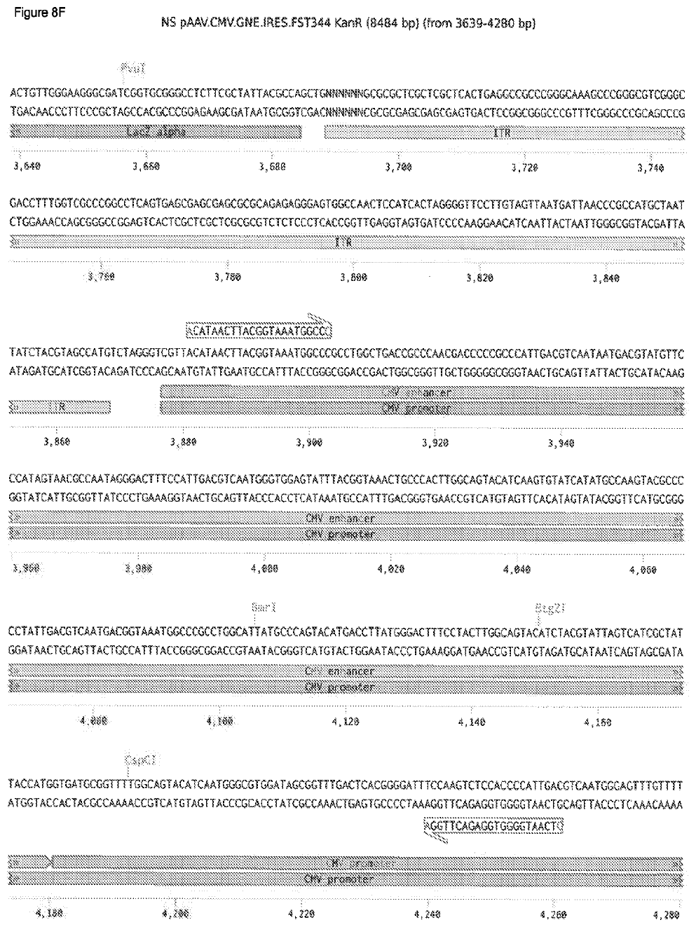

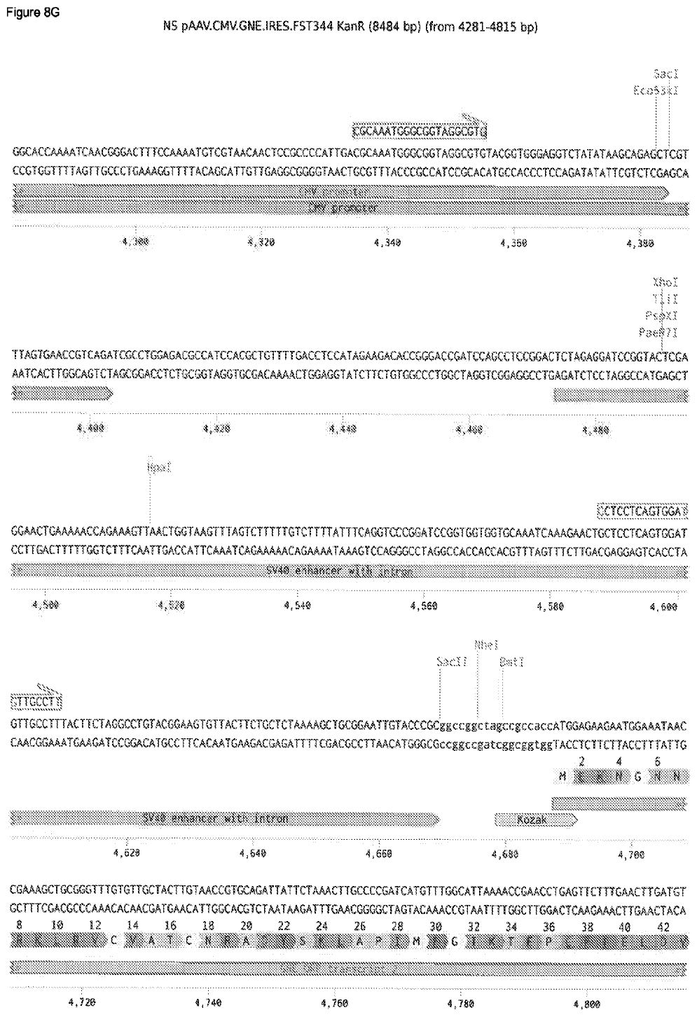

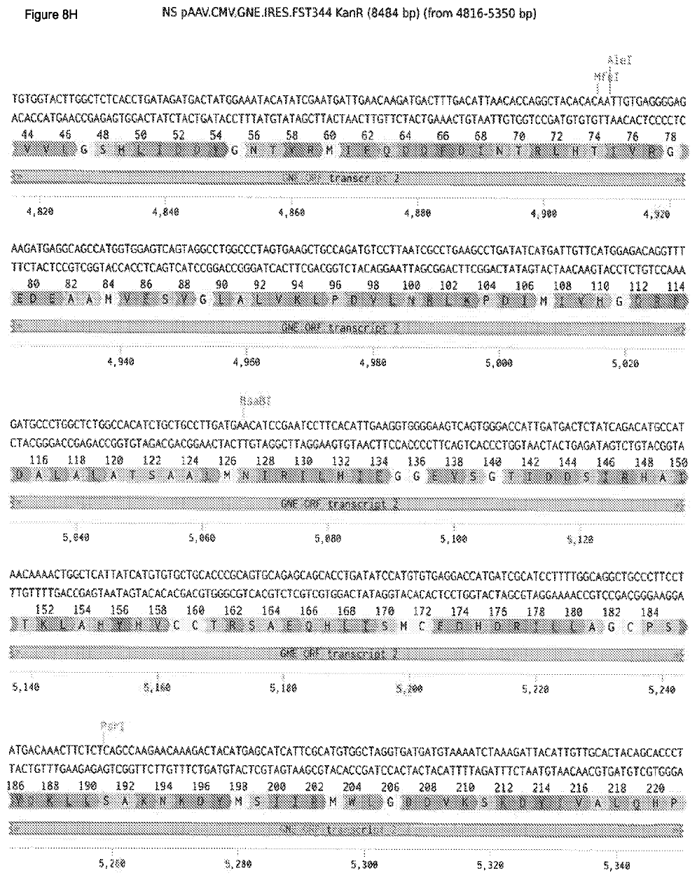

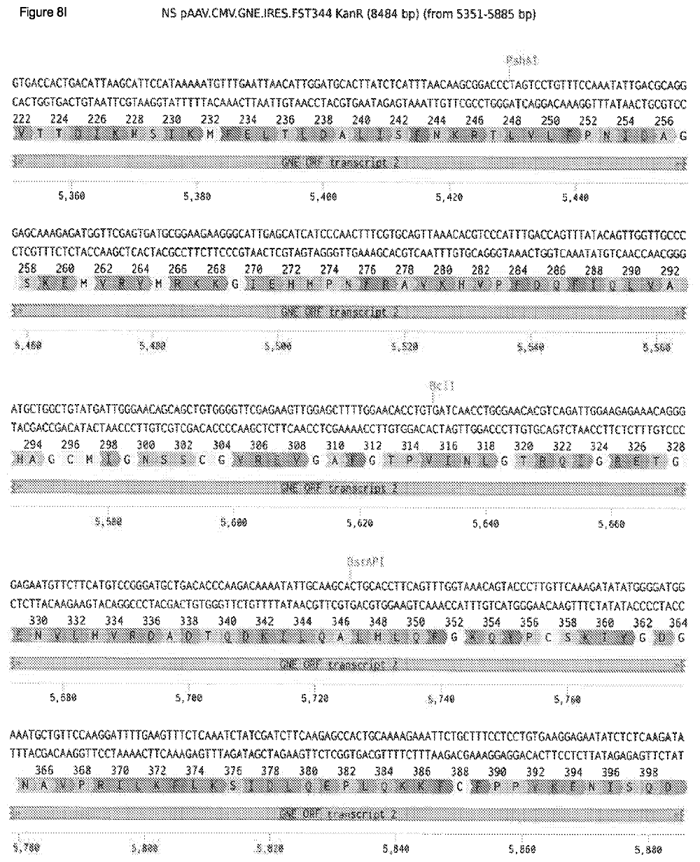

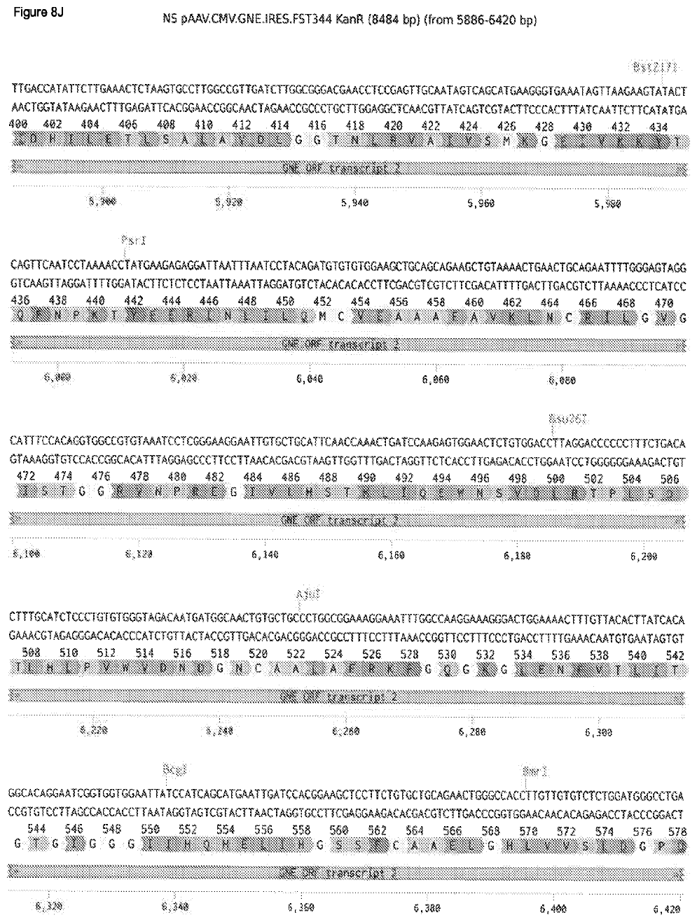

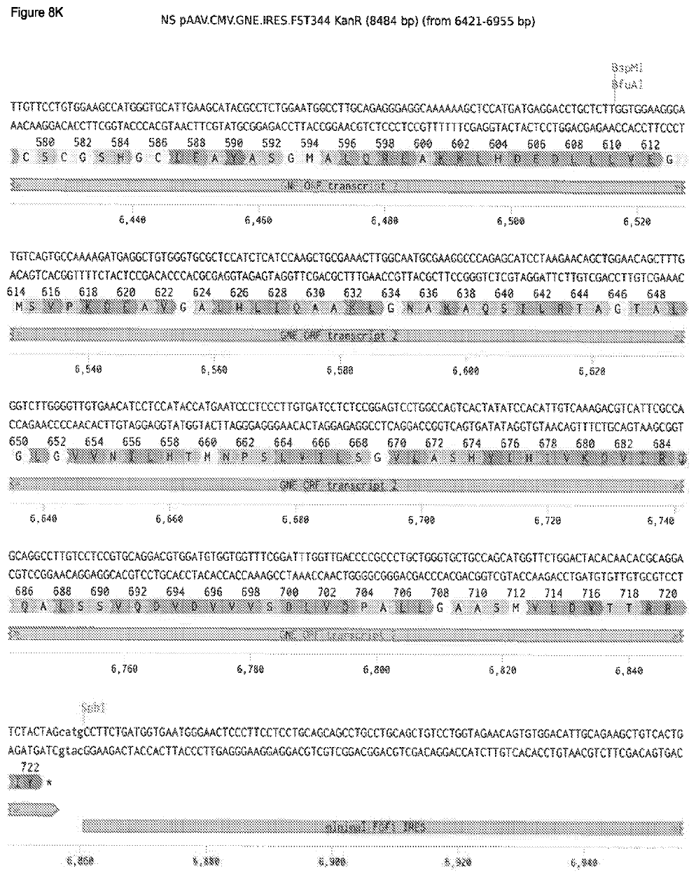

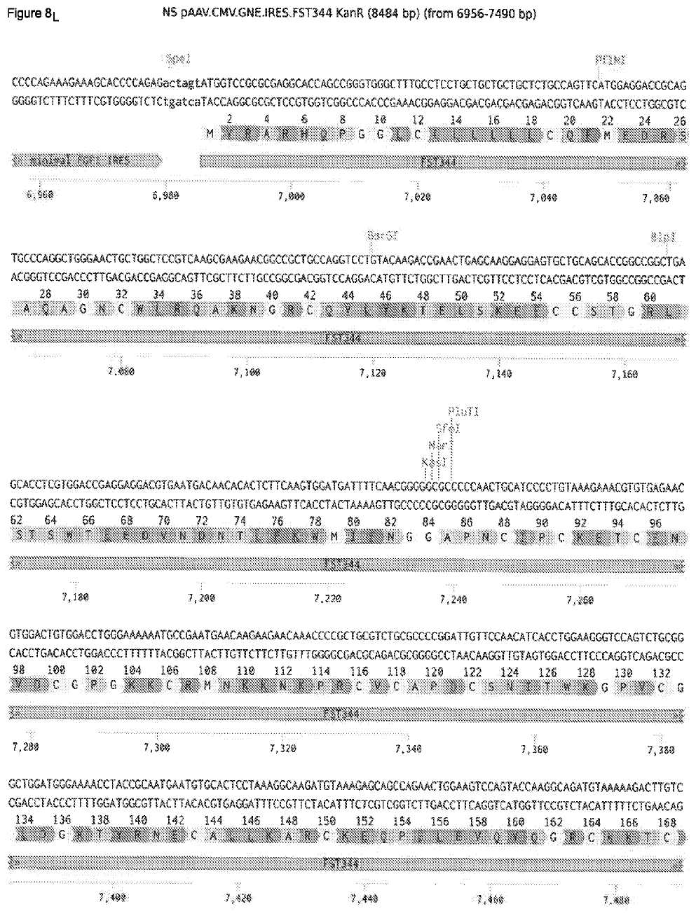

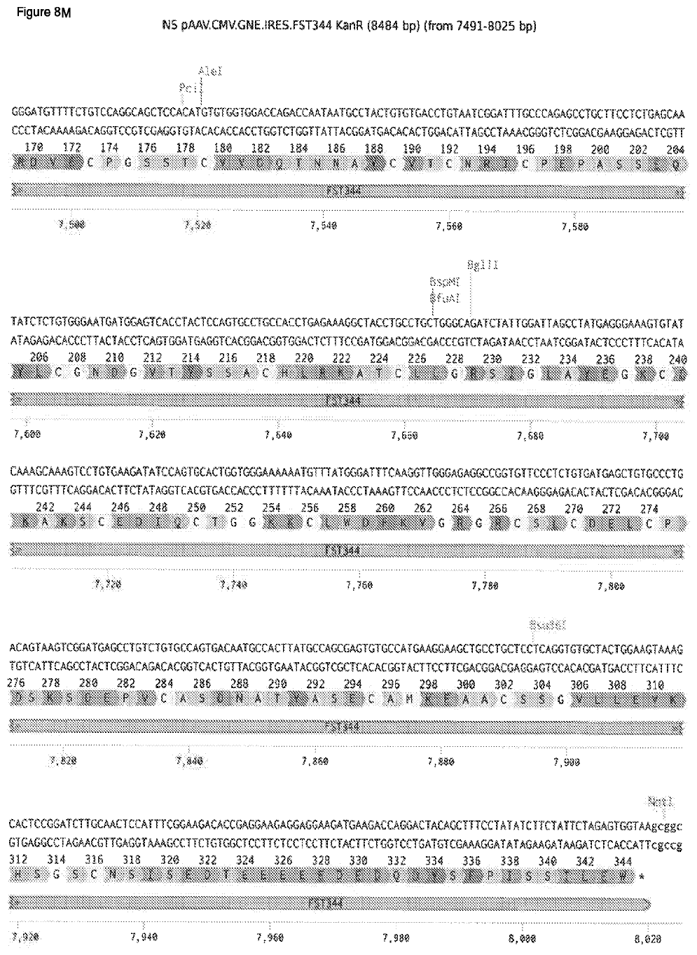

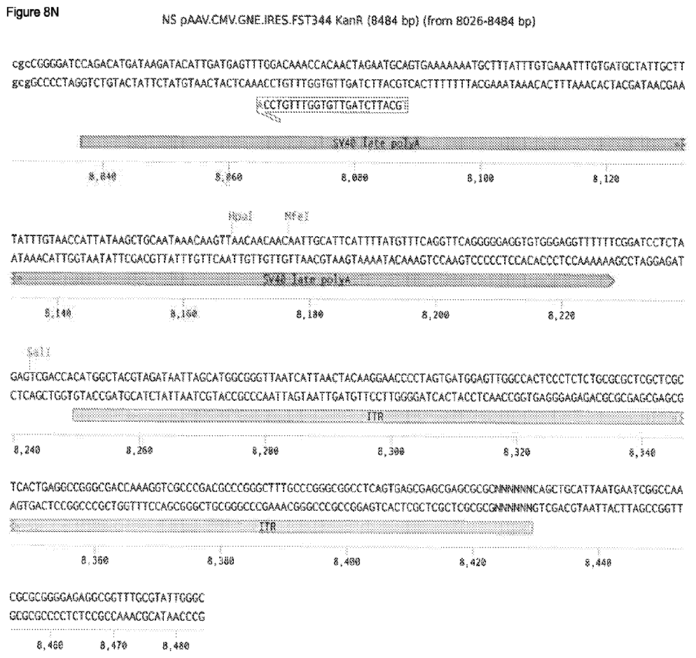

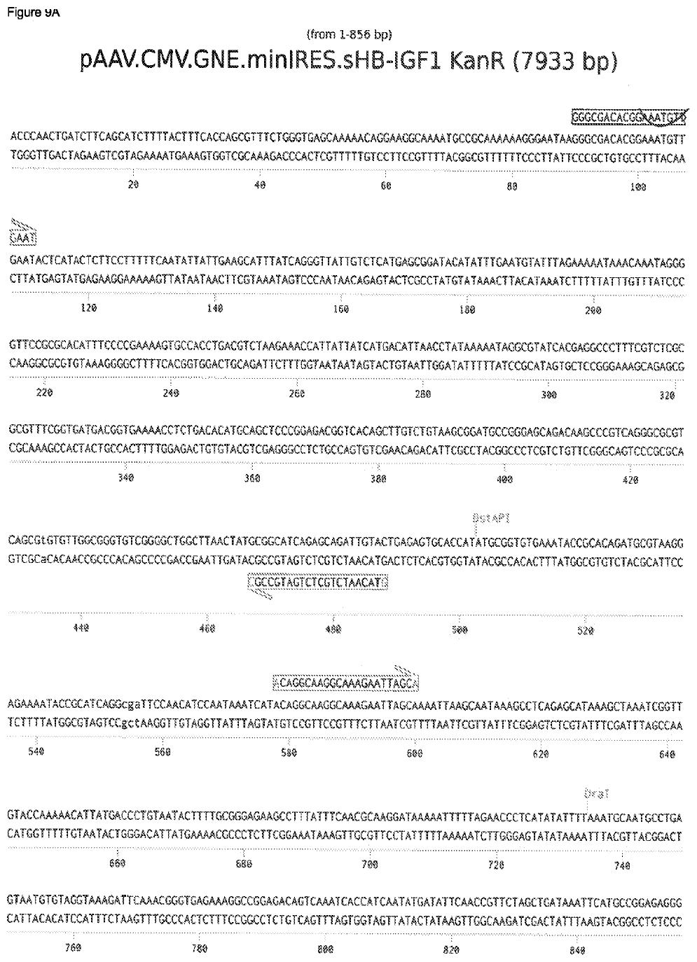

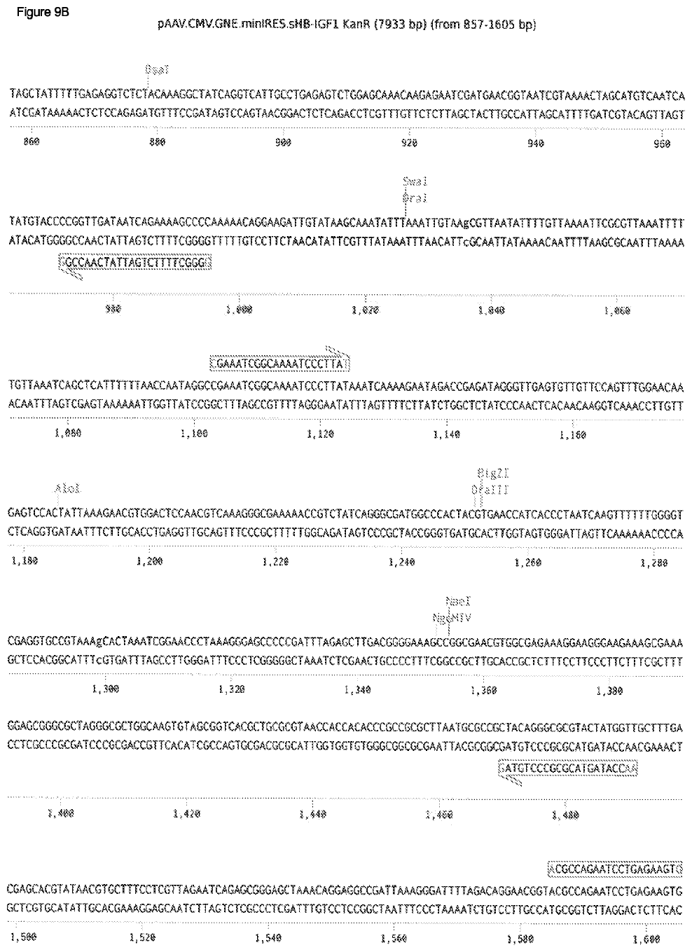

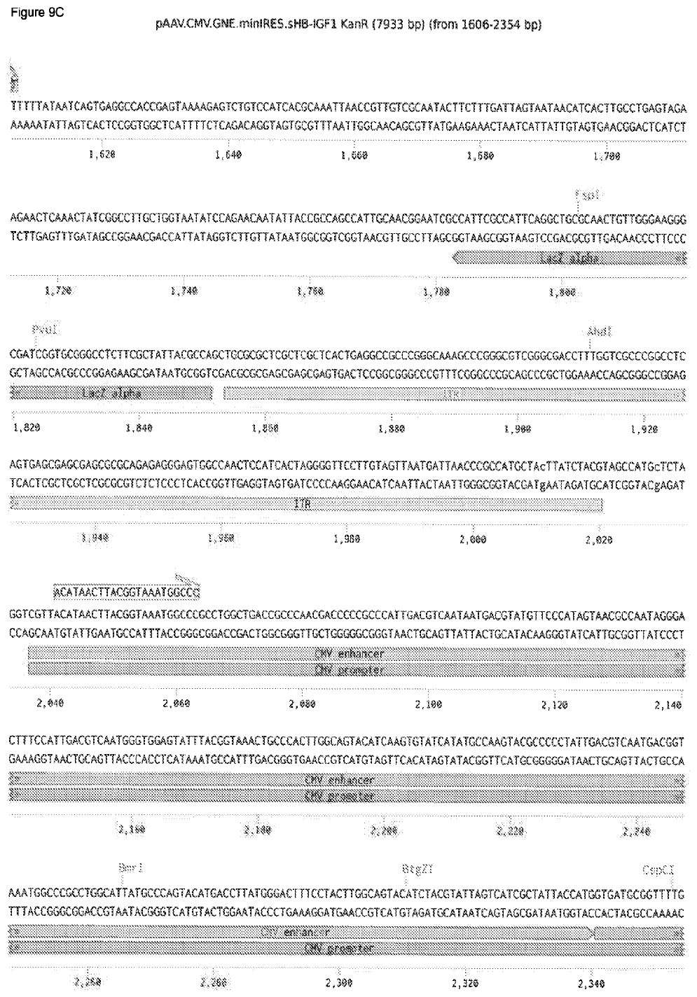

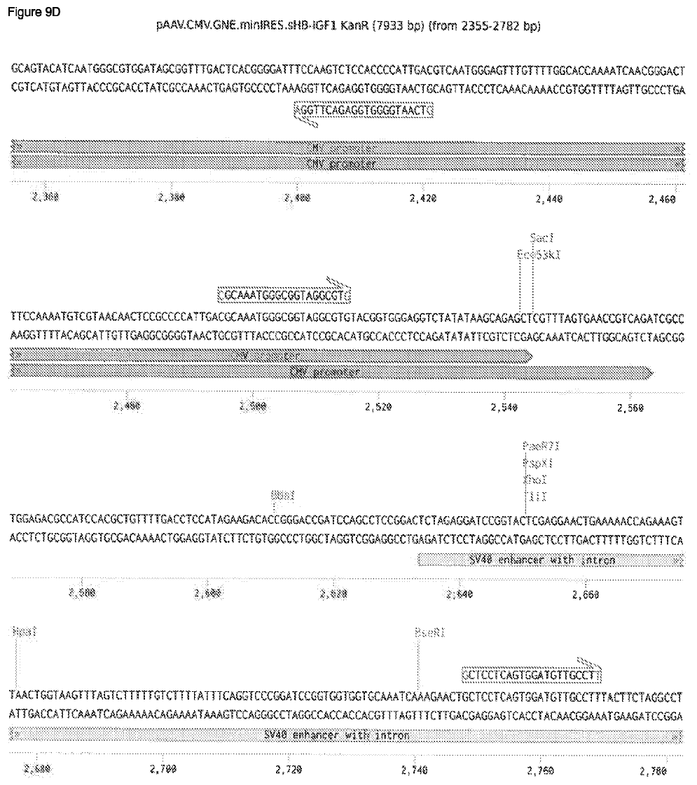

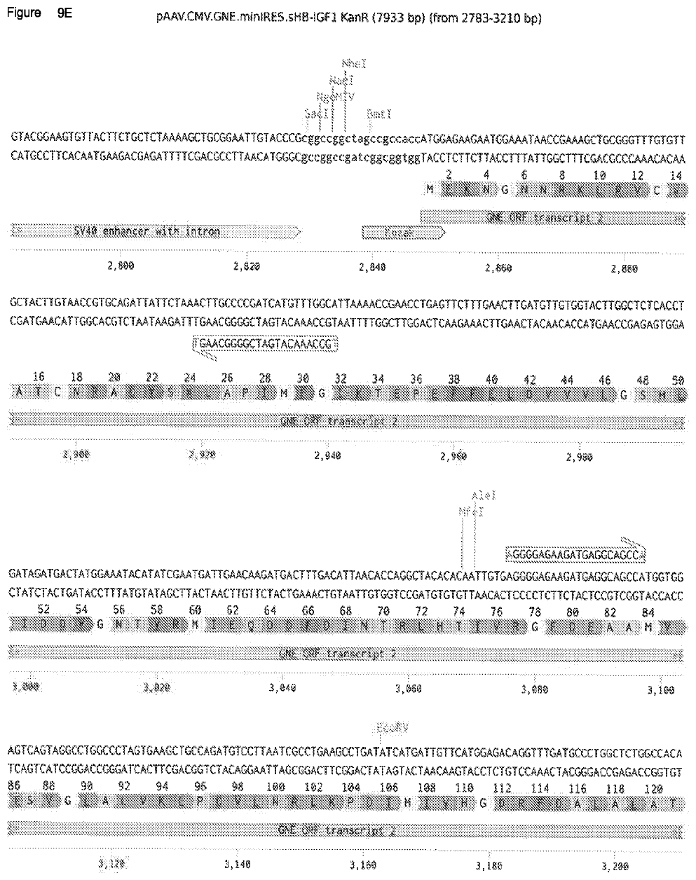

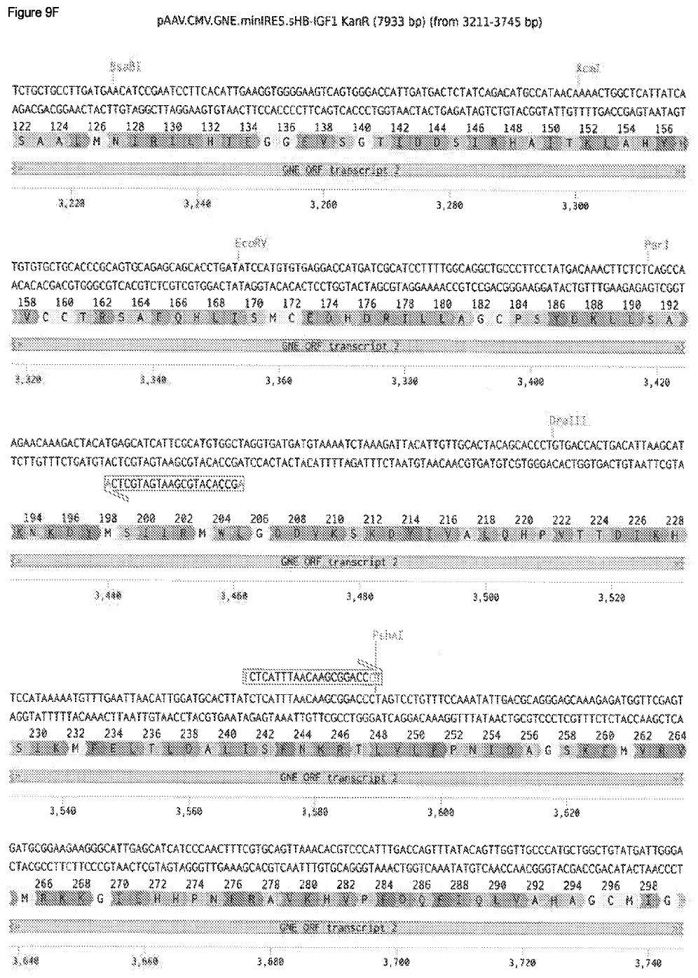

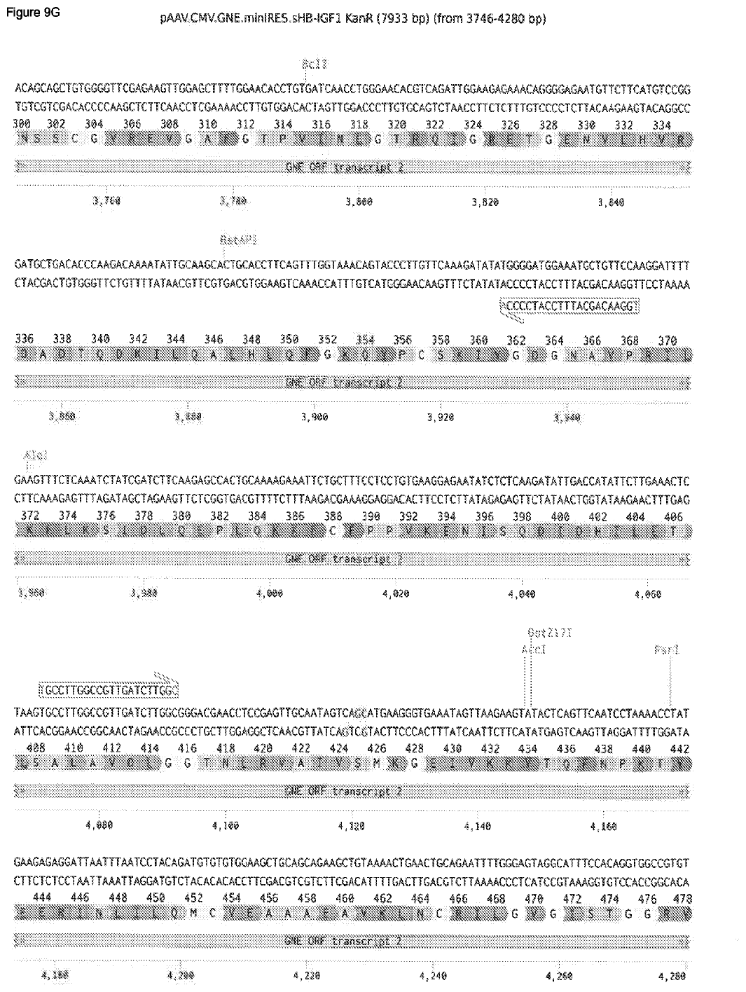

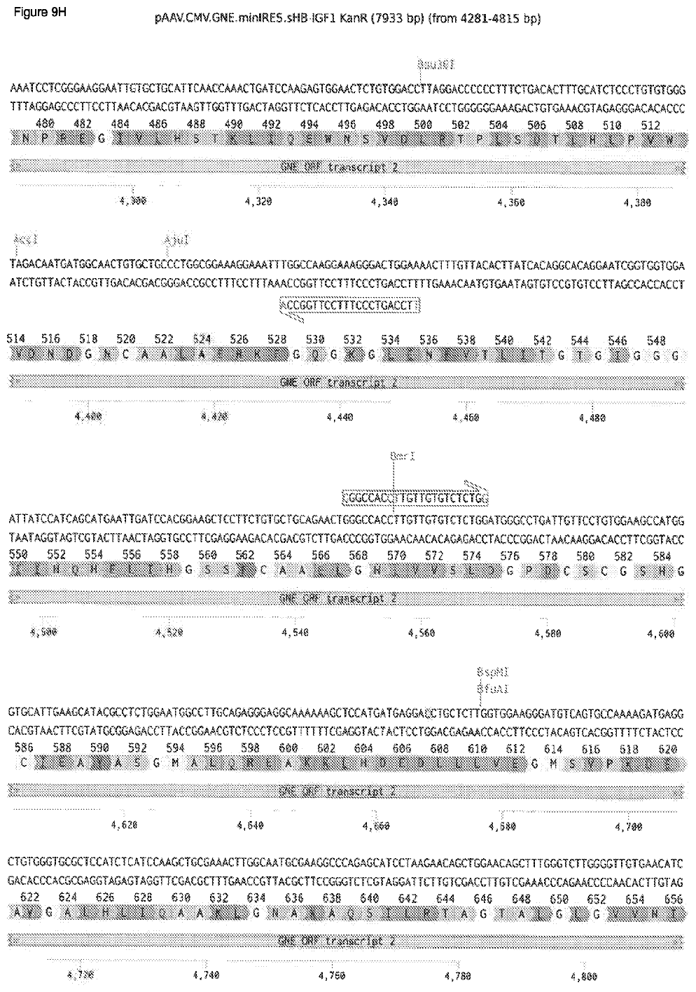

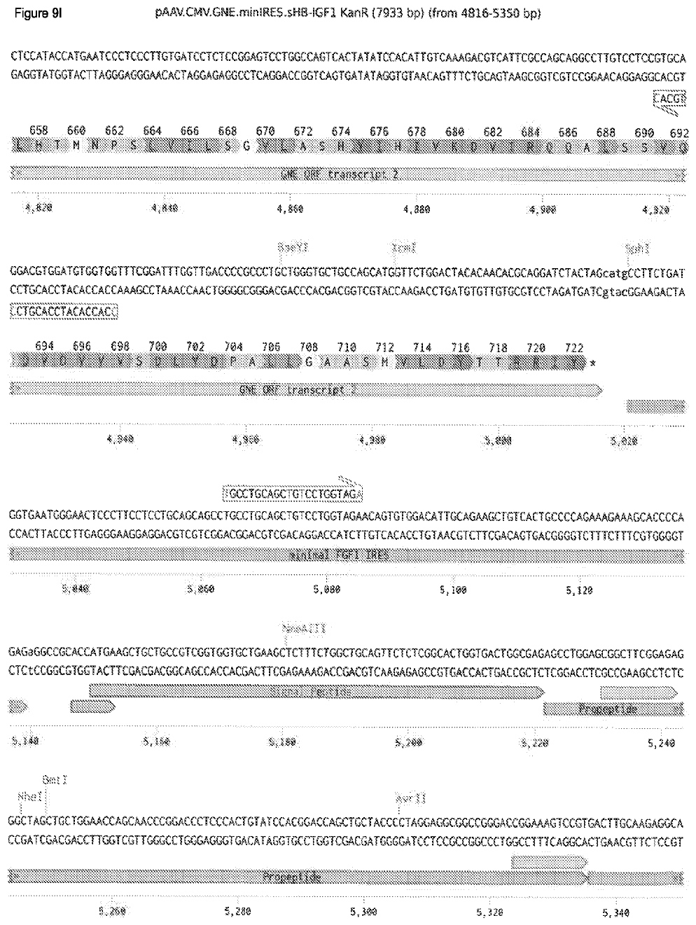

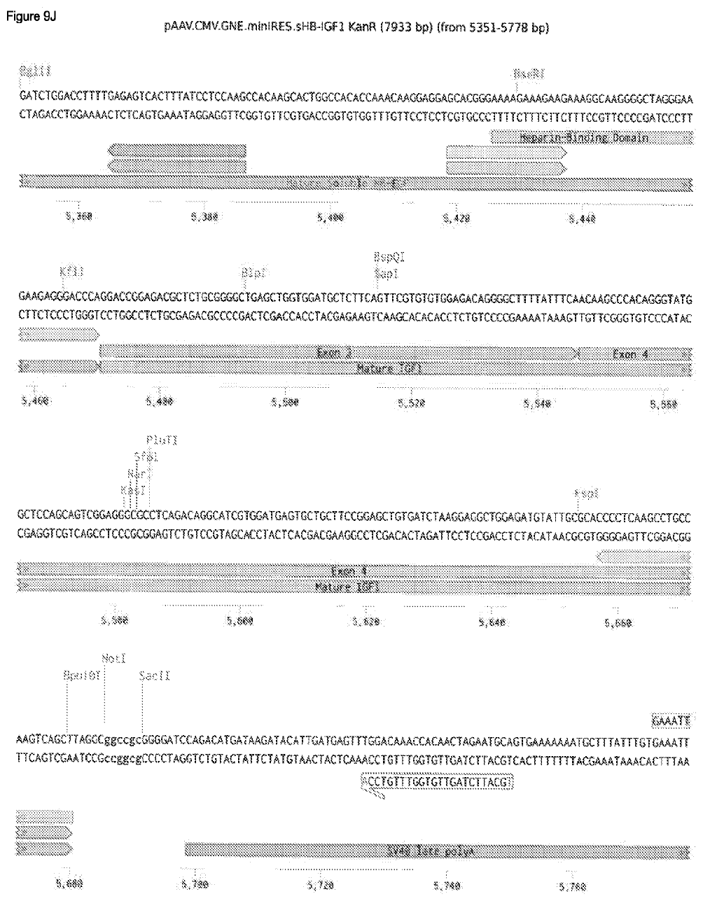

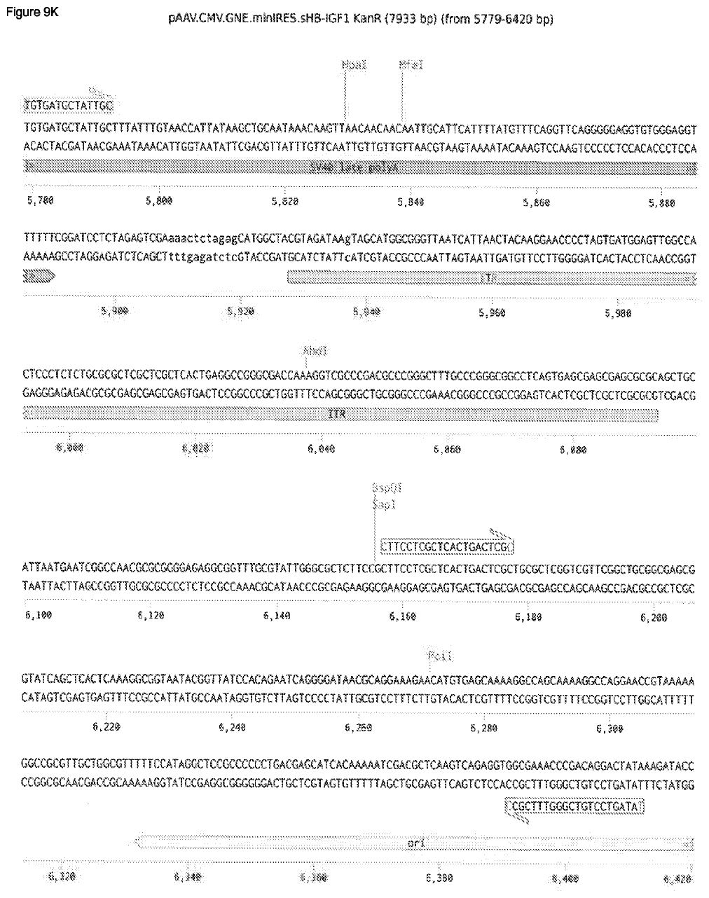

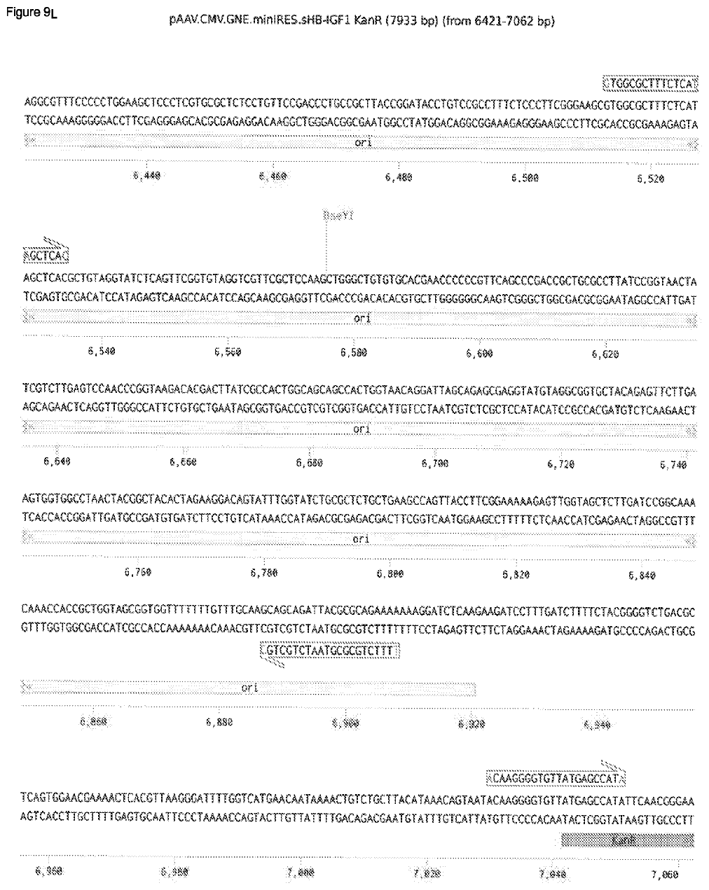

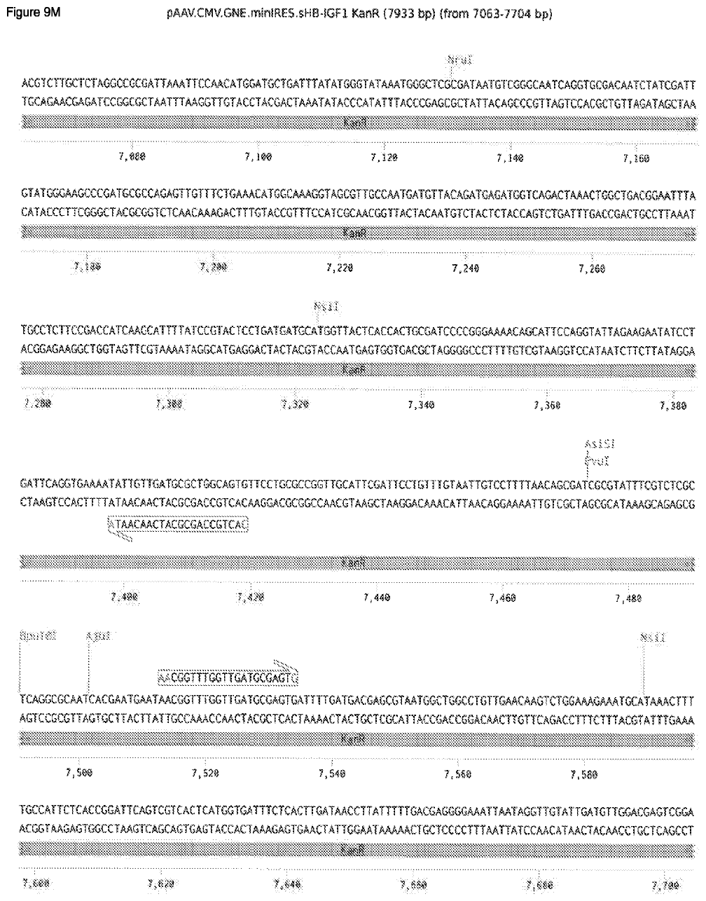

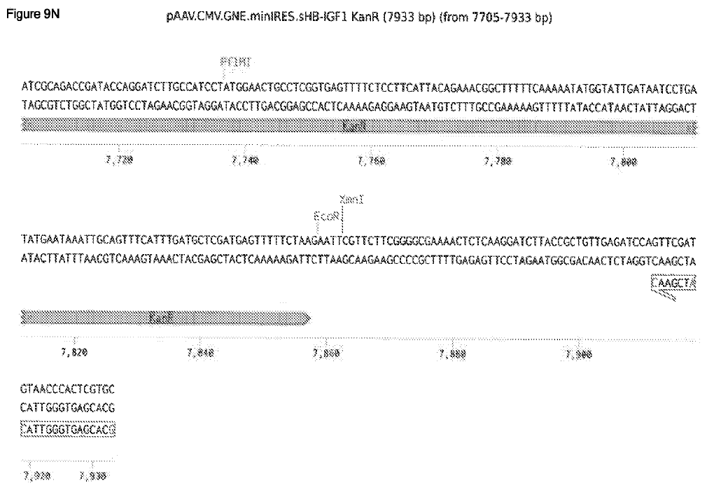

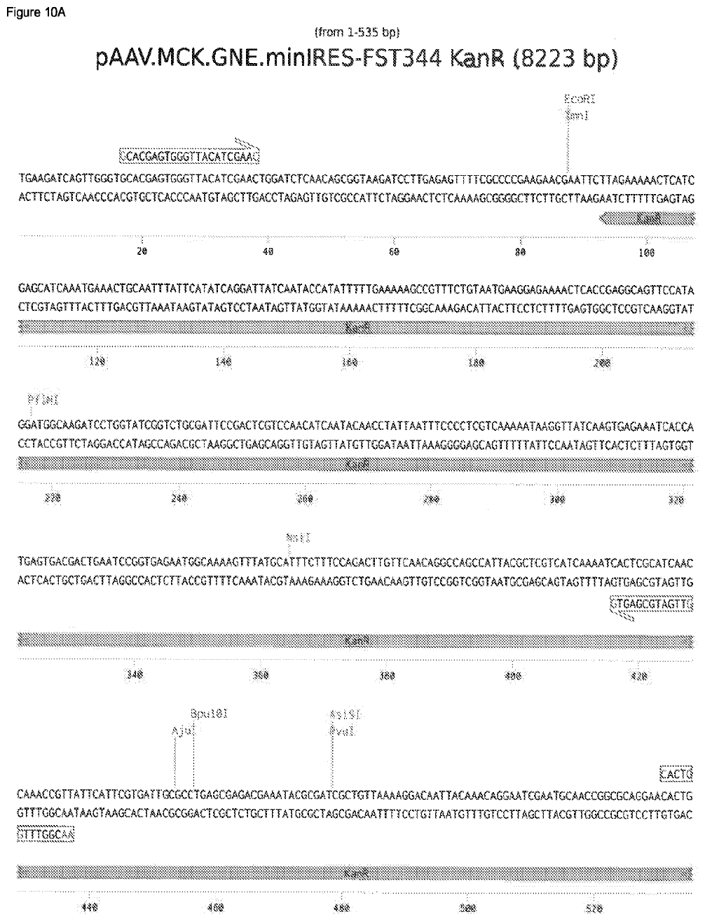

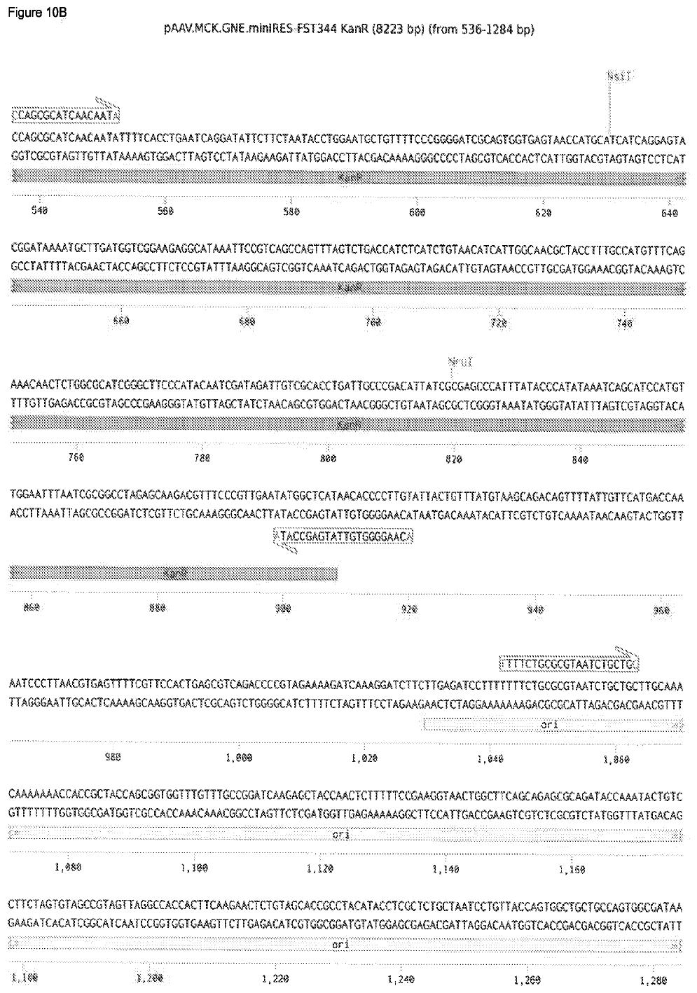

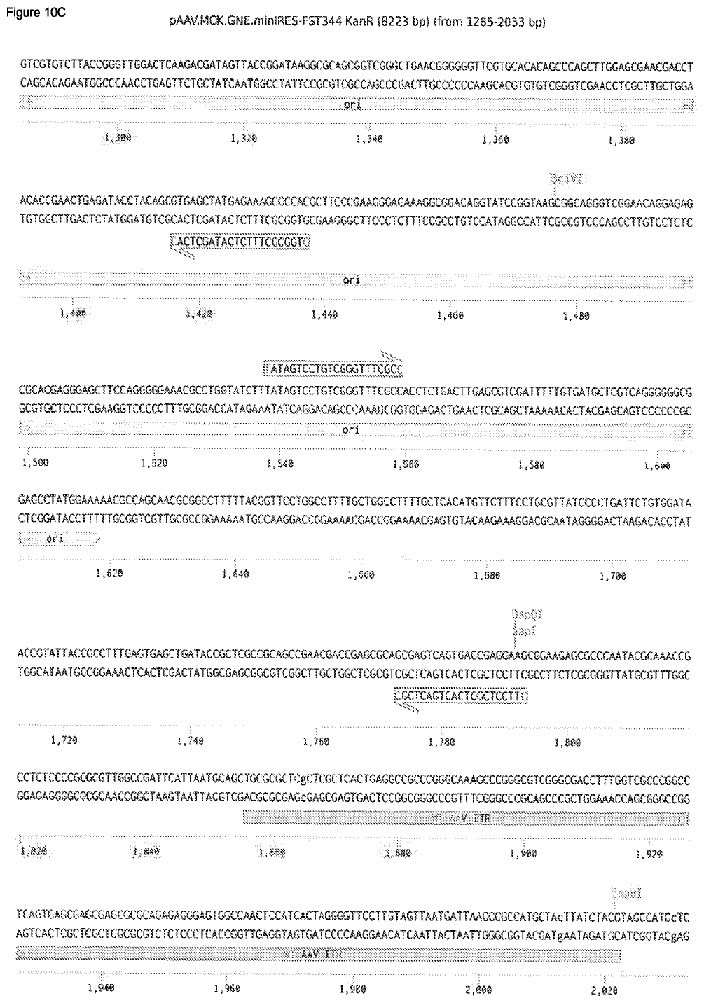

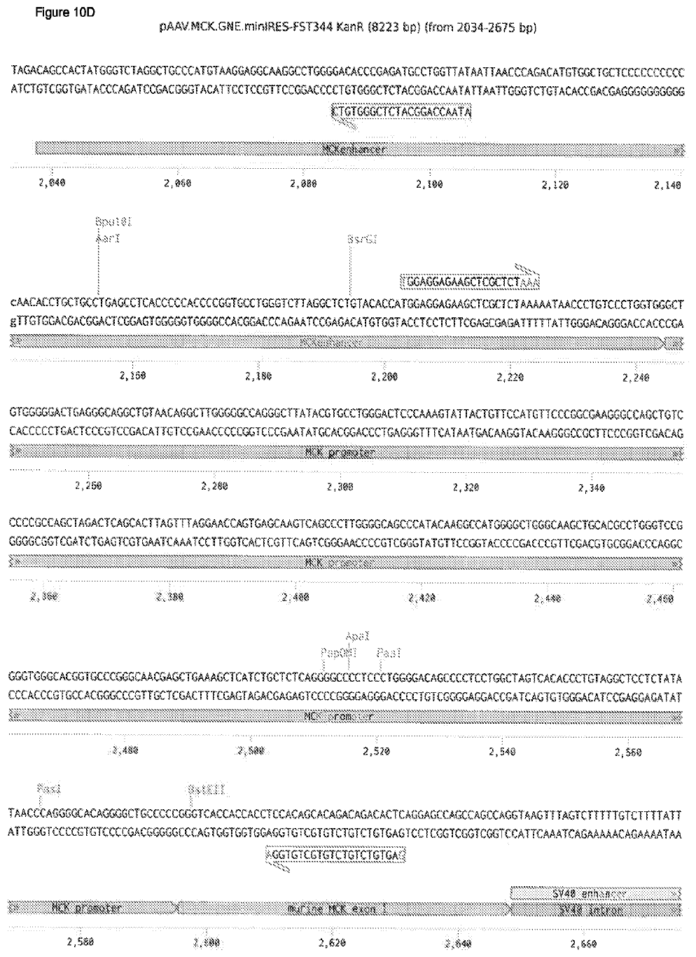

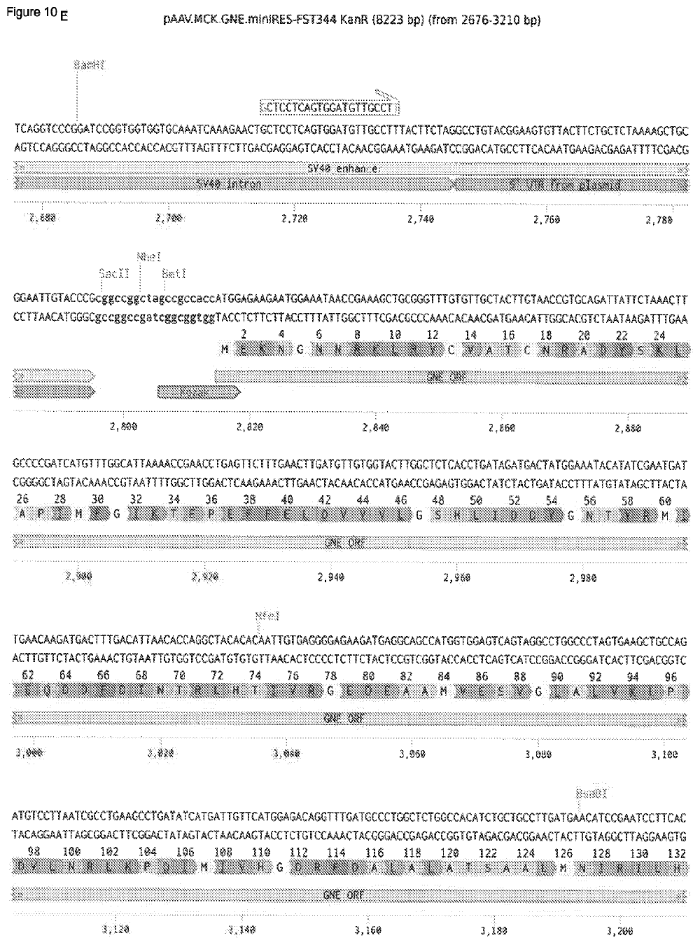

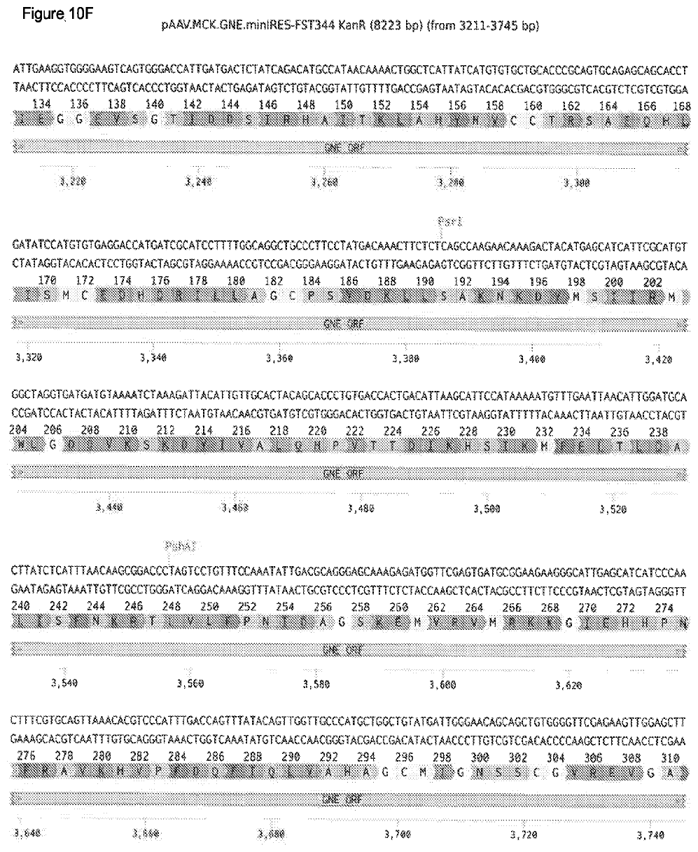

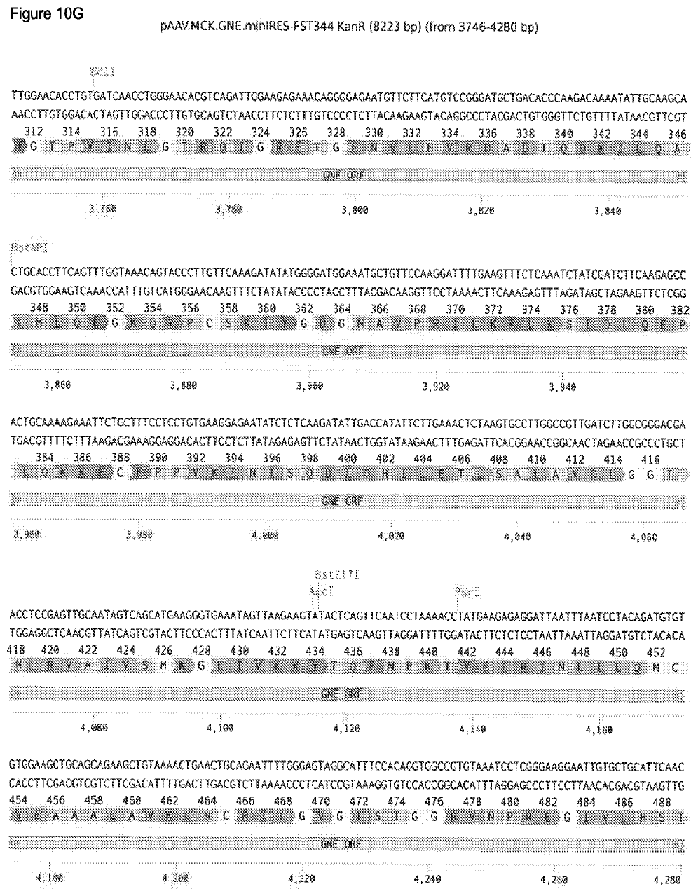

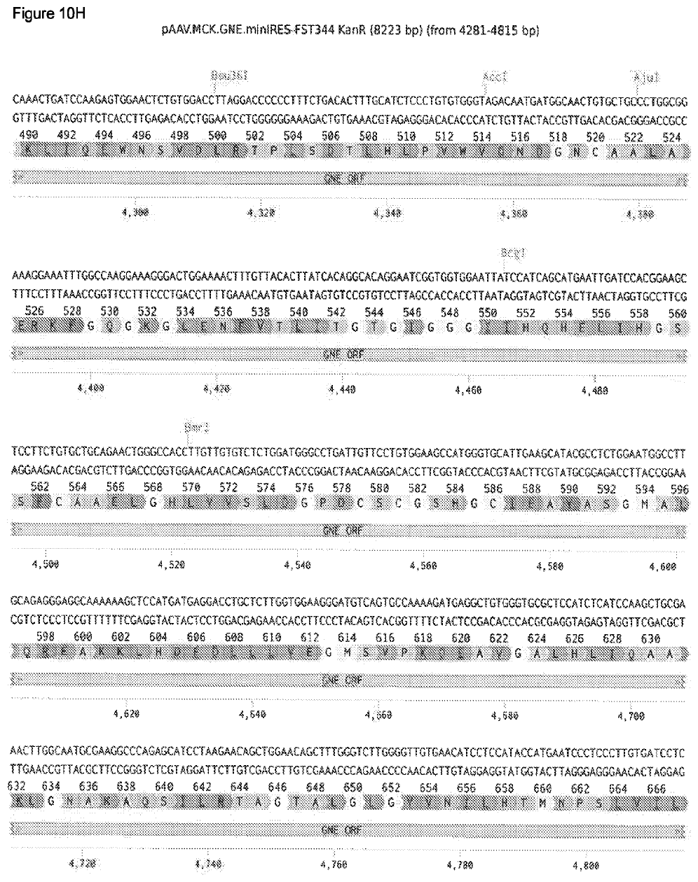

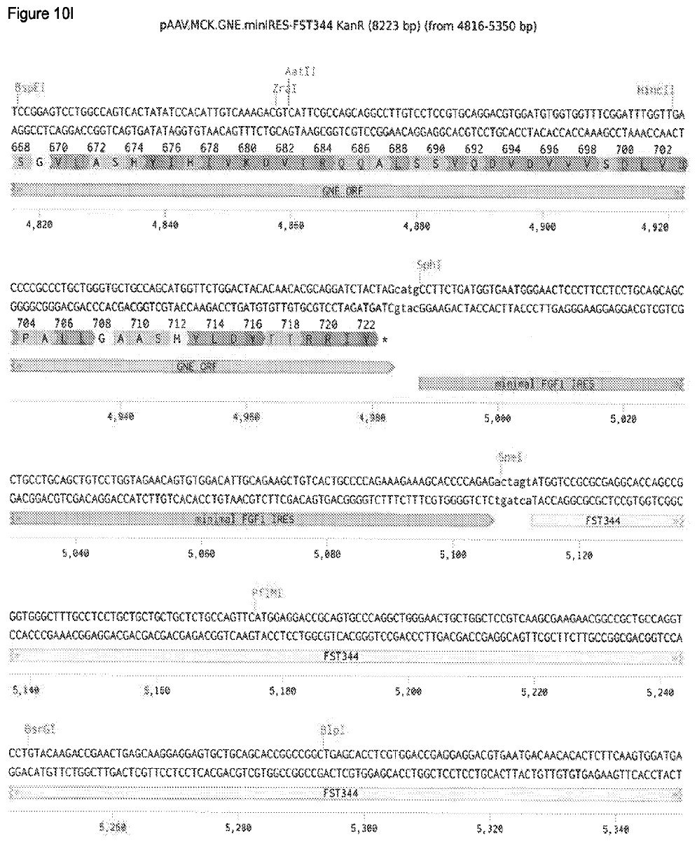

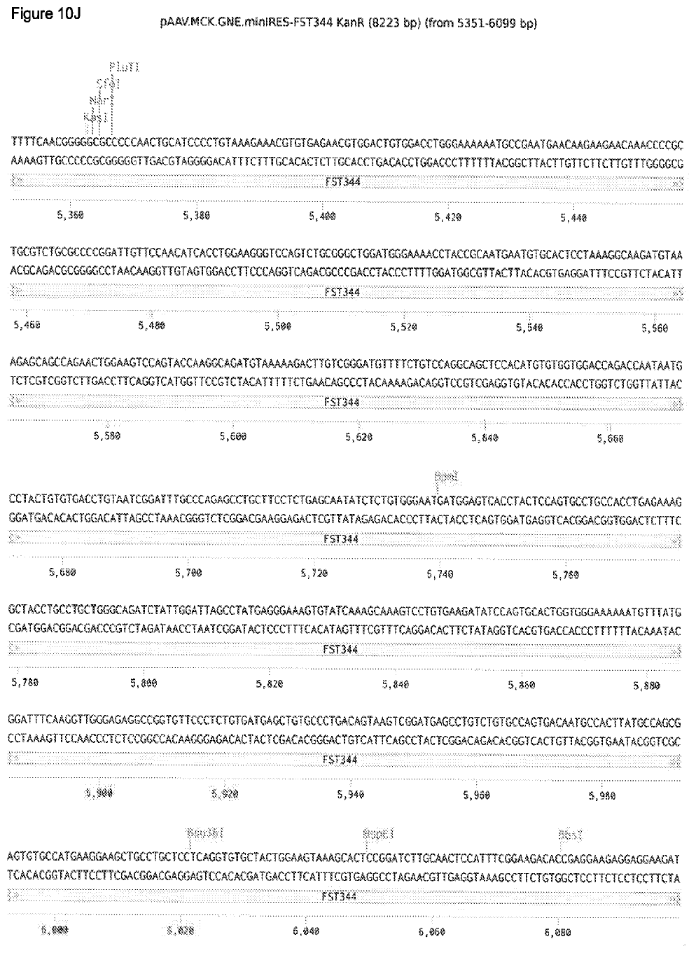

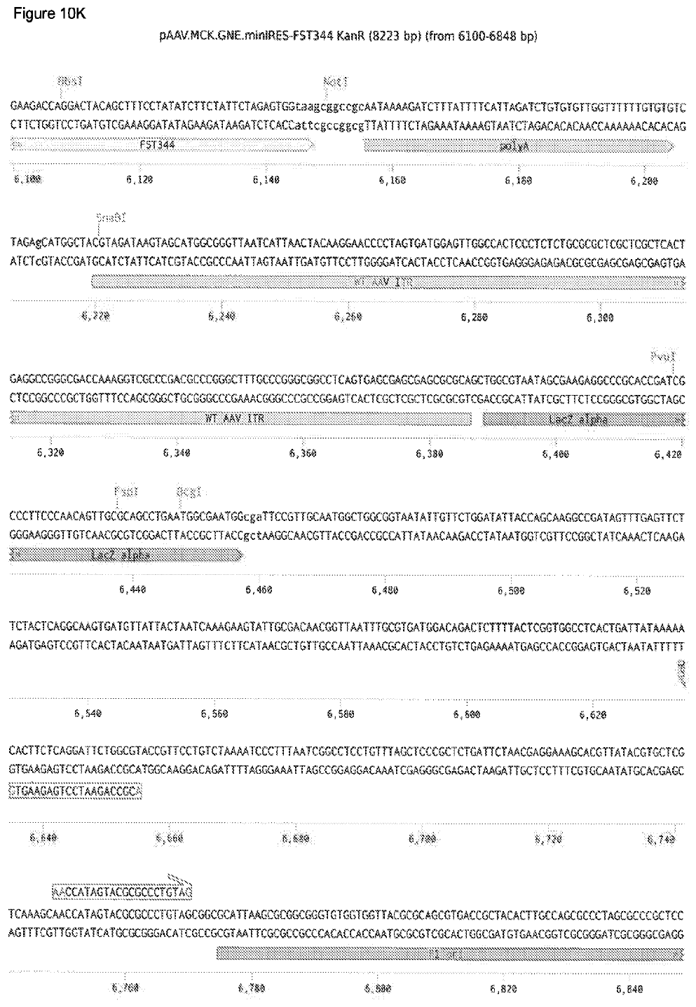

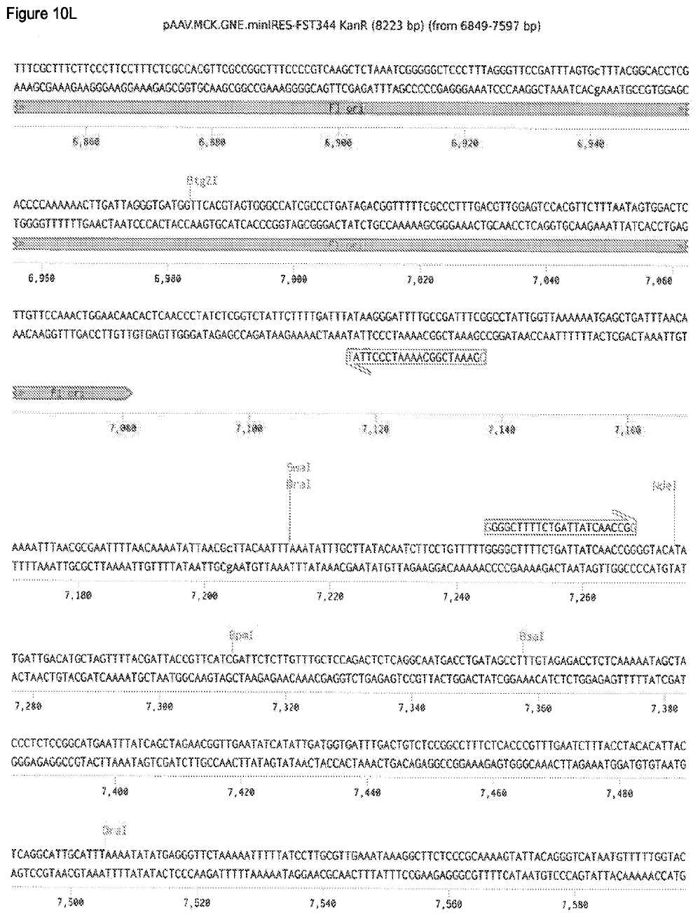

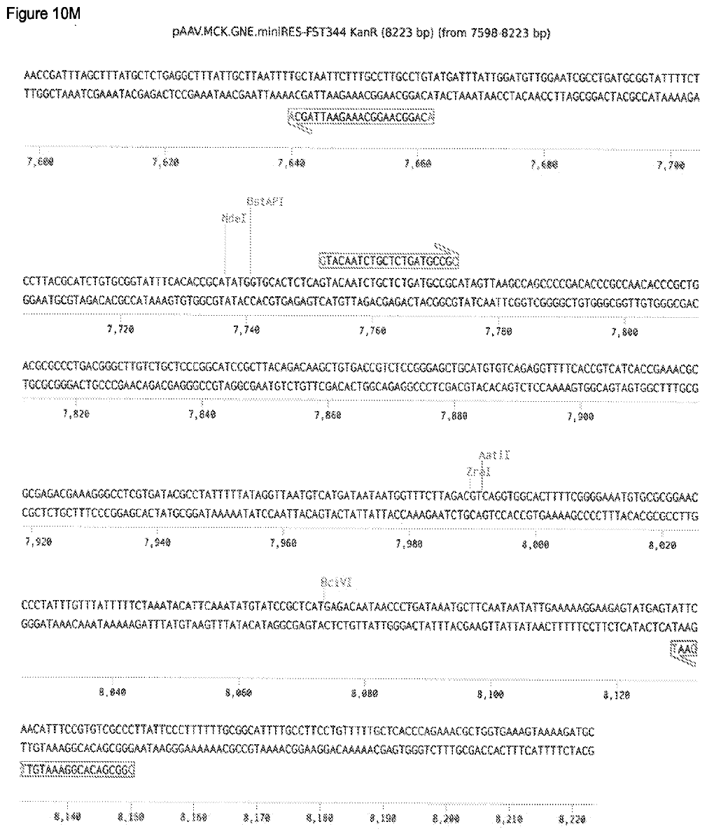

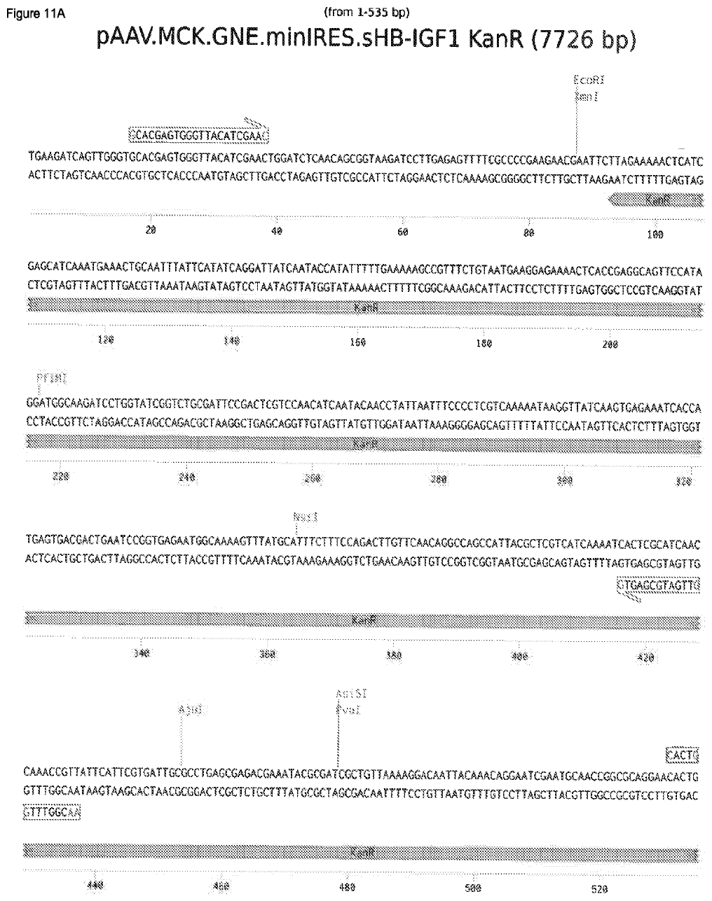

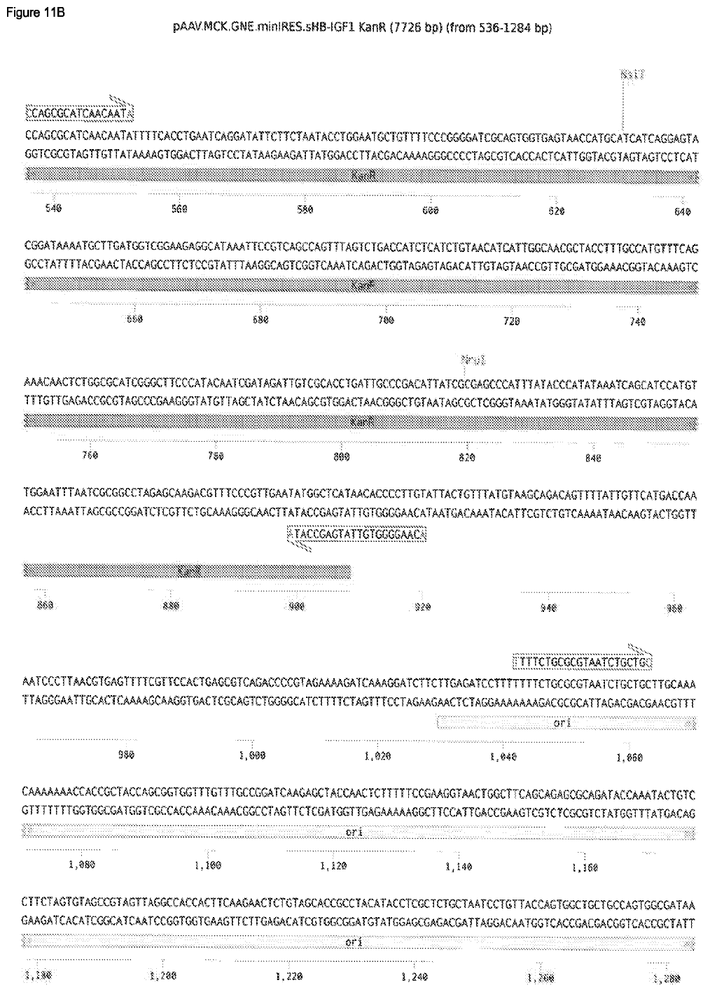

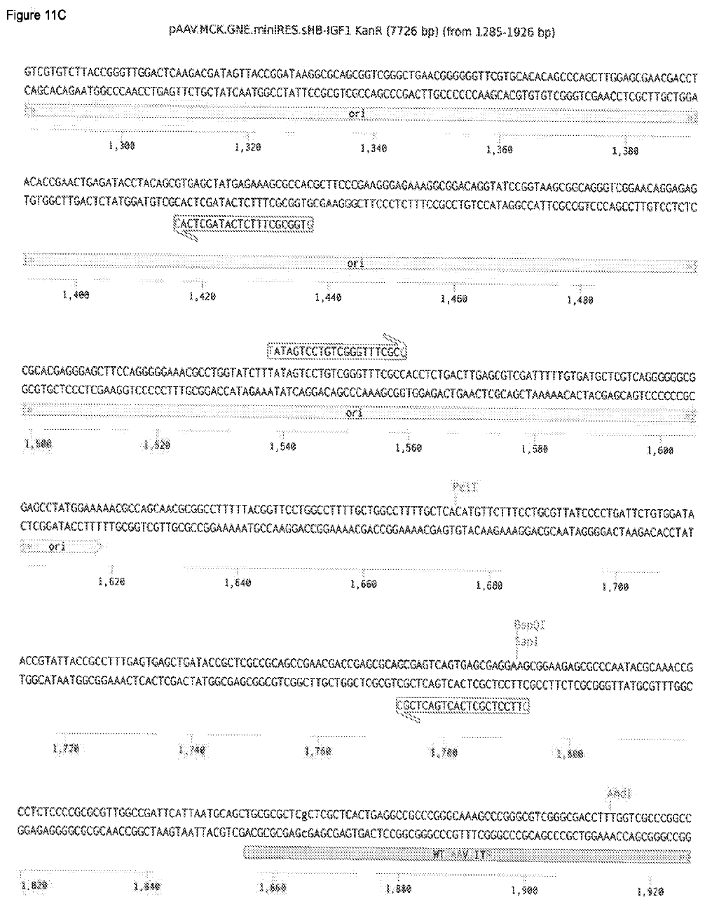

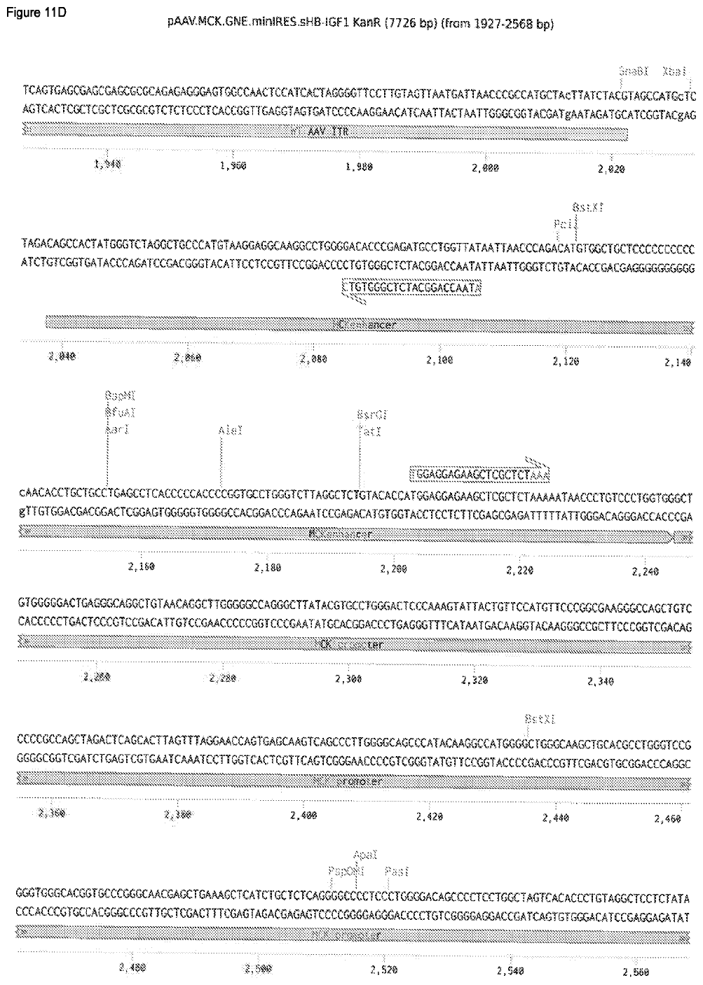

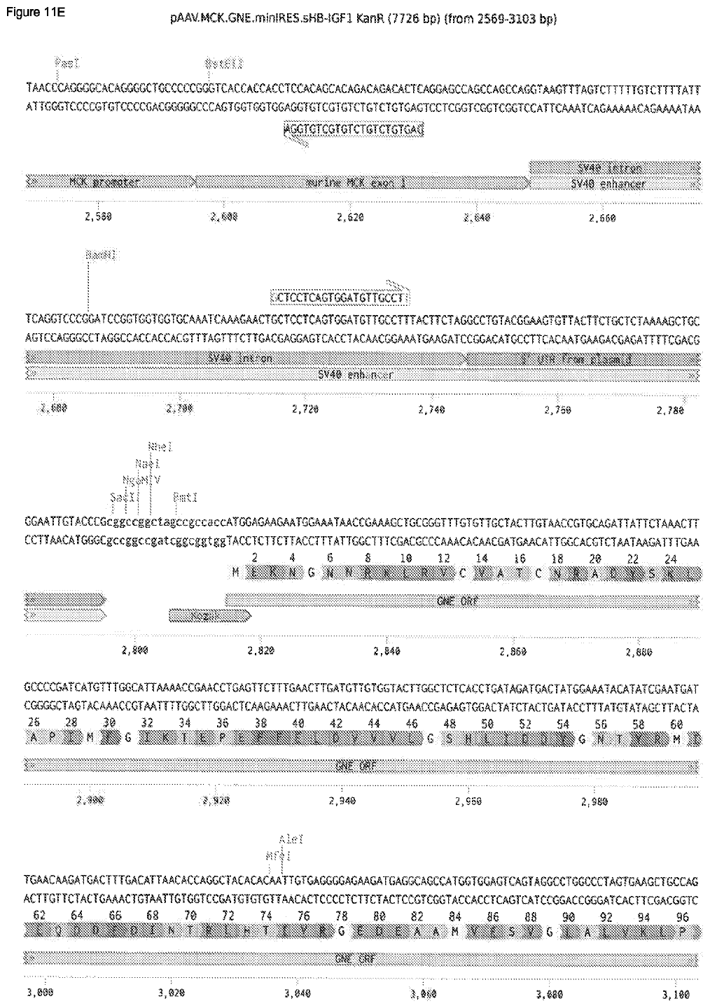

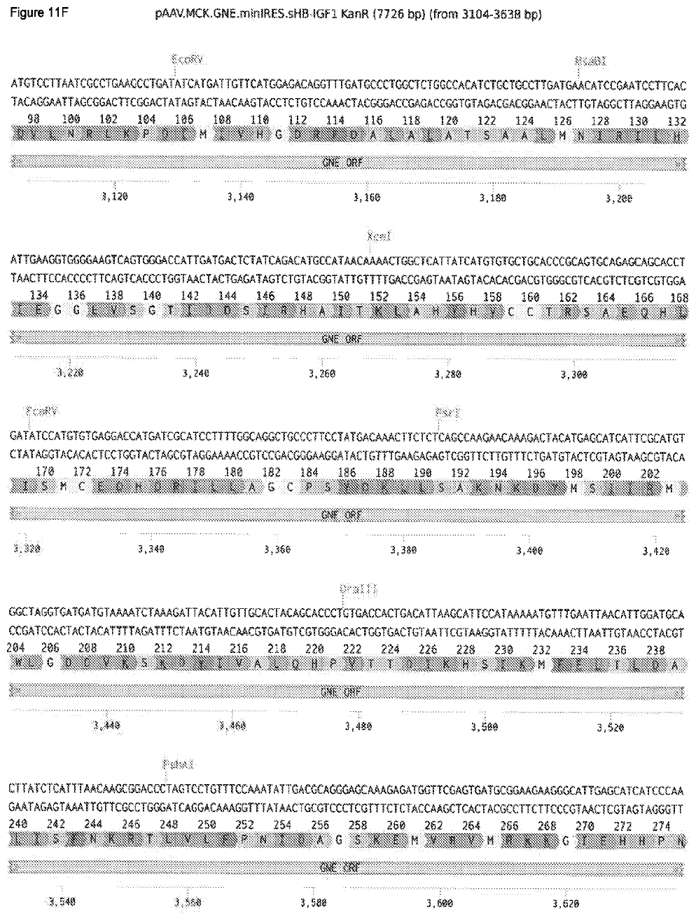

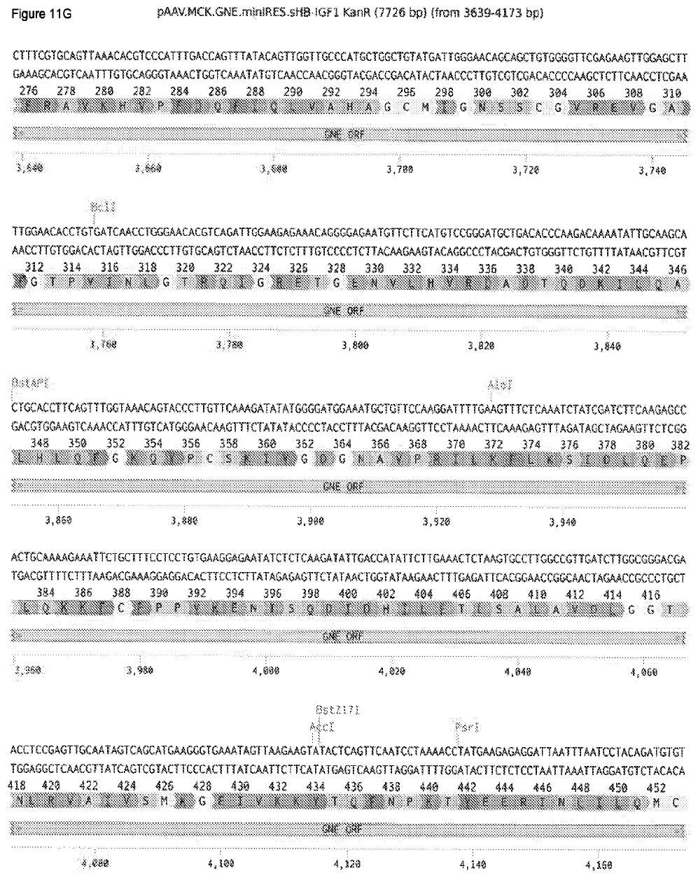

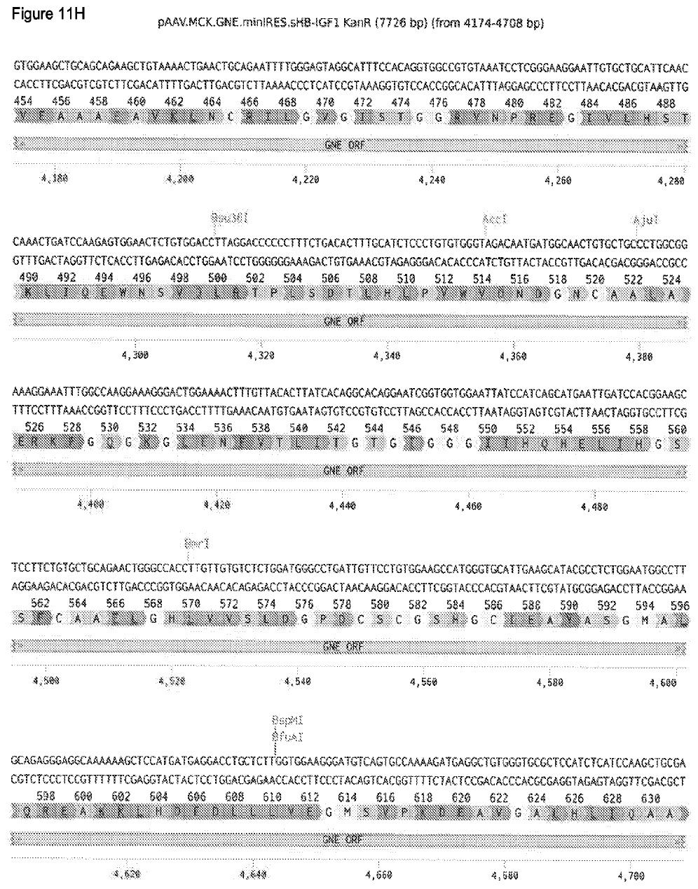

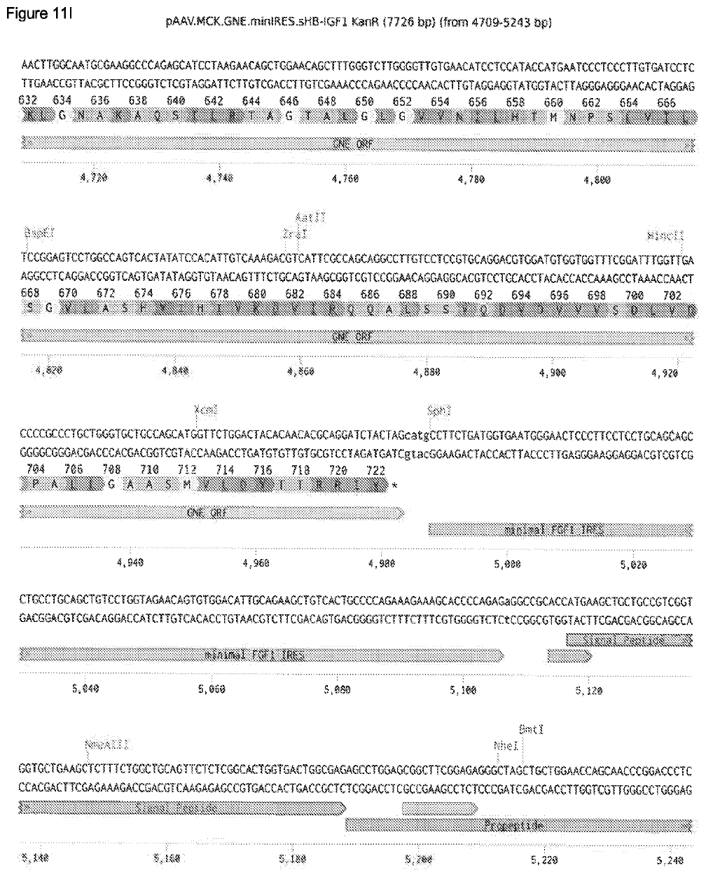

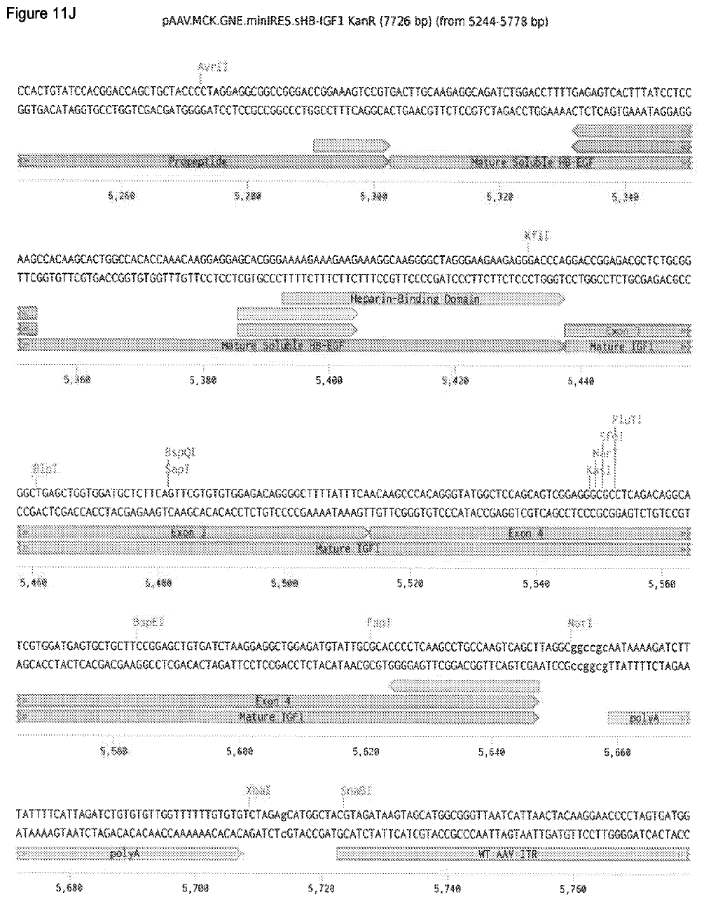

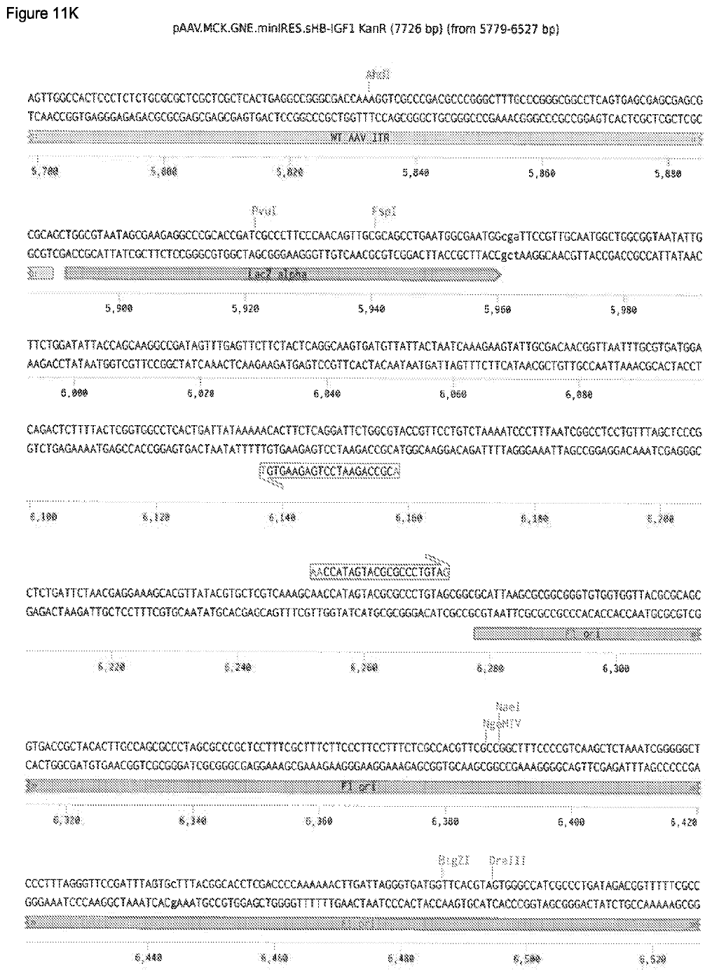

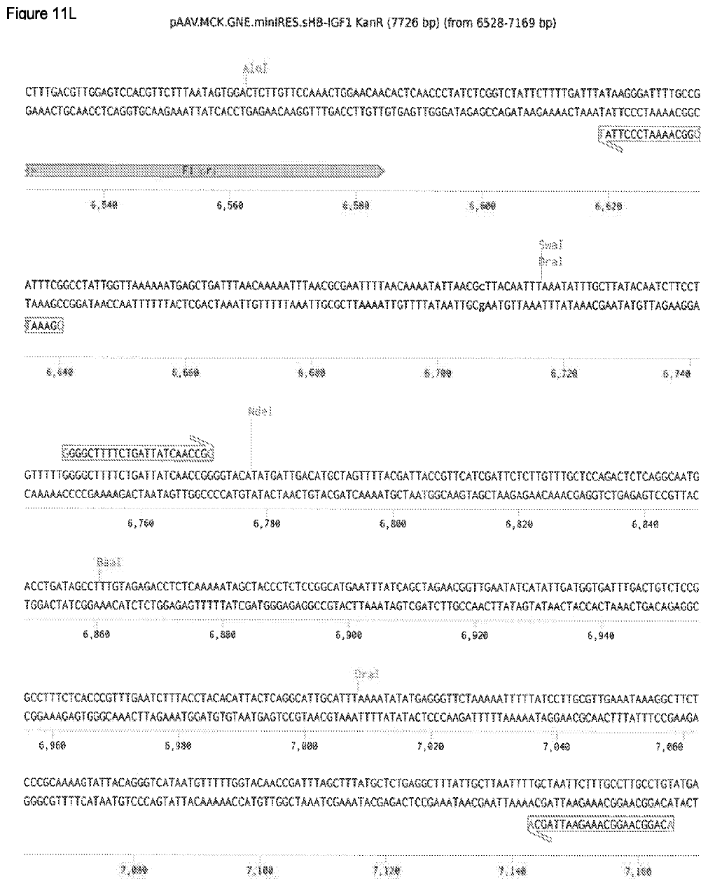

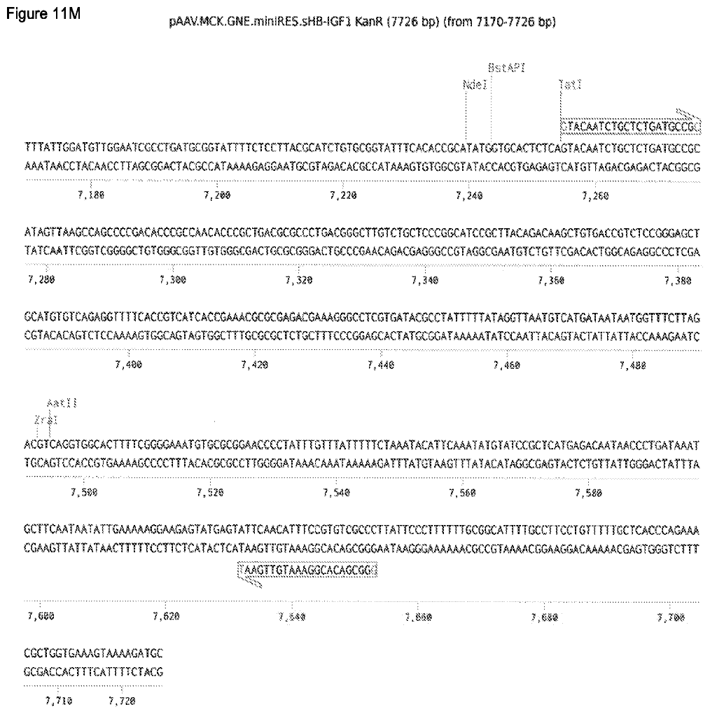

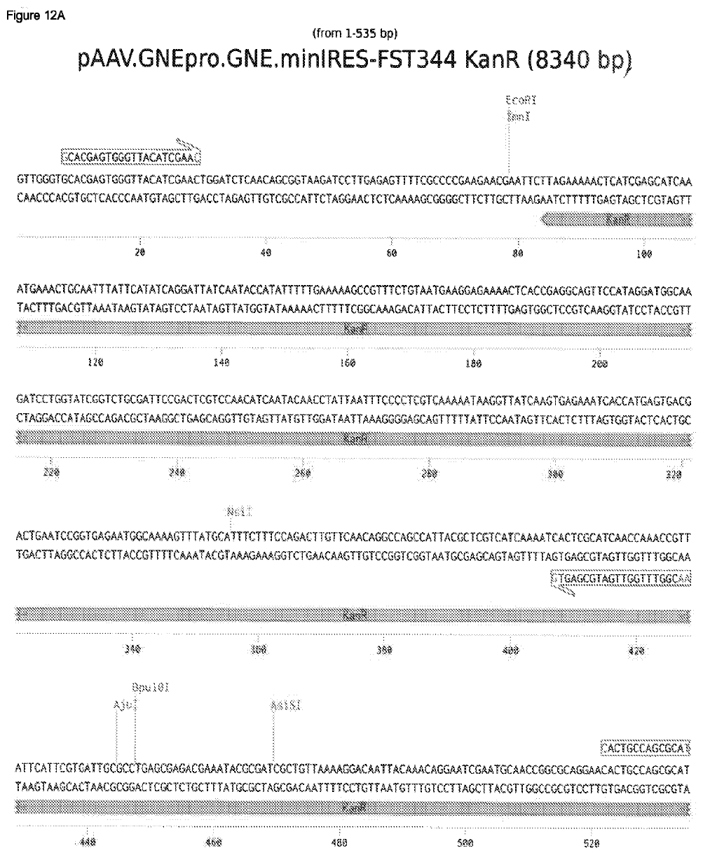

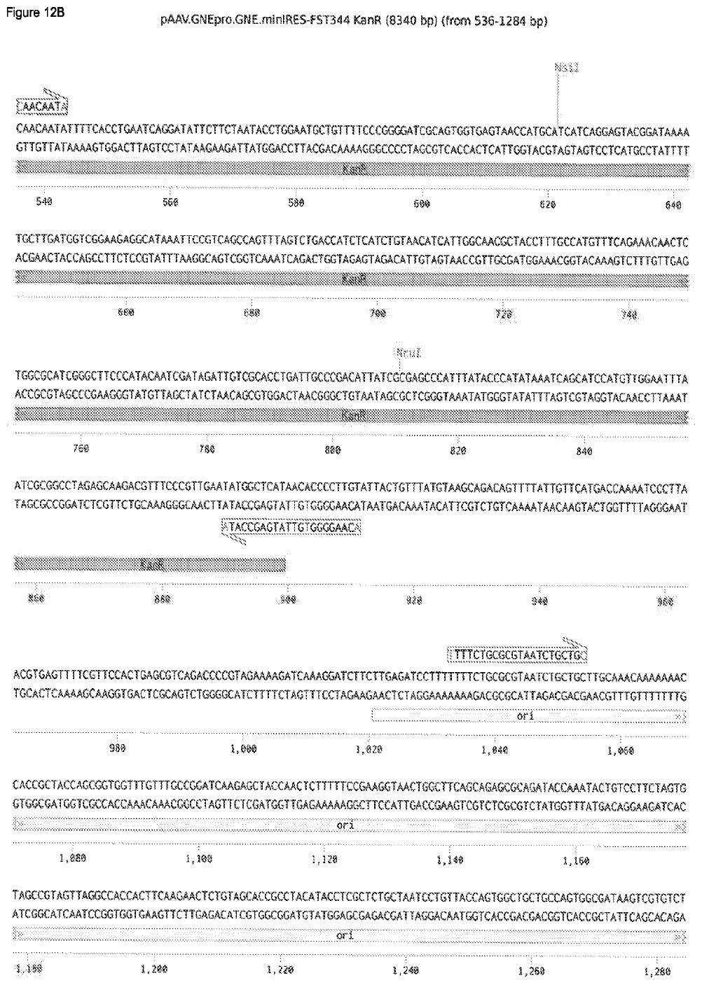

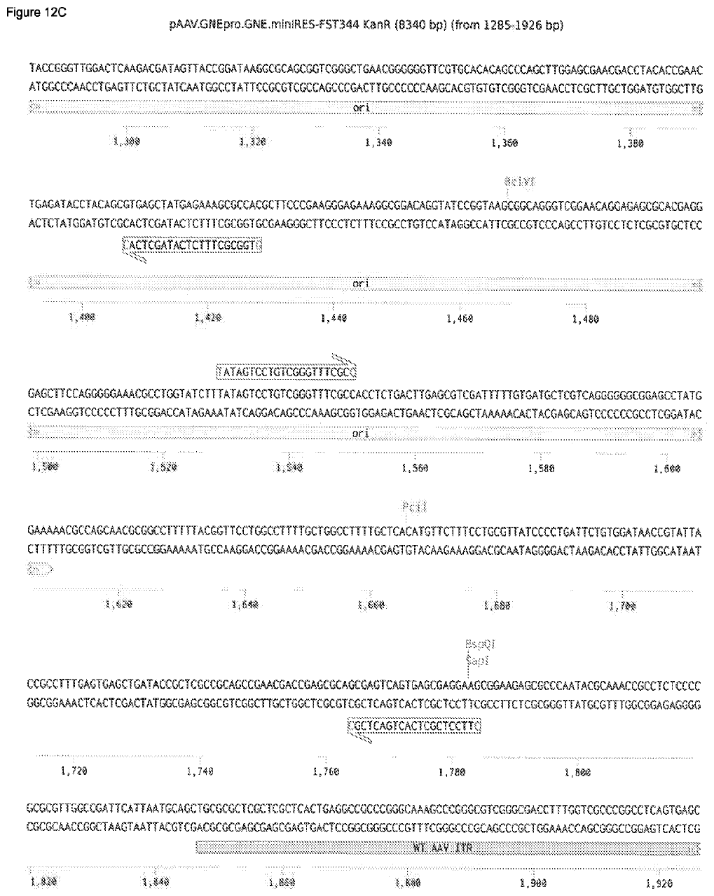

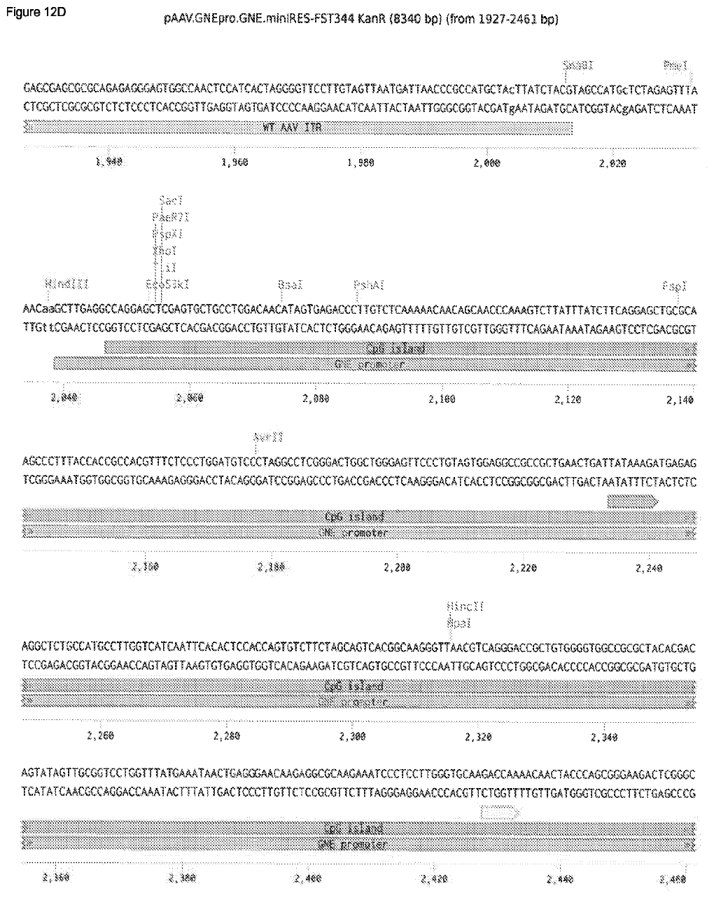

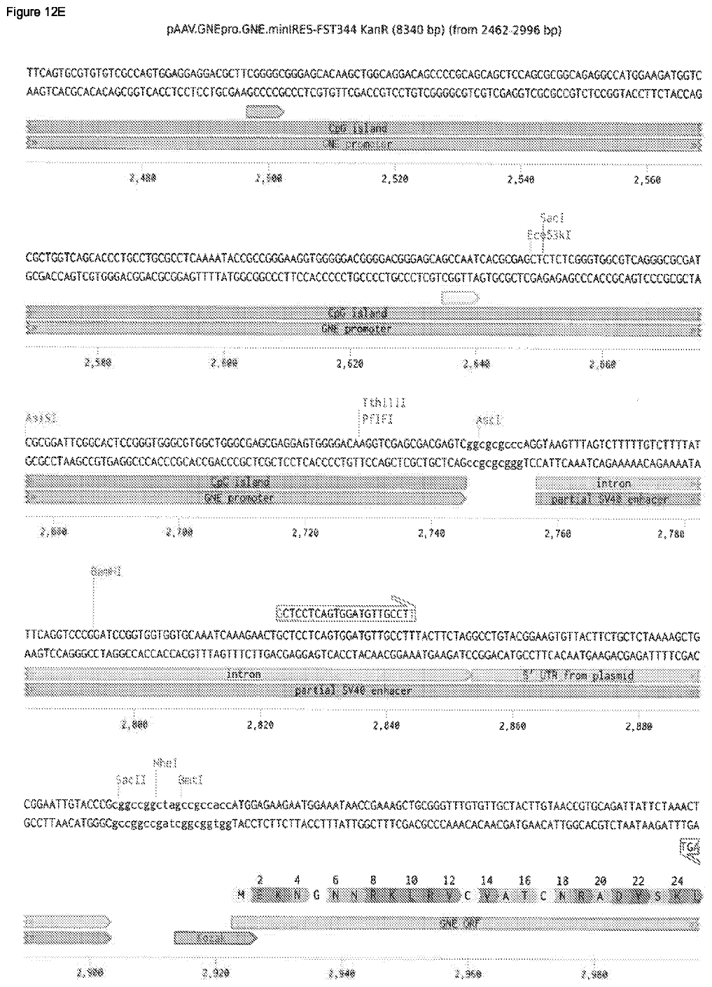

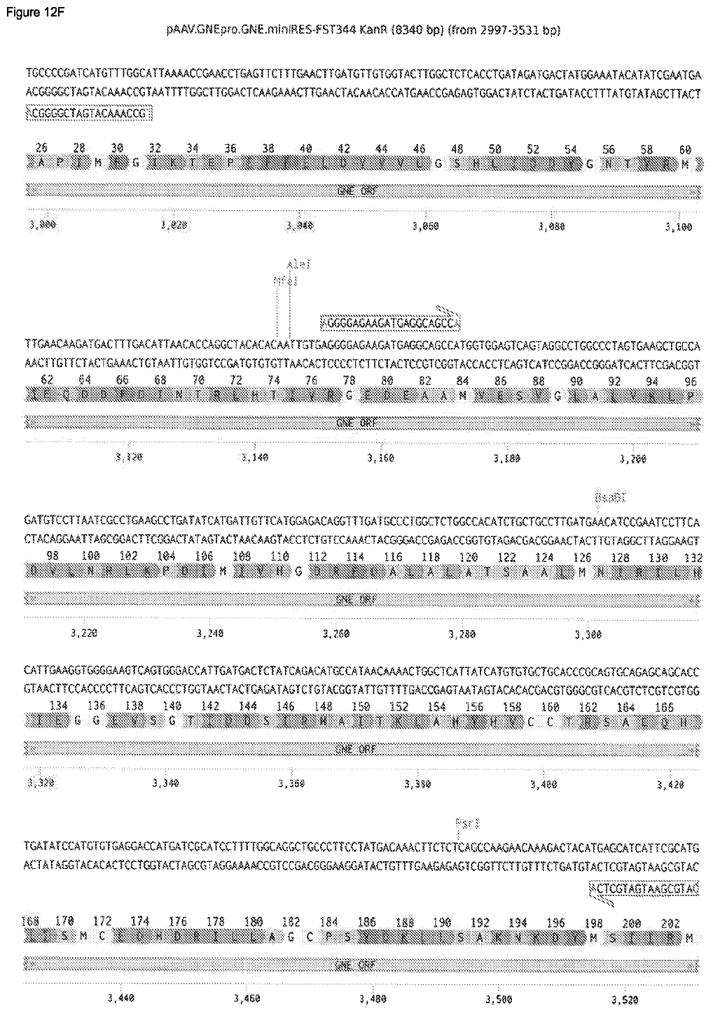

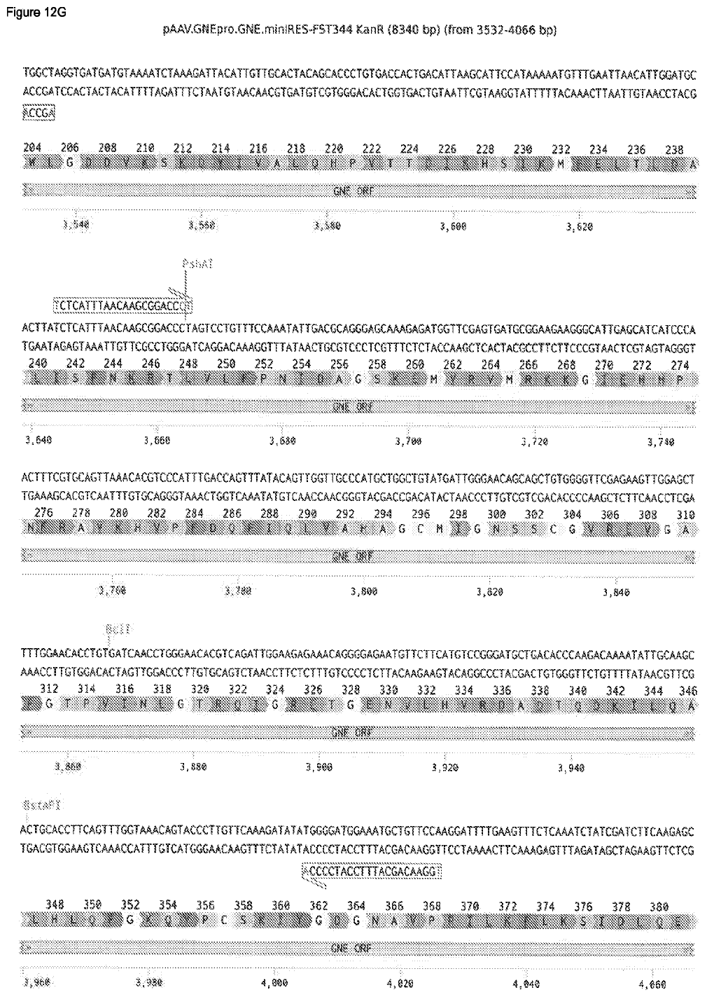

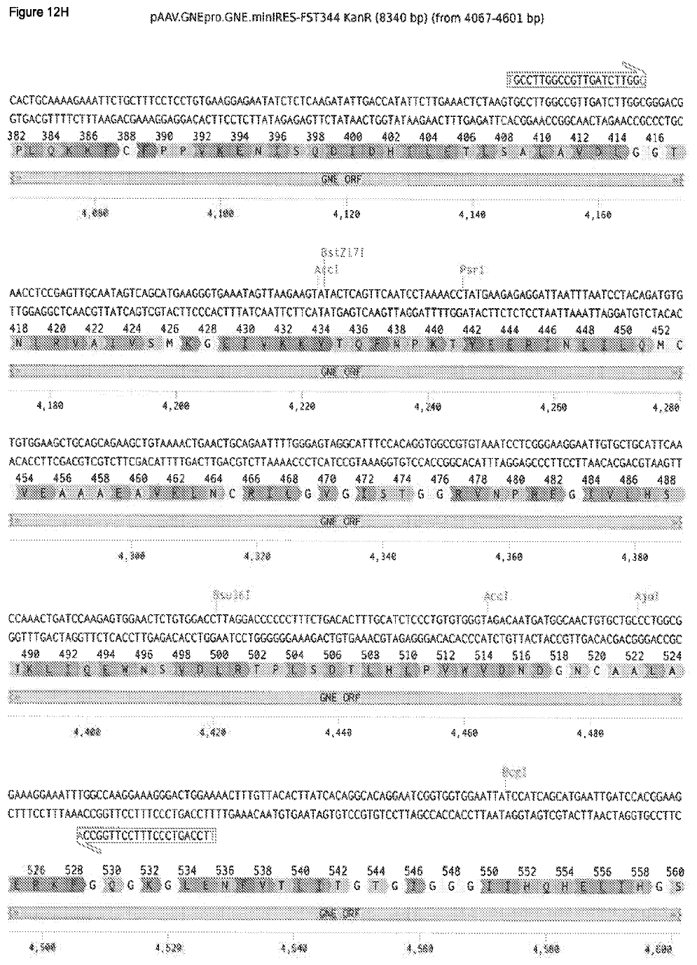

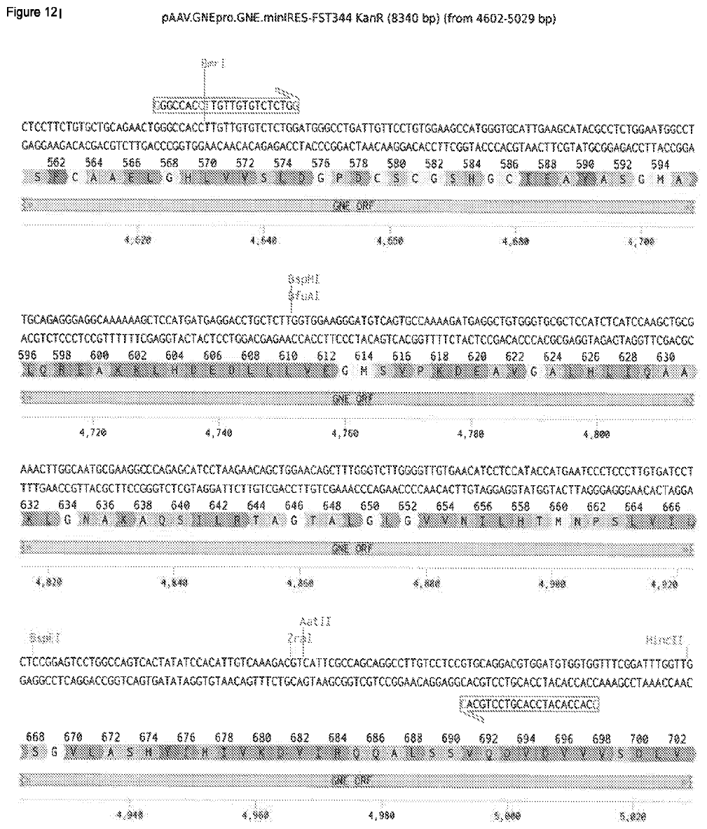

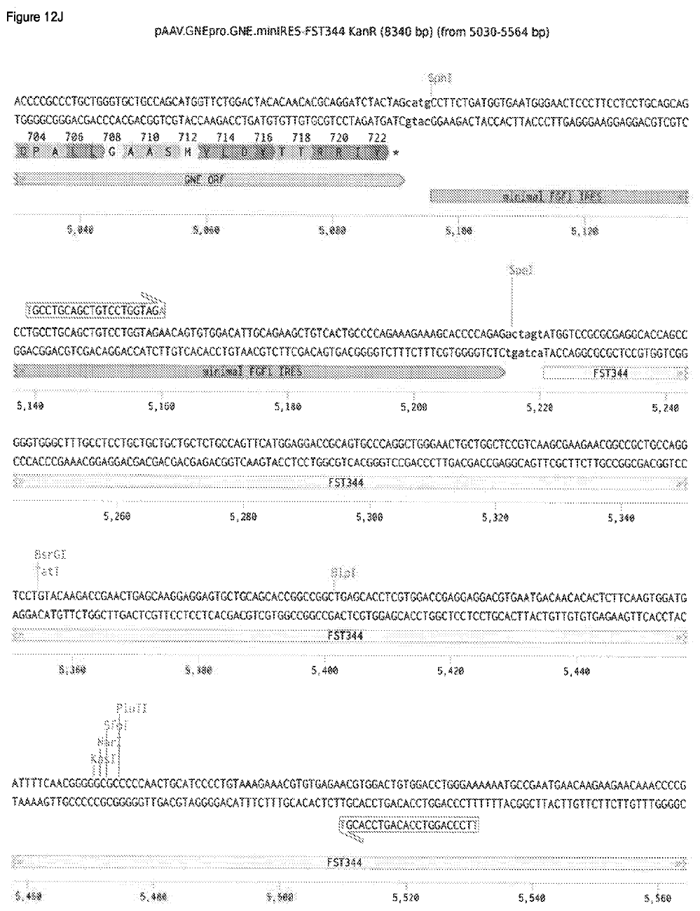

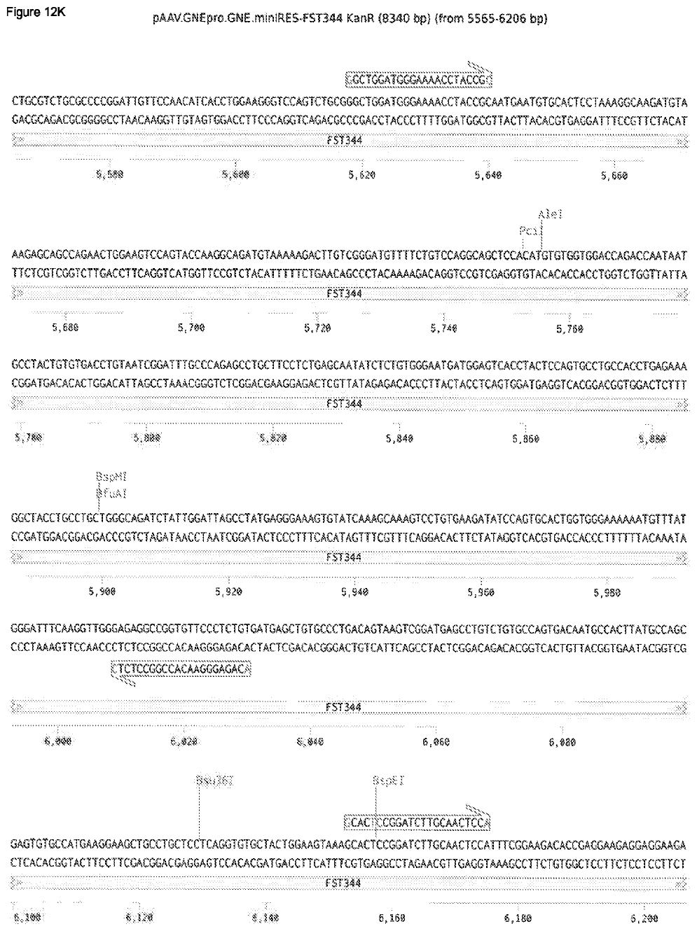

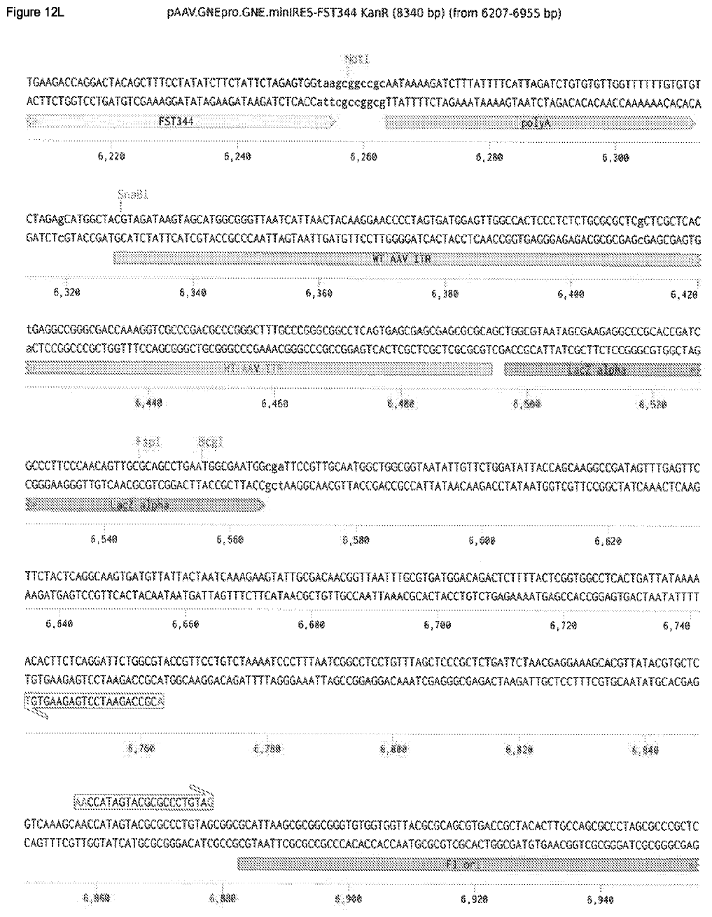

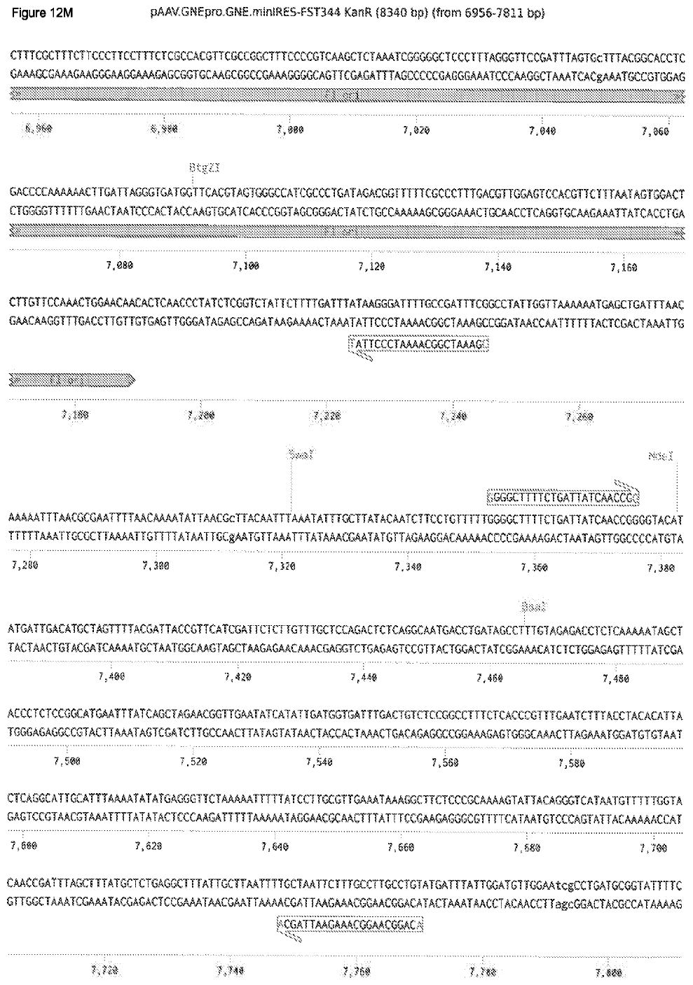

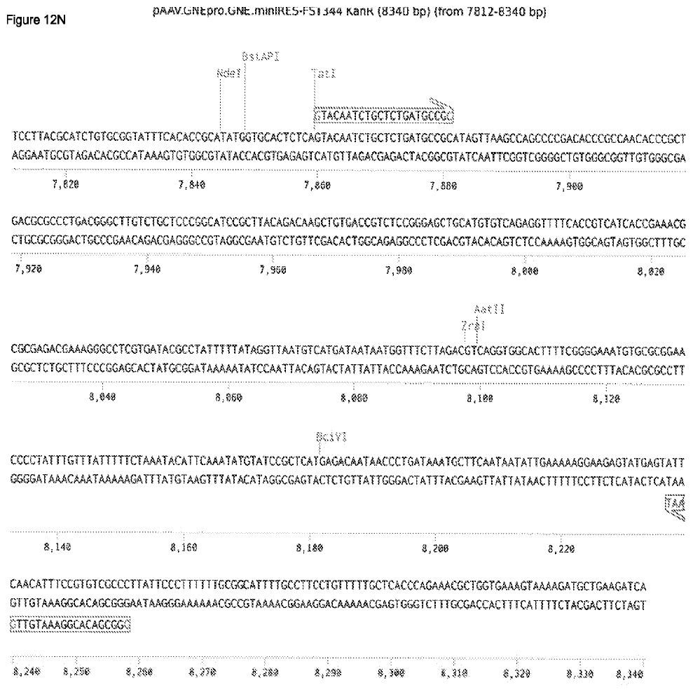

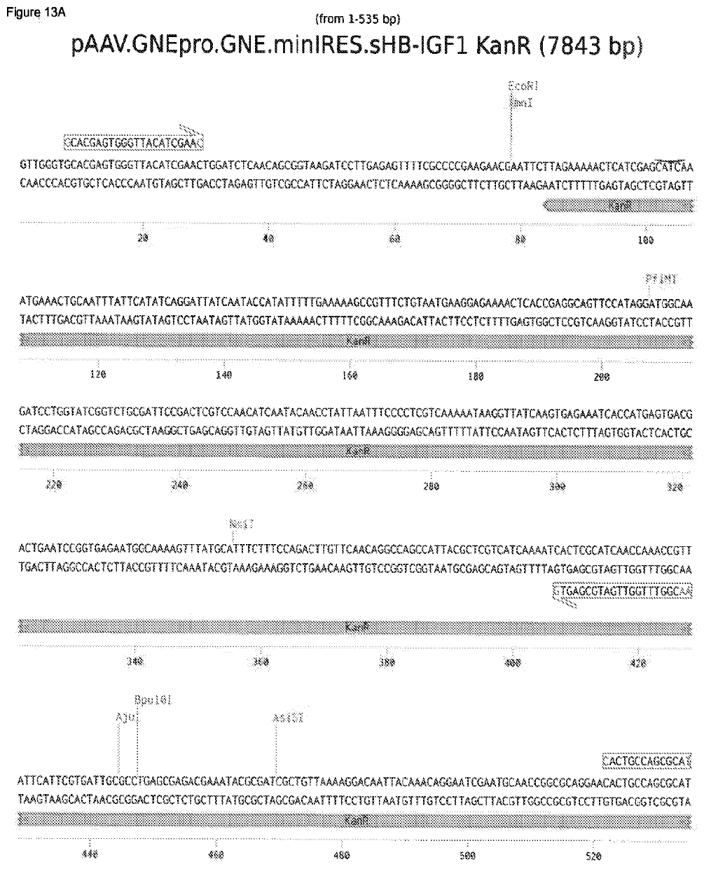

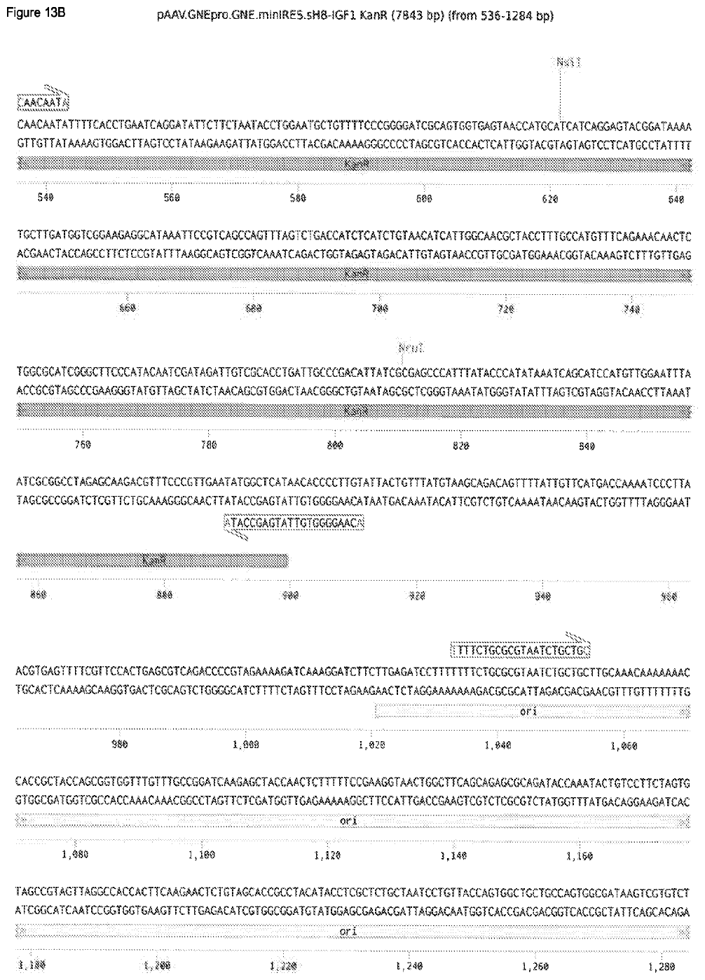

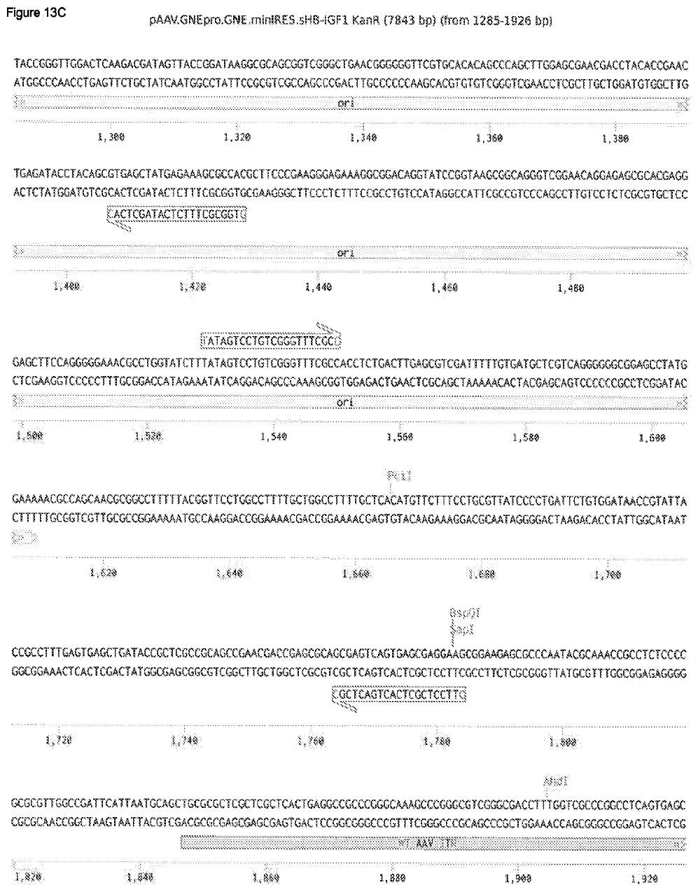

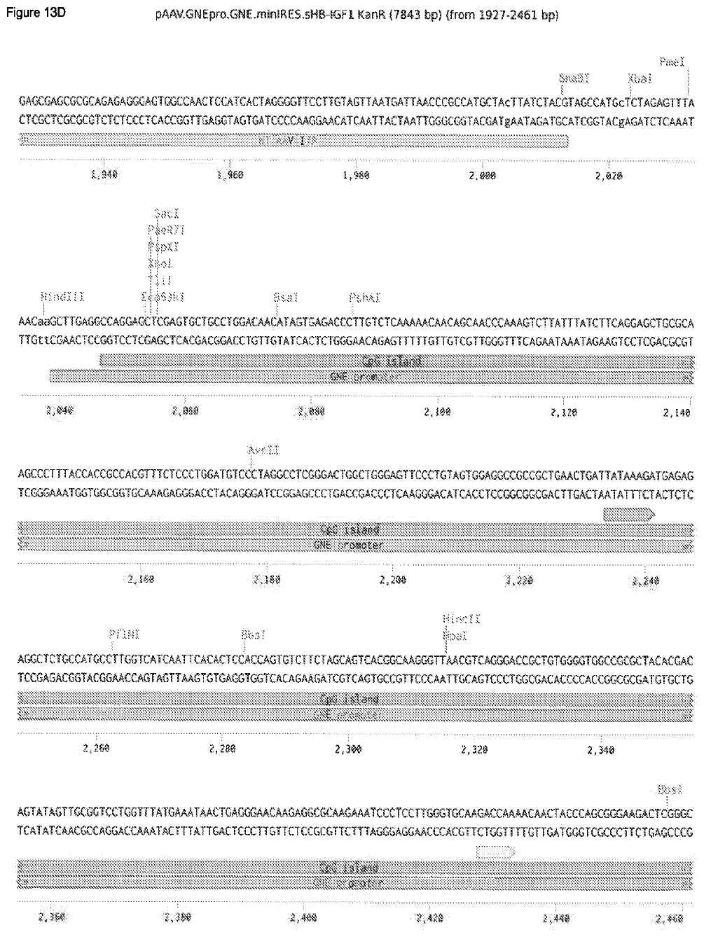

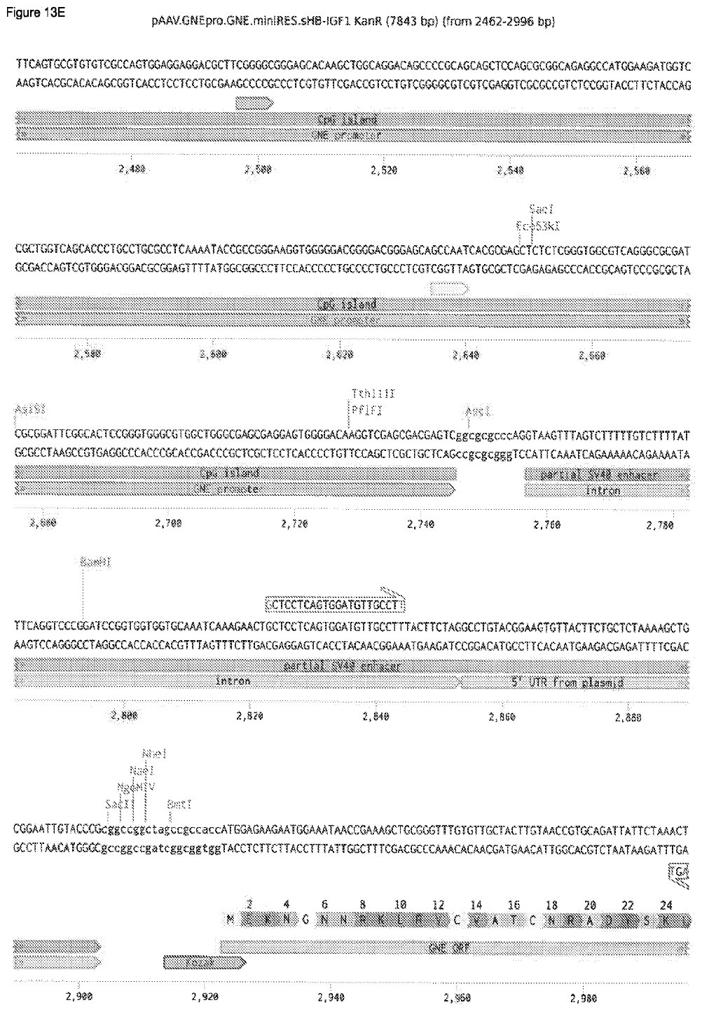

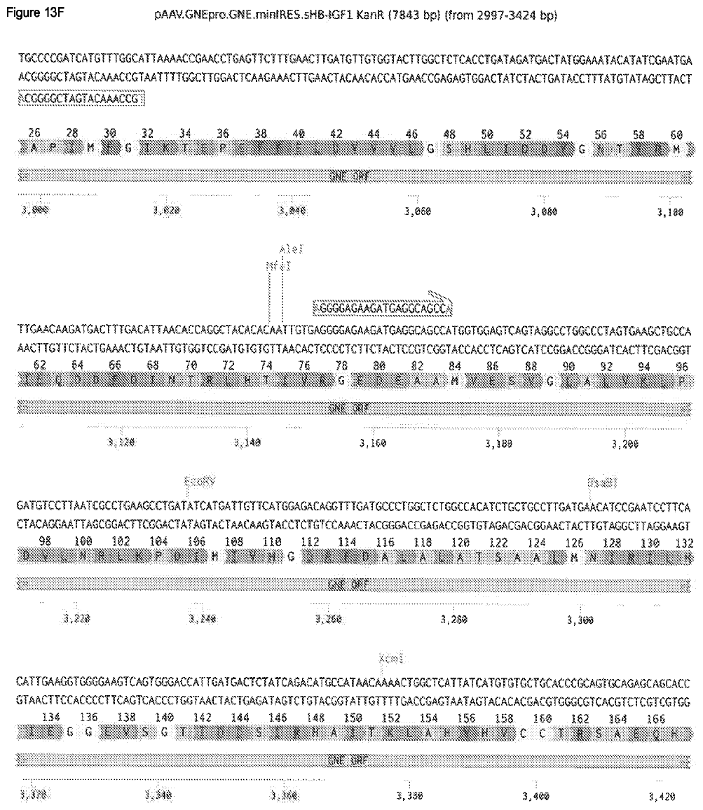

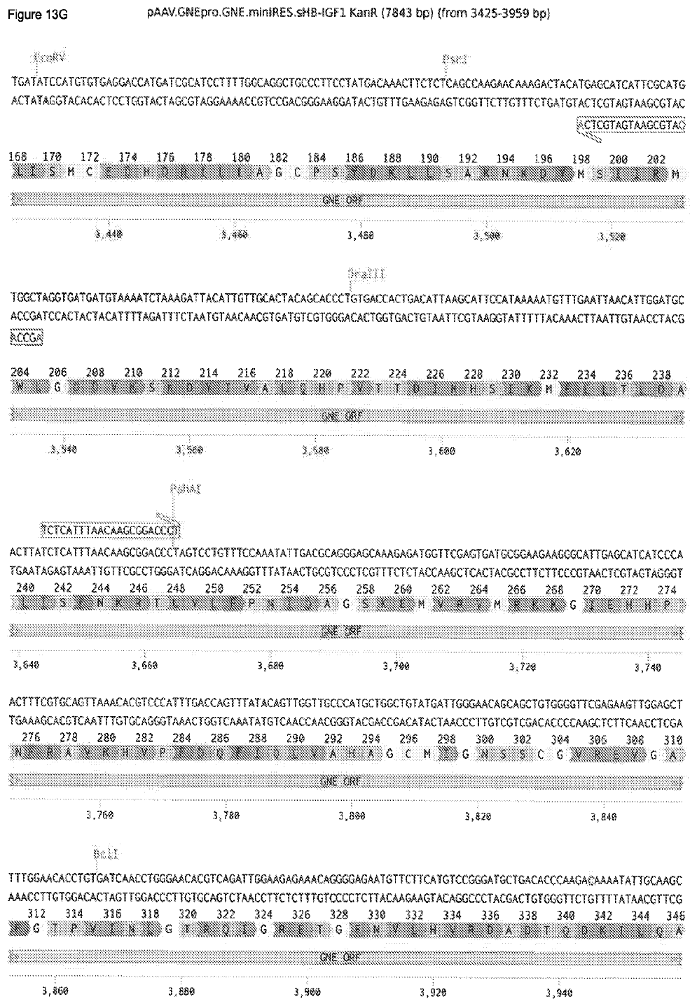

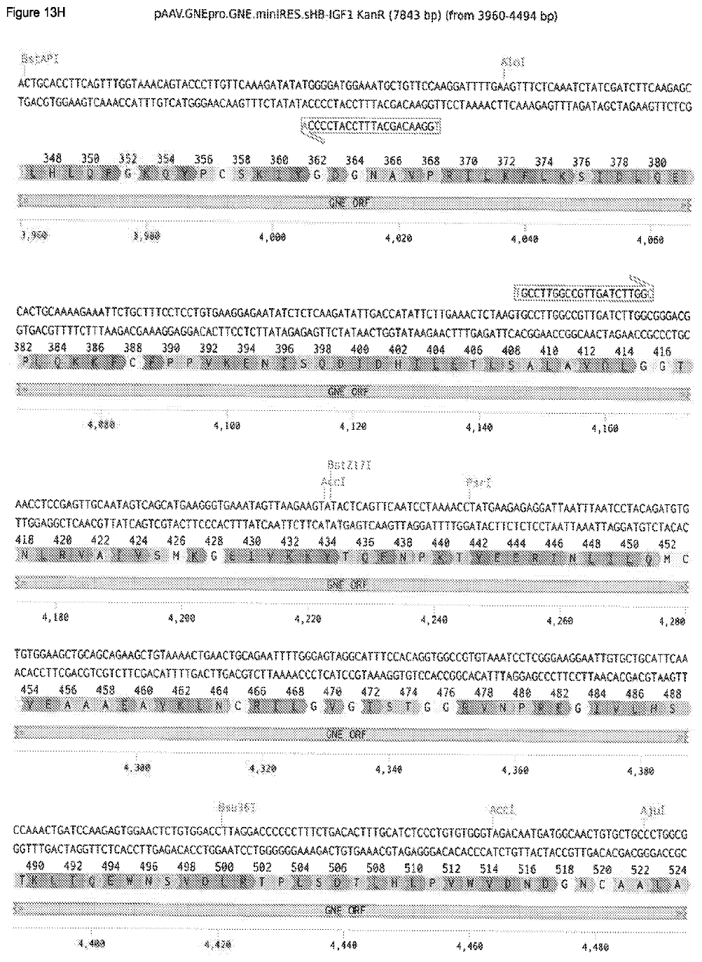

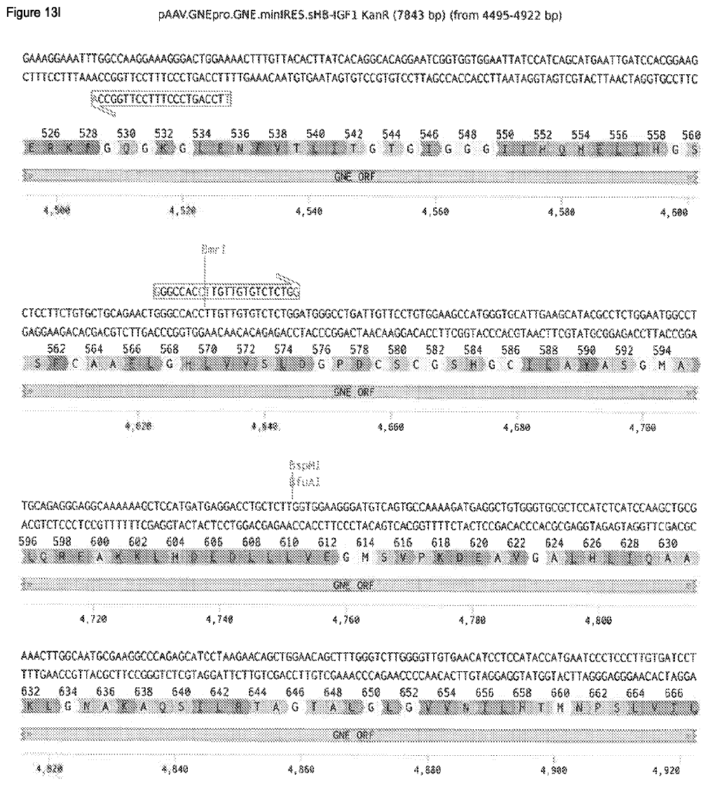

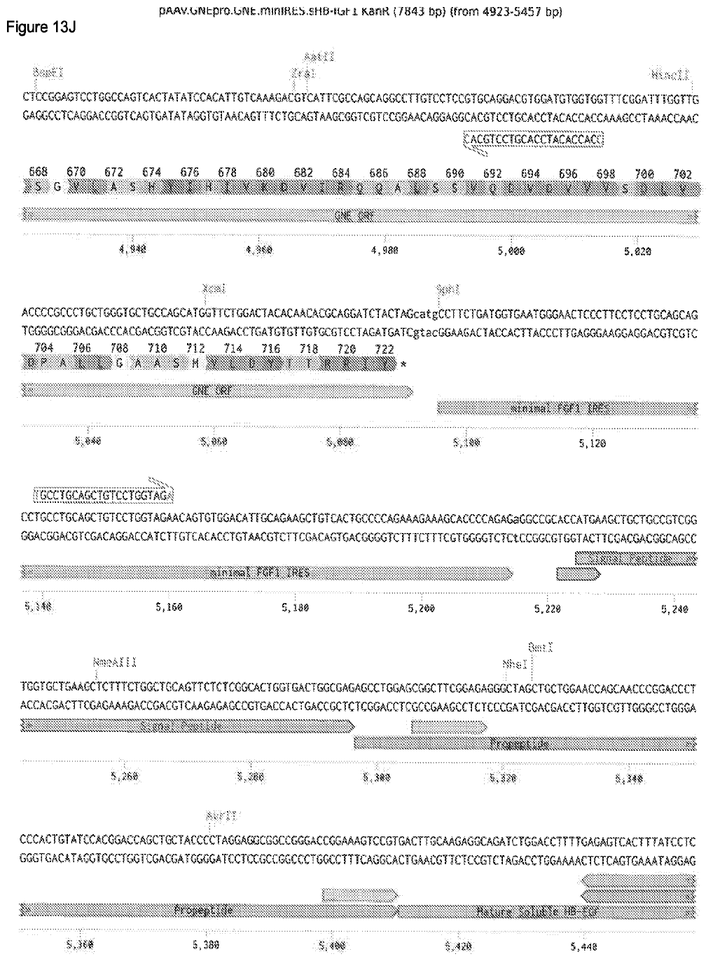

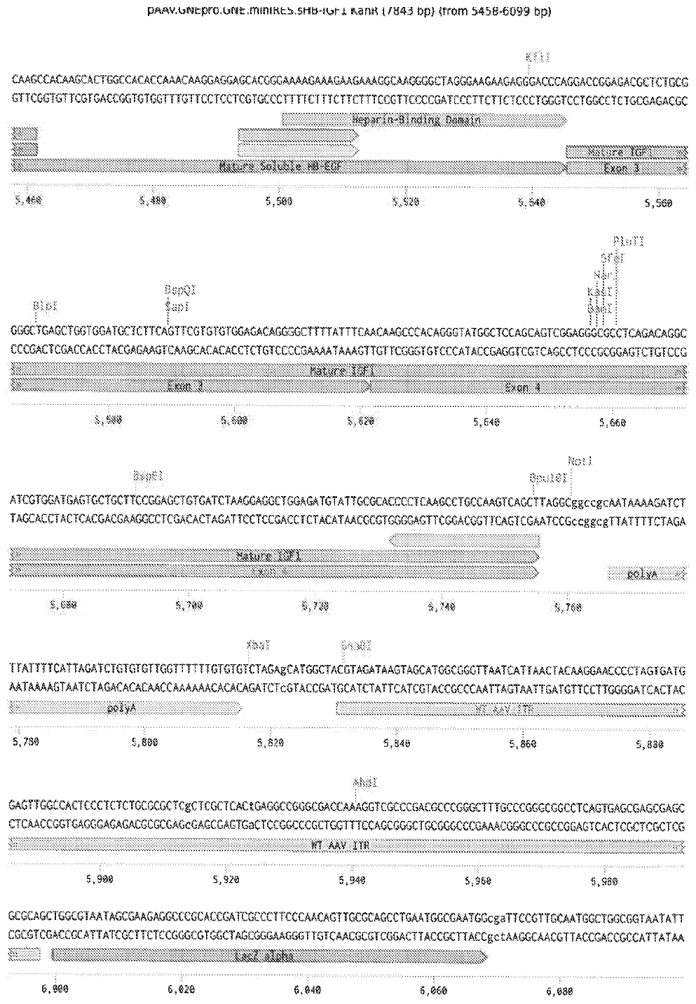

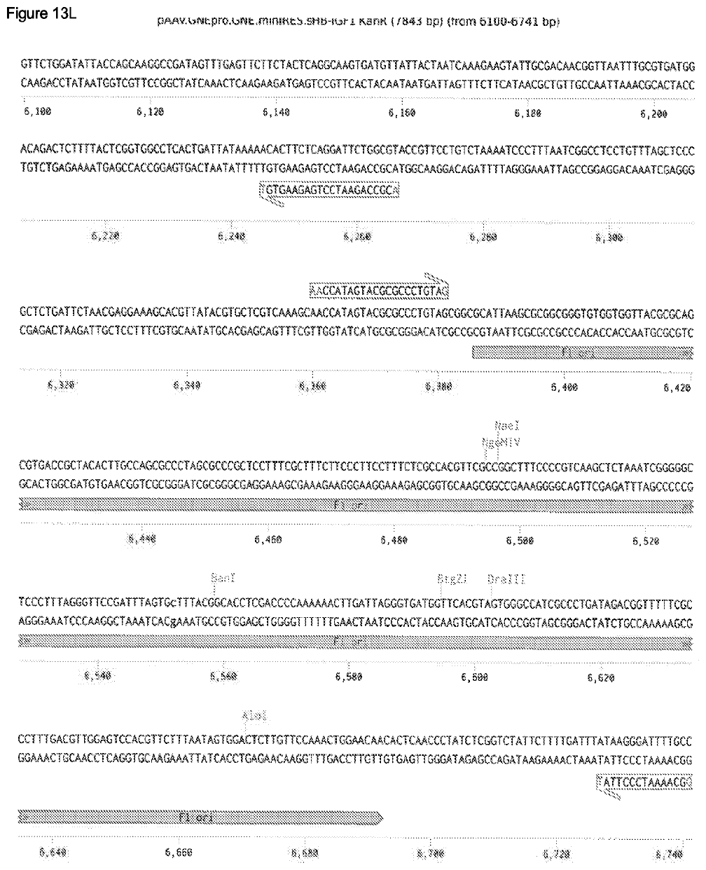

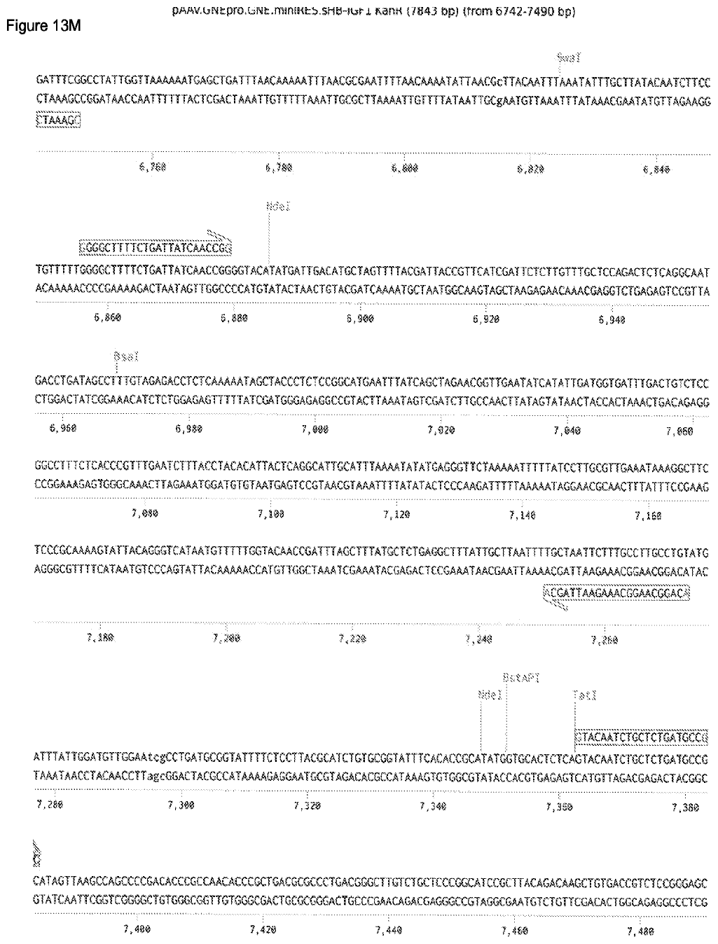

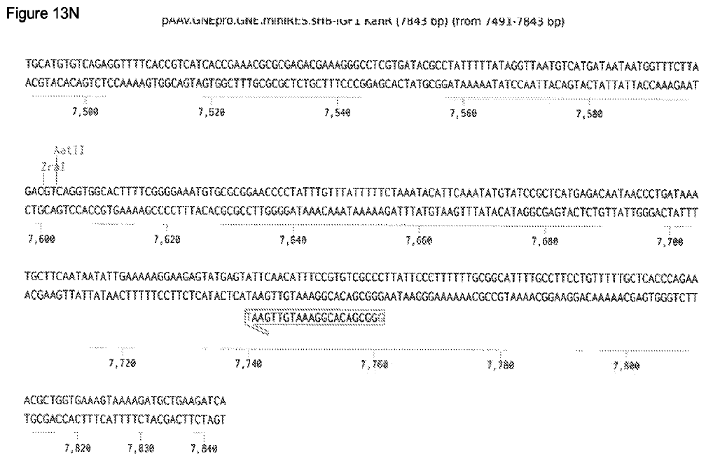

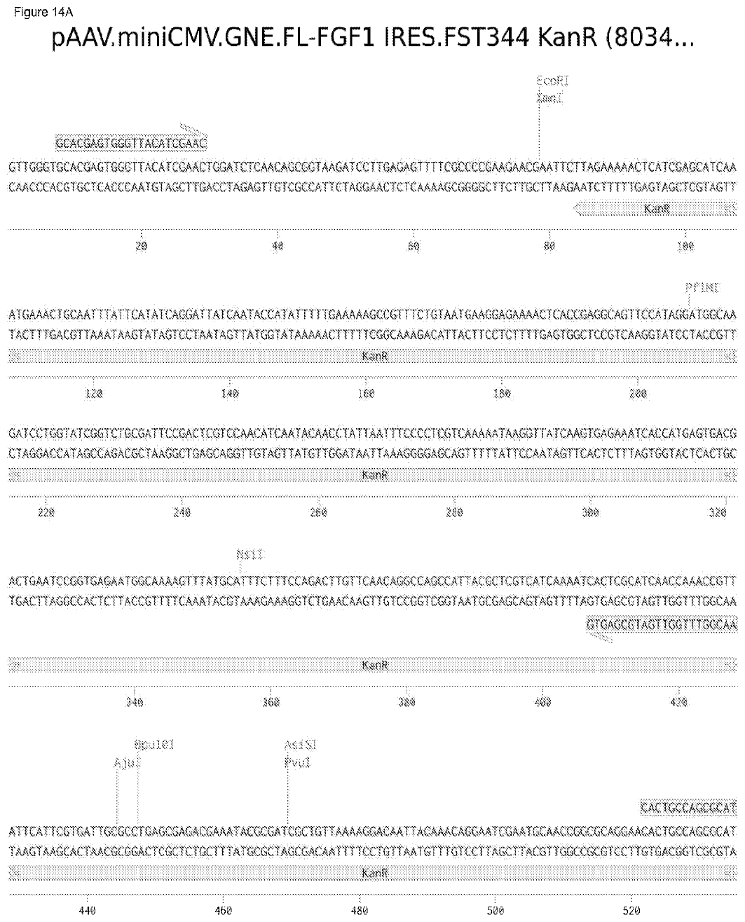

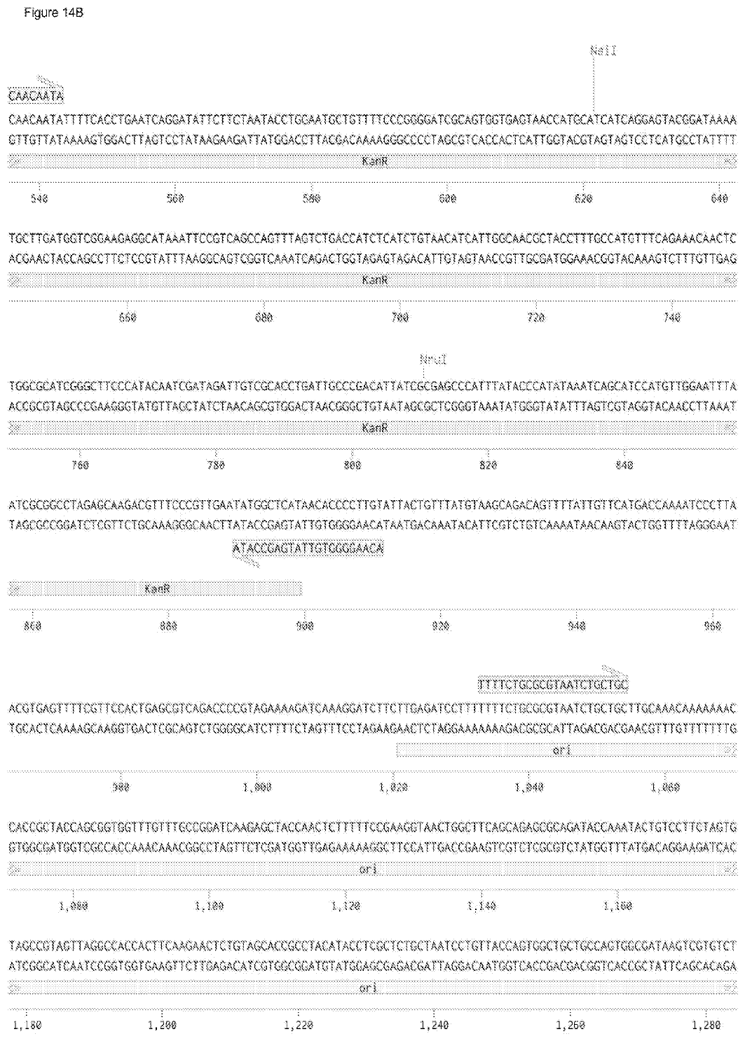

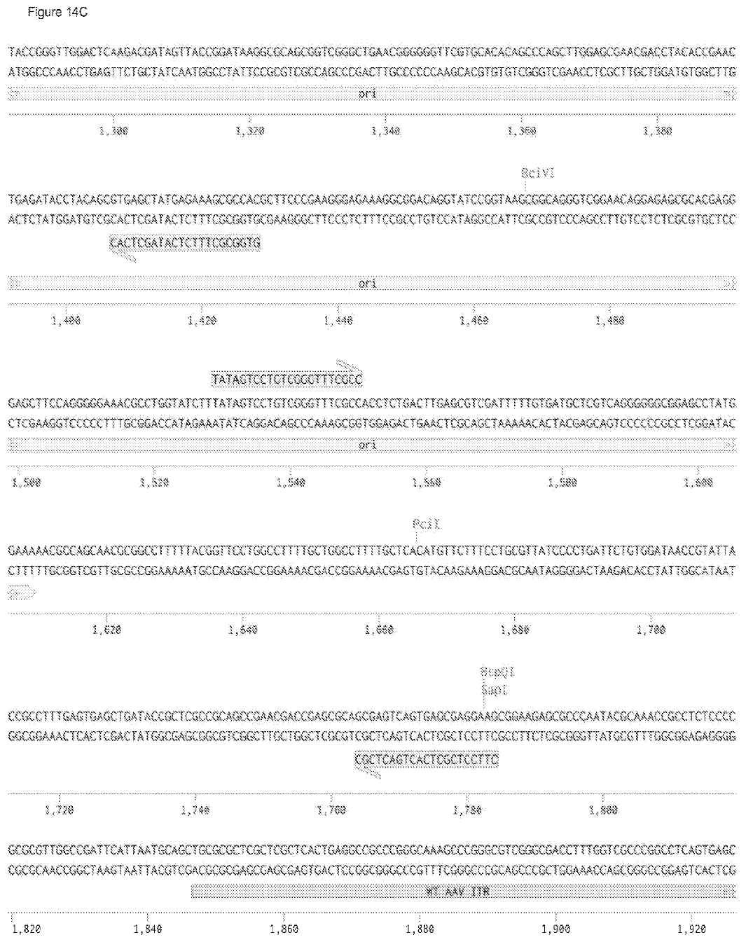

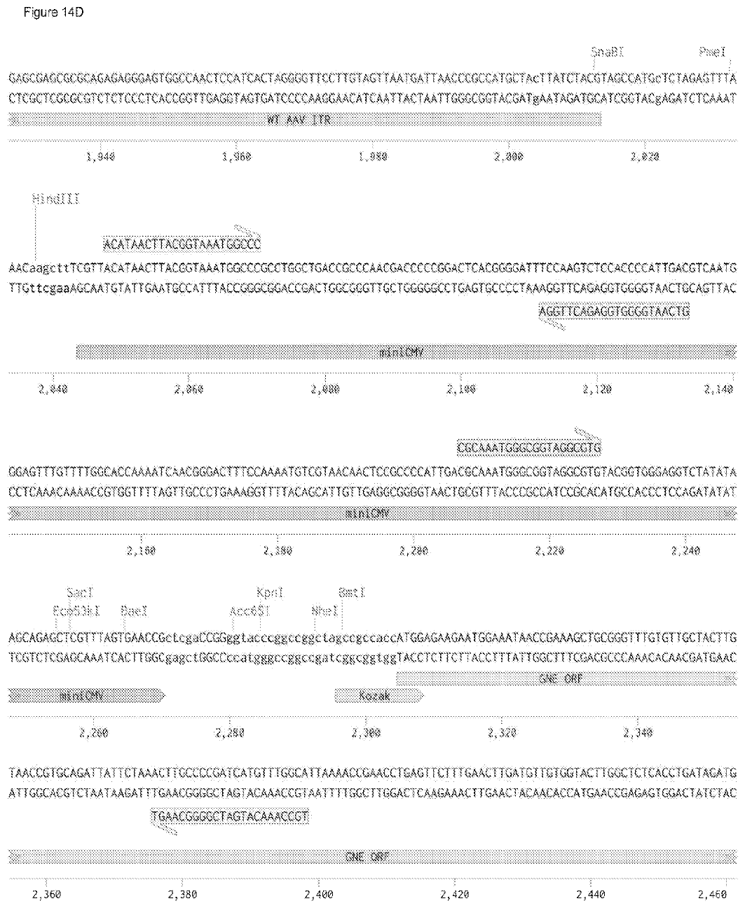

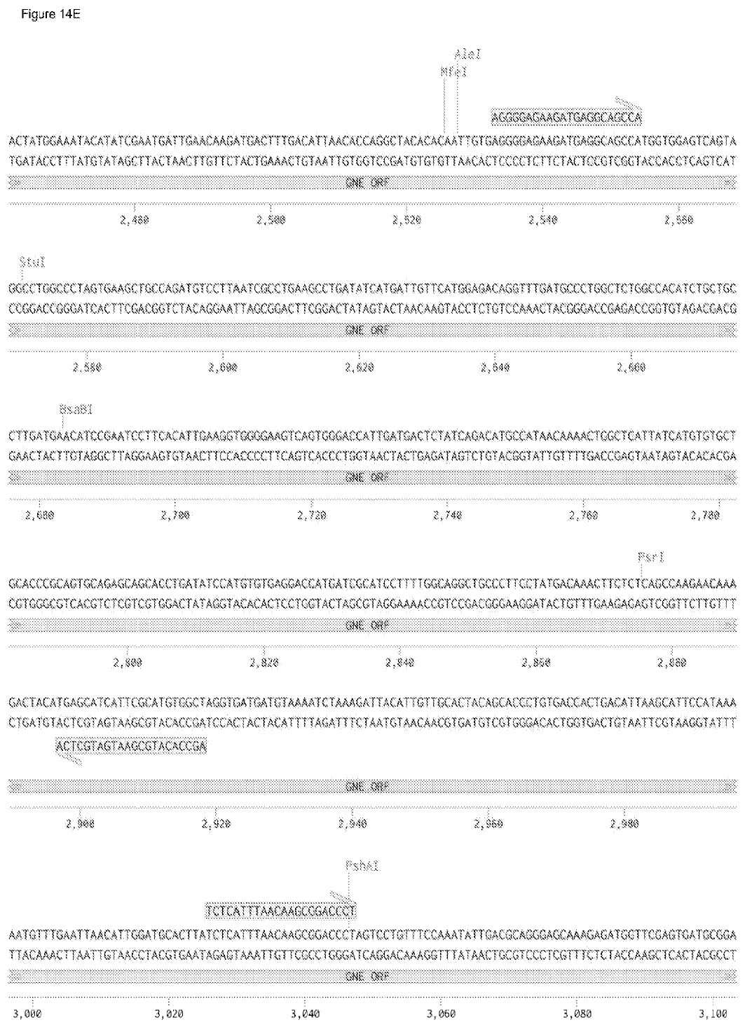

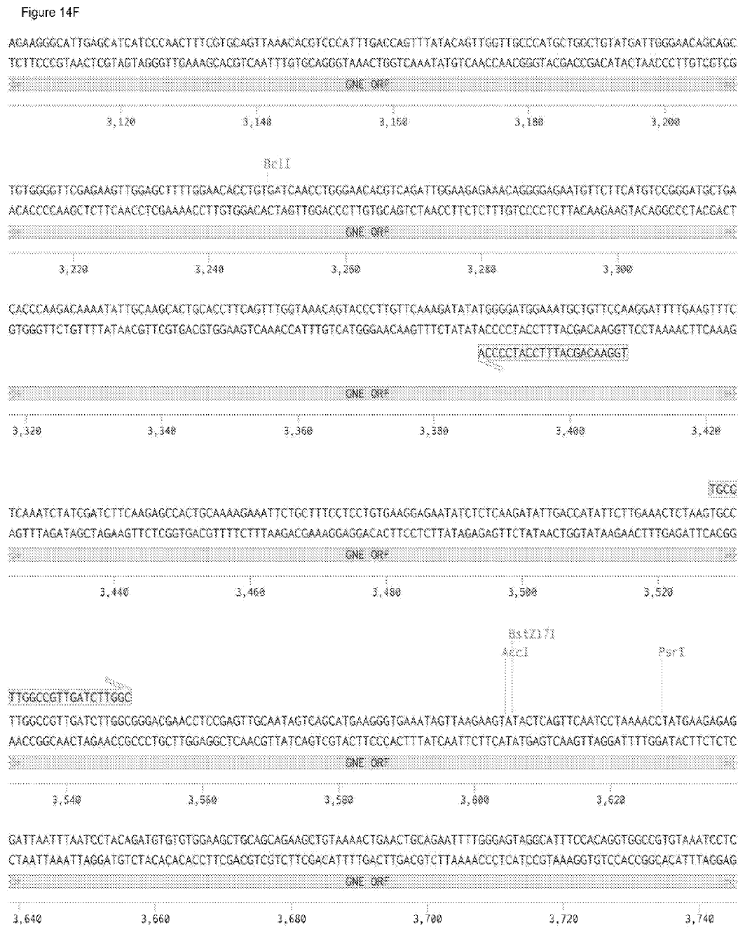

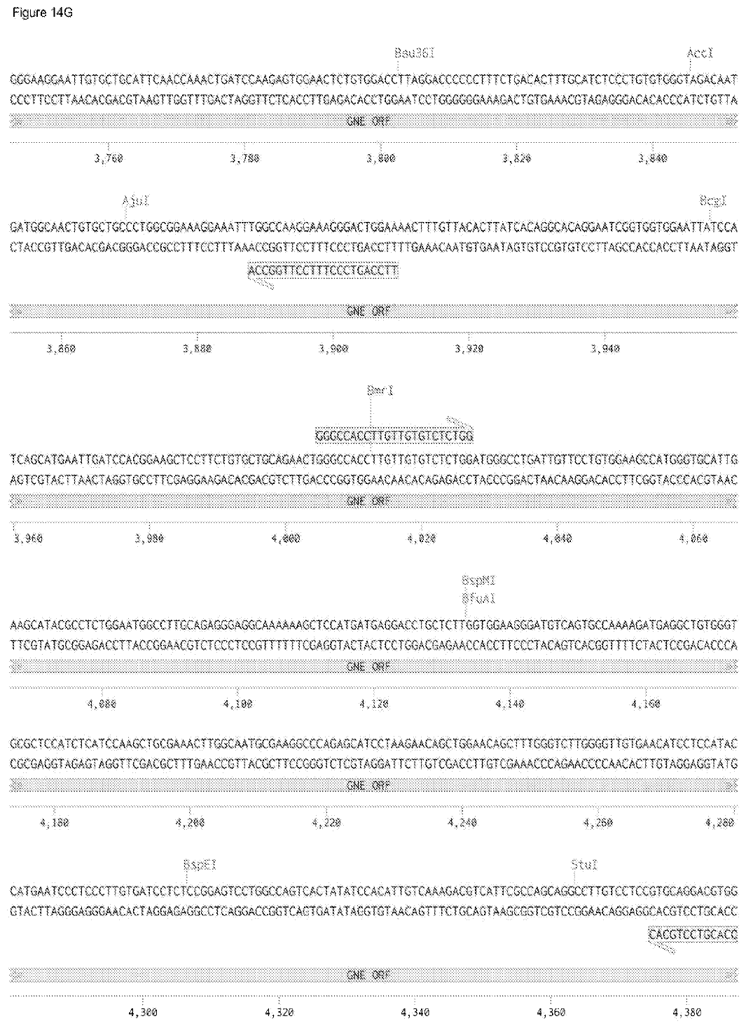

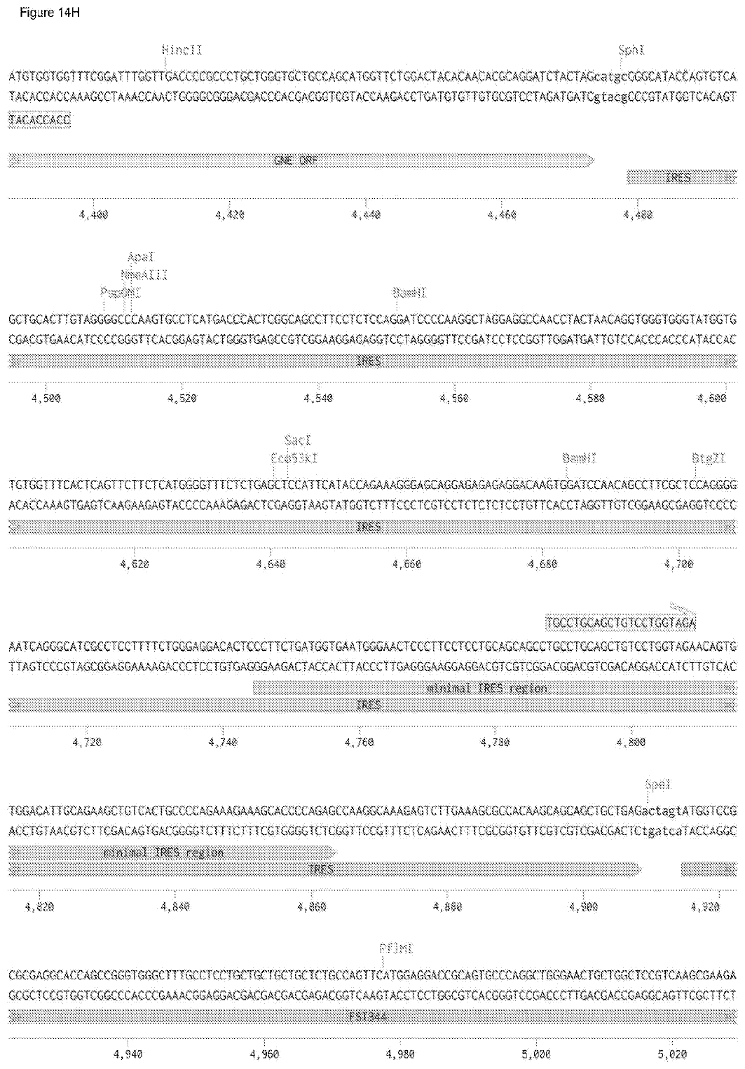

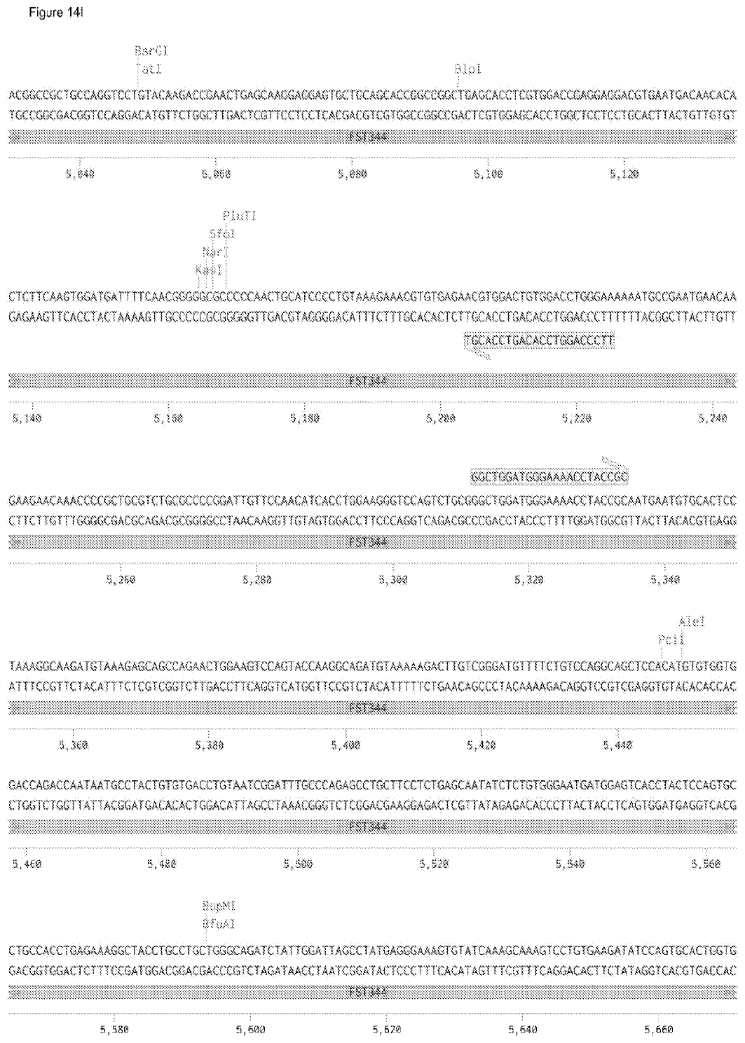

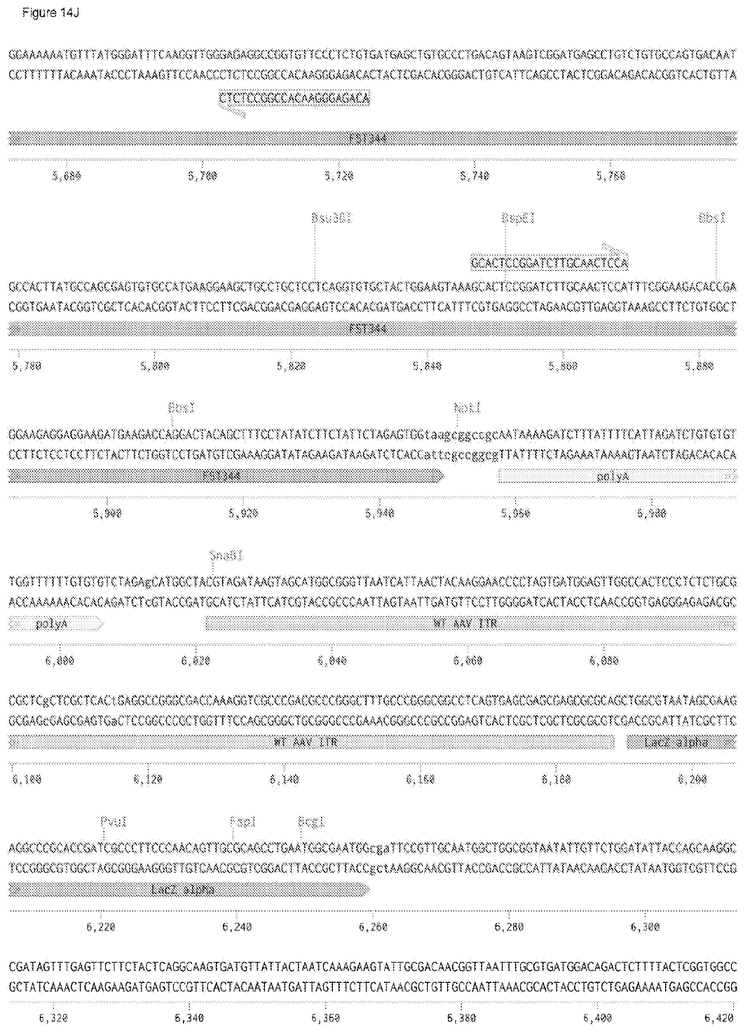

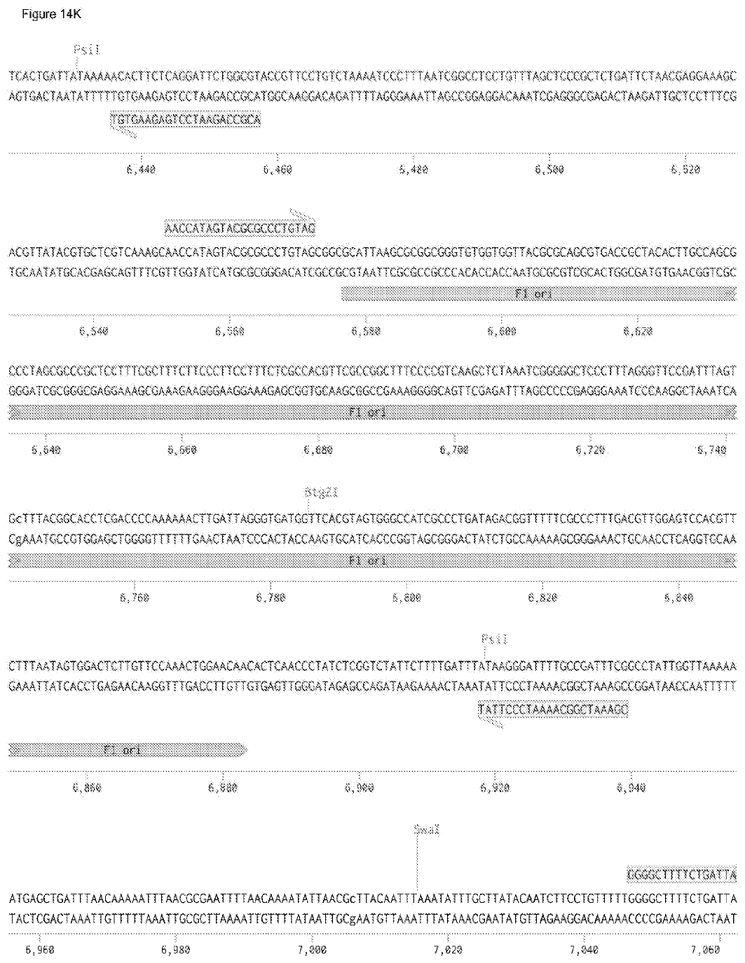

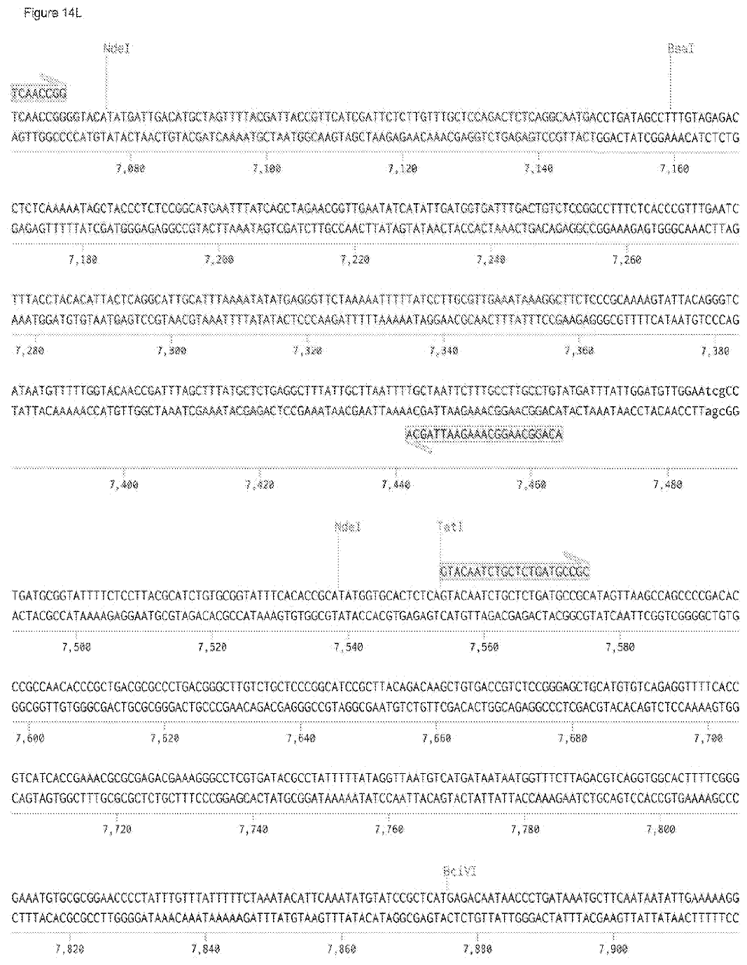

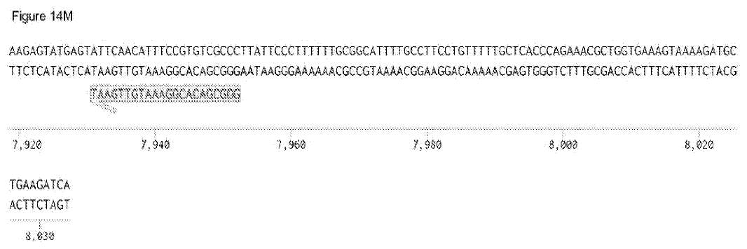

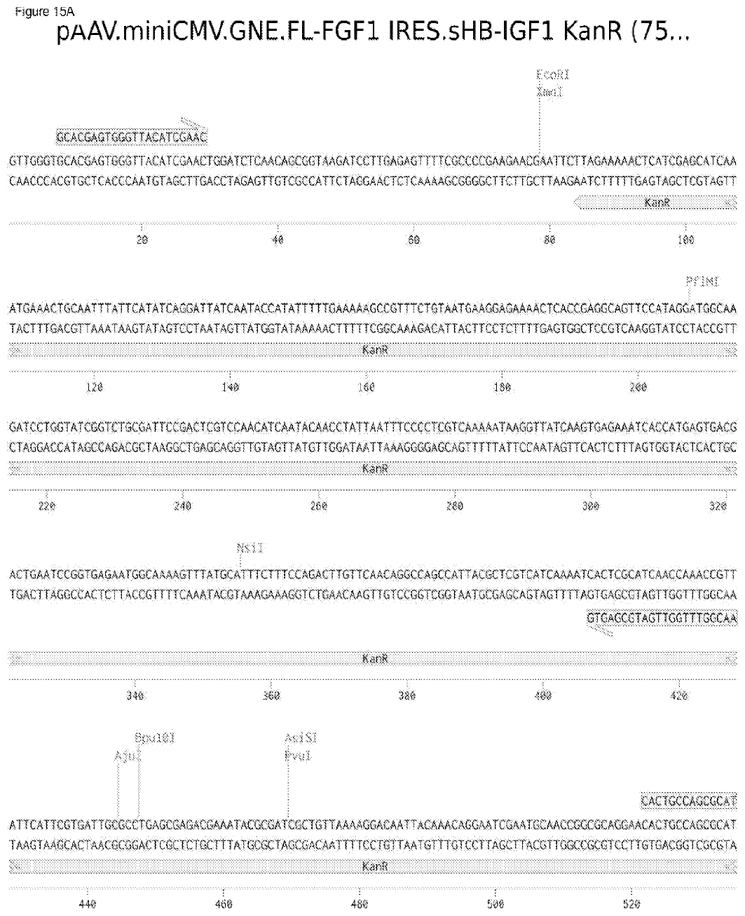

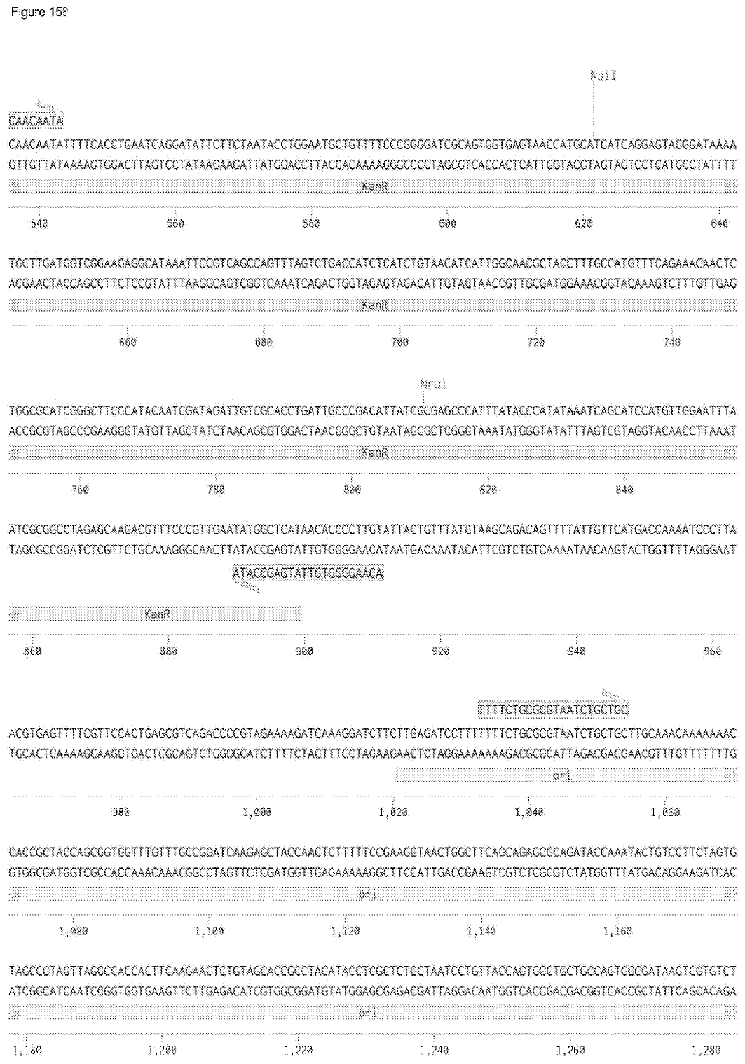

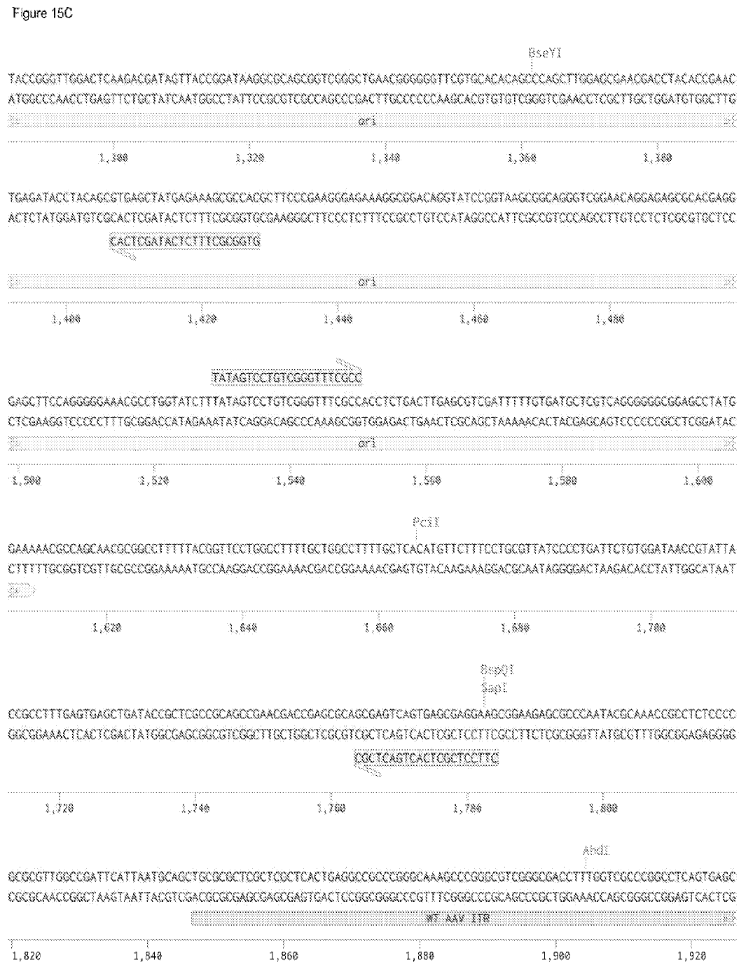

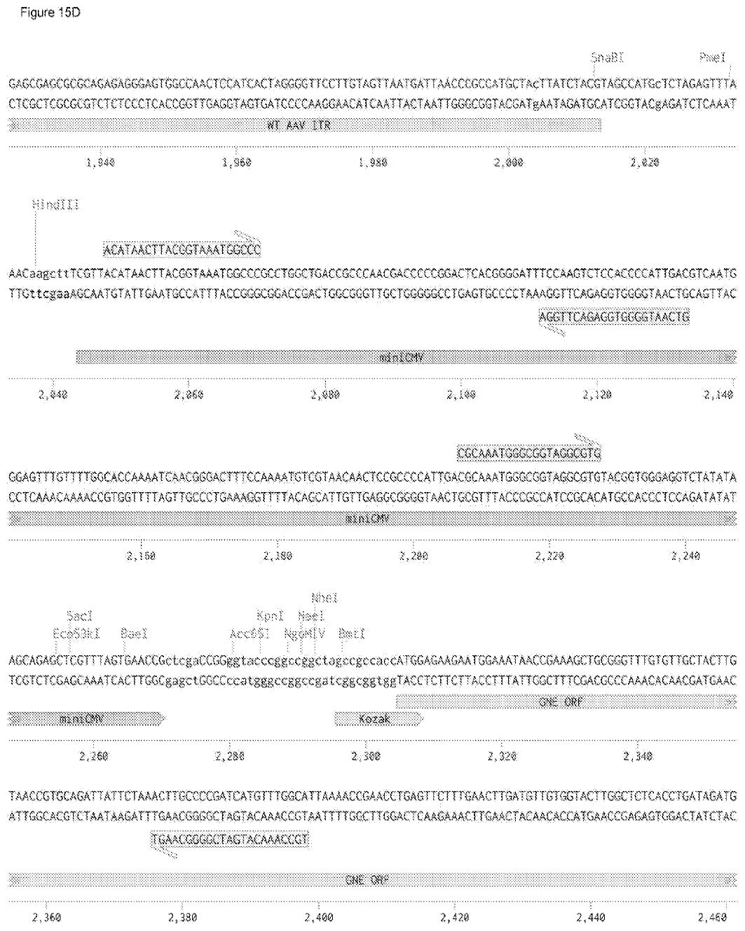

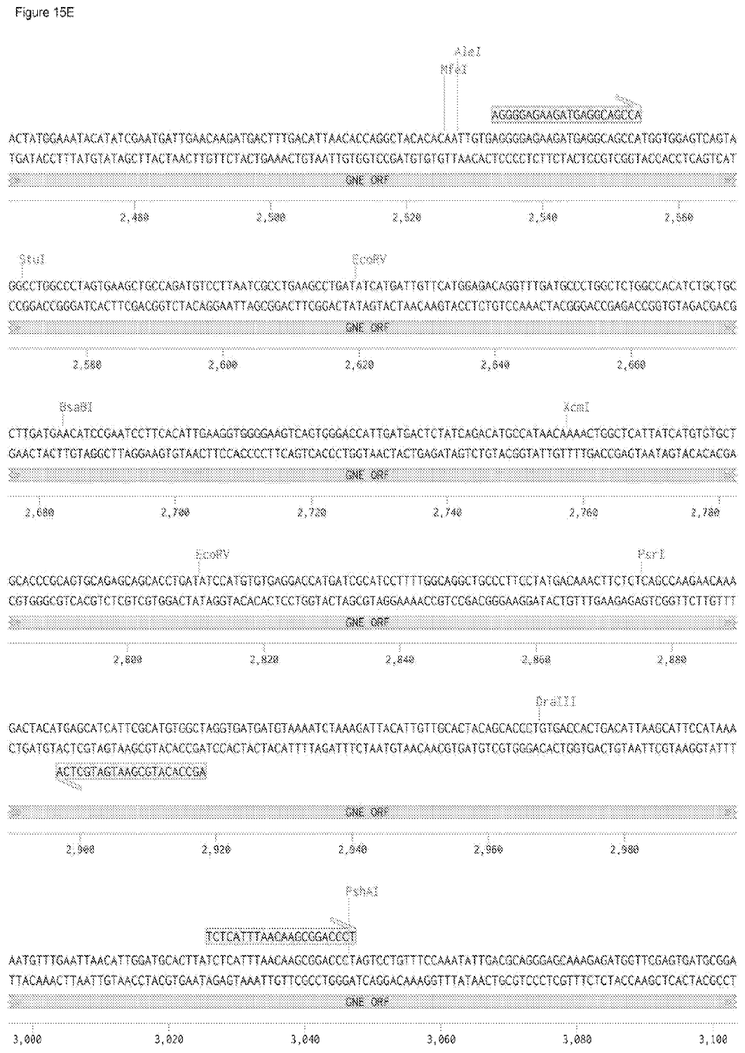

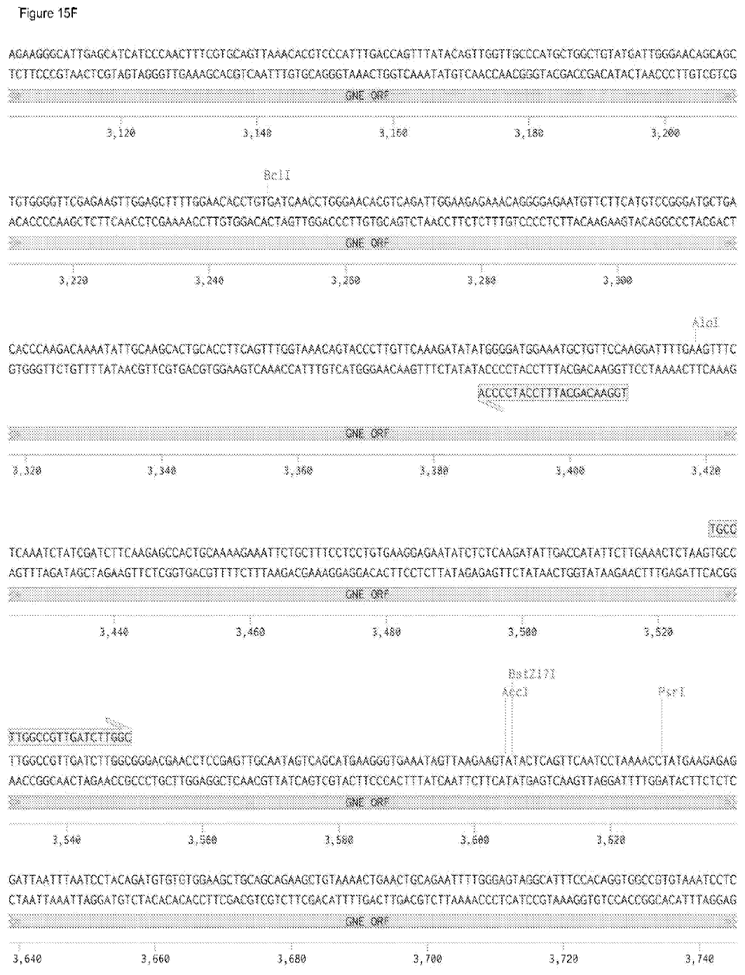

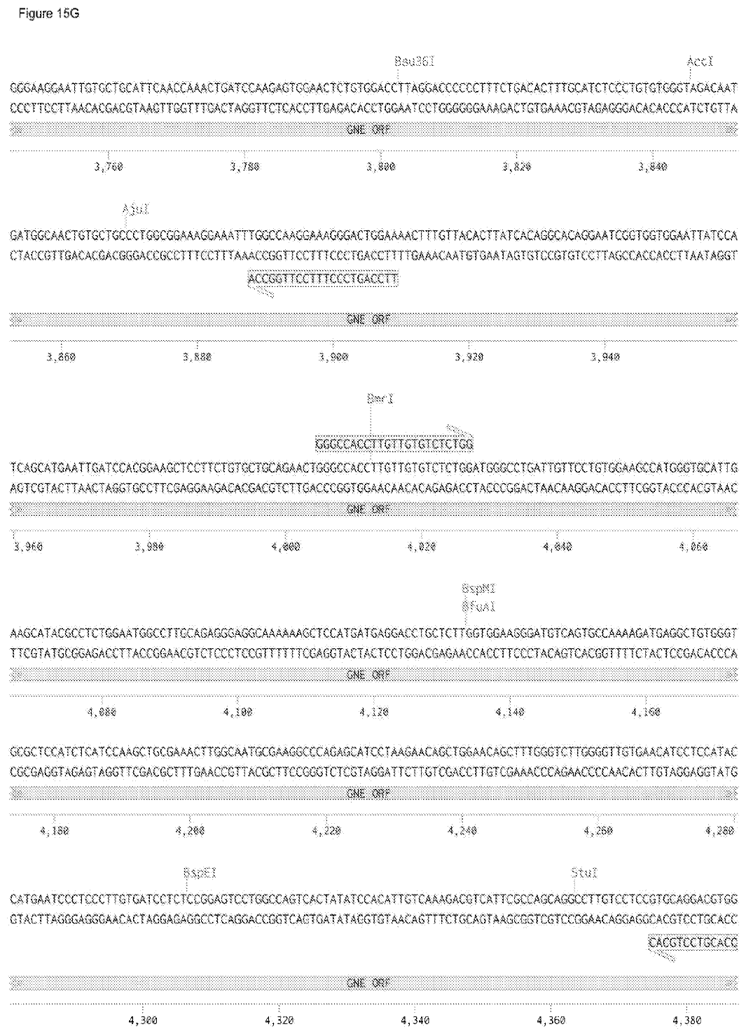

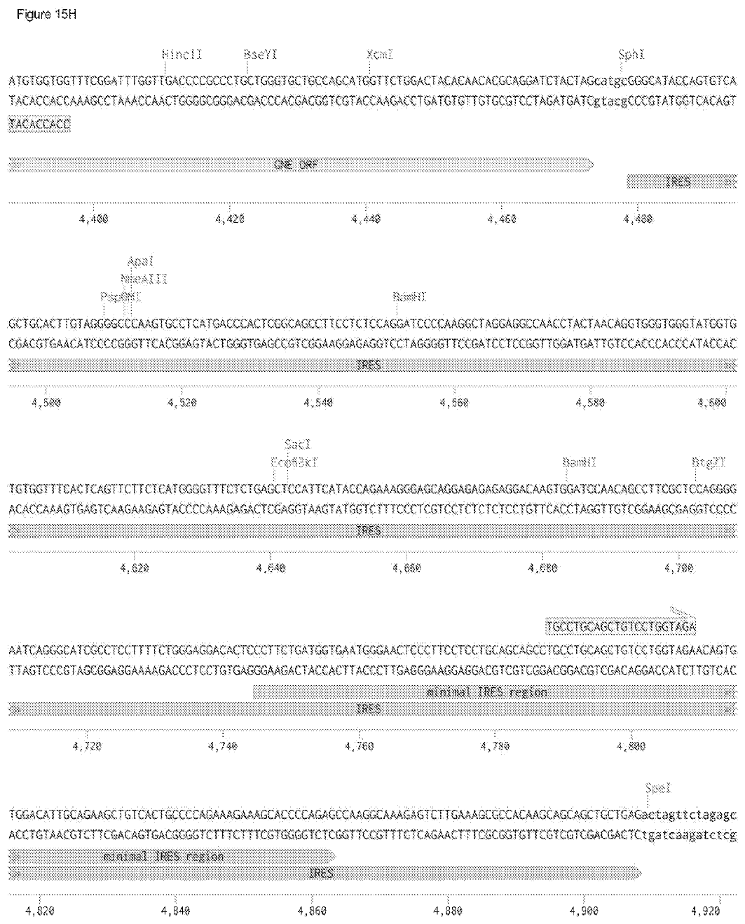

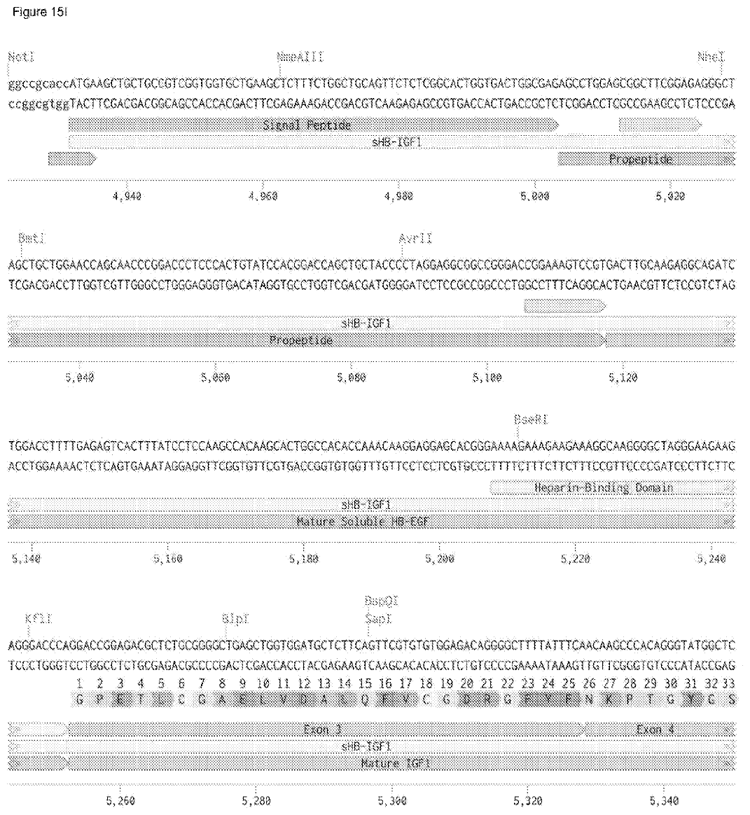

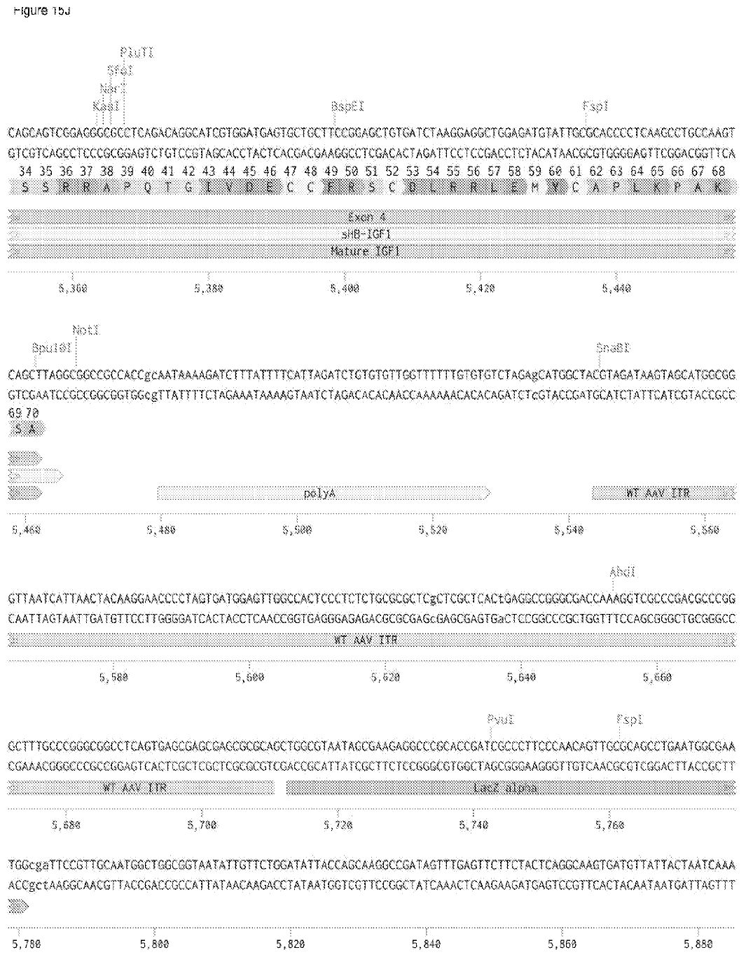

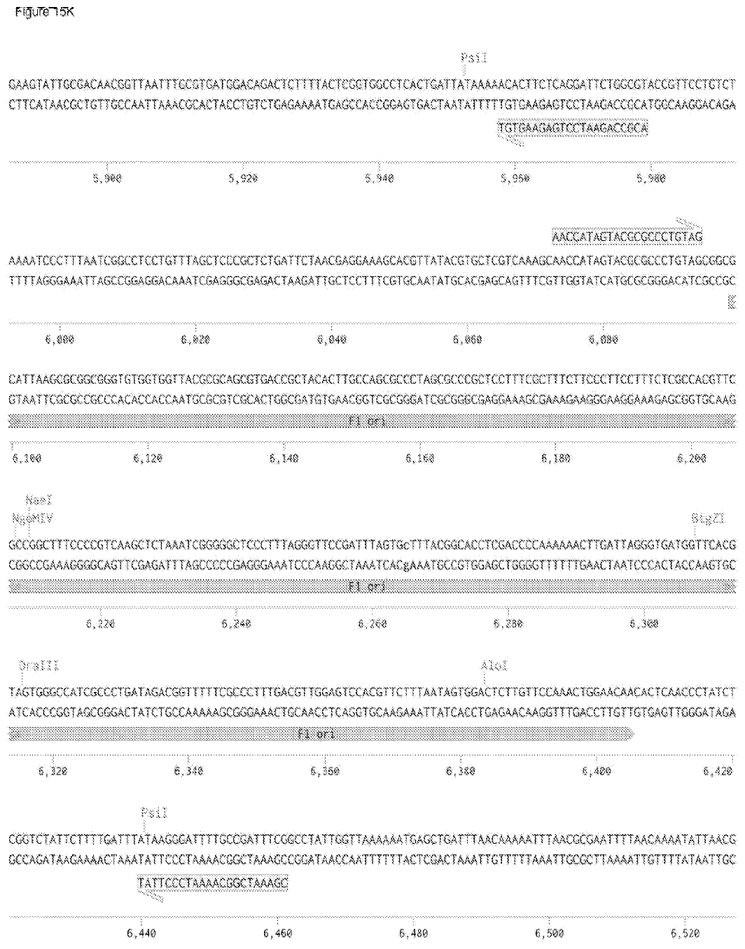

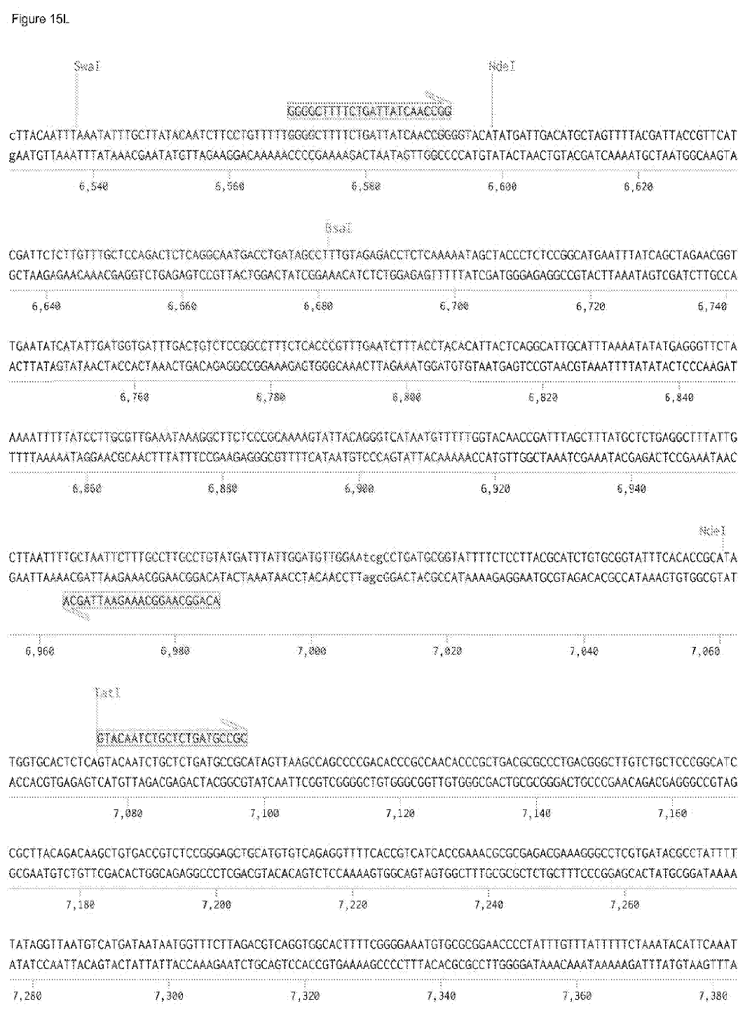

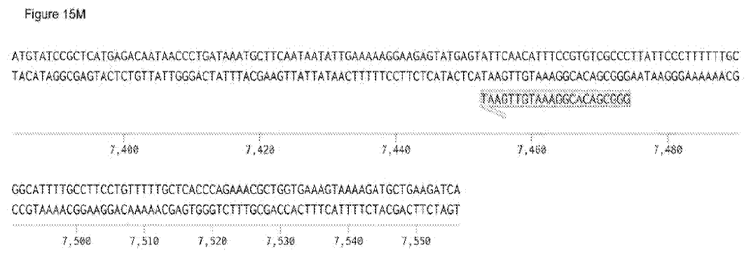

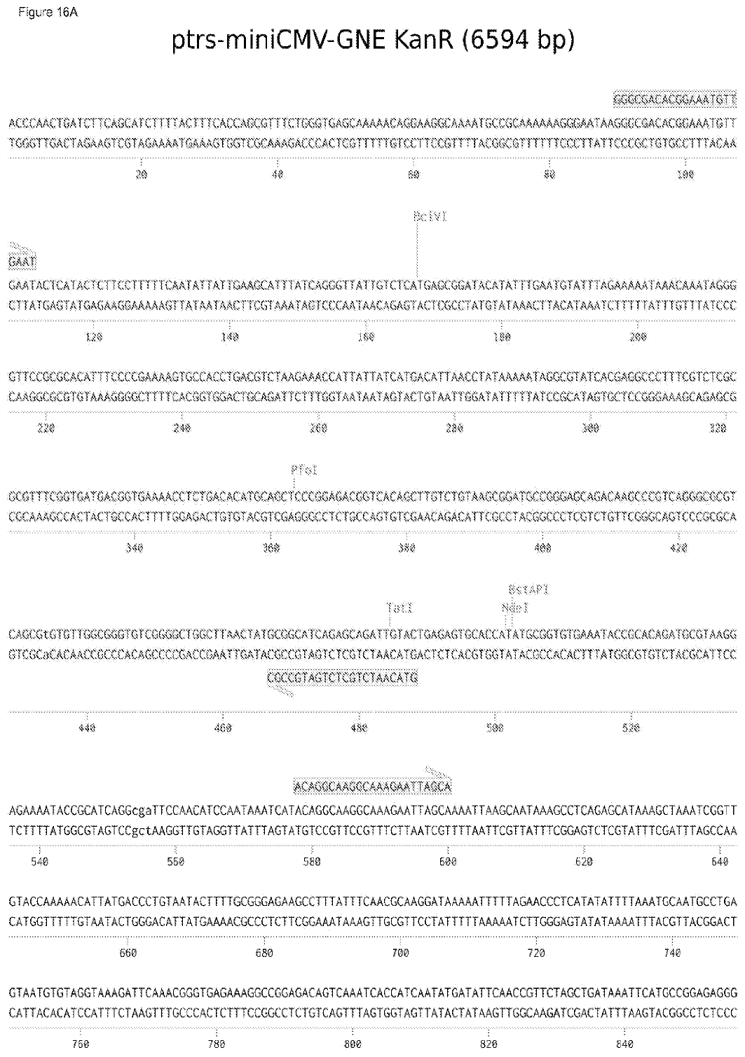

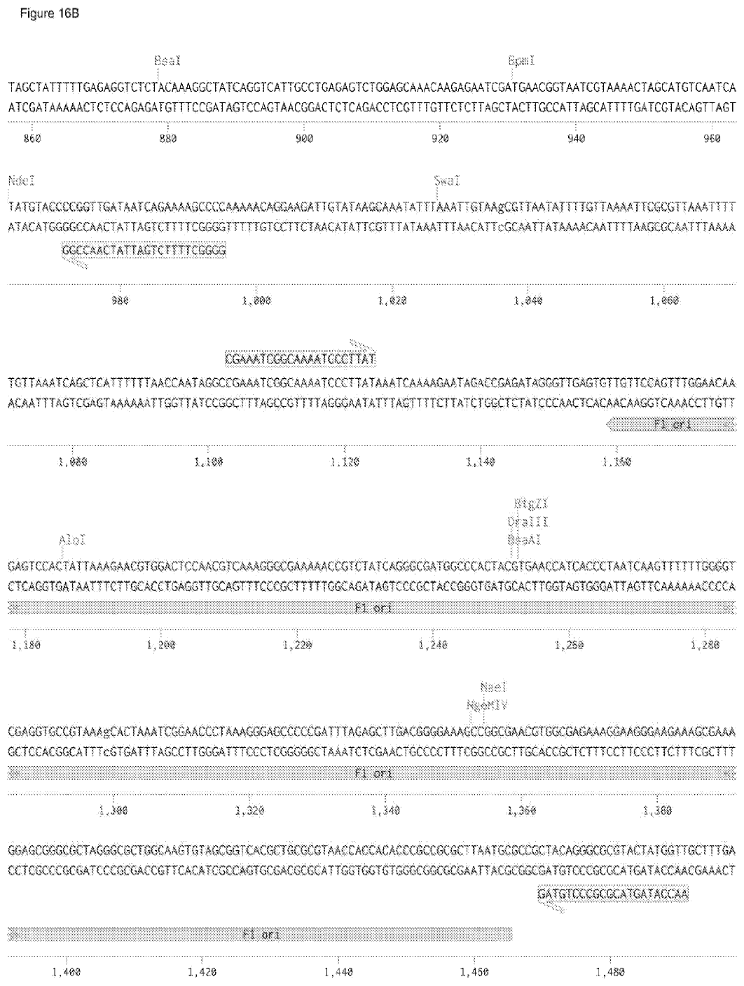

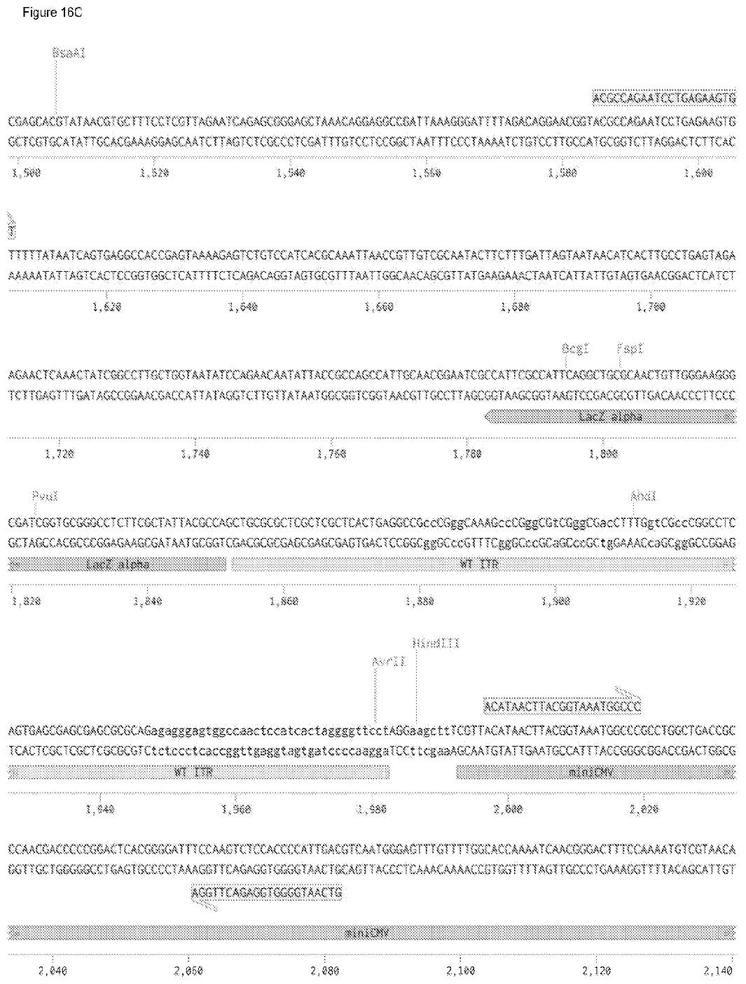

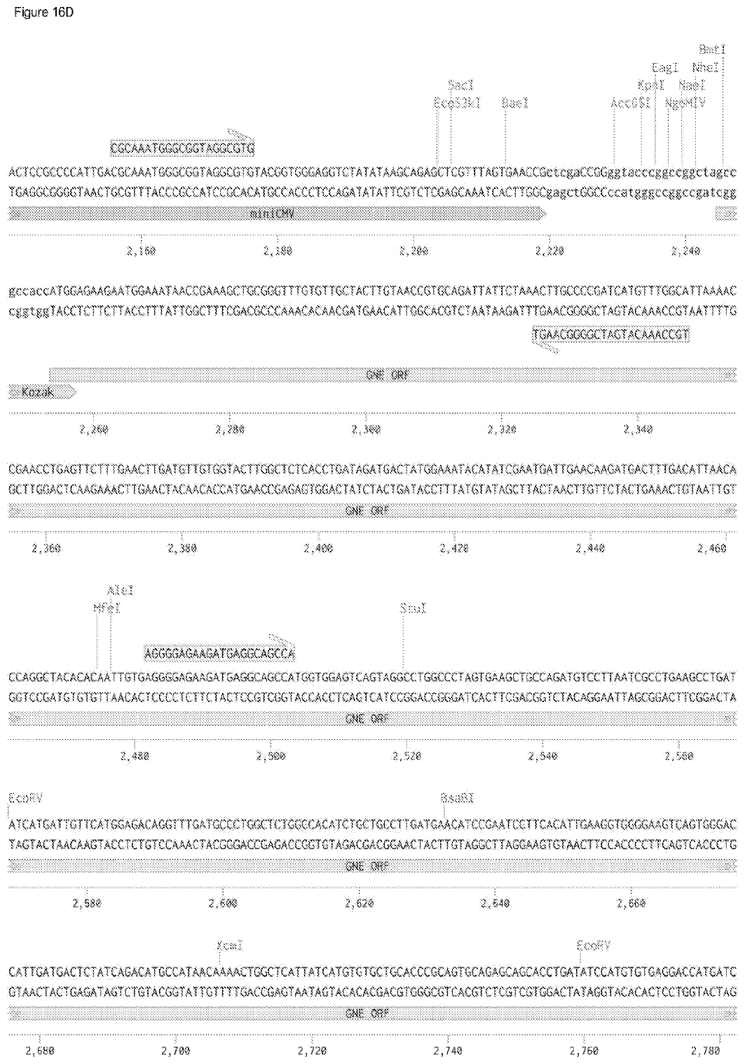

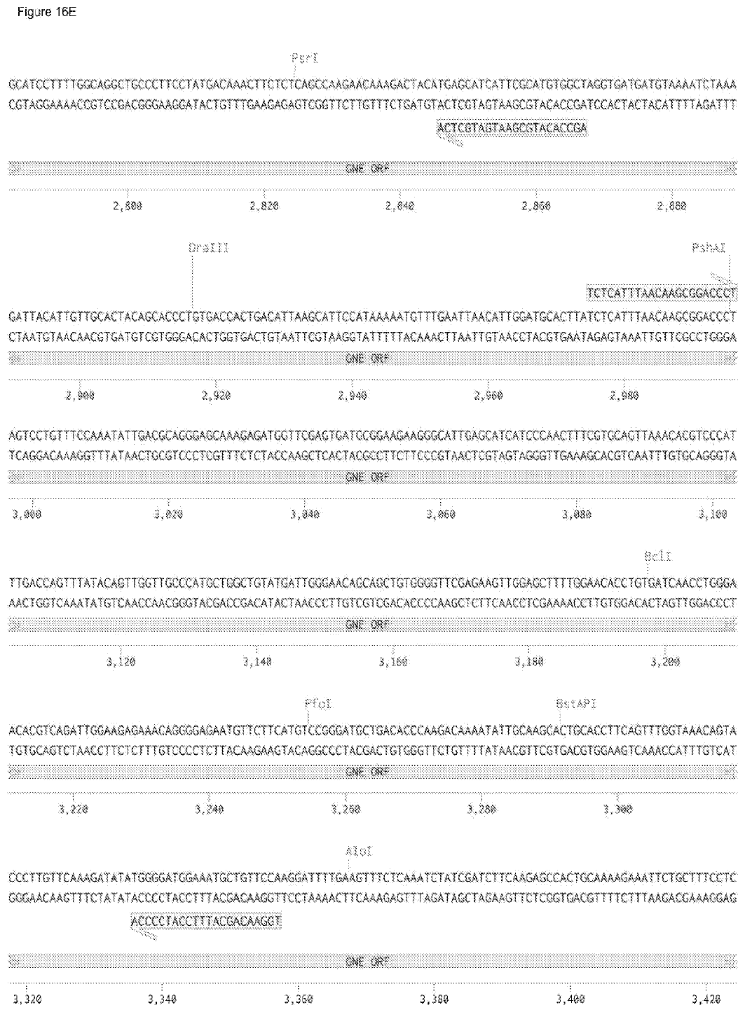

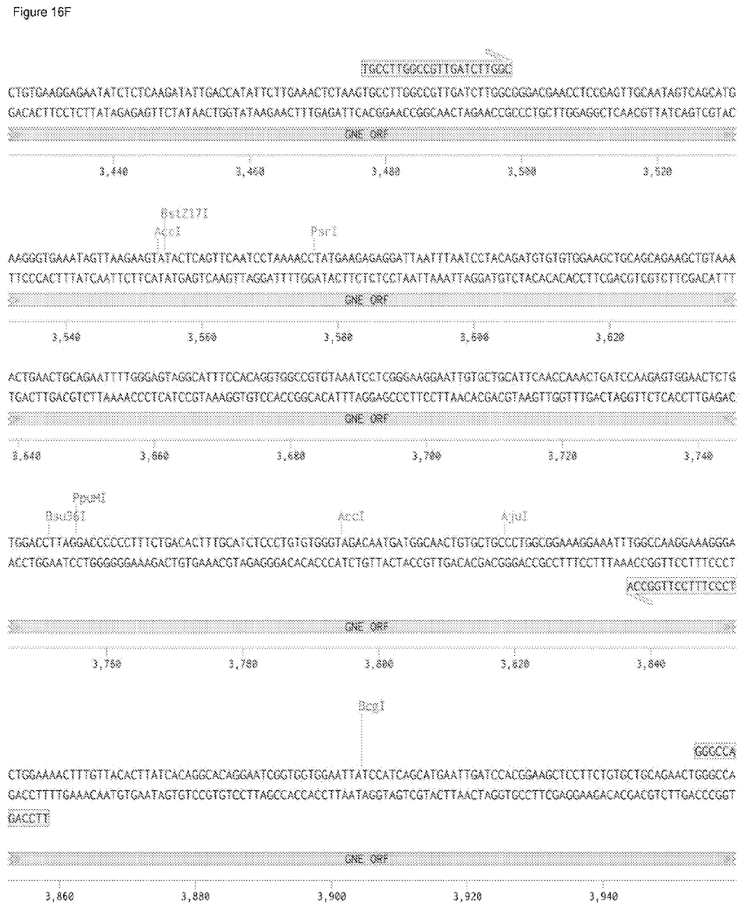

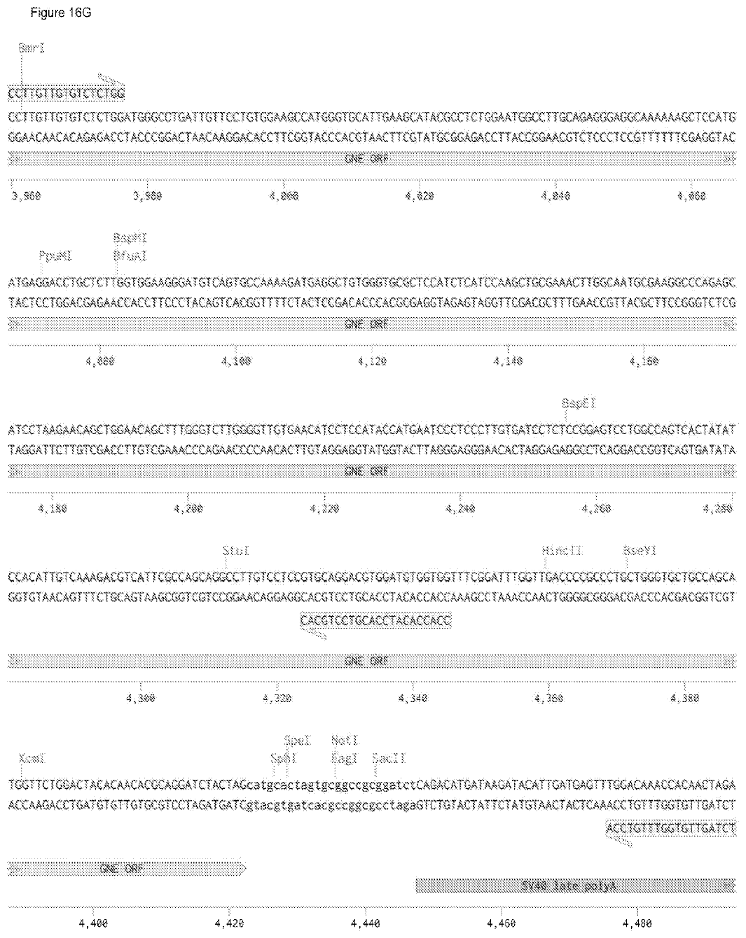

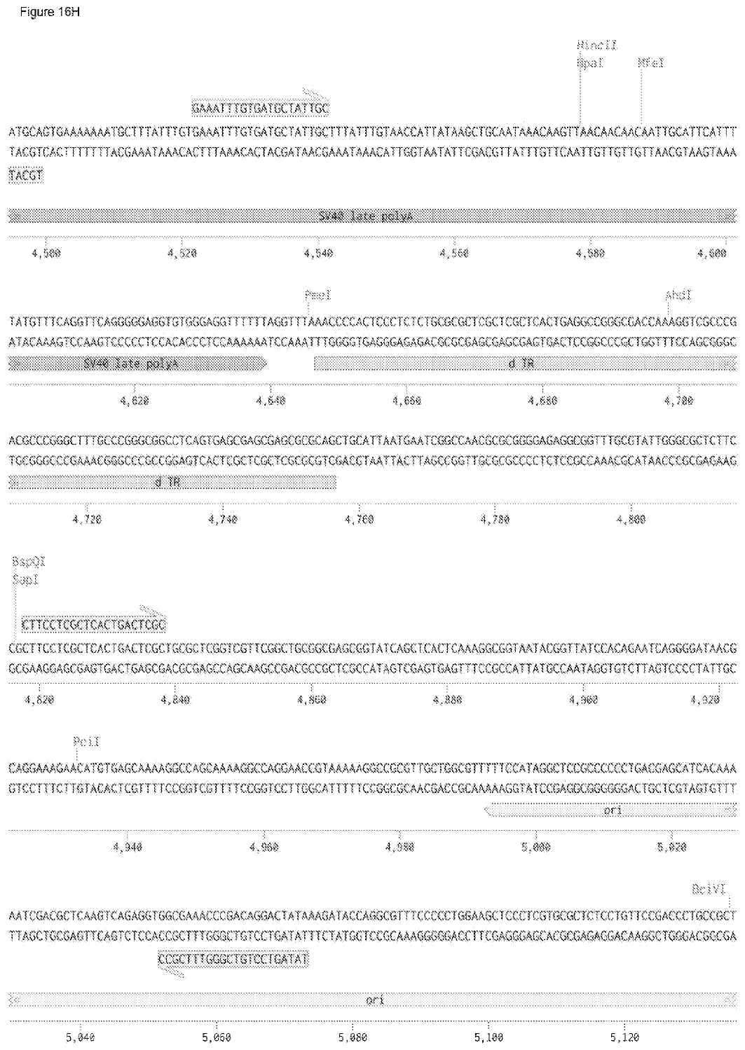

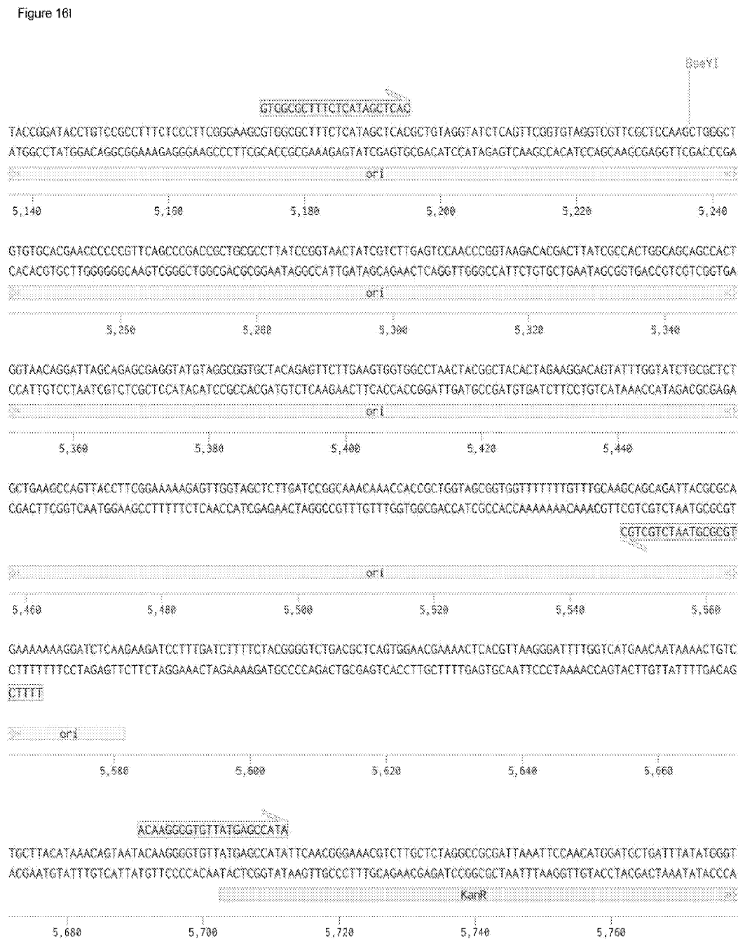

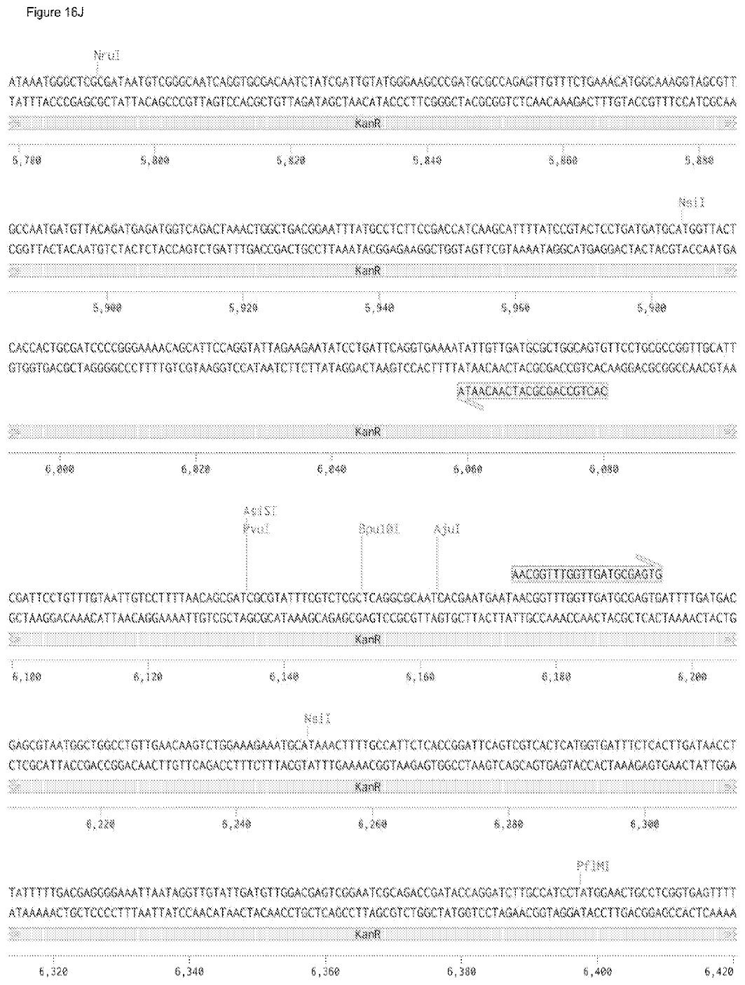

The following exemplary DNA constructs encoding UDP-GIcNAc-epimerase/ManNAc-6 kinase were generated as follows:
rAAVrh74.CMV.GNE (variant 2) set out in FIG. 1A and encoded by the polynucleotide of FIG. 2 (SEQ ID NO: 12).
rAAVrh74.MCK.GNE (variant 2) set out in FIG. 1B and encoded by the polynucleotide of FIG. 3 (SEQ ID NO: 13).
rAAVrh74.MHCK7.GNE (variant 2) set out in FIG. 1C and encoded by the polynucleotide of FIG. 4 (SEQ ID NO: 14).
rAAVrh74.GNE promoter.GNE (variant 2) set out in FIG. 1D and encoded by the polynucleotide of FIG. 5 (SEQ ID NO: 15).
rAAVrh74.MHCK7.GNE(variant 2).FGFIIRES.FS344 set out in FIG. 1E and encoded by the polynucleotide of FIG. 6 (SEQ ID NO: 16).
rAAVrh74.MHCK7.GNE(variant2).FGF1 IRES.HB-IGF1 set out in FIG. 1F and encoded by the polynucleotide of FIG. 7 (SEQ ID NO: 17).
rAAVrh74.CVM.GNE(variant 2).FGF1IRES.FS344 set out in FIG. 1G and encoded by the polynucleotide of FIG. 8 (SEQ ID NO: 18).
rAAVrh74.CMV.GNE(variant 2).FGF1 IRES.HB-IGF1 set out in FIG. 1H and encoded by the polynucleotide of FIG. 9 (SEQ ID NO: 19).
rAAVrh74.MCK.GNE(variant 2).FGF1IRES.FS344 set out in FIG. 1I and encoded by the polynucleotide of FIG. 10 (SEQ ID NO: 20).
rAAVrh74.MCK.GNE(variant2).FGF1 IRES.HB-IGF1 set out in FIG. 1J and encoded by the polynucleotide of FIG. 11 (SEQ ID NO: 21).
rAAVrh74.GNE promoter.GNE(variant 2).FGFIIRES.FS344 set out in FIG. 1K and encoded by the polynucleotide of FIG. 12 (SEQ ID NO: 22).
rAAVrh74.GNE promoter.GNE(variant 2).FGF1 IRES.HB-IGFI set out in FIG. 1L and encoded by the polynucleotide of FIG. 13 (SEQ ID NO: 23).
rAAVrh74.mimiCMV.GNE set out in FIG. 1M and encoded by the polynucleotide of FIG. 14 (SEQ ID NO: 24).
rAAVrh74.mimiCMV.GNE(variant 2).FGF1IRES.FS344 set out in FIG. 1N and encoded by the polynucleotide of FIG. 15 (SEQ ID NO: 25).
rAAVrh74,miniCMV.GNE(variant 2).FGF1.IRES.HB-IGF1 set out in FIG. 1O and encoded by the polynucleotide of FIG. 16 (SEQ ID NO: 26).

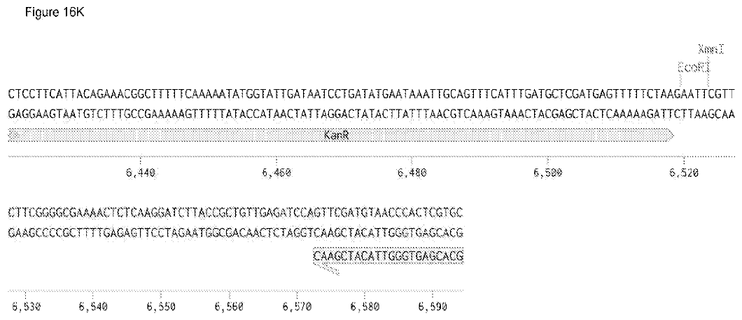

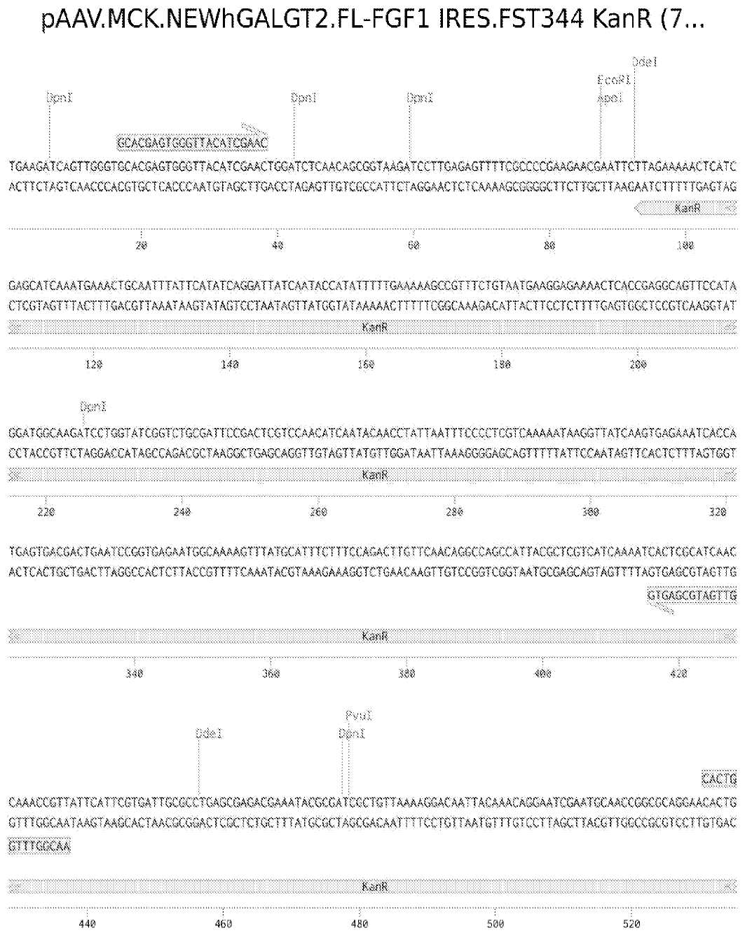

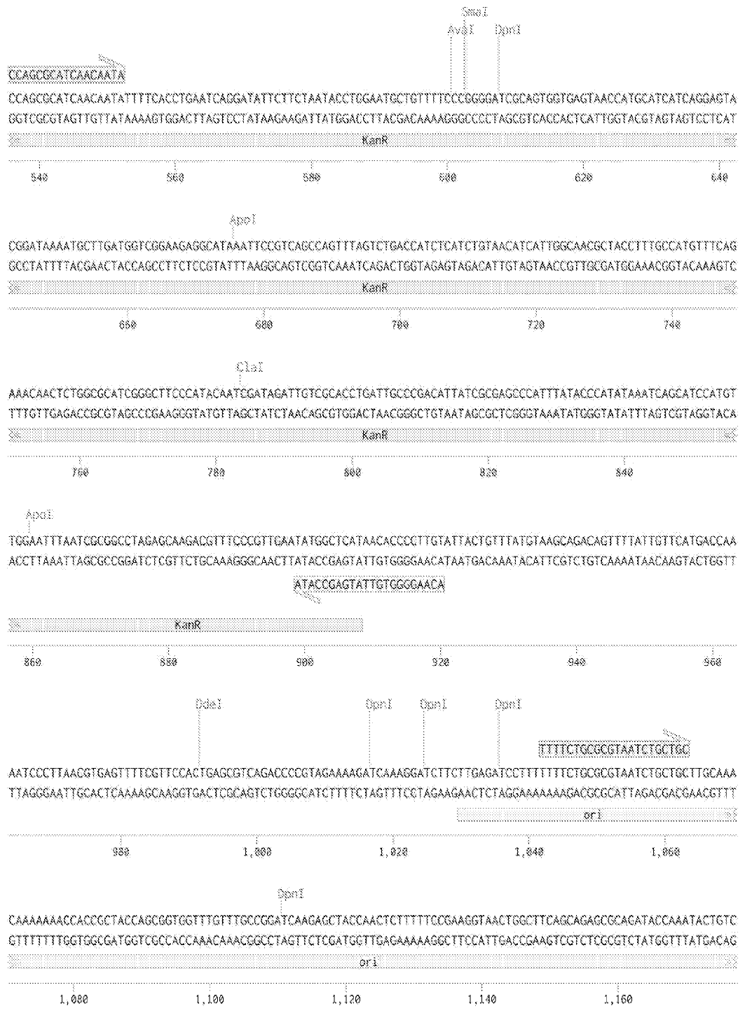

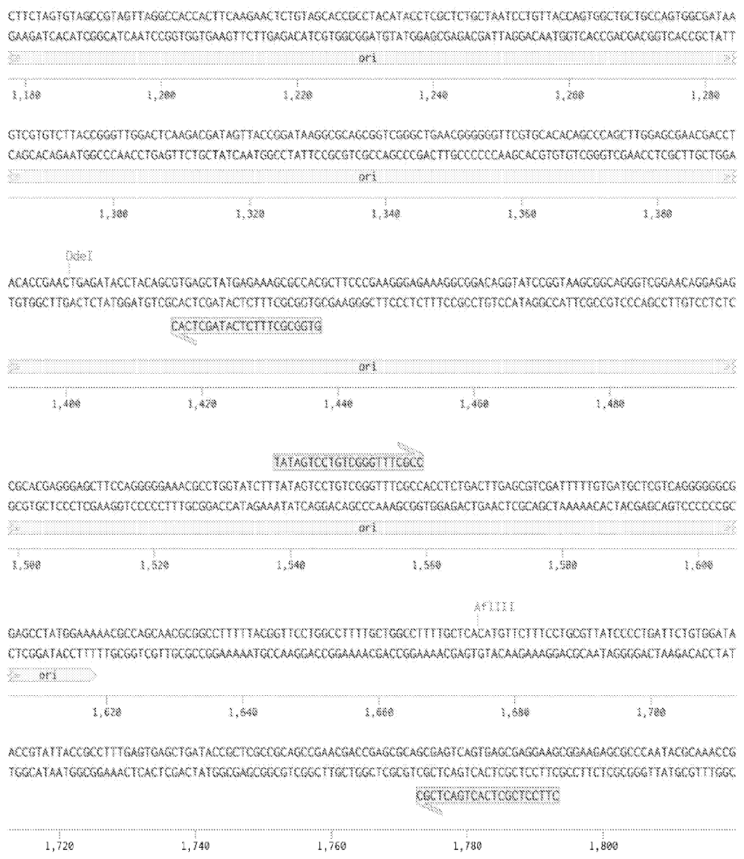

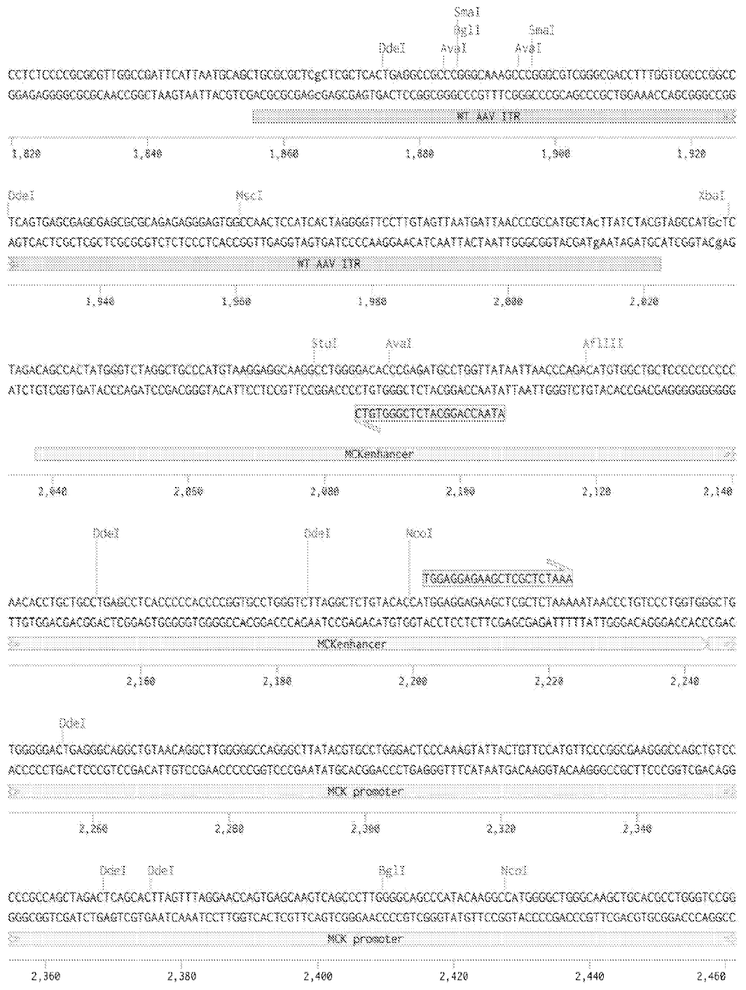

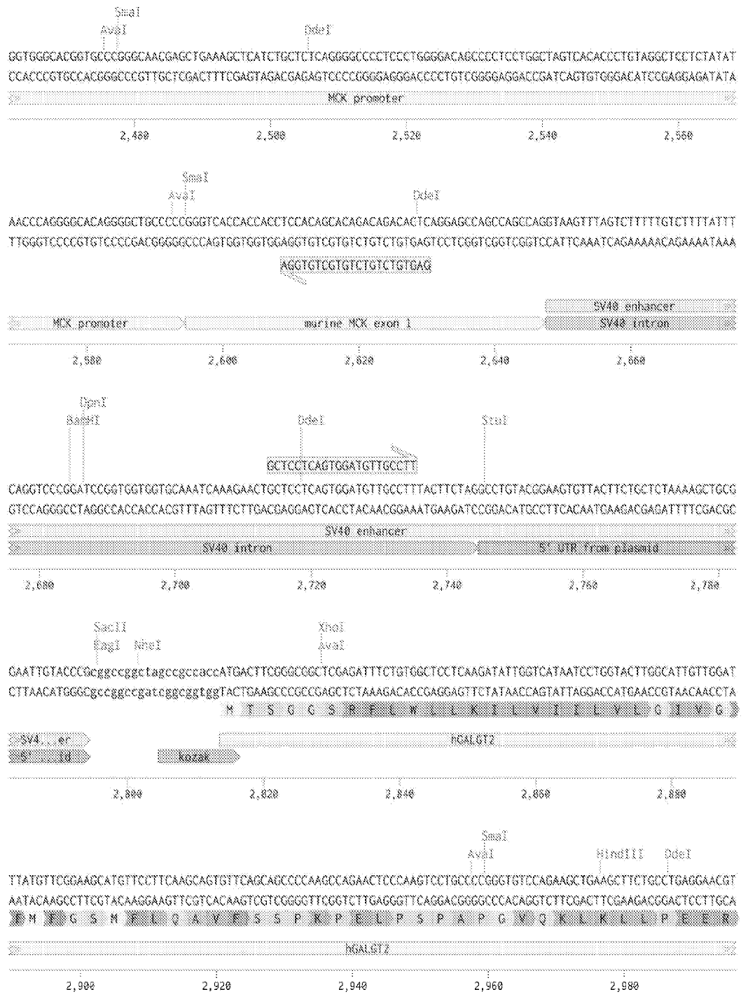

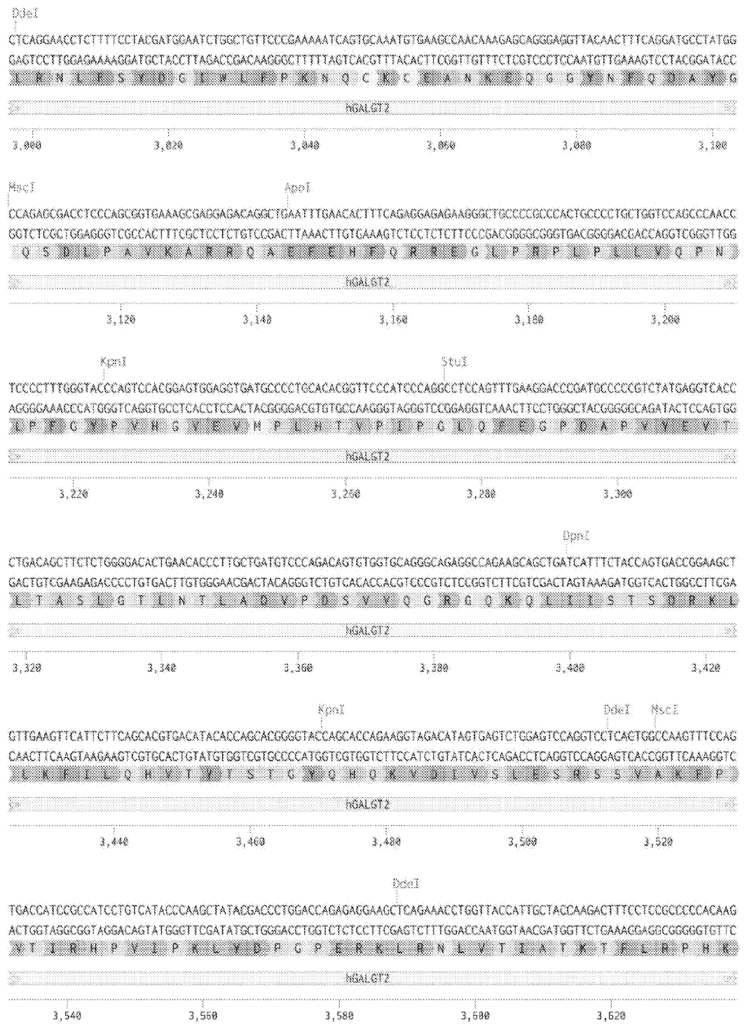

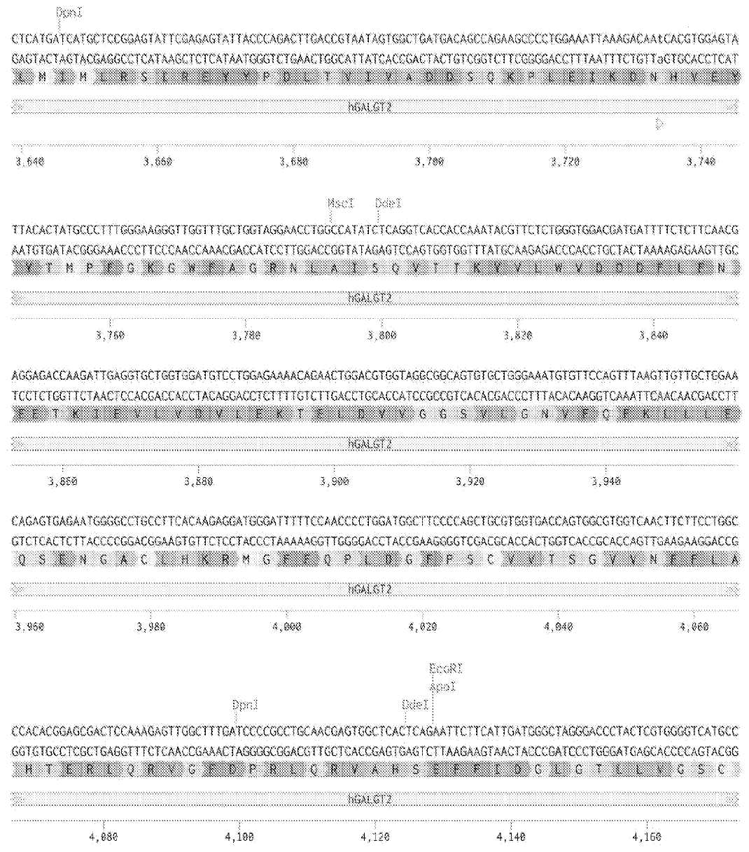

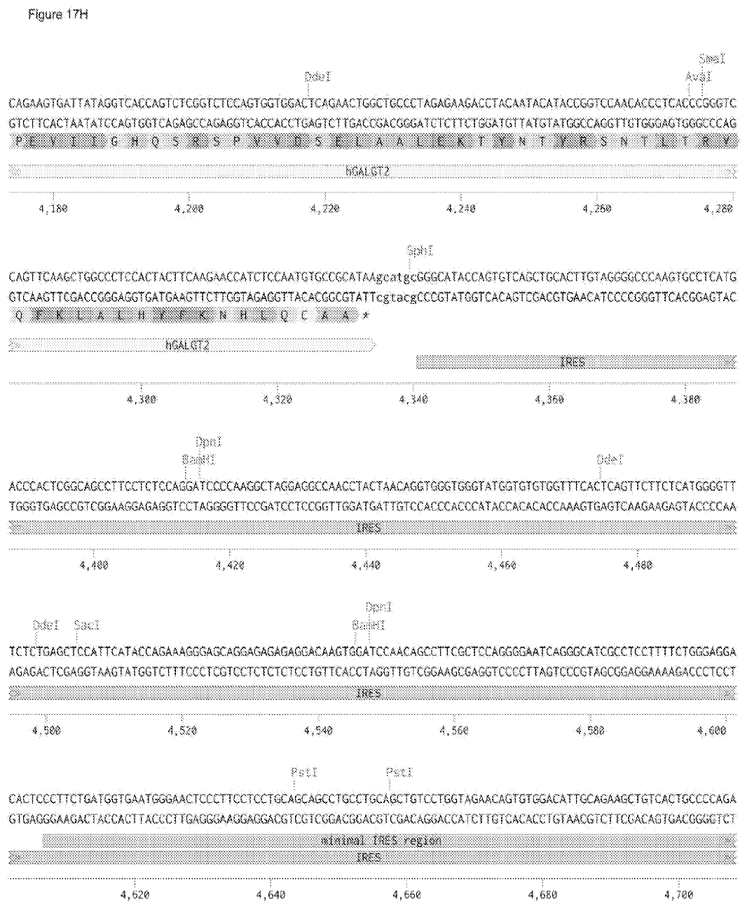

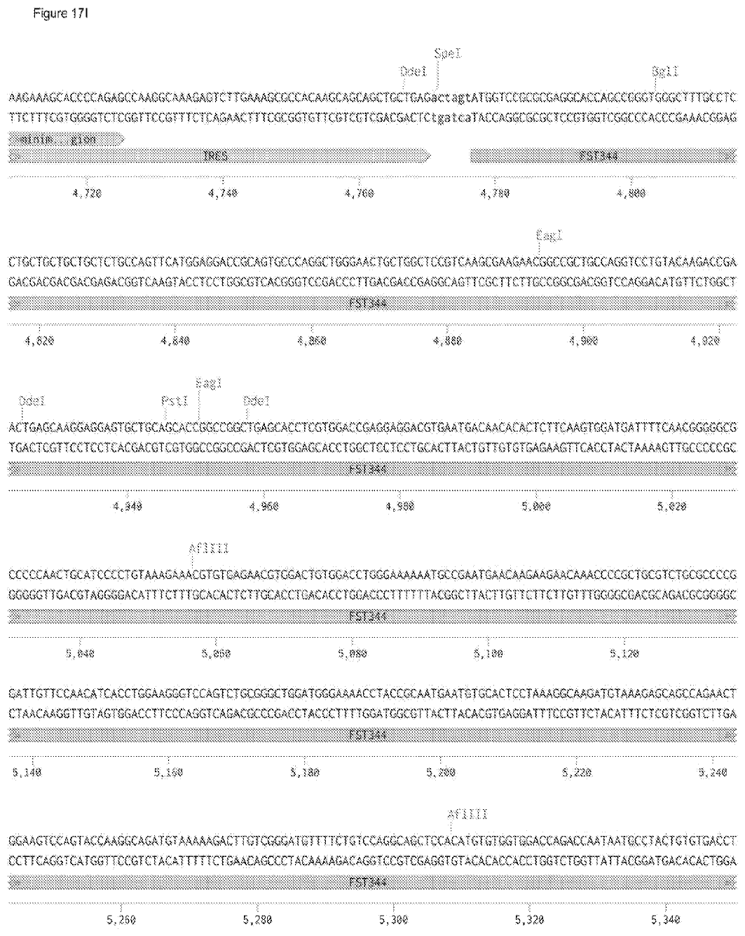

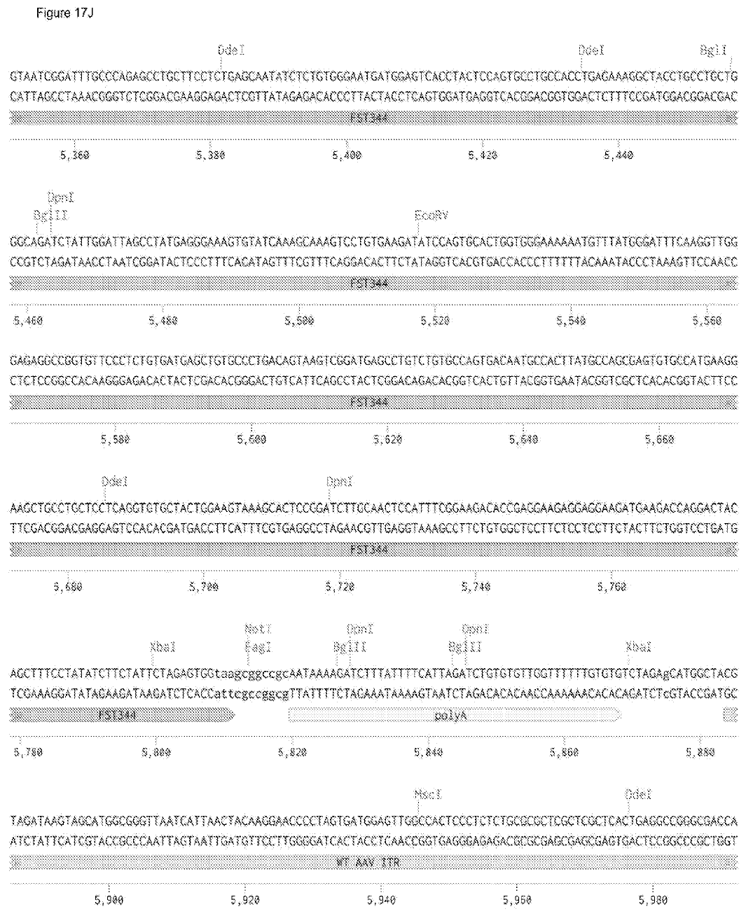

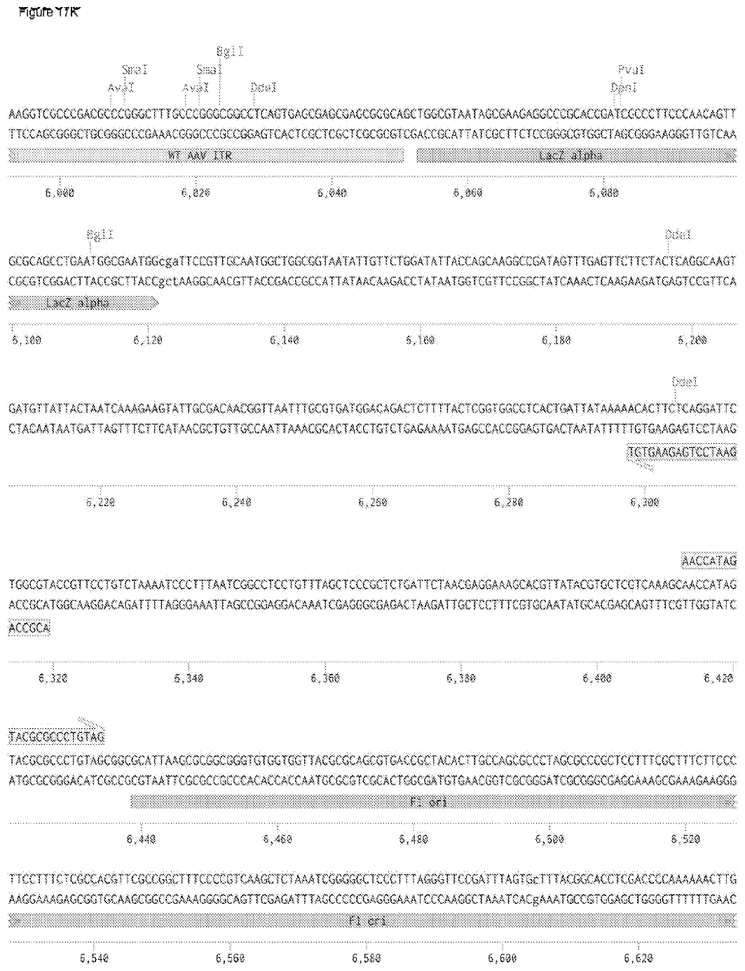

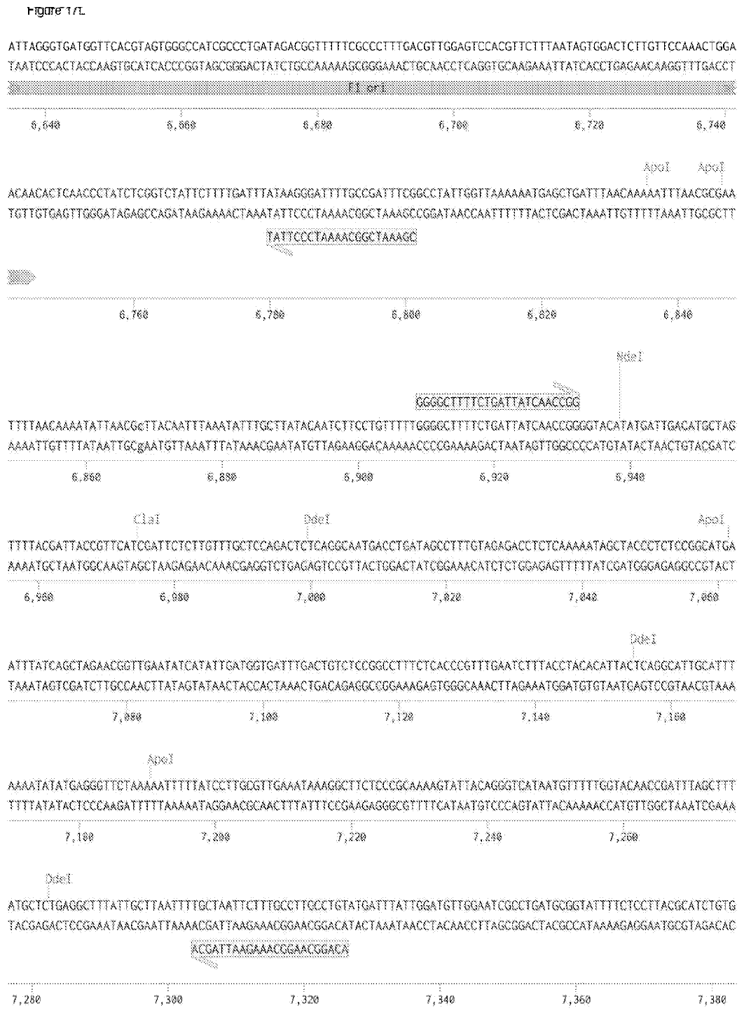

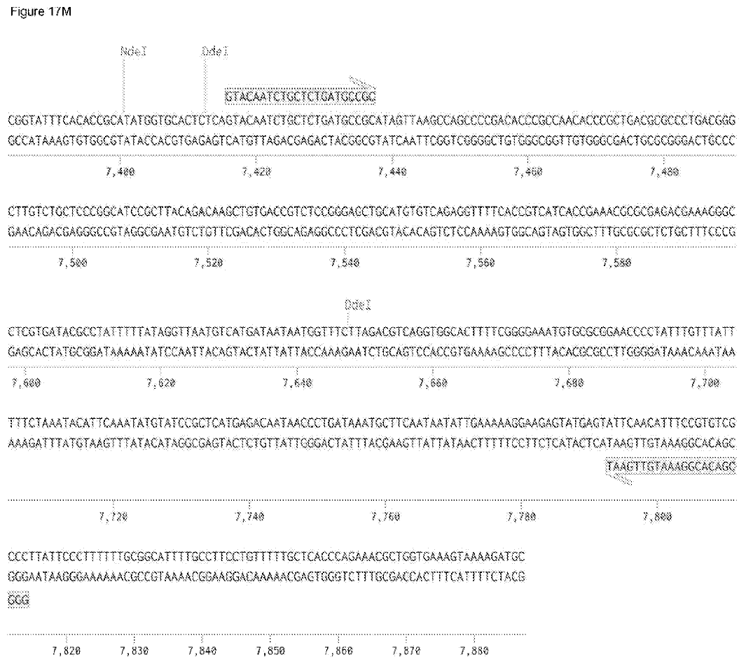

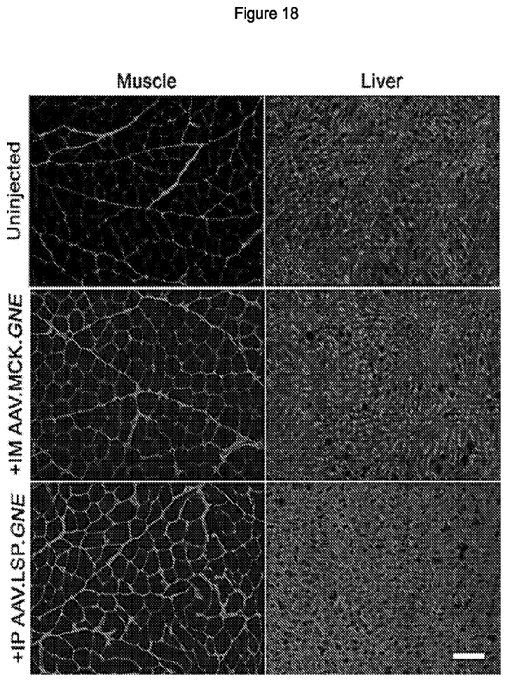

In addition, the exemplary DNA construct encoding GalNAc transferase rAAVrh74.MCK.GALGT2.FGF1IRES.FS344 set out in FIG. 1P and encoded by the polynucleotide of FIG. 17 (SEQ ID NO: 38) was generated as follows.

The disclosed plasmid contains a human GNE cDNA or GATGT2 expression cassette flanked by AAV2 inverted terminal repeat sequences (ITR), these expression cassettes may also comprise a FGFIIRES and a second transgene that induces muscle growth such as Follistatin 344 or HB-IGF1. The expression of the GIcNAc epimerase/ManNAc kinase protein or GalNAc transferase protein is guided by either the CMV, MCK, MHCK7, miniCMV or the GNE promoter. CMV is the cytomegalovirus promoter (SEQ ID NO: 3). MCK is the muscle creatine kinase promoter (CK7-like) (SEQ ID NO: 4). MHCK7 is the MCK promoter with additional enhancer (SEQ ID NO: 5). MiniCMV is a smaller version of the CMV promoter (SEQ ID NO: 7). GNE variant 2 is the GIcNAc epimerase/ManNAc kinase gene cDNA variant 2, which encodes a 722 amino acid protein beginning within exon 3 (NM_005476; SEQ ID NO: 1). GALGT2 is the GALGT2 (or B4GALNT2) gene cDNA (Genbank Accession #AJ517771; SEQ ID NO: 36). miniFGF1IRES represents a minimal FGFI internal ribosomal entry site (SEQ ID NO: 8). FS344 is follistatin 344 amino acid form (SEQ ID NO: 10). HB-IGF1 is the signal peptide and pre-pro-peptide domains of human heparin binding Epidermal Growth Factor-like growth factor linked to exons 1˜4 of Insulin like growth factor 1 (SEQ ID NO: 11). GNE promoter (SEQ ID NO: 6) represents the indicated sequence elements immediately 5′ of exon 2, which should be used to drive expression of variant 2 GNE transcripts.

Wild type human GNE is a 2.2 kB cDNA, so a shortened FGF1A IRES may be required for some embodiments. This shortened FGF1A IRES can be as small as 100 bp, to fit FST (1.3 kB) into the 4.7 kB packaging limit of AAV. A shortened CMV promoters (220 bp instead of 800 bp) is denoted herein as the miniCMV, that works very well if this is an issue, which would allow for a longer IRES sequence to be used.

The GNE cDNA expression cassette or the GATGT2 cDNA expression cassette had a Kanamycin resistance gene, and an optimized Kozak sequence an optimized Kozak sequence, which allows for more robust transcription. rAAV vectors were produced by a modified cross-packaging approach whereby the AAV type 2 vector genome can be packaged into multiple AAV capsid serotypes [Rabinowitz et al., J Virol. 76 (2):791-801 (2002)]. Production was accomplished using a standard three plasmid DNA/CaPO4 precipitation method using HEK293 cells. HEK293 cells were maintained in DMEM supplemented with 10% fetal bovine serum (FBS) and penicillin and streptomycin. The production plasmids were: (i) plasmids encoding the therapeutic proteins, (ii) rep2-capX modified AAV helper plasmids encoding cap serotype AAVrh74 isolate, and (iii) an adenovirus type 5 helper plasmid (pAdhelper) expressing adenovirus E2A, E4 ORF6, and VA I/II RNA genes. A quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)]. A final titer (vg m1⁻¹) was determined by quantitative reverse transcriptase PCR using the specific primers and probes utilizing a Prism 7500 Real-time detector system (PE Applied Biosystems, Grand Island, N.Y., USA). Aliquoted viruses were kept at −80° C. until

All plasmids used to make AAV genomes to be packaged also contain a Kanamycin resistance gene (KanR) outside of the ITR sequences used for packaging of the genome. This allows for the DNA encoding the AAV genome to be transformed into bacteria to produce large amounts of DNA in the presence of Kanamycin, which will kill all non-transformed bacteria. KanR is not packaged into the AAV capsid in the AAV genome used to treat patients, but its presence allows for DNA production in bacteria.

### Example 2

### Expression and Testing

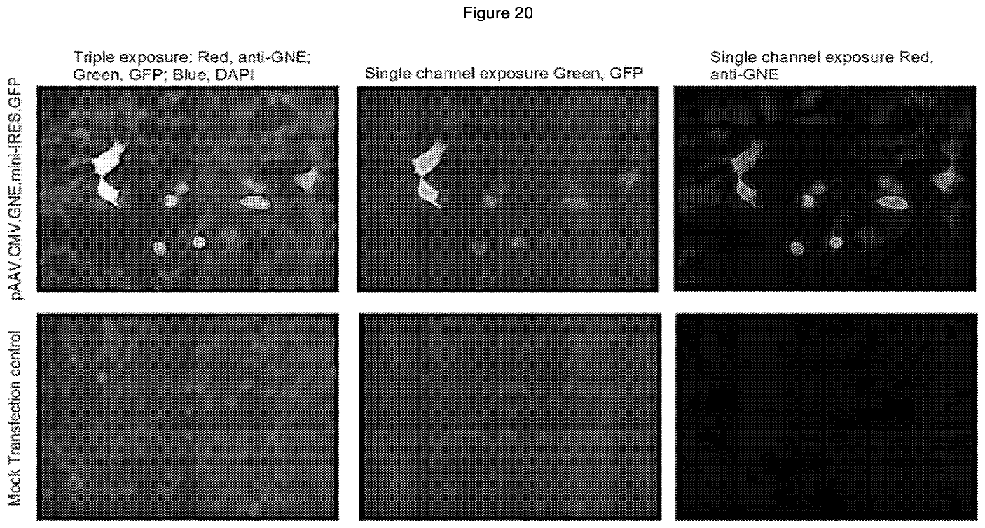

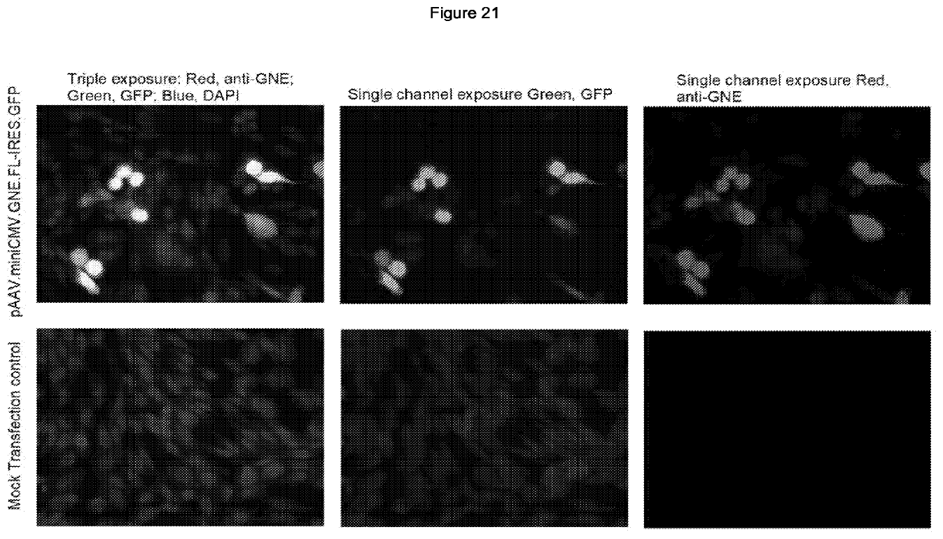

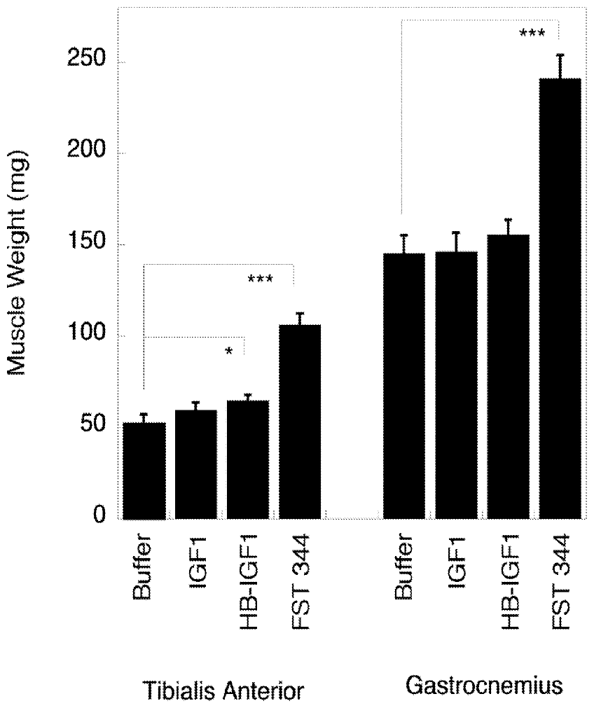

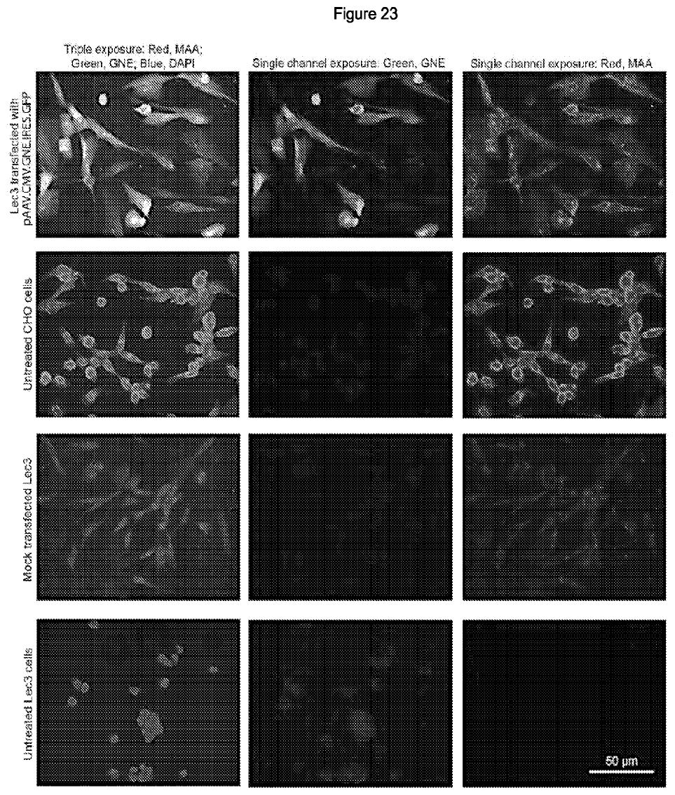

The vector genomes for AAV vectors rAAV.CMV.GNE.mini-IRES.GFP and rAAV.miniCMV.GNE.Full-length (FL)-IRES.GFP were tested by transfecting them into GNE-deficient Lec3 CHO cells (Lec3) cells to demonstrate that the vectors described in Example 1 express both GFP and a second protein. The miniIRES is a further shortened version of the IRES and it is set out as SEQ ID NO: 7. As shown in FIGS. 20, the presence of the mini-IRES in the vector genome allowed for expression of the second protein downstream of the IRES (GFP). In FIG. 20, the GFP shows endogenous fluorescence, while GNE expression is demonstrated by immunostaining. As shown in FIG. 21, the full-length IRES also allowed for expression of the second gene (GFP). FIG. 23 shows that GNE can allow for sialic acid production when introduced into Gne-deficient Lec3 cells at the same time that the IRES produces a second protein, in this case GFP.

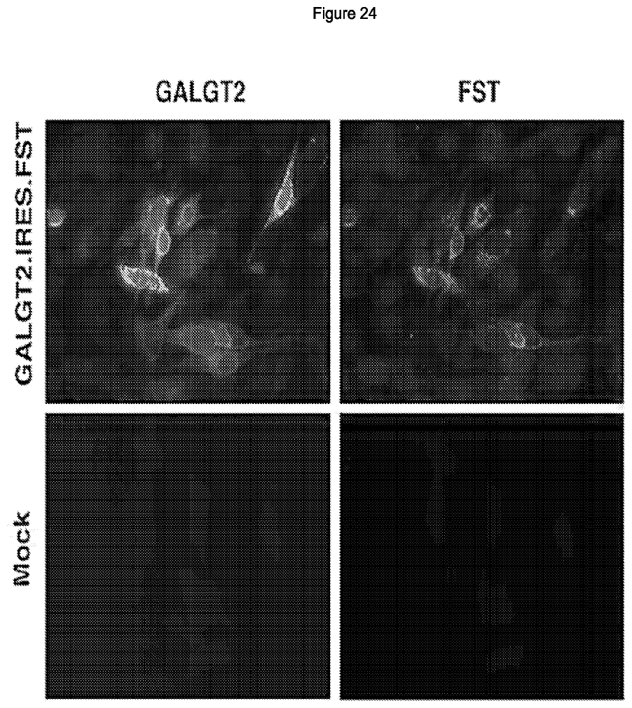

FIG. 24 shows that any transgene of an appropriate size can be in the first position as a gene replacement or surrogate gene replacement. C2C12 cells were transfected with the AAV vector rAAV.MCK.GALGT2.IRES.FS344, which expresses GALGT2, a surrogate gene replacement for dystrophin in Duchenne Muscular Dystrophy. Expression of both GALGT2 (stained green) and FST (stained in red) was observed in the same cell. Inclusion of the IRES allows for production of a muscle growth factor in the same cells, in this case follistatin (FS344 or FST).

For additional analysis, any of the AAV vectors described in Example 1 are tested in muscle cells and in GNE-deficient CHO cells (Lec3) cells to demonstrate their function. AAV vectors are added at different doses, from 10 MOI (multiplicity of infection) to 10,000 MOI in log increments. High MOI are typically needed for AAVs to infect cells in culture, as AAV works far better in vivo than in vitro. C2C12 myoblast and C2C12 myotube cultures as well as CHO-K1 (wild type) cells and Lec3 cells, a CHO cell variant that lacks Gne activity are infected with the provided AAV vectors.

In vivo tests of function are carried out in Gne deficient mice, where GNE gene correction is tested either by demonstration of UDP-GlcNAc epimerase enzyme activity or by measurement of free or membrane bound sialic acid. These measurements are carried out either by gas chromatorgraphy-mass spectrometry using known standards or by quantitative lectin staining using Maackia amurensis agglutinin or Sambuca Nigra agglutinin, which bind sialic acid. Assays of Gne enzyme activity, for example UDP-GlcNAc epimerase activity, may also define gene replacement. FST and IGF1 induction of muscle growth is assayed by weighing limb muscles and comparing them to total animal weight (e.g., see FIG. 22), by sectioning muscles and measuring the area and number of skeletal myofibers present using hematoxylin and eosin staining of thin sections, coupled with morphometric software, or by physiological measures of muscle strength, including grip strength, ambulation, and ex vivo measures of specific force, for example in the tibialis anterior or extensor digitorum longus muscle.

Cells are stained for MAA or SNA (conjugated to Cy3) to assess sialylation, and with antibodies to GNE, FST, or IGF1 to assess protein co-expression. The same constructs are infected in larger cell cultures to assess protein expression by Western blotting and ELISA, as previously described (Haidet et al., Proc. Natl. Acad. Sci. 105(11): 4318-22, 2008; Hennebry et al., J. Endocrinolgy 234: 187-200, 2008). Changes in signaling, in particular reduced phosphor-Smad 2 levels for FST and increased phosphor-Akt (for IGF1) is assessed with immunostaining and Western blotting, as done previously (Chandraskeharen et al. Muscle Nerve 39(1):25-41, 2008; Cramer et al., Mol. Cell. Biol. 39(14), 2019). In all cases, for gene expression is assessed by qRT-PCR and for AAV biodistribution by qPCR, as previously described in Xu et al. (Mol. Ther. 2019). We have already identified the ideal IGF1 splice form for muscle growth (ns).

The bicistronic vectors described in Example 1 allow for GNE protein expression and either follistatin or IGF1 protein expression from the same mRNA. Infection of muscle cultures allows for greater IRES-mediated bicistronic expression, as the FGF1A IRES shows much greater effects in muscle than in non-muscle cell lines. GNE expression in Lec3 cells increase sialylation, as these cells are deficient in Gne enzyme activity, and this is equal to or exceeds SA levels in normal CHO-K1 cells.

As shown in FIG. 4, both muscle and liver specific expression of GNE contributed to muscle SA expression. Sialic acid staining of liver and muscle after intramuscular injection of rAAVrh74.MCK.GNE or IP injection of rAAVrh74.LSP.GNE in GNED176V TgGne^{-/-} mice was carried out. Sialic acid staining in muscle and liver was shown for time-matched images 6 months after IM injection of a muscle-specific GNE gene therapy vector in muscle or IP delivery of a liver-specific GNE gene therapy vector in liver, both at a dose of 5×10¹¹ vg. qRT-PCR showed a 30-fold increase in muscle expression for MCK, with no expression in liver, while LSP showed an 8-fold increase in liver expression, with no increase in muscle (ns). After 6 months, MCK increased muscle SA, but LSP increased it even more so, likely the result deposition of serum glycoprotein secreted by the liver in the muscle extracellular matrix.

To demonstrate that transduction of muscles cells using a rAAV vector results in muscle growth, the tibialis anterior (TA) muscle of C57Bl/6J mice as injected with 1×10¹¹ vg (vector genomes) and the gastrocnemius (Gastroc) muscle was injected with 5×10¹¹ vg of AAV expressing Insulin-like growth factor 1 (IGF1, muscle form Ea), HB-IGF1, or follistatin (FST) form 344. Muscles were dissected and weighed at 2 months post-injection, showing significant increases for HB-IGF1 and FST344 in the TA and for FST344 in the Gastroc compared to injection of buffer alone (see FIG. 21).

### Example 3

### Mouse Model for GNE Function in Adult Mice

A mouse model of GNE myopathy is generated by introducing a floxed Gne allele into exon 3 of the mouse Gne gene, and introduction of this allelle is sufficient to allow for Cre-mediated deletion, yielding a GNE myopathy-like phenotype. The field of GNE myopathy research has been plagued by the inadequacies of the diseases models that have been made. GNED176VTgGne^{-/-} mice were first reported to be a good late onset model for GNE myopathy (Malicdan et al., Hum. Mol. Ther. 16(22): 2669-82, 2007; Malicdan et al Nat. Med. 15(6): 690-5, 2009), but upon further breeding these mice have lost much of their phenotype, while a mouse knock-in of the GNE_{M712T} (now GNE M743T) Persian Jewish mutation led to lethality[10], in part due to kidney dysfunction, while other strains of the same line show no phenotype at all (Sela et al., Neuromolecular medicine 15(1): 180-91, 201311. As Gne is essential in mice, leading to lethality between E8.5 and E9.5, creation of a floxed allele to delete the gene in the adult mouse should allow for creation of a robust body-wide or muscle-specific phenotypes using Cre-mediated deletion. This, in turn, allows for more reproducible demonstrations of therapeutic efficacy.

Cas9-CRISPR is used to make a deletion in exon 3 on the mouse Gne gene, the exon where the functional domain for UDP-GlcNAc epimerase begins and which contains the translation start site for the Gne gene. Fertilized oocytes are injected with Cas9-CRISPR, relevant guide RNAs, and a long DNA oligonucleotide that allows for recombination to create a new exon 3 flanked by loxP recombination sites. Founders are bred out over two generations and then shipped from vendor (Mouse Biology Program at UC Davis) for subsequent analysis.

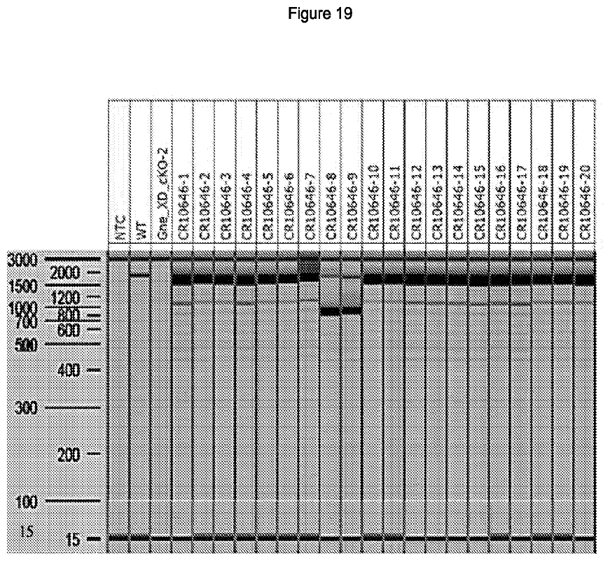

An 80-mouse injection session has yielded two Gne deletion exon 3 deletion founders (though no floxed founders) from 26 live mice (FIG. 19). This is followed by another injection round of 160 mice. If successful, rAAVrh74.CMV.Cre-GFP is used to express Cre systemically via IV tail vein injection, or use rAAVrh74.MCK.Cre-GFP is used to delete Gne only in skeletal muscle (and heart). These experiments provide a means of understanding how deletion of Gne in the adult mouse cause disease phenotypes. While qPCR results showed no floxed allele was present bordering exon 3 in these founders, they can nevertheless be used to make Gne^{-/-} mice. These mice also demonstrate that the guide RNAs used do allow for Cas9-CRISPR deletion of Gne exon 3.

Assays for detecting disease phenotypes are currently available. For example, to understand loss of sialylation MAA and SNA lectin staining is used to visualize sialic acid expression (with endogenous Cre-GFP used to see which cells Cre is expressed in), which bind α2,3- and α2,6-linked SAs respectively. qRT-PCR is used to understand loss of Gne gene expression (and increase in Cre-GFP gene expression). qPCR is used to understand the number of vector genomes present per nucleus in each muscle tissue and the extent of gene deletion. For methods see Kim et al. (Mol. Cell Neurosci. 39(3): 452-64, 2008) and Xu et al., (Mol. Ther. 2019). The GC-MS/MS method is also used to measure total free sialic acid and total glycoprotein conjugated N- and O-linked sialic acid, see Yoon et al., (PLoS Currents 2013). Last, Gne enzyme activity, either UDP-GlcNAc epimerase activity or ManNAc 6 kinase activity, may be used to measure the degree of functional gene replacement.

Muscle pathology analysis includes staining of thin sections with hematoxylin and eosin, trichrome, and Congo Red. Measures include numbers of inclusion bodies, myofiber size, central nuclei, variance in myofiber size, fibrosis, and non-muscle area (wasting), see Chandraskeharen et al. (Muscle Nerve 39(1):25-41, 2008). If inclusion bodies are found, their ultrastructure using electron microscopy is assessed. Muscle function is determined by measuring grip strength, ambulation (treadmill walking), open field tests, and ex vivo specific force and force drop during repeated contractions (in TA and EDL), see (Chandraskeharen et al. (Muscle Nerve 39(1):25-41, 2008; Martin et al., Am. J. Physiol. Cell Physiol., 296:C476-88, 2009).

Floxed Gne mice are mock-injected (control) or 1×10¹⁴ vg/kg AAV.CMV.Cre-GFP or AAV.MCK.Cre-GFP at 2 months of age, with analysis at 1, 2 and 4 months post-injection. Six mice (3 males and 3 females) per group are injected, and age-matched mock-injected mice and wild type mice are used as controls.

If the above experiments do not generate any floxed mouse founders from these injection sessions, two Gne deletion founders are breto homozygosity in the presence of 2 g/kg/day ManNAc, which rescues sialylation and lethality in the GNE_{M743T} model and in Gne^{-/-} model. Here, mice are given ManNAc at 2-4 g/kg/day in water from conception onward. Once the pups are weaned, ManNAc can be removed and gene therapies tested, essentially creating an inducible Gne knock-out model. These mice do not allow for a muscle-specific Gne deletion, one could rescue such mice at the time of ManNAc withdrawal with AAV.CMV.GNE_{M712T} or AAV.CMV.GNE_{D207V} and test for a muscle-specific disease if needed. If needed, one could also down-regulate endogenous Gne gene expression in wild type or in Gne^{+/-} mice using a micro-RNA or siRNA targeted to the mouse and/or human GNE allele. While such experiments would be subject to the same issues as the previous transgenic and knock-in models, the ability to dose the mouse with different amounts of the GNE mutant allows for more control.

### Example 4

### In Vitro AAV.GNE Potency Assay

An MAA-HRP ELISA allows for a comparison of sialic acid levels between Gne-expressing CHO cells and Gne-deficient Lec3 cells, and this assay should be sufficient to define AAV.GNE potency after infecting Lec3 cells with different concentrations of AAV.GNE.

Any gene therapy clinical development plan must contain a potency assay that effectively describes the biological activity of the AAV vector to be used, in this case a AAV.GNE gene therapy vector. This assay will be carried out annually on clinical lots of AAV to demonstrate that activity has not been lost, and it will be carried out to demonstrate that the AAV to be used in patients has the necessary biological activity when it is administered.

Infection of different amounts of AAV.GNE into Gne-deficient Lec3 (mutant CHO) cells (Hong et al. J. Biol. Chem. 278:53045-530454, 2003) is carried out to bring Lec3 sialylation up to a defined amount found in equivalent numbers of normal CHO cells, thus demonstrating the potency of the AAV vector's biological activity. This is carried out using Maackia amurensis agglutinin (MAA), which binds a2,3-linked sialic acids (Song et al. 286: 31610-31622, 2011). Such an assay could be applied to any number of GNE-containing gene therapy vectors.

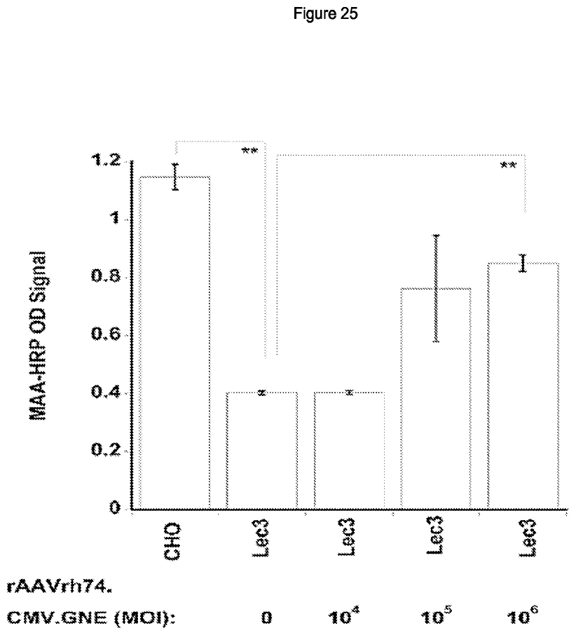

Lec3 cells fed 10% serum-containing media did not show a difference from normal CHO cells in MAA-HRP-binding ELISA assays (ns), but feeding of Lec3 cells for 3 days in Opti-MEM media, a defined serum-free media, eliminated most MAA binding, while CHO cells maintain their MAA signal (FIG. 25). This is because free sialic acid (SA) from serum was taken up into cells and incorporated into lipids and glycoproteins, bypassing the Gne deficiency in Lec3 cells. This bypass can only be removed by eliminating serum from the media used to feed the cells. For example, infection of rAAVrh74.CMV.GNE into Lec3 cells fed in Opti-MEM for two days allowed for partial recovery of a MAA-binding signal at 10⁵ or 10⁶ MOI (multiplicity of infection) doses (FIG. 25). Some additional optimization work, (i.e., varying time of AAV infection, varying time of Lec3 cells in Opti-MEM, or varying AAV dose used) may need to be carried out to expand signal differences in this assay. Regardless, this assay is able to determine the potency of AAV.GNE vectors by adding different amounts of AAV to Lec3 cells and defining potency as the dose required to recover a normal (or half-normal) CHO cell signal. As shown in FIG. 23. when an AAV plasmid containing CMV.GNE was transfected into Lec3 cells and co-stain for GNE protein and MAA, we find that GNE-expressing Lec3 cells actually secrete sialylated glycoproteins that MAA can bind on non-GNE expressing cells. Thus, this potency assay may be more sensitive than assays where GNE protein or gene levels are used as the standard due to trans effects from secreted SA-containing proteins. To test this assay, CHO cells and Lec3 cells are transferred at 10,000 cells/well into 96-well ELISA plates, with triplicate wells being used for each condition. Cells are fed Opti-MEM for one day, after which cells will be re-fed Opti-MEM and allowed to grow for two more days with or without AAV. During that period, some cells are infected with different doses of an rAAV comprising the GNE cDNA. Note that any serotype of AAV could be used in these assays. The conventional measure of MOI is used to carry out different levels of AAV infectivity, including 1×10⁴, 5×10⁴, 1×10⁵, 5×10⁵, 1×10⁶, 5×10⁶, and 1×10⁷. It is important to note that AAV is not very efficient at infecting cells grown in culture. This differs very significantly from its robust ability to infect cells in tissues. As such, a relatively large concentration of virus needs to be used. Because so few cells need to be infected, however, this assay still utilizes only a very small amount of virus per assay.

After infection, cells are washed in phosphor-buffered saline (PBS), fixed in 4% paraformaldehyde in PBS for 20 minutes, and washed again in PBS. Cells are then blocked in PBS containing 1% fish gelatin (which contains no sialic acid) for 1 hour, incubated with 2 mg/mL Maackia ameurensus agglutinin linked to horseradish peroxidase (MAA-HRP) for one hour, and washed 3 times for 10 minutes each in PBS. Bound MAA-HRP is detected using standard HRP activity (OPD) colorimetric assay, which are developed for 20 minutes followed by quenching in acid for 10 minutes. Absorbance (color) is read at 450 nm on a SprectraMax plate reader.

A concentration curve to determine the optimal MAA-HRP concentration to use in this assay (2 μg/mL) has been generated. This MAA-HRP concentration yields OD readings at or above 1.0 for CHO cells and significantly reduced OD levels for Lec3 cells (e.g., see FIG. 25). The concentration curve is used to compare measures for uninfected Lec3 cells, which will have a low signal, AAV.GNE-infected Lec3 cells, which should show a dose-responsive increase in signal, and CHO cell levels, which should have a high signal that is our standard for full biological activity. The MOI that achieves a signal at the signal found in CHO cells (or half that signal, depending on ease of reproducibility) is the dose defined as giving potency. These measurements are repeated at least 6 times, using triplicate measures per data point, and determine intra- and inter-assay variability of repeated measures. AAV concentrations are adjusted as needed to more narrowly define the MOI required to give full potency if necessary.

When a rAAV vector comprises a muscle specific promoter. e.g. MCK, and the GNE cDNA sequence, a myoblast cell line where GNE has been deleted may be used. Other “muscle-specific” promoters, e.g. MHCK7, will work in CHO cells, but MCK does not. Gne-deficient myoblasts could be obtained from other NDF investigators, or if necessary, such a cell line is generated by deleting GNE in human cells using Cas9-CRISPR. Gne-deficient myoblasts may also be generated from primary cells cultured from Gne-deficient mice using methods described in Xia et al., Dev. Biol. 242: 58-73, 2002. Positive controls from normal wild type mice may also be used in this assay. It is important to understand that the cells used for the potency assay need not be human cells, just cells where sialic acid is defined as being absent or very reduced compared to a control as the result of Gne gene deficiency.

### Example 5

### In Vivo AAV.GNE Potency Assay

Wild type mice are used to define AAV.GNE potency in tissues using a measure of UDP-GlcNAc epimerase activity. Any gene therapy clinical development plan must also contain a potency assay that effectively describes the biological activity of the AAV.GNE vector to be used in tissues. Because GNE enzyme activity displays product inhibition from CMP-Neu5Ac when the enzyme is overexpressed, measures of sialic acid will saturate at normal levels and not increase further. As such, measures of UDP-GlcNAc epimerase activity in tissue lysates, which show increases beyond normal levels in tissue lysates, is one of the best means of assessing total GNE activity. An UDP-GlcNAc epimerase assay that can be used to measure GNE enzyme activity in mouse and human tissues is an in vivo potency assay for the GNE gene therapy vectors described herein. A dose-response study in wild type (C57Bl/6J) mice with AAV.GNE vector is carried out to assess the dose and level of vector genome transduction needed to provide a one-fold elevation in GNE enzyme activity, which is defined as the amount required for functional gene replacement. This information may be used to help define dosage even in the absence of proof of concept studies in a GNE disease model.

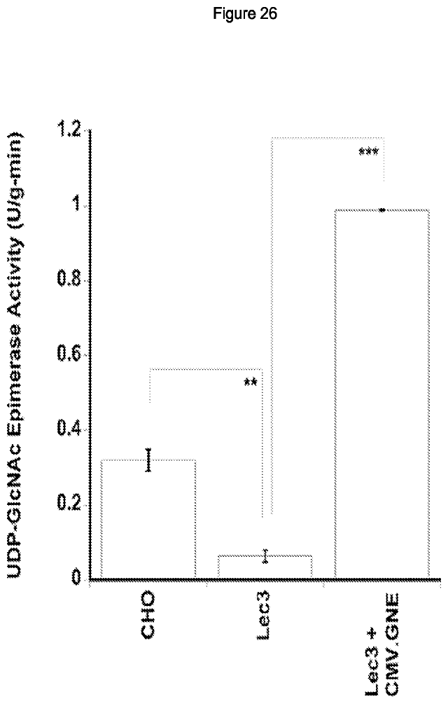

GNE enzyme activity (UDP-GlcNAc epimerase activity) was measured and compared in CHO cell lysates, Lec3 cell lysates (which are deficient in Gne enzyme activity[2]), and Lec3 cells transfected with pAAV.CMV.GNE plasmid. GNE enzyme activity was demonstrated in CHO cells, while almost no GNE enzyme activity was observed in Lec3 cells, and supernormal enzyme activity was observed in Lec3 cells transfected with pAAV.CMV.GNE (FIG. 26). In vivo measures of GNE enzyme activity are superior to MAA assay of sialic acid because of the absence of feedback inhibition in this assay, which will increase the assay's linear read-out. In addition, significantly more material is required to carry out the UDP-GlcNAc epimerase enzyme assay (millions to tens of millions of CHO cells instead of 10,000 CHO cells used for the MAA-HRP ELISA (FIG. 25)). As such, this enzyme activity assay should only be used with tissues (while MAA binding can be used for Lec3 cell ELISAs). This UDP-GlcNAc epimerase assay also works in mouse tissues (e.g., in liver).

As the GNE gene and protein are expressed in almost all organs, the changed GNE enzyme activity (UDP-GlcNAc epimerase activity) is measured in tissues throughout the body plan (liver, kidney, spleen, heart, lung, colon, brain). However, skeletal muscles throughout the body plan (including diaphragm, biceps brachii, triceps brahii, gastrocnemius, quadriceps and tibialis anterior) are a focus for this analysis, as muscle pathology causes disease in GNE myopathy. Tissue lysates from 6 mice (3 male and 3 female) are analyzed, allowing for determinations of reproducibility while accounting for possible gender differences. 30-50 mg of tissue will be cut and homogenized using a TissueLyser (4 30 Hz pulses of 30 seconds each) and allowed to shake on ice for 30 minutes. Once lysed, protein levels are measured by standard Bradford assay and to allow enzyme activity to be normalized to total protein.

UDP-GlcNAc epimerase activity is assayed using the Morgan-Eslon DMAB (4-di-methylamino benzaldeyde) colorimetric method[6] with a 30-minute incubation time, where ManNAc production will be measured by product absorbance on a spectrophotometer at 578 nm. 300 μg of total protein will be used per assay. ManNAc produced by the enzyme is determined by comparison with a ManNAc standard curve undergoing the same DMAB chemical modification protocol, using concentrations of 0, 0.5, 1, 2.5, 5, 10, 25,50 and 75 μg/mL. Next, IV injection of rAAVrh74.CMV.GNE in age and gender-matched wild type mice is carried out to determine the dose required to double endogenous GNE enzyme activity in tissues throughout the body plan. A linear increase in GNE enzyme activity is expected as the dose of AAV increases. Dose of 1×1011 vg/kg, 1×1012 vg/kg, and 1×1013 vg/kg doses are compared. The amount of virus in each tissue is quantified by standard qPCR measures and the amount of GNE gene expression will be measured by qRT-PCR, as we have done previously (Xu et al., Mol. Ther.). Protein levels are also be compared by Western blot should reagents become available.

Most investigators have defined transduction of GNE gene therapy vectors by measuring the amount of GNE cDNA introduced into a tissue or the level of induction of GNE mRNA expression, but neither of these are functional measures of GNE biological activity. The assay described herein which measures GNE enzyme activity (UDP-GlcNAc epimerase activity) allows for a robust functional measure that can be normalized to the amount of total protein used in the assay, and that this assay will be reproducible between mice. It is also expected that by introducing GNE gene therapy at different doses, will demonstrate increases in GNE potency using this assay, and the minimal dose needed to provide an endogenous level of GNE enzyme activity (i.e., a doubling of enzyme activity found in normal tissue) will be defined. This assay provides data needed to determine levels of functional GNE overexpression required for gene replacement in all organs and the number of vector genomes that must be transduced to accomplish such changes.

### Example 6

### Functional Assessment of Bistronic GALGT2 and Follistatin Gene Therapy

The mdx model of muscular dystrophy was used to assess the function of the bistronic rAAV gene therapy expressing GALGT2 and follistatin 344 (FST). It is known that GALGT2 overexpression in skeletal muscle of mdx mice has been reported to prevent muscle damage and inhibit muscle disease (Xu et al., Neuromuscul. Disord. 17: 209-220 (2007); Martin et al. Am. J. Physiol. Cell. Physiol., 296: C476-488 (2009); Nguyen et al., Proc. Natl. Acad. Sci. USA, 99: 5616-5621 (2002), GALGT2 expression in mdx mice has induced improvement equal to that of micro-dystrophin gene transfer even though only half the number of fibers were transduced (Martin et al. (2009), supra).

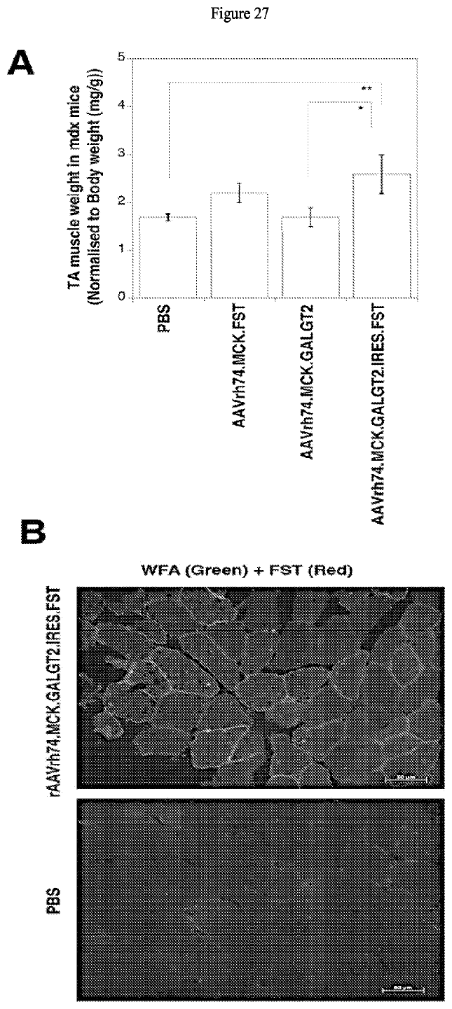

In the present experiment, 2-month-old mdx were injected in the TA with 1×10¹¹ vg of rAAVrh74.MCK.GALGT2.IRES.FST or with single gene vectors (rAAVrh74.MCK.GALGT2 or rAAVrh74.MCK.FST) at the same dose. Phosphobuffered saline (PBS) was injected as a negative control. Two months after injection, the mice were euthanized and muscles weighed, relative to total body weight. As shown in FIG. 27A, both single gene FST and bicistronic GALGT2/FST gene injection led to an increase in muscle size, showing that the placement of the FST gene in the second position of bicistronic vectors leads allows for significant FST function in inducing muscle growth.

After euthanization, the TA muscles were sectioned, fixed in acetone, and stained with antibodies to FST and WFA (to recognize GalNAc made by GALGT2) after injection. As shown in FIG. 27B, injection with the bicistronic vector (rAAVrh74.MCK.GALGT2.IRES.FST) led to functional expression of both GALGT2, which induces glycosylation on the muscle membrane (shown by WFA staining), and FST, which is expressed in the Golgi apparatus, from where it is ultimately secreted outside the muscle cell. Note that the myofibers expressing GALGT2 show normal muscle morphology, showing no signs of muscular dystrophy, a function known for GALGT2 gene overexpression. Thus, this single bicistronic AAV vector can both inhibit muscle pathology, which results from GALGT2 overexpression, and increase muscle size, which results from FST gene expression, allowing a dual function therapy.

1. A polynucleotide comprising a
a) a promoter element,
b) a transgene,
c) internal ribosomal entry site (IRES), and
d) a nucleotide sequence encoding a muscle growth factor or a muscle transdifferentiation factor.

2. A polynucleotide of claim 1 wherein the promoter element is operably linked to the transgene.

3. A polynucleotide of claim 1 wherein the IRES is operably linked to the nucleotide sequence encoding a muscle growth factor or a muscle transdifferentiation factor.

4. polynucleotide comprising
a) one or more promoter elements and
b) a GNE cDNA sequence.

5. A polynucleotide comprising
a) one or more promoter elements,
b) a GNE cDNA sequence or a GALGT2 cDNA sequence,
c) internal ribosomal entry site (IRES), and
d) a nucleotide sequence that encodes a muscle growth factor or muscle transdifferentation factor.

6. A polynucleotide of claim 5 wherein the promoter element is operably linked to the GNE cDNA sequence or the GALGT2 cDNA sequence.

7. A polynucleotide of claim 5 wherein the IRES is operably linked to the nucleotide sequence that encodes a muscle growth factor or muscle transdifferentiation factor.

8. The polynucleotide of claim 1 wherein the promoter element is a constitutive promoter or a muscle-specific promoter.

9. The polynucleotide of claim 1 wherein the promoter element is the CMV promoter, the MCK promoter, the MHCK7 promoter, the miniCMV promoter or the GNE promoter.

10. The polynucleotide of claim 5 wherein the GNE cDNA sequence is a variant 2 GNE wild type human GNE gene comprising the nucleic acid sequence of SEQ ID NO: 1.

11. The polynucleotide sequence of claim 5, further comprising the human GNE promoter element found between exons 1 and 2 to drive expression of the GNE cDNA.

12. The polynucleotide sequence of claim 5 wherein
a) the GALGT2 cDNA sequence comprises the nucleic acid sequence of SEQ ID NO: 36,
b) the IRES comprises the nucleotide sequence of SEQ ID NO: 30 or a fragment thereof
c) the IRES comprises the nucleotide sequence of SEQ ID NO: 8.

13-15. (canceled)

16. The polynucleotide of claim 5, wherein the nucleotide sequence encodes a follistatin, SMAD7 or an Insulin Growth Factor 1 (IGF1) variant.

17. (canceled)

18. (canceled)

19. A recombinant adeno-associated virus (rAAV) having a genome comprising a polynucleotide sequence of claim 1, wherein the polynucleotide is in a single rAAV genome.

20. The rAAV of claim 19 wherein the genome comprises
a) CMV promoter and a variant 2 wild type human GNE cDNA,
b) a MHCK promoter and a variant 2 wild type human GNE cDNA,
c) the GNE promoter and a variant 2 wild type human GNE cDNA
d) a miniCMV promoter and a variant 2 wild type human GNE cDNA,
e) the GNE promoter and a variant 2 wild type human GNE cDNA,
f) a miniCMV promoter and a variant 2 wild type human GNE cDNA,
g) the MCK7 promoter, a variant 2 wild type human cDNA, a FGF1 IRES and a nucleic acid sequence encoding follistatin 344,
h) the MHCK7 promoter, a variant 2 wild type human GNE cDNA, a FGF1 IRES and a nucleic acid sequence encoding HB-IGF1,
i) the CMV promoter, a variant 2 wild type human GNE cDNA, a FGF1 IRES and nucleic acid sequence encoding follistatin 344,
j) the CMV promoter, a variant 2 wild type human GNE cDNA, a FGF1 IRES and nucleic acid sequence encoding HB-IGF1,
k) the MCK promoter, a variant 2 wild type human GNE cDNA, a FGF1 IRES and a nucleic acid sequence encoding follistatin 344,
l) the MCK promoter, a variant 2 wild type human GNE cDNA, a FGF1 IRES and nucleic acid sequence encoding HB-IGF1,
m) the MCK promoter, a variant 2 wild type human GNE cDNA, a FGF1 IRES and nucleic acid sequence encoding HB-IGF1,
n) the GNE promoter, a variant 2 wild type human GNE cDNA, a FGF1 IRES and a nucleic acid sequence encoding follistatin 344,
o) the GNE promoter, a variant 2 wild type human GNE cDNA, a FGF1 IRES and a nucleic acid sequence encoding HB-IGF1,
p) the miniCMV promoter, a variant 2 wild type human GNE cDNA, FGF1 IRES and a nucleic acid sequence encoding follistatin 344,
q) the miniCMV promoter, a variant 2 wild type human GNE cDNA, a FGF1 IRES and a nucleic acid sequence encoding HB-IGF1,
r) MHCK7 promoter, a variant 2 wild type human GNE cDNA, a FGF1 IRES and a nucleic acid sequence encoding SMAD7,
s) CMV promoter, a variant 2 wild type human GNE cDNA, a FGF1 IRES and a nucleic acid sequence encoding SMAD7,
t) MCK promoter, a variant 2 wild type human GNE cDNA, a FGF1 IRES and a nucleic acid sequence encoding SMAD7,
u) GNE promoter, a variant 2 wild type human GNE cDNA, a FGF1 IRES and a nucleic acid sequence encoding SMAD7,
v) miniCMV promoter, a variant 2 wild type human GNE cDNA, a FGF1 IRES and a nucleic acid sequence encoding SMAD7,
w) the MCK promoter, the GALGT2 cDNA, a FGFR1 IRES and a nucleic acid encoding follistatin 344,
x) the MCK promoter, the GALGT2 cDNA, a FGFR1 IRES and a nucleic acid encoding HB-IGF1 or
y) the MCK promoter, a the GALGT2 cDNA, a FGF1 IRES and a nucleic acid sequence encoding SMAD7.

21-42. (canceled)

43. The rAAV of claim 19 wherein the rAAV is of the serotype rAAVrh.74.

44. An rAAV particle comprising the rAAV of claim 19.

45. A method of treating GNE myopathy in a human subject in need thereof comprising the step of administering an rAAV particle of claim 44.

46. (canceled)

47. (canceled)

48. A method of treating muscular dystrophy in a human subject in need thereof comprising the step of administering an rAAV particle of claim 44.

49. (canceled)

50. (canceled)

51. The method of claim 48 wherein the muscular dystrophy is Duchene muscular dystrophy, Limb Girdle Muscular Dystrophy 2D or Congenital Muscular Dystrophy 1A.


SEQUENCE LISTING (40 sequences)

SEQ ID NO: 1 - DNA, 2169 bp, Homo Sapiens
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 2 - protein, 753 aa, Homos Sapiens
METYGYLQRESCFQGPHELYFKNLSKRNKQIMEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 3 - DNA, 407 bp, Homo Sapiens
ctcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttg

SEQ ID NO: 4 - DNA, 564 bp, Homo Sapiens
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 5 - DNA, 810 bp, Homo Sapiens
gtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgccc

SEQ ID NO: 6 - DNA, 707 bp, Homo Sapiens
gcttgaggccaggagctcgagtgctgcctggacaacatagtgagacccttgtctcaaaaacaacagcaacccaaagtcttatttatcttcaggagctgcgcaagccctttaccaccgccacgtttctccctggatgtccctaggcctcgggactggctgggagttccctgtagtggaggccgccgctgaactgattataaagatgagagaggctctgccatgccttggtcatcaattcacactccaccagtgtcttctagcagtcacggcaagggttaacgtcagggaccgctgtggggtggccgcgctacacgacagtatagttgcggtcctggtttatgaaataactgagggaacaagaggcgcaagaaatccctccttgggtgcaagaccaaaacaactacccagcgggaagactcgggcttcagtgcgtgtgtcgccagtggaggaggacgcttcggggcgggagcacaagctggcaggacagccccgcagcagctccagcgcggcagaggccatggaagatggtccgctggtcagcaccctgcctgcgcctcaaaataccgccgggaaggtgggggacggggacgggagcagccaatcacgcgagctctctcgggtggcgtcagggcgcgatcgcggattcggcactccgggtgggcgtggctgggcgagcgaggagtggggacaaggtcgagcgacgagtc

SEQ ID NO: 7 - DNA, 228 bp, Homo Sapiens
tcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 8 - DNA, 119 bp, Homo Sapiens
ccttctgatggtgaatgggaactcccttcctcctgcagcagcctgcctgcagctgtcctggtagaacagtgtggacattgcagaagctgtcactgccccagaaagaaagcaccccagag

SEQ ID NO: 9 - DNA, 1032 bp, Homo Sapiens
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtgg

SEQ ID NO: 10 - protein, 344 aa, Homo Sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 11 - DNA, 534 bp, Homo Sapiens
gaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagcttaggc

SEQ ID NO: 12 - DNA, 7277 bp, Homo Sapiens
acccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgtgtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgcactagtgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaaaactctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgttcttcggggcgaaaactctcaaggatcttaccgctgttgagatccagttcgatgtaacccactcgtgc

SEQ ID NO: 13 - DNA, 7070 bp, Homo Sapiens
tgaagatcagttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacagccactatgggtctaggctgcccatgtaaggaggcaaggcctggggacacccgagatgcctggttataattaacccagacatgtggctgctcccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgcactagtgcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgc

SEQ ID NO: 14 - DNA, 7270 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgcactagtgcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 15 - DNA, 7187 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgaggccaggagctcgagtgctgcctggacaacatagtgagacccttgtctcaaaaacaacagcaacccaaagtcttatttatcttcaggagctgcgcaagccctttaccaccgccacgtttctccctggatgtccctaggcctcgggactggctgggagttccctgtagtggaggccgccgctgaactgattataaagatgagagaggctctgccatgccttggtcatcaattcacactccaccagtgtcttctagcagtcacggcaagggttaacgtcagggaccgctgtggggtggccgcgctacacgacagtatagttgcggtcctggtttatgaaataactgagggaacaagaggcgcaagaaatccctccttgggtgcaagaccaaaacaactacccagcgggaagactcgggcttcagtgcgtgtgtcgccagtggaggaggacgcttcggggcgggagcacaagctggcaggacagccccgcagcagctccagcgcggcagaggccatggaagatggtccgctggtcagcaccctgcctgcgcctcaaaataccgccgggaaggtgggggacggggacgggagcagccaatcacgcgagctctctcgggtggcgtcagggcgcgatcgcggattcggcactccgggtgggcgtggctgggcgagcgaggagtggggacaaggtcgagcgacgagtcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgcactagtgcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 16 - DNA, 8423 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagactagtatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 17 - DNA, 7928 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagactggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagcttaggcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 18 - DNA, 8430 bp, Homo Sapiens
acccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgtgtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagactagtatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaaaactctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgttcttcggggcgaaaactctcaaggatcttaccgctgttgagatccagttcgatgtaacccactcgtgc

SEQ ID NO: 19 - DNA, 7937 bp, Homo Sapiens
acccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgtgtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagactagggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagcttaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaaaactctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgttcttcggggcgaaaactctcaaggatcttaccgctgttgagatccagttcgatgtaacccactcgtgc

SEQ ID NO: 20 - DNA, 8223 bp, Homo Sapiens
tgaagatcagttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacagccactatgggtctaggctgcccatgtaaggaggcaaggcctggggacacccgagatgcctggttataattaacccagacatgtggctgctcccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagactagtatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgc

SEQ ID NO: 21 - DNA, 7726 bp, Homo Sapiens
tgaagatcagttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacagccactatgggtctaggctgcccatgtaaggaggcaaggcctggggacacccgagatgcctggttataattaacccagacatgtggctgctcccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagaggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagcttaggcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgc

SEQ ID NO: 22 - DNA, 8340 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgaggccaggagctcgagtgctgcctggacaacatagtgagacccttgtctcaaaaacaacagcaacccaaagtcttatttatcttcaggagctgcgcaagccctttaccaccgccacgtttctccctggatgtccctaggcctcgggactggctgggagttccctgtagtggaggccgccgctgaactgattataaagatgagagaggctctgccatgccttggtcatcaattcacactccaccagtgtcttctagcagtcacggcaagggttaacgtcagggaccgctgtggggtggccgcgctacacgacagtatagttgcggtcctggtttatgaaataactgagggaacaagaggcgcaagaaatccctccttgggtgcaagaccaaaacaactacccagcgggaagactcgggcttcagtgcgtgtgtcgccagtggaggaggacgcttcggggcgggagcacaagctggcaggacagccccgcagcagctccagcgcggcagaggccatggaagatggtccgctggtcagcaccctgcctgcgcctcaaaataccgccgggaaggtgggggacggggacgggagcagccaatcacgcgagctctctcgggtggcgtcagggcgcgatcgcggattcggcactccgggtgggcgtggctgggcgagcgaggagtggggacaaggtcgagcgacgagtcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagactagtatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 23 - DNA, 7843 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgaggccaggagctcgagtgctgcctggacaacatagtgagacccttgtctcaaaaacaacagcaacccaaagtcttatttatcttcaggagctgcgcaagccctttaccaccgccacgtttctccctggatgtccctaggcctcgggactggctgggagttccctgtagtggaggccgccgctgaactgattataaagatgagagaggctctgccatgccttggtcatcaattcacactccaccagtgtcttctagcagtcacggcaagggttaacgtcagggaccgctgtggggtggccgcgctacacgacagtatagttgcggtcctggtttatgaaataactgagggaacaagaggcgcaagaaatccctccttgggtgcaagaccaaaacaactacccagcgggaagactcgggcttcagtgcgtgtgtcgccagtggaggaggacgcttcggggcgggagcacaagctggcaggacagccccgcagcagctccagcgcggcagaggccatggaagatggtccgctggtcagcaccctgcctgcgcctcaaaataccgccgggaaggtgggggacggggacgggagcagccaatcacgcgagctctctcgggtggcgtcagggcgcgatcgcggattcggcactccgggtgggcgtggctgggcgagcgaggagtggggacaaggtcgagcgacgagtcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgccttctgatggtgaatgggaactcccttcctcctgcagcagcctgcctgcagctgtcctggtagaacagtgtggacattgcagaagctgtcactgccccagaaagaaagcaccccagagaggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagcttaggcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 24 - DNA, 6594 bp, Homo Sapiens
acccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgtgtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggggtacccggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgcactagtgcggccgcggatctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaggtttaaaccccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgttcttcggggcgaaaactctcaaggatcttaccgctgttgagatccagttcgatgtaacccactcgtgc

SEQ ID NO: 25 - DNA, 8340 bp, Homo Sapiens; identical to SEQ ID NO: 22
  misc_feature (6)..(6): n is a, c, g, or t
  misc_feature (15)..(15): n is a, c, g, or t
  misc_feature (20)..(20): n is a, c, g, or t

SEQ ID NO: 26 - DNA, 7539 bp, Homo Sapiens
gttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggggtacccggccggctagccgccaccatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagcatgcgggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagactggccgcaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccaggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagcttaggcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatca

SEQ ID NO: 27 - protein, 178 aa, Homo Sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPGPETLCGAELVDALQFVCGDRGFYFNKPTGYGSSSRRAPQTGIVDECCFRSCDLRRLEMYCAPLKPAKSAV

SEQ ID NO: 28 - DNA, 2549 bp, Homo Sapiens
gtccattgaatcgcgcgggcggccggcggcgagcgcggggctgcgccgggatcgctgcgccctccgccgctggcctctgcgacgcgcgccgctcgcccgagccacccgccgccgcgccggctccccgcgccgctgcgctcctcgccccgcgcctgcccccaggatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactgaatctgcccgtaaaacctgagccattgattcttcagaactttctgcagtttttgacttcatagattatgctttaaaaaaatttttttaacttattgcataacagcagatgccaaaaacaaaaaaagcatctcactgcaagtcacataaaaatgcaacgctgtaatatggctgtatcagagggctttgaaaacatacactgagctgcttctgcgctgttgttgtccgtatttaaacaacagctcccctgtattcccccatctagccatttcggaagacaccgaggaagaggaggaagatgaagaccaggactacagctttcctatatcttctattctagagtggtaaactctctataagtgttcagtgttgacatagcctttgtgcaaaaaaaaaaaaaaaaaaaaagaaaaagaaaaaaagaaaaatatattgtccatactgtaaataagtgtatgcttatttatttggggggaaaactatacattaaaggacctttgtcctaaagctctctcccaggccaccttgttactcattggacacggagaggcattcattgtgaggtctactggatgaggcccatagttgagacttgtagacatttatttatactgtgtcatgttttataatttatacataaaatgtctggttgactgtataccttgtttttgaagaaatttattcgtgaaaggaagagcagttgttatttattgtgaggtctcttgcttgtaaagtaaaagctttttttccttgtaaaccatttaagtccattccttactattcactcactcatctgtctcccttcatttcactgttagactcttttccactttcaacaaacttgcatgtcagtttctgtcatgtttatttattggattctctgctgcctgatctgtacatacatgatccctcgggttttgtttacaaggaaccttgactgaccaaaaggcattataactctgactcaaatacaaggtacagaagataagcatctttgaggaaactcctacttcagttcttttgttatgatgaagacatttgtgagagaggagatgattagaattctagtaatgtacttttaagatgttacagatacaaagaaatgatgtgggtgtcaggagactaaaggatgttgaaggctacacattcaaccttttgttaggtgtttcctttaagctactcagctgtaccttttaaattagttctttttcaaccagtatatcactaaaagttatatcaaagctttatcagttcaagtttcttgcttttcataatacttttttctgatgcaattttatattttcaaacatggcaagttaaaatataaattcatttaaatatatagttttgtacttttctaccatgtaaatgtgcaatgtatataaaagttataatgtgtatttgtaaataaatgatgagtgaaaaaataaaaaaa

SEQ ID NO: 29 - protein, 317 aa, Homo Sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 30 - DNA, 427 bp, Homo Sapiens
gggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctcgagcagcctgcctgcagctgtcctggtagaacagtgtggacattgcagaagctgtcactgccccagaaagaaagcaccccagagccaaggcaaagagtcttgaaagcgccacaagcagctgctgag

SEQ ID NO: 31 - protein, 319 aa, Homo Sapiens [sequence omitted]

SEQ ID NO: 32 - DNA, 2501 bp, Homo Sapiens
aagtcggtgccgctgccgtctctgcgttcgccatgcgtcccggggcgccagggccactctggcctctgccctggggggccctggcttgggccgtgggcttcgtgagctccatgggctcggggaaccccgcgcccggtggtgtttgctggctccagcagggccaggaggccacctgcagcctggtgctccagactgatgtcacccgggccgagtgctgtgcctccggcaacattgacaccgcctggtccaacctcacccacccggggaacaagatcaacctcctcggcttcttgggccttgtccactgccttccctgcaaagattcgtgcgacggcgtggagtgcggcccgggcaaggcgtgccgcatgctggggggccgcccgcgctgcgagtgcgcgcccgactgctcggggctcccggcgcggctgcaggtctgcggctcagacggcgccacctaccgcgacgagtgcgagctgcgcgccgcgcgctgccgcggccacccggacctgagcgtcatgtaccggggccgctgccgcaagtcctgtgagcacgtggtgtgcccgcggccacagtcgtgcgtcgtggaccagacgggcagcgcccactgcgtggtgtgtcgagcggcgccctgccctgtgccctccagccccggccaggagctttgcggcaacaacaacgtcacctacatctcctcgtgccacatgcgccaggccacctgcttcctgggccgctccatcggcgtgcgccacgcgggcagctgcgcaggcacccctgaggagccgccaggtggtgagtctgcagaagaggaagagaacttcgtgtgagcctgcaggacaggcctgggcctggtgcccgaggccccccatcatcccctgttatttattgccacagcagagtctaatttatatgccacggacactccttagagcccggattcggaccacttggggatcccagaacctccctgacgatatcctggaaggactgaggaagggaggcctgggggccggctggtgggtgggatagacctgcgttccggacactgagcgcctgatttagggcccttctctaggatgccccagcccctaccctaagacctattgccggggaggattccacacttccgctcctttggggataaacctattaattattgctactatcaagagggctgggcattctctgctggtaattcctgaagaggcatgactgcttttctcagccccaagcctctagtctgggtgtgtacggagggtctagcctgggtgtgtacggagggtctagcctgggtgagtacggagggtctagcctgggtgagtacggagggtctagcctgggtgagtacggagggtctagcctgggtgtgtatggaggatctagcctgggtgagtatggagggtctagcctgggtgagtatggagggtctagcctgggtgtgtatggagggtctagcctgggtgagtatggagggtctagcctgggtgtgtatggagggtctagcctgggtgagtatggagggtctagcctgggtgtgtacggagggtctagtctgagtgcgtgtggggacctcagaacactgtgaccttagcccagcaagccaggcccttcatgaaggccaagaaggctgccaccattccctgccagcccaagaactccagcttccccactgcctctgtgtgcccctttgcgtcctgtgaaggccattgagaaatgcccagtgtgccccctgggaaagggcacggcctgtgctcctgacacgggctgtgcttggccacagaaccacccagcgtctcccctgctgctgtccacgtcagttcatgaggcaacgtcgcgtggtctcagacgtggagcagccagcggcagctcagagcagggcactgtgtccggcggagccaagtccactctgggggagctctggcggggaccacgggccactgctcacccactggccccgaggggggtgtagacgccaagactcacgcatgtgtgacatccggagtcctggagccgggtgtcccagtggcaccactaggtgcctgctgcctccacagtggggttcacacccagggctccttggtcccccacaacctgccccggccaggcctgcagacccagactccagccagacctgcctcacccaccaatgcagccggggctggcgacaccagccaggtgctggtcttgggccagttctcccacgacggctcaccctcccctccatctgcgttgatgctcagaatcgcctacctgtgcctgcgtgtaaaccacagcctcagaccagctatggggagaggacaacacggaggatatccagcttccccggtctggggtgaggaatgtggggagcttgggcatcctcctccagcctcctccagcccccaggcagtgccttacctgtggtgcccagaaaagtgcccctaggttggtgggtctacaggagcctcagccaggcagcccaccccaccctggggccctgcctcaccaaggaaataaagactcaa

SEQ ID NO: 33 - protein, 263 aa, Homo Sapiens
MRPGAPGPLWPLPWGALAWAVGFVSSMGSGNPAPGGVCWLQQGQEATCSLVLQTDVTRAECCASGNIDTAWSNLTHPGNKINLLGFLGLVHCLPCKDSCDGVECGPGKACRMLGGRPRCECAPDCSGLPARLQVCGSDGATYRDECELRAARCRGHPDLSVMYRGRCRKSCEHVVCPRPQSCVVDQTGSAHCVVCRAAPCPVPSSPGQELCGNNNVTYISSCHMRQATCFLGRSIGVRHAGSCAGTPEEPPGGESAEEEENFV

SEQ ID NO: 34 - DNA, 3417 bp, Homo Sapiens
agaggcgccagcagcctgcctgtgacaggcatcaggttagctggctcccactcgggtggcgcgcccaggatataaatccgggcgcgggcccctgctgtggctcctctccctgcacactcaggagagggagcttccttctaaagacctttcttttatctgaagccgcacagcccggcaggctgtgctgacttggtggaggcagcagcggcagagcagcctgagcagcagcctgagcaggaaacctgctggggtggggagggcaggtgtctgcagcccctgagaagaaggccctggtgggccccagaccctggcatcgtttcaggggaggtctctagccgccccagcctgcaccatgtgggccccaaggtgtcgccggttctggtctcgctgggagcaggtggcagcgctgctgctgctgctgctactgctcggggtgcccccgcgaagcctggcgctgccgcccatccgctattcccacgccggcatctgccccaacgacatgaatcccaacctctgggtggacgcacagagcacctgcaggcgggagtgtgagacggaccaggagtgtgagacctatgagaagtgctgccccaacgtatgtgggaccaagagctgcgtggcggcccgctacatggacgtgaaagggaagaagggcccagtgggcatgcccaaggaggccacatgtgaccacttcatgtgtctgcagcagggctctgagtgtgacatctgggatggccagcccgtgtgtaagtgcaaagaccgctgtgagaaggagcccagctttacctgcgcctcggacggcctcacctactataaccgctgctacatggatgccgaggcctgctccaaaggcatcacactggccgttgtaacctgccgctatcacttcacctggcccaacaccagccccccaccacctgagaccaccatgcaccccaccacagcctccccagagacccctgagctggacatggcggcccctgcgctgctcaacaaccctgtgcaccagtcggtcaccatgggtgagacagtgagcttcctctgtgatgtggtgggccggccccggcctgagatcacctgggagaagcagttggaggatcgggagaatgtggtcatgcggcccaaccatgtgcgtggcaacgtggtggtcaccaacattgcccagctggtcatctataacgcccagctgcaggatgctgggatctacacctgcacggcccggaacgtggctggggtcctgagggctgatttcccgctgtcggtggtcaggggtcatcaggctgcagccacctcagagagcagccccaatggcacggctttcccggcggccgagtgcctgaagcccccagacagtgaggactgtggcgaagagcagacccgctggcacttcgatgcccaggccaacaactgcctgaccttcaccttcggccactgccaccgtaacctcaaccactttgagacctatgaggcctgcatgctggcctgcatgagcgggccgctggccgcgtgcagcctgcccgccctgcaggggccctgcaaagcctacgcgcctcgctgggcttacaacagccagacgggccagtgccagtcctttgtctatggtggctgcgagggcaatggcaacaactttgagagccgtgaggcctgtgaggagtcgtgccccttccccagggggaaccagcgctgtcgggcctgcaagcctcggcagaagctcgttaccagcttctgtcgcagcgactttgtcatcctgggccgagtctctgagctgaccgaggagcctgactcgggccgcgccctggtgactgtggatgaggtcctaaaggatgagaaaatgggcctcaagttcctgggccaggagccattggaggtcactctgcttcacgtggactgggcatgcccctgccccaacgtgaccgtgagcgagatgccgctcatcatcatgggggaggtggacggcggcatggccatgctgcgccccgatagctttgtgggcgcatcgagtgcccgccgggtcaggaagcttcgtgaggtcatgcacaagaagacctgtgacgtcctcaaggagtttcttggcttgcactgaagccccccacccctccctgccccctccctggccttcttccacctatccaccccaatgcctctcagcaaactgggcgaggtcagattagacaggcttgggacagcagggaaacatcaaccgacgtgtcacagaaaaagccacagaaggtctcagatcagcatctattctttgggttcaataaggggttcatatcttttttagctgagggggacaagaggagaagtcagtggacacatggaagttactcgtgaccaccagcttgctcagatattctcctcctcccctcactggccccacacccctggctctcccagtcaccctcccctagccagtctcccagcaagggtttaagagatggccgctgtgtgctggtcacaggaagtgttgaatggattggcttgcaaagggggtaggtggggagagataggagggcccagggactcatgggacacctttcccacagcctcctcgattgctgtgagcagaggccactcggagttaggggcatgggcaatagcaagctggcggcagagtccagcccagcatatgacttgccctgaatggaagctgctgaaacgggtgcctttgggtggtggtcggcttgcctctgaggccaccacggcaccagcagaatacgtatttcttctccttggctgcactggtttgtcgatctagttcagttcaactcagtggatgttctctgaatgcttactgggtgccaggaccacagagagatgttagtcactgcccagttcttagagccccaacacagataccctcatcccagggcccccagacacacccctccgctggactcacaactgtctggagtttctgtctgatggatggtgtgctttcatatgccactggcttccttggacatagatcagacaaaagccccgggatctgtttggtagcaggagaaatgaaggaagatgaaaaagcaggcagggaagggggtagtaaaggactgagagaggagggaggtggctggagaaggaaaaggaacattgctcgatgctcccatctggtggcggcctcaggaacccacgggaacctggaaggaggctctttgtgagacctgggcaaaggatggggcagctcgtcgatgatttttttgtgtttccaggcttcctgtgtgatcctggccctccggccgctagagagaggattgggaaaccccactgtcagctctgcatctgcccccactaccctcctctgccctattctgtccctgcccctccaagctgaagaaggtccttgtggggcgtcctcatttcttcctcaaatataaggaggaagataccaattaaaagctcatagtatcaactgcc

SEQ ID NO: 35 - protein, 576 aa, Homo Sapiens
MWAPRCRRFWSRWEQVAALLLLLLLLGVPPRSLALPPIRYSHAGICPNDMNPNLWVDAQSTCRRECETDQECETYEKCCPNVCGTKSCVAARYMDVKGKKGPVGMPKEATCDHFMCLQQGSECDIWDGQPVCKCKDRCEKEPSFTCASDGLTYYNRCYMDAEACSKGITLAVVTCRYHFTWPNTSPPPPETTMHPTTASPETPELDMAAPALLNNPVHQSVTMGETVSFLCDVVGRPRPEITWEKQLEDRENVVMRPNHVRGNVVVTNIAQLVIYNAQLQDAGIYTCTARNVAGVLRADFPLSVVRGHQAAATSESSPNGTAFPAAECLKPPDSEDCGEEQTRWHFDAQANNCLTFTFGHCHRNLNHFETYEACMLACMSGPLAACSLPALQGPCKAYAPRWAYNSQTGQCQSFVYGGCEGNGNNFESREACEESCPFPRGNQRCRACKPRQKLVTSFCRSDFVILGRVSELTEEPDSGRALVTVDEVLKDEKMGLKFLGQEPLEVTLLHVDWACPCPNVTVSEMPLIIMGEVDGGMAMLRPDSFVGASSARRVRKLREVMHKKTCDVLKEFLGLH

SEQ ID NO: 36 - DNA, 1521 bp, Homo Sapiens
atgacttcgggcggctcgagatttctgtggctcctcaagatattggtcataatcctggtacttggcattgttggatttatgttcggaagcatgttccttcaagcagtgttcagcagccccaagccagaactcccaagtcctgccccgggtgtccagaagctgaagcttctgcctgaggaacgtctcaggaacctcttttcctacgatggaatctggctgttcccgaaaaatcagtgcaaatgtgaagccaacaaagagcagggaggttacaactttcaggatgcctatggccagagcgacctcccagcggtgaaagcgaggagacaggctgaatttgaacactttcagaggagagaagggctgccccgcccactgcccctgctggtccagcccaacctcccctttgggtacccagtccacggagtggaggtgatgcccctgcacacggttcccatcccaggcctccagtttgaaggacccgatgcccccgtctatgaggtcaccctgacagcttctctggggacactgaacacccttgctgatgtcccagacagtgtggtgcagggcagaggccagaagcagctgatcatttctaccagtgaccggaagctgttgaagttcattcttcagcacgtgacatacaccagcacggggtaccagcaccagaaggtagacatagtgagtctggagtccaggtcctcagtggccaagtttccagtgaccatccgccatcctgtcatacccaagctatacgaccctggaccagagaggaagctcagaaacctggttaccattgctaccaagactttcctccgcccccacaagctcatgatcatgctccggagtattcgagagtattacccagacttgaccgtaatagtggctgatgacagccagaagcccctggaaattaaagacaaccacgtggagtattacactatgccctttgggaagggttggtttgctggtaggaacctggccatatctcaggtcaccaccaaatacgttctctgggtggacgatgattttctcttcaacgaggagaccaagattgaggtgctggtggatgtcctggagaaaacagaactggacgtggtaggcggcagtgtgctgggaaatgtgttccagtttaagttgttgctggaacagagtgagaatggggcctgccttcacaagaggatgggatttttccaacccctggatggcttccccagctgcgtggtgaccagtggcgtggtcaacttcttcctggcccacacggagcgactccaaagagttggctttgatccccgcctgcaacgagtggctcactcagaattcttcattgatgggctagggaccctactcgtggggtcatgcccagaagtgattataggtcaccagtctcggtctccagtggtggactcagaactggctgccctagagaagacctacaatacataccggtccaacaccctcacccgggtccagttcaagctggccctccactacttcaagaaccatctccaatgtgccgcataa

SEQ ID NO: 37 - protein, 506 aa, Homo Sapiens
MTSGGSRFLWLLKILVIILVLGIVGFMFGSMFLQAVFSSPKPELPSPAPGVQKLKLLPEERLRNLFSYDGIWLFPKNQCKCEANKEQGGYNFQDAYGQSDLPAVKARRQAEFEHFQRREGLPRPLPLLVQPNLPFGYPVHGVEVMPLHTVPIPGLQFEGPDAPVYEVTLTASLGTLNTLADVPDSVVQGRGQKQLIISTSDRKLLKFILQHVTYTSTGYQHQKVDIVSLESRSSVAKFPVTIRHPVIPKLYDPGPERKLRNLVTIATKTFLRPHKLMIMLRSIREYYPDLTVIVADDSQKPLEIKDNHVEYYTMPFGKGWFAGRNLAISQVTTKYVLWVDDDFLFNEETKIEVLVDVLEKTELDVVGGSVLGNVFQFKLLLEQSENGACLHKRMGFFQPLDGFPSCVVTSGVVNFFLAHTERLQRVGFDPRLQRVAHSEFFIDGLGTLLVGSCPEVIIGHQSRSPVVDSELAALEKTYNTYRSNTLTRVQFKLALHYFKNHLQCAA

SEQ ID NO: 38 - DNA, 7887 bp, Homo Sapiens
tgaagatcagttgggtgcacgagtgggttacatcgaactggatctcaacagcggtaagatccttgagagttttcgccccgaagaacgaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccggctagccgccaccatgacttcgggcggctcgagatttctgtggctcctcaagatattggtcataatcctggtacttggcattgttggatttatgttcggaagcatgttccttcaagcagtgttcagcagccccaagccagaactcccaagtcctgccccgggtgtccagaagctgaagcttctgcctgaggaacgtctcaggaacctcttttcctacgatggaatctggctgttcccgaaaaatcagtgcaaatgtgaagccaacaaagagcagggaggttacaactttcaggatgcctatggccagagcgacctcccagcggtgaaagcgaggagacaggctgaatttgaacactttcagaggagagaagggctgccccgcccactgcccctgctggtccagcccaacctcccctttgggtacccagtccacggagtggaggtgatgcccctgcacacggttcccatcccaggcctccagtttgaaggacccgatgcccccgtctatgaggtcaccctgacagcttctctggggacactgaacacccttgctgatgtcccagacagtgtggtgcagggcagaggccagaagcagctgatcatttctaccagtgaccggaagctgttgaagttcattcttcagcacgtgacatacaccagcacggggtaccagcaccagaaggtagacatagtgagtctggagtccaggtcctcagtggccaagtttccagtgaccatccgccatcctgtcatacccaagctatacgaccctggaccagagaggaagctcagaaacctggttaccattgctaccaagactttcctccgcccccacaagctcatgatcatgctccggagtattcgagagtattacccagacttgaccgtaatagtggctgatgacagccagaagcccctggaaattaaagacaatcacgtggagtattacactatgccctttgggaagggttggtttgctggtaggaacctggccatatctcaggtcaccaccaaatacgttctctgggtggacgatgattttctcttcaacgaggagaccaagattgaggtgctggtggatgtcctggagaaaacagaactggacgtggtaggcggcagtgtgctgggaaatgtgttccagtttaagttgttgctggaacagagtgagaatggggcctgccttcacaagaggatgggatttttccaacccctggatggcttccccagctgcgtggtgaccagtggcgtggtcaacttcttcctggcccacacggagcgactccaaagagttggctttgatccccgcctgcaacgagtggctcactcagaattcttcattgatgggctagggaccctactcgtggggtcatgcccagaagtgattataggtcaccagtctcggtctccagtggtggactcagaactggctgccctagagaagacctacaatacataccggtccaacaccctcacccgggtccagttcaagctggccctccactacttcaagaaccatctccaatgtgccgcataagcatgcgggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagactagtatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggtaagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgc

SEQ ID NO: 39 - DNA, 3341 bp, Homo Sapiens
attgcctgcttctccccacccccaaattaagttgcttagcaagggggaaagaggctttttccttcttccaacagcccagccgaacgcctttcgttttttgcccccgcggaccttccatgtaggaagccgaggctggcgagcccgacattcgggagccactgcgggggggcctctttttggggaggcgccgacgggggcaggctcggccgtccccagggaagcggcggccgggttcctccggggcgcgccggggccggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccacccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccatcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgtttttttctatgggtgttatcacctagctgaatgtttttctaaaggagtttatgttccattaaacgatttttaaaatgtacacttga

SEQ ID NO: 40 - protein, 426 aa, Homo Sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR